(19) United States
(12) Patent Application Publication
(10) Pub. No.: US 2025/0051854 A1
(43) Pub. Date: Feb. 13, 2025

(54) METHODS FOR PREDICTING TUMOR CONTENT USING EPIGENETIC SIGNATURES

(71) Applicant: Flagship Pioneering Innovations VI, LLC, Cambridge, MA (US)

(72) Inventors: Jocelyn Charlton, London (GB); James Xin Sun, Needham Heights, MA (US); Caitlin M. Gilley, Boston, MA (US)

(21) Appl. No.: 18/749,470

(22) Filed: Jun. 20, 2024

Related U.S. Application Data
(60) Provisional application No. 63/515,034, filed on Jul. 21, 2023.
(60) Provisional application No. 63/509,105, filed on Jun. 20, 2023.

(51) Int. Cl. C12Q 1/6886; G16B 20/20

(52) U.S. Cl. CPC C12Q 1/6886; G16B 20/20; C12Q 2600/106; C12Q 2600/154

(57) ABSTRACT

Disclosed herein are methods and compositions for predicting tumor content e.g., tumor content in a sample and tumor burden of an individual. Generally, methods and compositions involve determining methylation statuses of two or more sequential CpG sites in one or more genomic locations using nucleic acids of the obtained sample. The methylation statuses of the two or more sequential CpG in genomic locations are used to distinguish between reads of nucleic acids that are likely derived from cancer and reads of other nucleic acids that are unlikely to be derived from cancer. Distinguishing reads that are likely derived from cancer enables the prediction of tumor content in a sample.

### CROSS-REFERENCE TO RELATED APPLICATIONS

This application claims priority to, and the benefit of, U.S. Prov. App. No. 63/509,105 filed Jun. 20, 2023, and U.S. Prov. App. No. 63/515,034, filed Jul. 21, 2023, each of which are incorporated herein in their entirety.

### SEQUENCE LISTING

The instant application contains a Sequence Listing which has been submitted electronically in XML format and is hereby incorporated by reference in its entirety. Said Sequence Listing, created on Nov. 4, 2024, is named FLG-016US_SL. and is 1,482,612 bytes in size.

### BACKGROUND

Cancer detection methods have traditionally relied on the detection of somatic mutations, often in the form of single nucleotide variants (SNVs). However, SNVs are generally low-recurring or non-recurring across various cancers (either same cancers or different cancers), thereby rendering the use of SNVs as universal predictors of cancer challenging. Due to the lack of a universal signal, using SNVs to detect cancer often requires reference biopsy samples. Therefore, new methods of detecting cancer signatures with improved predictability are needed.

### SUMMARY

Disclosed herein are methods of detecting cancer using epigenetic signatures in the form of methylation variants (MVs). In particular, the methods disclosed herein involve identifying methylation variants at conserved genomic locations, herein referred to as universal genomic locations, and using the detection of methylation variants at the universal genomic locations to determine the presence of cancer e.g., in a sample obtained from a subject.

Disclosed herein is a method of determining tumor DNA content in a biological sample comprising: a) obtaining cell-free DNA fragments from the sample; b) determining methylation statuses of methylation variants each representing two or more sequential CpG sites in a genomic location from the cell-free DNA fragments; and c) quantifying a proportion of fully methylated methylation variants, wherein the quantification in step c) is performed without requiring a matched tissue sample. In various embodiments, the methylation variants are conserved across two or more, three or more, four or more, or five or more cancers. In various embodiments, each methylation variant comprises 3 or more sequential CpG sites, 4 or more sequential CpG sites, or 5 or more sequential CpG sites. In various embodiments, each methylation variant comprises 5 sequential CpG sites. In various embodiments, each methylation variant refers to a range of genomic locations and corresponding CpG sites identified in Table 1 or Table 2. In various embodiments, determining methylation statuses of methylation variants comprises performing one or more assays on the cell-free DNA fragments. In various embodiments, the one or more assays comprises bisulfite conversion, nucleic acid amplification, polymerase chain reaction (PCR), methylation-specific PCR, bisulfite pyrosequencing, single-strand conformation polymorphism (SSCP) analysis, methylation-sensitive single-strand conformation analysis, high resolution melting analysis, methylation-sensitive single-nucleotide primer extension, restriction analysis, microarray technology, next generation methylation sequencing, nanopore sequencing, endonuclease digestion, affinity enrichment, target enrichment, hybrid capture, or enzymatic conversion.

In various embodiments, determining methylation statuses of methylation variants comprises determining methylation statuses of about 5 to about 5000 methylation variants. In various embodiments, determining methylation statuses of methylation variants comprises determining methylation statuses of 1043 methylation variants. In various embodiments, the proportion of fully methylated methylation variants relative to a total number of methylation variants is from about 0.00001 to about 0.9. In various embodiments, the proportion of fully methylated methylation variants relative to a total number of methylation variants is from about 0.0001 to about 0.001. In various embodiments, methods disclosed herein further comprise determining whether the sample is positive for cancer if the proportion of fully methylated methylation variants relative to a total number of methylation variants is greater than a threshold proportion. In various embodiments, the threshold proportion is from about 0.0001 to about 0.001. In various embodiments, the cell-free DNA fragments are detected with a sensitivity of at least 85% when the proportion of fully methylated methylation variants relative to a total number of methylation variants is greater than 0.0001, optionally wherein the cell-free DNA fragments are cancer-derived cell-free DNA fragments. In various embodiments, the proportion of fully methylated methylation variants is associated with tumor content, tumor size, or tumor burden. In various embodiments, the proportion of fully methylated methylation variants is indicative of a cancer type. In various embodiments, the cancer type comprises carcinomas, adenocarcinomas, blastomas, leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer, lung cancer, and the metastases thereof.

In various embodiments, the proportion of fully methylated methylation variants is associated with a cancer stage. In various embodiments, the cancer stage comprises stage 0, stage 1, stage 2, stage 3, or stage 4 cancer. In various embodiments, the sample is a blood sample. In various embodiments, determining methylation statuses of methylation variants comprises: providing at least one oligonucleotide probe designed to be complementary to a sequence of a binding site comprising two or more sequential CpG sites of a cell-free DNA fragment; using the at least one oligonucleotide probe, performing nucleic acid enrichment of the sequence of the binding site comprising two or more sequential CpG sites of the cell-free DNA fragment. In various embodiments, the at least one oligonucleotide probe is a DNA oligonucleotide probe or a RNA oligonucleotide probe. In various embodiments, providing at least one oligonucleotide probe comprises providing a primer pair. In various embodiments, performing nucleic acid enrichment comprises performing one or more of hybrid capture, nucleic acid amplification, or CRISPR-based enrichment. In various embodiments, determining methylation statuses of methylation variants comprises sequencing one or more nucleic acid sequences comprising the sequence of the binding site comprising two or more sequential CpG sites, or a complement thereof.

In various embodiments, determining methylation statuses of methylation variants further comprises: obtaining and aligning sequence reads to a reference genome, wherein at least a subset of the aligned sequence reads are aligned to genomic locations comprising two or more sequential CpG sites represented by methylation variants; for each methylation variant, determining methylation status of each CpG site of the two or more sequential CpG sites of the methylation variant. In various embodiments, determining methylation statuses of methylation variants further comprises: responsive to the determination that every CpG site of the two or more sequential CpG sites of the methylation variant was of a methylated state, identifying the methylation variant as fully methylated. In various embodiments, determining methylation statuses of methylation variants further comprises: responsive to the determination that not every CpG site of the two or more sequential CpG sites of the methylation variant was of a methylated state, identifying the methylation variant as not fully methylated.

Additionally disclosed herein is a method of determining tumor DNA content in a biological sample comprising: a) obtaining cell-free DNA fragments from the sample; b) determining methylation statuses of methylation variants each representing two or more sequential CpG sites in a genomic location from the cell-free DNA fragments; and c) quantifying a proportion of fully methylated methylation variants, wherein the cell-free DNA fragments are detected with a sensitivity of at least 85% when the proportion of the cell-free DNA fragments in which the sequential CpG sites of the genomic locations are methylated is greater than 0.0001. In various embodiments, the methylation variants are conserved across two or more, three or more, four or more, or five or more cancers. In various embodiments, each methylation variant comprises 3 or more sequential CpG sites, 4 or more sequential CpG sites, or 5 or more sequential CpG sites. In various embodiments, each methylation variant comprises 5 sequential CpG sites. In various embodiments, each methylation variant refers to a range of genomic locations and corresponding CpG sites identified in Table 1 or Table 2. In various embodiments, determining methylation statuses of methylation variants comprises performing one or more assays on the cell-free DNA fragments.

In various embodiments, the genomic assay comprises bisulfite conversion, nucleic acid amplification, polymerase chain reaction (PCR), methylation-specific PCR, bisulfite pyrosequencing, single-strand conformation polymorphism (SSCP) analysis, methylation-sensitive single-strand conformation analysis, high resolution melting analysis, methylation-sensitive single-nucleotide primer extension, restriction analysis, microarray technology, next generation methylation sequencing, nanopore sequencing, endonuclease digestion, affinity enrichment, target enrichment, hybrid capture, or enzymatic conversion. In various embodiments, determining methylation statuses of methylation variants comprises determining methylation statuses of about 5 to about 5000 methylation variants. In various embodiments, determining methylation statuses of methylation variants comprises determining methylation statuses of 1043 methylation variants. In various embodiments, the proportion of fully methylated methylation variants relative to a total number of methylation variants is from about 0.00001 to about 0.9 In various embodiments, the proportion of fully methylated methylation variants relative to a total number of methylation variants is from about 0.0001 to about 0.001. In various embodiments, methods disclosed herein further comprise determining whether the sample is positive for cancer if the proportion of fully methylated methylation variants relative to a total number of methylation variants is greater than a threshold proportion. In various embodiments, the threshold proportion is from about 0.0001 to about 0.001. In various embodiments, the proportion of fully methylated methylation variants is associated with tumor content, tumor size, or tumor burden. In various embodiments, the proportion of fully methylated methylation variants is indicative of a cancer type. In various embodiments, the cancer type comprises carcinomas, adenocarcinomas, blastomas, leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer, lung cancer, and the metastases thereof.

In various embodiments, the proportion of fully methylated methylation variants is associated with a cancer stage. In various embodiments, the cancer stage comprises stage 0, stage 1, stage 2, stage 3, or stage 4 cancer. In various embodiments, the sample is a blood sample.

Additionally disclosed herein is a method of identifying a methylation variant comprising: a) obtaining cell-free DNA fragments from a first plurality of samples; b) identifying a first set of genomic locations each comprising two or more sequential CpG sites that are methylated; c) obtaining cell-free DNA fragments from a second plurality of samples; d) identifying a second set of genomic locations each comprising two or more sequential CpG sites that are methylated in the second plurality of samples; and e) determining the methylation variant as the overlap between the first set of genomic locations and the second set of genomic locations, wherein the first set of genomic locations is present in at least 10% of the first plurality of samples and the second set of genomic locations is present in the second plurality of samples with a frequency of less than 0.010%. In some embodiments, the first set of genomic locations may be present in at least about 10% to about 80%, about 10% to about 70%, about 10% to about 60%, about 10% to about 50%, about 10% to about 40%, about 10% to about 30%, or about 10% to about 20% of the first plurality of samples. In some embodiments, the second set of genomic locations may be resent in the second plurality of sample with a frequency of less than about 0.009%, less than about 0.008%, less than about 0.007%, less than about 0.006%, less than about 0.005%, less than about 0.004%, less than about 0.003%, less than about 0.002%, or less than about 0.001%. In various embodiments, the methylation variants are conserved across two or more, three or more, four or more, or five or more cancers. In various embodiments, the first plurality of samples comprises at least 300 cancer biopsy samples. In various embodiments, the second plurality of samples comprises at least 300 cancer-free samples. In various embodiments, identifying the first and second set of genomic locations comprises sequencing nucleic acids derived from the cell-free DNA fragments from the first and second pluralities of samples. In various embodiments, the methylation variant comprises 3 or more sequential CpG sites, 4 or more sequential CpG sites, or 5 or more sequential CpG sites. In various embodiments, the methylation variant comprises 5 sequential CpG sites. In various embodiments, the methylation variant refers to a range of genomic locations and corresponding CpG sites identified in Table 1 or Table 2.

Additionally disclosed herein is a composition, comprising: a bisulfite-converted sequence from one of a range of genomic locations identified in Table 1 or Table 2 or the complement thereof; and an oligonucleotide complementary to a binding site of the bisulfite-converted sequence from one of the range of genomic locations identified in Table 1 or Table 2 or the complement thereof. In various embodiments, the oligonucleotide is a DNA oligonucleotide probe. In various embodiments, compositions disclosed herein further comprise a second oligonucleotide complementary to a second binding site. In various embodiments, the oligonucleotide is an RNA oligonucleotide probe. In various embodiments, the RNA oligonucleotide is a guide RNA. In various embodiments, the bisulfite-converted sequence comprises a sequence in which methylated cytosines have not been converted to thymine and unmethylated cytosines have been converted to uracil or thymine. In various embodiments, each bisulfite-converted sequence comprises from about 5 CpG sites to about 200 CpG sites.

Additionally disclosed herein is a non-transitory computer readable medium comprising instructions that, when executed by a processor, cause the processor to: obtain and align sequence reads to a reference genome, wherein at least a subset of the aligned sequence reads are aligned to genomic locations comprising two or more sequential CpG sites represented by methylation variants; for each methylation variant, determine methylation status of each CpG site of the two or more sequential CpG site of the methylation variant; and quantify a proportion of fully methylated methylation variants, wherein the cell-free DNA fragments are detected with a sensitivity of at least 85% when the proportion of the cell-free DNA fragments in which the sequential CpG sites of the genomic locations are methylated is greater than 0.0001. In various embodiments, the instructions that cause the processor to quantify a proportion of fully methylated methylation variants further comprises instructions that, when executed by the processor, cause the processor to: determine a quantity of sequence reads with fully methylated methylation variants; determine a quantity of total sequence reads comprising two or more sequential CpGs; and determine the proportion of fully methylated methylation variants as a ratio between the quantity of sequence reads with fully methylated methylation variants and the quantity of total sequence reads comprising two or more sequential CpGs.

In various embodiments, the instructions that cause the processor to determine a quantity of total sequence reads comprising two or more sequential CpGs further comprises instructions that, when executed by the processor, cause the processor to determine a quantity of total sequence reads comprising five sequential CpGs. In various embodiments, the methylation variants are conserved across two or more, three or more, four or more, or five or more cancers. In various embodiments, each methylation variant comprises 3 or more sequential CpG sites, 4 or more sequential CpG sites, or 5 or more sequential CpG sites. In various embodiments, each methylation variant comprises 5 sequential CpG sites. In various embodiments, each methylation variant refers to a range of genomic locations and corresponding CpG sites identified in Table 1 or Table 2. In various embodiments, the instructions that cause the processor to determine methylation statuses of methylation variants further comprises instructions that, when executed by the processor, cause the processor to determine methylation statuses of about 5 to about 5000 methylation variants. In various embodiments, the instructions that cause the processor to determine methylation statuses of methylation variants further comprises instructions that, when executed by the processor, cause the processor to determine methylation statuses of 1043 methylation variants.

In various embodiments, the proportion of fully methylated methylation variants relative to a total number of methylation variants is from about 0.00001 to about 0.9 In various embodiments, the proportion of fully methylated methylation variants relative to a total number of methylation variants is from about 0.0001 to about 0.001. In various embodiments, further comprising instructions that, when executed by a processor, cause the processor to: determine whether a sample, from which the sequence reads were derived, is positive for cancer if the proportion of fully methylated methylation variants relative to a total number of methylation variants is greater than a threshold proportion. In various embodiments, the threshold proportion is from about 0.0001 to about 0.001. In various embodiments, the cell-free DNA fragments are detected with a sensitivity of at least 85% when the proportion of fully methylated methylation variants relative to a total number of methylation variants is greater than 0.0001, optionally wherein the cell-free DNA fragments are cancer-derived cell-free DNA fragments. In various embodiments, the proportion of fully methylated methylation variants is associated with tumor content, tumor size, or tumor burden. In various embodiments, the proportion of fully methylated methylation variants is associated with a cancer stage. In various embodiments, the cancer stage comprises stage 0, stage 1, stage 2, stage 3, or stage 4 cancer.

Additionally disclosed herein is a non-transitory computer readable medium for identifying a methylation variant, the non-transitory computer readable medium comprising instructions that, when executed by a processor, cause the processor to: a) obtaining sequence reads derived from cell-free DNA fragments from a first plurality of samples; b) using the obtained sequence reads from the cell-free DNA fragments from a first plurality of samples, identifying a first set of genomic locations each comprising two or more sequential CpG sites that are methylated; c) obtaining sequence reads derived from cell-free DNA fragments from a second plurality of samples; d) using the obtained sequence reads from the cell-free DNA fragments from the second plurality of samples, identifying a second set of genomic locations each comprising two or more sequential CpG sites that are methylated; e) determining the methylation variant as an overlap between the first set of genomic locations and the second set of genomic locations, wherein the first set of genomic locations is present in at least 10% of the first plurality of samples and the second set of genomic locations is present in the second plurality of samples with a frequency of less than 0.01%. In various embodiments, the methylation variants are conserved across two or more, three or more, four or more, or five or more cancers. In various embodiments, the first plurality of samples comprises at least 300 cancer biopsy samples. In various embodiments, the second plurality of samples comprises at least 300 cancer-free samples. In various embodiments, identifying the first and second set of genomic locations comprises sequencing nucleic acids derived from the cell-free DNA fragments from the first and second pluralities of samples. In various embodiments, the methylation variant comprises 3 or more sequential CpG sites, 4 or more sequential CpG sites, or 5 or more sequential CpG sites. In various embodiments, the methylation variant comprises 5 sequential CpG sites. In various embodiments, the methylation variant refers to a range of genomic locations and corresponding CpG sites identified in Table 1 or Table 2.

Additionally disclosed herein is a method for preparing and sequencing nucleic acids, the method comprising: a) providing a sample of cell-free deoxyribonucleic acid (cfDNA) molecules from a subject; b) reacting the plurality of the cfDNA molecules with a deaminating agent to generate converted cfDNA molecules; c) amplifying a plurality of the converted cfDNA molecules by polymerase chain reaction (PCR)-based amplification to provide amplified nucleic acids; d) enriching the amplified nucleic acids for a panel of genomic regions, wherein the genomic regions each comprise a methylation locus comprising at least a portion of a range of genomic locations selected from Table 1 or Table 2; e) sequencing the enriched set of amplified nucleic acids to generate sequence reads, wherein the sequencing is performed at a sequence read depth of at least 5 sequence reads per base; f) determining methylation statuses of the genomic regions; and g) quantifying sequencing reads having two or more sequentially methylated CpG sites in the genomic regions. In various embodiments, the sequencing is performed at a sequence read depth of at least 10, at least 20, at least 50, at least 100, at least 100, at least 500, at least 1000, at least 2000, at least 3000, at least 4000, at least 5000, at least 10,000, at least 20,000, at least 30,000, at least 40,000, or at least 50,000 sequence reads per base. In various embodiments, each methylation locus comprising at least a portion of a range of genomic locations selected from Table 1 or Table 2 comprises two or more sequential CpG sites. In various embodiments, the two or more sequential CpG sites comprise three, four, or five sequential CpG sites.

Additionally disclosed herein is a method of detecting methylation markers in a human subject suspected of having cancer, the method comprising: determining a methylation status of each of at least 5 methylation markers identified in a sample obtained from the human subject suspected of having cancer, wherein the sample comprises cell-free DNA that is isolated from blood or plasma of the human subject, wherein each of the at least 5 methylation markers each comprise a methylation locus comprising at least a portion of a range of genomic locations selected from the group consisting of the range of genomic locations in Table 1 or Table 2. In various embodiments, determining the methylation status of each of at least 5 methylation markers comprises determining methylation status of each of at least 10, at least 20, at least 30, at least 40, at least 50, at least 100, at least 100, at least 500, at least 1000, at least 2000, at least 3000, at least 4000, at least 5000, at least 10,000, at least 20,000, at least 30,000, at least 40,000, or at least 50,000 methylation markers. In various embodiments, each methylation locus comprising at least a portion of a range of genomic locations selected from Table 1 or Table 2 comprises two or more sequential CpG sites. In various embodiments, the two or more sequential CpG sites comprise three, four, or five sequential CpG sites.

Additionally disclosed herein is a method of performing longitudinal tracking of tumor content across two or more samples comprising: a) obtaining cell-free DNA fragments from a sample obtained from a subject at a timepoint; b) determining methylation statuses of methylation variants each representing two or more sequential CpG sites in a genomic location from the cell-free DNA fragments; c) quantifying a proportion of fully methylated methylation variants without requiring a matched tissue sample; d) determining whether the sample is positive for cancer if the proportion of fully methylated methylation variants relative to a total number of methylation variants is greater than a threshold proportion; e) repeating steps (a)-(d) for an additional sample of the two or more samples obtained from the subject at a different timepoint; and f) determining a total number or proportion of samples of the two or more samples that are positive for cancer.

In various embodiments, the methylation variants are conserved across two or more, three or more, four or more, or five or more cancers. In various embodiments, each methylation variant comprises 3 or more sequential CpG sites, 4 or more sequential CpG sites, or 5 or more sequential CpG sites. In various embodiments, each methylation variant comprises 5 sequential CpG sites. In various embodiments, each methylation variant refers to a range of genomic locations and corresponding CpG sites identified in Table 1 or Table 2. In various embodiments, determining methylation statuses of methylation variants comprises performing one or more assays on the cell-free DNA fragments. In various embodiments, the one or more assays comprises bisulfite conversion, nucleic acid amplification, polymerase chain reaction (PCR), methylation-specific PCR, bisulfite pyrosequencing, single-strand conformation polymorphism (SSCP) analysis, methylation-sensitive single-strand conformation analysis, high resolution melting analysis, methylation-sensitive single-nucleotide primer extension, restriction analysis, microarray technology, next generation methylation sequencing, nanopore sequencing, endonuclease digestion, affinity enrichment, target enrichment, hybrid capture, or enzymatic conversion. In various embodiments, determining methylation statuses of methylation variants comprises determining methylation statuses of about 5 to about 5000 methylation variants. In various embodiments, determining methylation statuses of methylation variants comprises determining methylation statuses of 1043 methylation variants.

In various embodiments, the proportion of fully methylated methylation variants relative to a total number of methylation variants is from about 0.00001 to about 0.9. In various embodiments, the proportion of fully methylated methylation variants relative to a total number of methylation variants is from about 0.0001 to about 0.001. In various embodiments, methods disclosed herein further comprise determining whether the sample is positive for cancer if the proportion of fully methylated methylation variants relative to a total number of methylation variants is greater than a threshold proportion. In various embodiments, the threshold proportion is determined from a limit of detection (LOD) value. In various embodiments, the threshold proportion is from about 0.0001 to about 0.001. In various embodiments, the cell-free DNA fragments are detected with a sensitivity of at least 85% when the proportion of fully methylated methylation variants relative to a total number of methylation variants is greater than 0.0001, optionally wherein the cell-free DNA fragments are cancer-derived cell-free DNA fragments. In various embodiments, the proportion of fully methylated methylation variants is associated with tumor content, tumor size, or tumor burden. In various embodiments, the proportion of fully methylated methylation variants is indicative of a cancer type. In various embodiments, the cancer type comprises carcinomas, adenocarcinomas, blastomas, leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer, lung cancer, and the metastases thereof. In various embodiments, the proportion of fully methylated methylation variants is associated with a cancer stage. In various embodiments, the cancer stage comprises stage 0, stage 1, stage 2, stage 3, or stage 4 cancer.

In various embodiments, the sample is a blood sample. In various embodiments, determining methylation statuses of methylation variants comprises: providing at least one oligonucleotide probe designed to be complementary to a sequence of a binding site comprising two or more sequential CpG sites of a cell-free DNA fragment; using the at least one oligonucleotide probe, performing nucleic acid enrichment of the sequence of the binding site comprising two or more sequential CpG sites of the cell-free DNA fragment. In various embodiments, the at least one oligonucleotide probe is a DNA oligonucleotide probe or a RNA oligonucleotide probe. In various embodiments, providing at least one oligonucleotide probe comprises providing a primer pair. In various embodiments, performing nucleic acid enrichment comprises performing one or more of hybrid capture, nucleic acid amplification, or CRISPR-based enrichment. In various embodiments, determining methylation statuses of methylation variants comprises sequencing one or more nucleic acid sequences comprising the sequence of the binding site comprising two or more sequential CpG sites, or a complement thereof. In various embodiments, determining methylation statuses of methylation variants further comprises: obtaining and aligning sequence reads to a reference genome, wherein at least a subset of the aligned sequence reads are aligned to genomic locations comprising two or more sequential CpG sites represented by methylation variants; for each methylation variant, determining methylation status of each CpG site of the two or more sequential CpG site of the methylation variant. In various embodiments, determining methylation statuses of methylation variants further comprises: responsive to the determination that every CpG site of the two or more sequential CpG sites of the methylation variant was of a methylated state, identifying the methylation variant as fully methylated. In various embodiments, determining methylation statuses of methylation variants further comprises: responsive to the determination that not every CpG site of the two or more sequential CpG sites of the methylation variant was of a methylated state, identifying the methylation variant as not fully methylated.

Additionally disclosed herein is a method of treating a subject, the method comprising: a) performing longitudinal tracking of tumor content across two or more samples obtained from the subject, wherein the performing comprises: i) obtaining cell-free DNA fragments from a sample obtained from a subject at a timepoint; ii) determining methylation statuses of methylation variants each representing two or more sequential CpG sites in a genomic location from the cell-free DNA fragments; iii) quantifying a proportion of fully methylated methylation variants without requiring a matched tissue sample; iv) determining whether the sample is positive for cancer if the proportion of fully methylated methylation variants relative to a total number of methylation variants is greater than a threshold proportion; v) repeating steps (i)-(iv) for an additional sample of the two or more samples obtained from the subject at a different timepoint; and f) determining a total number or proportion of samples of the two or more samples that are positive for cancer; and g) responsive to determining that at least a threshold of the two or more samples are positive for cancer, administering a treatment to treat the subject. In various embodiments, the subject has not been previously diagnosed with cancer. In various embodiments, the subject has not been previously been administered a treatment for cancer.

Additionally disclosed herein is a method of modifying a treatment regimen for a subject, the method comprising: a) performing longitudinal tracking of tumor content across two or more samples obtained from the subject, wherein the performing comprises: i) obtaining cell-free DNA fragments from a sample obtained from a subject at a timepoint; ii) determining methylation statuses of methylation variants each representing two or more sequential CpG sites in a genomic location from the cell-free DNA fragments; iii) quantifying a proportion of fully methylated methylation variants without requiring a matched tissue sample; iv) determining whether the sample is positive for cancer if the proportion of fully methylated methylation variants relative to a total number of methylation variants is greater than a threshold proportion; v) repeating steps (i)-(iv) for an additional sample of the two or more samples obtained from the subject at a different timepoint; and f) determining a total number or proportion of samples of the two or more samples that are positive for cancer; and g) responsive to determining that at least a threshold of the two or more samples are positive for cancer, modifying the treatment regimen by providing an additional treatment to the subject. In various embodiments, the subject was previously classified as one of a non-responder, partial responder, or complete responder to a first treatment of the treatment regimen. In various embodiments, the subject was previously classified as one of a non-responder, partial responder, or complete responder according to a method other than quantifying a proportion of fully methylated methylation variants.

Additionally disclosed herein is a method of identifying a subject as a candidate subject for treatment, the method comprising: a) performing longitudinal tracking of tumor content across two or more samples obtained from the subject, wherein the performing comprises: i) obtaining cell-free DNA fragments from a sample obtained from a subject at a timepoint; ii) determining methylation statuses of methylation variants each representing two or more sequential CpG sites in a genomic location from the cell-free DNA fragments; iii) quantifying a proportion of fully methylated methylation variants without requiring a matched tissue sample; iv) determining whether the sample is positive for cancer if the proportion of fully methylated methylation variants relative to a total number of methylation variants is greater than a threshold proportion; v) repeating steps (i)-(iv) for an additional sample of the two or more samples obtained from the subject at a different timepoint; and f) determining a total number or proportion of samples of the two or more samples that are positive for cancer; and g) responsive to determining that at least a threshold of the two or more samples are positive for cancer, identifying the subject as a candidate subject for treatment.

### BRIEF DESCRIPTION OF THE DRAWINGS

These and other features, aspects, and advantages of the present invention will become better understood with regard to the following description and accompanying drawings.

FIG. 1A an overall flow process for predicting tumor content, in accordance with an embodiment.

FIG. 1B is a schematic depicting the concept of tumor content in an exemplary embodiment. Cell-free DNA may be obtained from a plasma sample and the proportion of DNA fragments ascribed to as cancer DNA fragments compared to non-tumor derived DNA fragments yields the tumor content (p).

FIG. 2 depicts an example conversion of nucleic acids, in accordance with an embodiment.

FIG. 3A is an example flow chart for predicting tumor content in a sample, in accordance with an embodiment.

FIG. 3B is an example flow chart for identifying a universal cancer signature, in accordance with an embodiment.

FIG. 3C shows an exemplary flow process for predicting prognosis for a subject.

FIG. 4A and FIG. 4B show example compositions including a converted nucleic acid sequence and an oligonucleotide complementary to a binding site of the converted nucleic acid sequence, in accordance with two embodiments.

FIG. 5 illustrates an example computer for implementing the entities shown in FIGS. 1A-1B, 2, 3A-3B, and 4A-4B.

FIG. 6A is a graph showing the correlation between estimating tumor content (TC) using single nucleotide variants (SNVs) in cell-free DNA and estimating TC using methylation variants (MVs).

FIG. 6B is a graph showing the number of methylation variants across 70 biopsy samples.

FIG. 6C is a graph showing the lack of recurring SNVs across multiple samples in contrast to methylation variants.

FIG. 7 is a graph showing the correlation between estimating TC using MVs versus estimating TC using MVs that use a reference positive control cancer sample (e.g., matched biopsy).

FIG. 8 is a graph showing the estimated TC using MVs (e.g., without the use of a matched biopsy) in cancer samples and in normal samples.

FIG. 9 is a graph showing differences in the range of estimated TC across different cancer indications. BLCA refers to bladder cancer. BRCA refers to breast cancer. CESC refers to cervical squamous cancer. COADREAD refers to colorectal cancer. ESCA refers to esophageal carcinoma. HNSC refers to head and neck squamous cell carcinoma. KIPAN refers to kidney cancer. LUNG refers to lung cancer. MISC refers to other cancers. NonCancer refers to a normal sample. OV refers to ovarian cancer. PAAD refers to pancreatic adenocarcinoma. PRAD refers to prostate adenocarcinoma. SKCM refers to skin cutaneous melanoma. STAD refers to stomach adenocarcinoma. THCA refers to thyroid cancer. UC refers to urothelial cancer.

FIG. 10 is a graph showing the relationship between TC determined using MVs and the stage of cancer.

FIG. 11A shows characteristics of cancer subjects and healthy subjects.

FIGS. 11B and 11C show box plots of estimated tumor content by cancer stage at timepoint 0.

FIG. 12A shows average estimated tumor content by RECIST response.

FIGS. 12B-12C show the endpoints of three stage IV prostate cancer partial responders and the corresponding estimated tumor content.

FIG. 13A shows an example prognosis score schematic.

FIG. 13B shows that high initial content values (e.g., >10%) can be associated with prognosis.

FIG. 13C shows that lower initial tumor content values and lack of association with prognosis.

FIG. 13D shows association between the number of samples obtained from patients across longitudinal timepoints that are above the LT and prognosis.

FIG. 14A shows the results of whole exome sequencing which was performed on matched biopsy and cfDNA so that SNVs could be used to estimate percentage cfDNA.

FIG. 14B shows the fraction of correctly classified cancer cfDNA samples by % cfDNA.

FIG. 15 shows a flowchart of a method of estimating tumor burden in an individual.

FIG. 16 is a flowchart of calculations that may be used in a method of estimating tumor burden in an individual.

### DETAILED DESCRIPTION

### Definitions

Terms used in the claims and specification are defined as set forth below unless otherwise specified.

The term “about” refers to a value that is within 10% above or below the value being described. For example, the term “about 5 nM” indicates a range of from 4.5 nM to 5.5 nM.

The terms “subject,” “patient,” and “individual” are used interchangeably and encompass a cell, tissue, or organism, human or non-human, male or female.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humor.

The terms “treating,” “treatment,” or “therapy” may be used interchangeably.

The term “CpG site” refers to a location of a genome that has cytosine and guanine separated by only one phosphate group, and is often denoted as “5′-C-phosphate-G-3′”, or “CpG” for short. Regions with a high frequency of CpG sites are commonly referred to interchangeably as “CG islands,” “CpG islands,” or “CGIs”.

The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

The terms “tumor content,” tumor fraction”, or “percent tumor in cfDNA” are used interchangeably and generally refer to a proportion of tumor derived nucleic acids in a sample. For example, tumor content may refer to a proportion of tumor derived nucleic acids in a sample relative to non-tumor derived nucleic acids. As another example, tumor content may refer to a proportion of tumor derived nucleic acids in a sample relative to the total nucleic acids in the sample.

The term “universal genomic location,” as used herein, refers to a recurring and/or substantially conserved (e.g., present in about 80% or more cancers) location and/or region in a genome wherein there are two or more sequential Cytosine-p-Guanine (CpG) sites. In some embodiments, the universal genomic locations are conserved in about 80% to about 90% (e.g., about 80%, about 81%, about 82%, about 83%, about 84%, about 85%, about 86%, about 87%, about 88%, about 89%, or about 90%) of two or more (e.g., three or more, four or more, five or more cancers, six or more, seven or more, eight or more, nine or more, ten or more, eleven or more, twelve or more, thirteen or more, fourteen or more, or fifteen or more) cancers.

The phrase “sequential CpG sites” refers to CpG sites within a range of genomic locations in which all CpG sites within the range of genomic locations are part of the sequential CpG sites. Sequential CpG sites include a neighboring CpG site i.e., a previous contiguous or next contiguous CpG site.

The phrase “methylation variant” refers to two or more sequential CpG sites within a genomic location that can be informative for detecting cancer in one or more samples. For example, methylation variants can be differentially methylated in cancer versus non-cancer samples. In various embodiments, a methylation variant refers to five sequential CpG sites, examples of which include five sequential CpG sites shown in Table 1 or Table 2.

The phrases “fully methylated methylation variant” and “universal cancer signature” are used interchangeably and generally refer to the fully methylated methylation status of two or more sequential CpG sites that are substantially conserved (e.g., methylated in about 80% or more of two or more cancers). In various embodiments, a fully methylated methylation variant or universal cancer signature refers to the fully methylated methylation status of five sequential CpG sites. Such fully methylated methylation variants are used to distinguish between nucleic acids (e.g., nucleic acid fragments) that are likely derived from cancer and other nucleic acids that are unlikely to be derived from cancer. For example, a fully methylated methylation variant that indicates that a DNA fragment originated from a tumor can be exemplified as five sequential CpG sites (e.g., five sequential CpG sites shown in Table 1 or Table 2, provided at the end of the Examples section) that are fully methylated.

The term “tumor burden” refers to a tumor size of a tumor. “Tumor burden” may include volumes, areas, lengths, and/or other measurements of a tumor.

It must be noted that, as used in the specification, the singular forms “a,” “an” and “the” include plural referents unless the context clearly dictates otherwise.

### Overview

Disclosed herein are methods and compositions for predicting tumor content, e.g., tumor content in a sample. Generally, methods and compositions disclosed herein involve obtaining a sample from a patient and determining methylation statuses of two or more sequential CpG sites in one or more genomic locations using nucleic acids of the obtained sample. The methylation status of two or more sequential CpG sites is referred to herein as a methylation variant. In various embodiments, the two or more sequential CpG sites appear on a common sequencing read. In particular embodiments, a methylation variant refers to methylation statuses of five sequential CpG sites. In such embodiments, the five sequential CpG sites appear on a common sequencing read. Such methylation variants are used to distinguish between nucleic acids (e.g., nucleic acid fragments) or sequence reads of nucleic acids that are likely derived from cancer and other nucleic acids that are unlikely to be derived from cancer. Quantifying proportions of nucleic acids (e.g., nucleic acid fragments) or sequence reads of nucleic acids that are likely derived from cancer enables the prediction of tumor content in a sample. As disclosed herein, methylation variants may be universal cancer signatures that are informative for determining tumor content of more than one cancer type. Predicting tumor content in a sample is useful for various purposes. In various embodiments, predicting tumor content is useful for identifying a presence of cancer in the sample. In various embodiments, predicting tumor content is useful for determining a cancer stage (e.g., stage 0, stage 1, stage 2, stage 3, or stage 4) of a cancer. In various embodiments, predicting tumor content is useful for determining a tissue of origin of a cancer. Altogether, methylation variants and/or universal cancer signatures disclosed can be applicable for various cancers (e.g., for identifying presence of one of a variety of different cancers in a sample, for determining a cancer stage for one of a variety of different cancers in a sample, or for determining a tissue of origin for one of a variety of different cancers).

In various embodiments, disclosed herein are methods for preparing and sequencing nucleic acids. In various embodiments, methods include providing a sample of cell-free deoxyribonucleic acid (cfDNA) molecules from a subject and reacting the plurality of the cfDNA molecules with a deaminating agent to generate converted cfDNA molecules. In various embodiments, methods include amplifying a plurality of the converted cfDNA molecules by polymerase chain reaction (PCR)-based amplification to provide amplified nucleic acids; enriching the amplified nucleic acids for a panel of genomic regions, wherein the genomic regions each comprise a methylation locus comprising at least a portion of a range of genomic locations selected from Table 1 or Table 2; and sequencing the enriched set of amplified nucleic acids to generate sequence reads, wherein the sequencing is performed at a sequence read depth of at least 5 sequence reads per base; determining methylation statuses of the genomic regions; and quantifying sequencing reads having two or more sequentially methylated CpG sites in the genomic regions.

In various embodiments, disclosed herein are methods for detecting methylation markers in a human subject suspected of having cancer, the method comprising: determining a methylation status of each of at least 5 methylation markers identified in a sample obtained from the human subject suspected of having cancer, wherein the sample comprises cell-free DNA that is isolated from blood or plasma of the human subject, wherein each of the at least 5 methylation markers each comprise a methylation locus comprising at least a portion of a range of genomic locations selected from the group consisting of the range of genomic locations in Table 1 or Table 2.

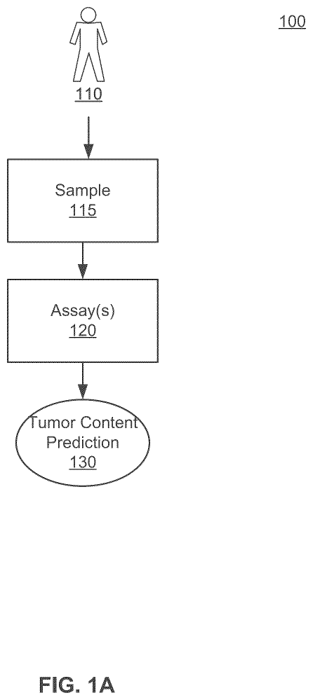

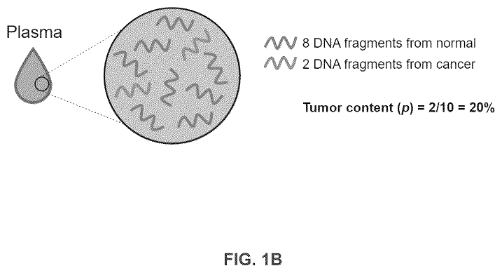

FIG. 1A an overall flow process 100 for predicting tumor content, in accordance with an embodiment. Although FIG. 1A shows the flow process in relation to a single subject 110, in various embodiments, the flow process can be performed for more than a single subject 110 (e.g., for thousands, millions, tens of millions, or hundreds of millions of subjects).

As shown in FIG. 1A, step 115 involves obtaining a sample 115 from the subject 110. In various embodiments, multiple samples 115 may be obtained from the subject 110 at multiple timepoints. In various embodiments, a sample is any of a blood sample, a plasma sample, a stool sample, a urine sample, a semen sample, an intraocular fluid sample, a peritoneal sample, a pleural fluid sample, an amniotic fluid sample, a cerebrospinal fluid sample, a mucous sample, or a saliva sample. In some embodiments, the biological sample may include a liquified biopsy obtained from a solid tissue and processed (e.g., through tissue trituration and/or enzymatic treatment) to produce a liquid sample. In particular embodiments, a sample obtained from the subject 110 is a blood sample, such as a liquid biopsy. The sample can be obtained by the subject or by a third party, e.g., a medical professional. Examples of medical professionals include physicians, emergency medical technicians, nurses, first responders, psychologists, phlebotomists, medical physics personnel, nurse practitioners, surgeons, dentists, and any other medical professional as would be known to one skilled in the art. In various embodiments, the sample 115 can be obtained from the subject 110 by a reference lab.

In some embodiments, the sample 115 may include nucleic acids that are informative for predicting tumor content in the sample 115. In various embodiments, the nucleic acids include cell-free DNA (cfDNA). In various embodiments, the nucleic acids include cell-free DNA fragments. In various embodiments, the cfDNA can be derived from tumor cells and is referred to herein as circulating tumor DNA (ctDNA). In particular embodiments, the nucleic acids include cfDNA fragments across a plurality of genomic locations. Genomic locations can include one or more CpG sites whose methylation statuses may be informative for predicting tumor content. Further details of exemplary genomic locations and CpG sites are described herein.

Next, one or more assays 120 are performed to generate the tumor content prediction 130. Generally, performing one or more assays 120 involves converting nucleic acids in the sample 115 obtained from the subject 110. In various embodiments, converting the nucleic acid involves converting unmethylated nucleotides (e.g., cytosines) to another nucleotide (a “converted nucleotide”, as used herein). In various embodiments, methylated cytosines are protected from conversion (e.g., deamination) during the conversion step. This enables subsequent downstream differentiation of methylated cytosines and unmethylated cytosines. Thus, methylation statuses of sequential CpG sites can be determined and used to predict tumor content in a sample. Further details of exemplary assays 120 are described herein.

The tumor content prediction 130 refers to a detection of tumor derived nucleic acids in a sample in relation to non-tumor derived nucleic acids in the sample. In various embodiments, the tumor content prediction 130 represents a proportion of tumor derived nucleic acids relative to the total nucleic acids in the sample. In various embodiments, tumor derived nucleic acids are identified as cell-free DNA fragments in which two or more sequential CpG sites of genomic locations are fully methylated. In particular embodiments, tumor derived nucleic acids are identified as cell-free DNA fragments in which five sequential CpG sites of genomic locations (e.g., five sequential CpG sites in ranges of genomic locations shown in Table 1 or Table 2) are fully methylated. In various embodiments, non-tumor derived nucleic acids are identified as cell-free DNA fragments in which two or more sequential CpG sites of genomic locations are not fully methylated. For example, for a DNA fragment, if a first CpG site is determined to be methylated and a second CpG is determined to be nonmethylated, the DNA fragment can be deemed a non-tumor derived nucleic acid.

Reference is made to FIG. 1B, which is a schematic depicting the concept of tumor content in an exemplary embodiment. Here, cell-free DNA fragments may be obtained from a plasma sample. Using the methods and compositions described herein, the cell-free DNA fragments can be identified as originating from cancer or normal tissue. The proportion of DNA fragments ascribed to as tumor derived DNA fragments compared to non-tumor derived DNA fragments yields the tumor content (p). In the example shown in FIG. 1B, a total of 8 DNA fragments are determined to originate from non-tumor tissue whereas a total of 2 DNA fragments are determined to originate from tumor tissue. Thus, in this example, the predicted tumor content is 20%. FIG. 1B is introduced merely for ease of explanation. In many scenarios, the proportion of DNA fragments originating from tumor tissue may be significantly lower than DNA fragments originating from non-tumor tissue (e.g., the proportion may be less than 1%, less than 0.1%, less than 0.01%, or less than 0.001%). The tumor content prediction 130 can then be useful for various purposes e.g., for determining whether the sample is positive for cancer, as is described in further detail herein.

### Example Assays

As discussed herein, FIG. 1A shows the step of performing one or more assays 120. In various embodiments, performing one or more assays 120 involves one or more steps of 1) converting nucleic acids from a sample, 2) enriching for target sequences of interest of the nucleic acids, and 3) determining and/or quantifying nucleic acids including two or more sequential CpG sites with particular methylation statuses (e.g., nucleic acids including two or more sequential CpG sites that are fully methylated). In various embodiments, performing one or more assays involves performing one or two of the aforementioned three steps. In particular embodiments, performing one or more assays 120 involves performing each of the aforementioned three steps. In various embodiments, performing one or more assays involves performing one or more of bisulfite conversion, nucleic acid amplification, polymerase chain reaction (PCR), methylation-specific PCR, bisulfite pyrosequencing, single-strand conformation polymorphism (SSCP) analysis, methylation-sensitive single-strand conformation analysis, high resolution melting analysis, methylation-sensitive single-nucleotide primer extension, restriction analysis, microarray technology, next generation methylation sequencing, nanopore sequencing, endonuclease digestion, affinity enrichment, target enrichment, hybrid capture, or enzymatic conversion.

Performing an assay can involve converting nucleic acids from the obtained sample 115. In various embodiments, converting nucleic acids includes treating the nucleic acids to capture methylation modifications. In various embodiments, converting nucleic acids involves converting one or more unmethylated nucleotides (e.g., cytosines) to another nucleotide (a “converted nucleotide”, as used herein), e.g., using chemical or enzymatic means. In certain embodiments, one or more unmethylated cytosines are converted to a nucleotide that pairs with adenine (e.g., the unmethylated cytosine may be converted to uracil). In certain embodiments, one or more unmethylated adenines are converted to a base that pairs with cytosine (e.g., the unmethylated adenine may be converted to inosine (I)). In certain embodiments, one or more methylated cytosines (e.g., a 5-methylcytosine (5mC)) is converted to a thymine, which pairs with adenine. In certain embodiments, methylated cytosines are protected from conversion (e.g., deamination) during the conversion step.

After a nucleic acid has been treated to convert unmethylated, or, in some cases, methylated nucleotides, into another nucleotide, the nucleic acid may be amplified. During amplification, the converted nucleotide pairs with its complementary nucleotide, and in the next round of amplification, the complementary nucleotide pairs with a replacement nucleotide. For example, following the conversion of an unmethylated cytosine to a uracil, the nucleic acid may be amplified such that an adenine pairs with the uracil in the first round of replication, and in the second round of replication, the adenine pairs with a thymine. Accordingly, the thymine replaces the uracil in the original nucleic acid sequence, and is referred to herein as a “replacement nucleotide”.

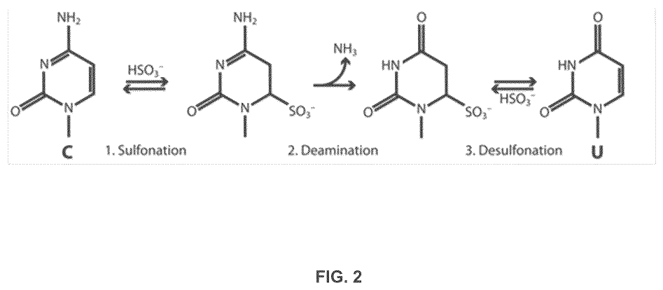

In various embodiments, the step of performing one or more assays 120 involves providing a sample of cell-free deoxyribonucleic acid (cfDNA) molecules from a subject and reacting the plurality of the cfDNA molecules with a deaminating agent to generate converted cfDNA molecules. In certain aspects, conversion of the nucleic acids involves using the deaminating agent to selectively deaminate nucleotides. FIG. 2 depicts an example conversion of nucleic acids, in accordance with an embodiment. Selective deamination refers to a process in which unmethylated cytosine residues are selectively deaminated over methylated cytosine (5-methylcytosine) residues. In certain embodiments, deamination of cytosine forms uracil, effectively inducing a C to T point mutation to allow for detection of methylated cytosines. Methods of deaminating cytosine are known in the art, and include chemical conversion (e.g., bisulfite conversion) and enzymatic conversion. In certain embodiments, the enzymatic conversion comprises subjecting the nucleic acid to TET2, which oxidizes methylated cytosines, thereby protecting them, and subsequent exposure to APOBEC, which converts unprotected (i.e., unmethylated) cytosines to uracils.

In some embodiments, the conversion, for example, bisulfite conversion or enzymatic conversion, uses commercially available kits. Bisulfite conversion can be performed using commercially available technologies, such as EZ DNA Methylation-Gold, EZ DNAMethylation-Direct or an EZ DNAMethylation-Lighting kit (Zymo Research Corp (Irvine, California)) or EpiTect Fast available from Qiagen (Germantown, MD). In another example a kit such as APOBECSeq (NEBiolabs) or OneStep qMethyl-PCR Kit (Zymo Research Corp (Irvine, California)) is used.

Bisulfite conversion is performed on DNA by denaturation using high heat, preferential deamination (at an acidic pH) of unmethylated cytosines, which are then converted to uracil by desulfonation (at an alkaline pH). Methylated cytosines remain unchanged on the single-stranded DNA (ssDNA) product.

In some embodiments the methods include treatment of the sample with bisulfite (e.g., sodium bisulfite, potassium bisulfite, ammonium bisulfite, magnesium bisulfite, sodium metabisulfite, potassium metabisulfite, ammonium metabisulfite, magnesium metabisulfite and the like). Unmethylated cytosine is converted to uracil through a three-step process during sodium bisulfite modification. As shown in FIG. 2, the steps are sulfonation to convert cytosine to cytosine sulphonate, deamination to convert cytosine sulphonate to uracil sulphonate and alkali desulfonation to convert uracil sulphonate to uracil. Conversion on methylated cytosine is much slower and is not observed at significant levels in a 4-16 hour reaction. (See Clark et al., Nucleic Acids Res., 22(15):2990-7 (1994).) If the cytosine is methylated it will remain a methylated cytosine. If the cytosine is unmethylated it will be converted to uracil. When the modified strand is copied, for example, through extension of a locus-specific primer, a random or degenerate primer or a primer to an adaptor, a G will be incorporated in the interrogation position (opposite the C being interrogated) if the C was methylated and an A will be incorporated in the interrogation position if the C was unmethylated and converted to U. When the double stranded extension product is amplified those Cs that were converted to Us and resulted in incorporation of A in the extended primer will be replaced by Ts during amplification. Those Cs that were not converted (i.e., the methylated Cs) and resulted in the incorporation of G will be replaced by unmethylated Cs during amplification.

In various embodiments, after conversion of nucleic acids, the converted nucleic acids undergo library construction. In various embodiments, converted nucleic acids can undergo end-repairing and/or addition of library or sequencing adapters. In various embodiments, converted nucleic acids can undergo biotinylation (e.g., addition of biotin moieties to converted nucleic acids). In various embodiments, barcodes can be incorporated into converted nucleic acids, thereby enabling subsequent sample demultiplexing (e.g., demultiplexing to identify sources of converted nucleic acids or demultiplexing to identify a common source from converted nucleic acids). As used herein, a “nucleic acid template” refers to a nucleic acid derived from the converted nucleic acid (e.g., any of a nucleic acid derived from a converted nucleic acid that underwent library construction, end-repairing, addition of library or sequencing adapters, biotinylation, barcode addition, or any combination thereof).

In various embodiments, the converted nucleic acids undergo nucleic acid amplification, an example of which includes polymerase chain reaction (PCR)-based amplification. Here, nucleic acid amplification results in the generation of amplified nucleic acids. Further examples of nucleic acid amplification assays, and in particular, PCR-based amplification, are described herein.

In various embodiments, performing the assay 120 described in FIG. 1A includes enriching for nucleic acid sequences of interest. In various embodiments, nucleic acid sequences of interest include sequences comprising ranges of genomic locations including two or more sequential CpG sites. In particular embodiments, nucleic acid sequences of interest include sequences comprising ranges of genomic locations including five sequential CpG sites (e.g., ranges of genomic locations including CpG sites shown in Table 1 or Table 2). In particular embodiments, the amplified nucleic acids are enriched for a panel of genomic regions. In various embodiments, one or more of the genomic regions comprise a methylation locus comprising at least a portion of a range of genomic locations selected from Table 1 or Table 2. In various embodiments, each genomic region comprises a methylation locus comprising at least a portion of a range of genomic locations selected from Table 1 or Table 2. In various embodiments, the portion of a range of genomic locations selected from Table 1 or Table 2 comprises two or more sequential CpG sites. In various embodiments, the two or more sequential CpG sites comprise three, four, or five sequential CpG sites. Example steps for enriching nucleic acid sequences of interest can include performing any of hybrid capture, nucleic acid amplification, or CRISPR-based enrichment methods.

In various embodiments, the amplified nucleic acids are enriched for a panel of genomic regions comprising at least 1000, at least 2000, at least 3000, at least 4000, at least 5000, at least 6000, at least 7000, at least 8000, at least 9000, or at least 10000 genomic regions. In various embodiments, the panel of genomic regions comprise cancer informative CG islands or “CGIs”. Example CGIs are disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety. In some embodiments, methylation statuses of a plurality of CpGs within a CGI may be analyzed. In some embodiments, at least a portion of the CpGs within a CGI may be analyzed. In other embodiments, all of the CpGs within a CGI may be analyzed.

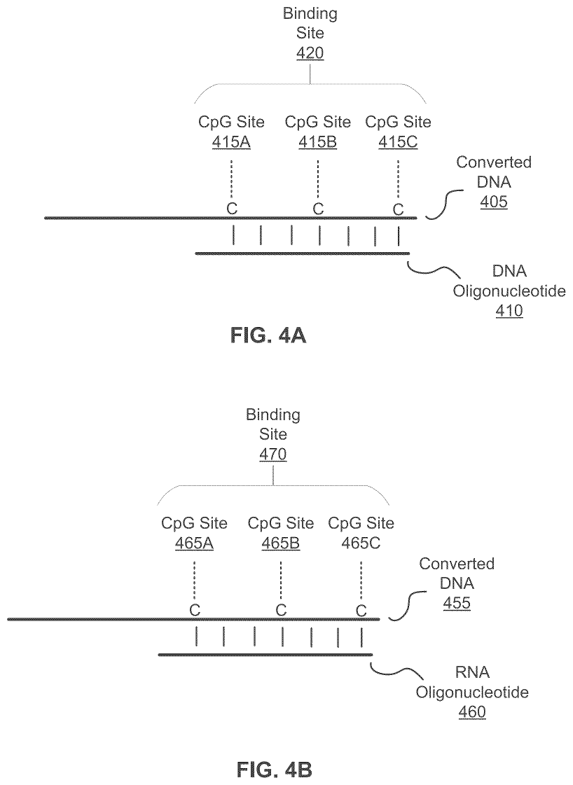

Referring to the method of hybrid capture, it may involve using a hybrid capture probe set. Here, a hybrid capture probe set can be generated such that probes of the probe set are complementary or substantially complementary to sequences of binding sites of converted nucleic acids. Examples of such hybrid capture probe sets include the KAPA HyperPrep Kit and SeqCAP Epi Enrichment System from Roche Diagnostics (Pleasanton, CA). For example, hybrid capture probe sets can be designed to hybridize with particular sequences of binding sites of converted nucleic acids (e.g., bisulfite converted DNA), thereby capturing and enriching the particular sequences. Further details of hybrid capture DNA oligonucleotide probes are described below in reference to FIG. 4A.

Referring to the method of nucleic acid amplification, in various embodiments, a nucleic acid amplification is “template-driven” in that base pairing of reactants, either nucleotides or oligonucleotides, have complements in a template polynucleotide that are required for the creation of reaction products. In one aspect, template-driven reactions are primer extensions with a nucleic acid polymerase, or oligonucleotide ligations with a nucleic acid ligase. Such reactions include, but are not limited to, polymerase chain reactions (PCR) assays, real-time PCR assays, quantitative real-time PCR (qPCR) assays, digital PCR (dPCR), allele-specific PCR assays, reverse-transcription PCR assays, reporter assays, linear polymerase reactions, nucleic acid sequence-based amplification (NASBAs), rolling circle amplifications, nicking endonuclease amplification (NEAR), transcription-mediated amplification (TMA), loop-mediated isothermal amplification (LAMP), helicase-dependent amplification (HAD), or strand displacement amplification (SDA) and the like, disclosed in the following references, each of which are incorporated herein by reference herein in their entirety: Mullis et al., U.S. Pat. Nos. 4,683,195; 4,965,188; 4,683,202; 4,800,159 (PCR); Gelfand et al., U.S. Pat. No. 5,210,015 (real-time PCR with “taqman” probes); Wittwer et al., U.S. Pat. No. 6,174,670; Kacian et al., U.S. Pat. No. 5,399,491 (“NASBA”); Lizardi, U.S. Pat. No. 5,854,033; Aono et al., Japanese patent publ. JP 4-262799 (rolling circle amplification); and the like. In one aspect, the amplification reaction is PCR. An amplification reaction may be a “real-time” amplification if a detection chemistry is available that permits a reaction product to be measured as the amplification reaction progresses, e.g., “real-time PCR”, or “real-time NASBA” as described in Leone et al., Nucleic Acids Research, 26: 2150-2155 (1998), and like references. For example, given a converted nucleic acid (e.g., bisulfite converted nucleic acid), primers designed to be complementary or substantially complementary to sequences of binding sites of the converted nucleic acid can be provided. Here, primers (e.g., PCR primers) are added to initiate the amplification of target sequences of binding sites of the converted nucleic acid. In various embodiments, the primers are whole genome primers that enable whole genome amplification. In various embodiments, the primers are gene-specific primers that result in amplification of sequences of specific genes. In various embodiments, the primers are allele-specific primers. For example, allele specific primers can target a range of genomic locations (e.g., a range of genomic locations shown in Table 1 or Table 2) which includes two or more sequential CpG sites. Therefore, performing nucleic acid amplification results in amplification of the range of genomic locations including the two or more sequential CpG sites.

In various embodiments, performing the assay 120 described in FIG. 1A includes determining and/or quantifying nucleic acids including two or more sequential CpG sites with particular methylation statuses. In various embodiments, determining and/or quantifying nucleic acids including two or more sequential CpG sites with particular methylation statuses is performed via quantitative methods such as by performing an ELISA, real-time PCR assay, quantitative PCR (qPCR) assay, allele-specific PCR assay, reverse-transcription PCR assay, or digital PCR (dPCR) assay. Therefore, the number of methylated, unmethylated, or partially methylated pre-selected genomic sequences can be quantified. In various embodiments, determining and/or quantifying nucleic acids including two or more sequential CpG sites with particular methylation statuses is performed via sequencing (e.g., by performing next generation sequencing). Thus, the sequenced reads can be aligned to a reference library and methylation sequence information including methylation statuses of two or more sequential CpG sites of genomic locations can be determined. Therefore, the number of methylated, unmethylated, or partially methylated genomic locations can be quantified via the sequenced reads. In various embodiments, the sequencing is performed at a sequence read depth of at least 5 sequence reads per base. In various embodiments, the sequencing is performed at a sequence read depth of at least 10, at least 20, at least 50, at least 100, at least 100, at least 500, at least 1000, at least 2000, at least 3000, at least 4000, at least 5000, at least 10,000, at least 20,000, at least 30,000, at least 40,000, or at least 50,000 sequence reads per base.

### Exemplary Methods

In various embodiments, the methods of the present disclosure involve determining tumor DNA content from a biological sample. In various embodiments, methods of the present disclosure involve preparing and sequencing nucleic acids. In various embodiments, methods of the present disclosure involve detecting methylation markers in a human subject suspected of having cancer. In various embodiments, methods for preparing and sequencing nucleic acids and/or methods for detecting methylation markers in a human subject can be useful e.g., for determining tumor DNA content in the human subject. Tumor DNA content is informative for, e.g., determining a presence of cancer in a sample and/or determining a risk of developing cancer. Generally, as described herein, one or more assays are performed to determine and/or quantify nucleic acids or reads of nucleic acids including two or more sequential CpG sites with particular methylation statuses (e.g., nucleic acids including two or more sequential CpG sites that are fully methylated). Thus, using the nucleic acids or reads of nucleic acids determined and/or quantified by performing the one or more assays, the nucleic acids or reads of nucleic acids originating from a tumor source and nucleic acids originating from a non-tumor source can be distinguished for purposes of calculating the predicted tumor content.

In various embodiments, methods involve performing the one or more assays to determine a proportion of the methylation variants in which two or more sequential CpG sites of genomic locations are methylated (e.g., fully methylated). In various embodiments, methods involve determining a proportion of the fully methylated methylation variants from the sample in which three or more, four or more, five or more, six or more, seven or more, eight or more, nine or more, or ten or more sequential CpG sites of genomic locations are methylated. In particular embodiments, the nucleic acids determined and/or quantified by performing the one or more assays are analyzed to determine a proportion of the methylation variants from the sample in which five sequential CpG sites of the genomic locations are methylated. In various embodiments, five sequential CpG sites of the genomic locations are considered methylated if every CpG site of the five sequential CpG sites is methylated. Thus, if less than five sequential CpG sites of the genomic locations are methylated (if at least one of the CpG sites is unmethylated), then the five sequential CpG sites of the genomic locations are considered not fully methylated.

In particular embodiments, the sequential CpG sites of the genomic locations refer to CpG sites of universal genomic locations as identified in Table 1 or Table 2. Specifically, Table 1 and Table 2 each shows human universal genomic locations and CpG sites mapped to human genome, hg19. The disclosure included herein is not meant to be limiting, and the person of ordinary skill in the art would recognize that other relevant regions and/or CpG sites of genomic locations can be identified in other versions of the human genome.

For example, the second row of Table 1 identifies a range of genomic locations between positions 36042792 and 36042826 of chromosome 1 (“chr1”). Five CpG sites are located at positions 36042792 (CpG1), 36042799 (CpG2), 36042816 (CpG3), 36042822 (CpG4) and 36042826 (CpG5). In this example, one or more sequence reads may be derived from bisulfite-converted DNA that includes a sequence including the range of genomic locations between positions 36042792 and 36042826 of chromosome 1. Thus, such sequence reads can be aligned to a reference genome to identify a sequence including the range of genomic locations between positions 36042792 and 36042826 of chromosome 1.

In various embodiments, the obtained sequence reads are aligned to a reference genome. Here, at least a subset of the aligned sequence reads are aligned to genomic locations comprising two or more sequential CpG sites. For example, a subset of the aligned sequence reads are aligned to ranges of genomic locations described in Table 1 or Table 2 with two or more sequential CpG sites (e.g., five sequential CpG sites as identified in Table 1 or Table 2). Predicting tumor content using aligned sequence reads enables analysis of sequential CpG sites that may not be present on a single cfDNA fragment. For example, a first cfDNA fragment may only encompass a portion of a range of genomic locations shown in Table 1 or Table 2. However, there may be additional cfDNA fragments that, taken together with the first cfDNA fragment, can provide aligned sequence reads that span the full range of genomic locations (e.g., range of genomic locations shown in Table 1 or Table 2).

The sequence reads are analyzed to determine the sequenced nucleotide corresponding to each of the five sequential CpG sites. For example, if the nucleotide at each of the five sequential CpG sites is a cytosine (or a complement thereof), then the methylation statuses of the five sequential CpG sites can be determined to be fully methylated. Thus, the cell-free DNA fragment corresponding to these fully methylated sequence reads can be categorized as likely originating from a tumor source.

As another example, if any of the nucleotides at the five sequential CpG sites is a thymine or uracil (or complement thereof), then the methylation statuses of the five sequential CpG sites can be determined to be not fully methylated (e.g., either partially methylated or fully nonmethylated). Thus, the cell-free DNA fragment corresponding to these not fully methylated sequence reads can be categorized as likely originating from a non-tumor source.

Table 1 and/or Table 2 further identifies additional ranges of genomic locations for additional chromosomes, such as chromosomes 1, 2, 3, 4, 5, 6, 7, 8, 9, 10, 11, 12, 13, 14, 15, 16, 17, 18, 19, 20, 21, and 22. The start and end of each universal genomic location is depicted using the nucleotide position. Additionally, for each universal genomic location, five CpG sites and the position of each CpG site is provided. Therefore, the description above in relation to the exemplary range of genomic locations between positions 36042792 and 36042826 of chromosome 1 can be similarly applicable to any of the additional ranges of genomic locations and CpG sites shown in Table 1 or Table 2.

In various embodiments, methods involve quantifying a proportion of cell-free DNA fragments comprising a range of genomic locations in which all CpG sites are methylated, and comparing the proportion to a threshold value. For example, the proportion can represent the tumor content (p) as described above in reference to FIG. 1B. The proportion can be the number of cell-free DNA fragments that are fully methylated are quantified in relation to a total number of cell-free DNA fragments. As another example, the proportion can be the number of cell-free DNA fragments that are fully methylated are quantified in relation to a number of cell-free DNA fragments that are not fully methylated. In various embodiments, if the quantified proportion of cell-free DNA fragments comprising a range of genomic locations in which all CpG sites are methylated is greater than a threshold value, then the cell-free DNA fragments comprising a range of genomic locations in which all CpG sites are methylated are deemed to have originated from a tumor source. In various embodiments, if the quantified proportion of cell-free DNA fragments comprising a range of genomic locations in which all CpG sites are methylated is less than a threshold value, then the cell-free DNA fragments comprising a range of genomic locations in which all CpG sites are methylated are deemed to not have originated from a tumor source.

In various embodiments, methods involve quantifying a proportion of fully methylated methylation variants and comparing the proportion to a threshold value. For example, the proportion can represent the tumor content (p). The proportion can be the number of methylation variants that are fully methylated in relation to a total number of methylation variants (e.g., methylated, partially methylated, and non-methylated methylation variants). As another example, the proportion can be the number of sequence reads having fully methylated methylation variants in relation to a number of sequence reads having any methylation variants (e.g., methylated, partially methylated, or non-methylated). In various embodiments, if the quantified proportion is greater than a threshold value, then the methylation variant is deemed to have originated from a tumor source. In various embodiments, if the quantified proportion is less than a threshold value, then the methylated variant is deemed to not have originated from a tumor source.

In various embodiments, the threshold value is between 0.00001 and 0.1. In various embodiments, the threshold value is between 0.00002 and 0.01, between 0.00004 and 0.005, between 0.00006 and 0.001, between 0.00008 and 0.0005, or between 0.00009 and 0.00025. In particular embodiments, the threshold value is about 0.0001.

In various embodiments, using the threshold value (e.g., threshold value of about 0.0001), cell-free DNA fragments originating from tumor sources (also referred to herein as circulating tumor DNA fragments) are successfully detected at a sensitivity of not less than 70%. In various embodiments, using the threshold value (e.g., threshold value of about 0.0001), circulating tumor DNA fragments) are successfully detected at a sensitivity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sensitivity. In particular embodiments, using the threshold value (e.g., threshold value of about 0.0001), circulating tumor DNA fragments) are successfully detected at a sensitivity of not less than 85%.

In various embodiments, using the threshold value (e.g., threshold value of about 0.0001), cell-free DNA fragments originating from tumor sources (also referred to herein as circulating tumor DNA fragments) are successfully detected at a specificity of at least 60%. In various embodiments, using the threshold value (e.g., threshold value of about 0.0001), cell-free DNA fragments originating from tumor sources (also referred to herein as circulating tumor DNA fragments) are successfully detected at a specificity of at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% specificity.

In various embodiments, using the threshold value (e.g., threshold value of about 0.0001), cell-free DNA fragments originating from tumor sources (also referred to herein as circulating tumor DNA fragments) are successfully detected at a particular sensitivity and a particular specificity. The combination of the sensitivity and specificity limits both the number of false positives and the number of false negatives. In various embodiments, using the threshold value (e.g., threshold value of about 0.0001), cell-free DNA fragments originating from tumor sources (also referred to herein as circulating tumor DNA fragments) are successfully detected at between 80% to 90% sensitivity and between 90% to 100% specificity. In various embodiments, using the threshold value (e.g., threshold value of about 0.0001), cell-free DNA fragments originating from tumor sources (also referred to herein as circulating tumor DNA fragments) are successfully detected at between 81% to 89% sensitivity and between 90% to 100% specificity. using the threshold value (e.g., threshold value of about 0.0001), cell-free DNA fragments originating from tumor sources (also referred to herein as circulating tumor DNA fragments) are successfully detected at between 82% to 88% sensitivity and between 90% to 100% specificity. In various embodiments, using the threshold value (e.g., threshold value of about 0.0001), cell-free DNA fragments originating from tumor sources (also referred to herein as circulating tumor DNA fragments) are successfully detected at between 83% to 87% sensitivity and between 90% to 100% specificity. In various embodiments, using the threshold value (e.g., threshold value of about 0.0001), cell-free DNA fragments originating from tumor sources (also referred to herein as circulating tumor DNA fragments) are successfully detected at between 84% to 86% sensitivity and between 90% to 100% specificity.

In various embodiments, for a sample with a corresponding tumor content prediction that indicates a presence of cancer in the sample, methods disclosed herein further involve providing an intervention to the subject from whom the sample was obtained. Example interventions include any a therapeutic intervention (e.g., a chemotherapeutic, a gene therapy, gene editing), a lifestyle intervention (e.g., change in behavior or habits), or a surgical intervention. In particular embodiments, methods disclosed herein involve administering one or more of a therapeutic agent, a radiotherapy, or a surgical intervention to the subject from whom the sample was obtained.

In various embodiments, methods disclosed herein involve detecting methylation markers in a human subject suspected of having cancer, the method comprising: determining a methylation status of each of at least 5 methylation markers identified in a sample obtained from the human subject suspected of having cancer, wherein the sample comprises cell-free DNA that is isolated from blood or plasma of the human subject, wherein each of the at least 5 methylation markers each comprise a methylation locus comprising at least a portion of a range of genomic locations selected from the group consisting of the range of genomic locations in Table 1 or Table 2. In various embodiments, methods involve detecting methylation markers of a subset of the 1043 genomic locations shown in Table 1. In various embodiments, methods involve detecting methylation markers of each of the 1043 genomic locations shown in Table 1. In various embodiments, methods involve detecting methylation markers of a subset of the 561 genomic locations shown in Table 2. In various embodiments, methods involve detecting methylation markers of each of the 561 genomic locations shown in Table 2. In various embodiments, methods involve detecting methylation markers of at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 of the 1043 genomic locations shown in Table 1. In various embodiments, methods involve detecting methylation markers of at least 10, at least 20, at least 30, at least 40, at least 50, at least 60, at least 70, at least 80, at least 90, at least 100, at least 200, at least 300, at least 400, or at least 500 of the 561 genomic locations shown in Table 1.

In various embodiments, determining the methylation status of each of at least 5 methylation markers comprises determining methylation status of each of at least 10, at least 20, at least 30, at least 40, at least 50, at least 100, at least 100, at least 500, at least 1000, at least 2000, at least 3000, at least 4000, at least 5000, at least 10,000, at least 20,000, at least 30,000, at least 40,000, or at least 50,000 methylation markers. In various embodiments, each methylation locus comprising at least a portion of a range of genomic locations selected from Table 1 or Table 2 comprises two or more sequential CpG sites. In various embodiments, the two or more sequential CpG sites comprise three, four, or five sequential CpG sites.

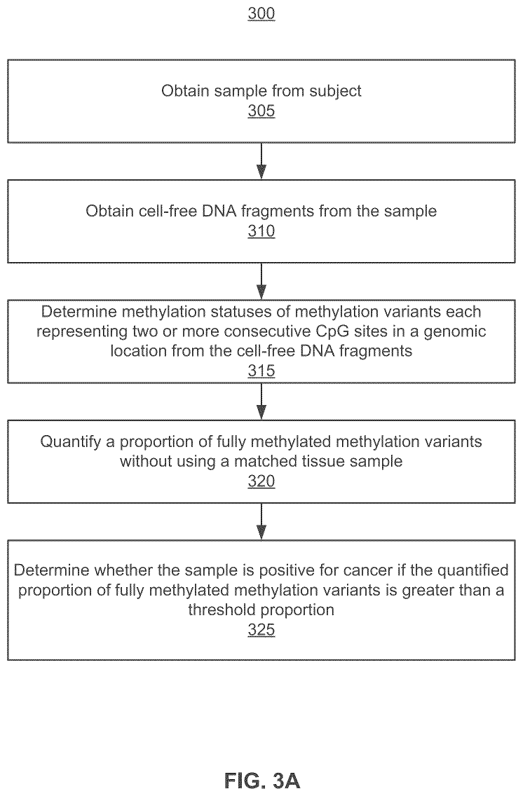

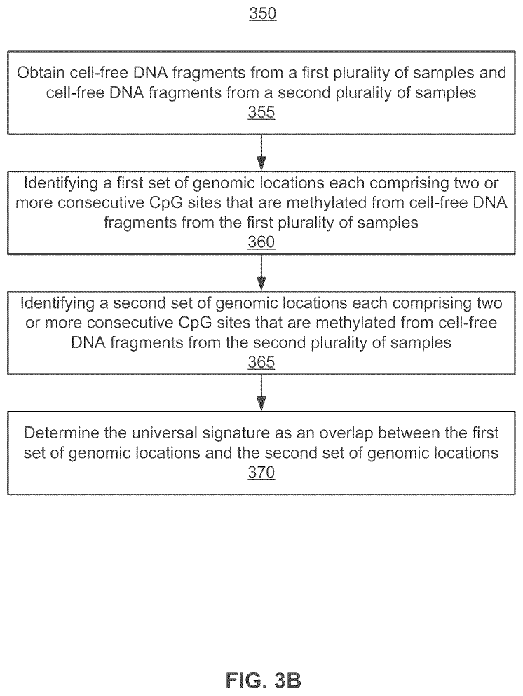

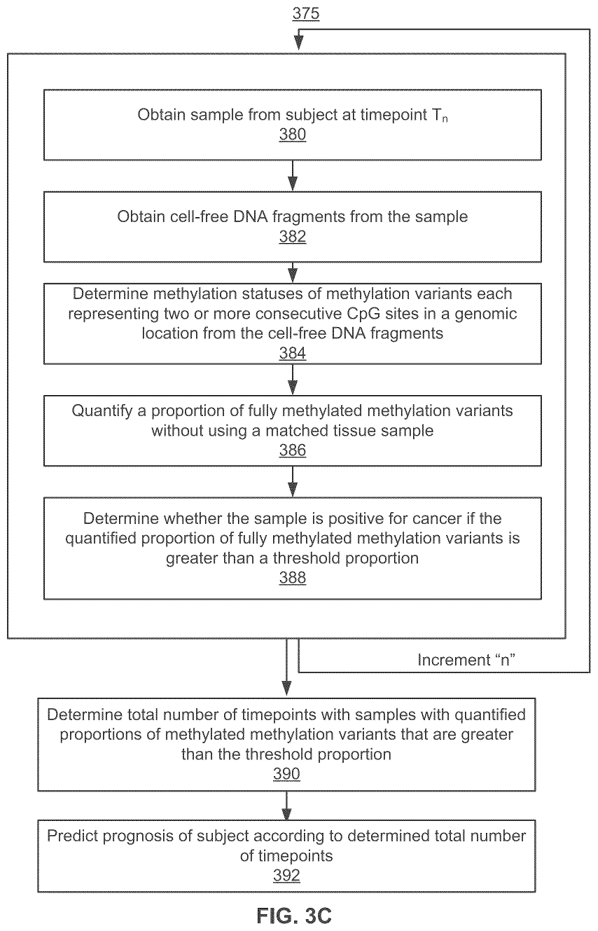

Reference is now made to FIG. 3A, which is a flow chart depicting an exemplary method for predicting tumor content in a sample, in accordance with an embodiment.

Step 305 involves obtaining a sample from a subject. In particular embodiments, step 305 involves obtaining a blood or plasma sample from the subject. Step 310 involves obtaining cell-free DNA fragments from the sample. Here, cell-free DNA fragments may derive from a tumor.

Step 315 involves determining methylation statuses of CpG sites in a plurality of genomic locations comprising two or more sequential CpG sites from the cell-free DNA fragments. In particular embodiments, step 315 involves determining methylation statuses of CpG sites in genomic locations that include five or more sequential CpG sites.

Step 320 involves quantifying a proportion of fully methylated methylation variants in which every CpG site of the sequential CpG sites is methylated. Here, the quantified proportion can represent the predicted tumor content. For example, step 320 can involves quantifying a proportion of methylation variants in which five sequential CpG sites are fully methylated. In particular embodiments, the particular CpG sites of the genomic locations are of interest because they were previously identified as universal cancer signatures which, when associated with cancer, are present in sufficient quantity in cancer biopsy samples and are present in low quantities in non-cancer samples. Thus, through the analysis of methylation statuses of universal cancer signatures, step 320 does not involve using a matched tissue sample.

In certain embodiments, step 325 involves determining whether the sample is positive for cancer using the predicted tumor content. For example, step 325 can involve determining whether the proportion of genomic locations in which the sequential CpG sites are methylated in relation to the total number of genomic locations in the cell-free DNA fragments is greater than a threshold proportion. If the genomic locations in which the sequential CpG sites are methylated is greater than the threshold proportion, the sample can be deemed to be positive for a cancer. Conversely, if the genomic locations in which the sequential CpG sites are methylated is less than the threshold proportion, the sample can be deemed to be negative for a cancer. In various embodiments, the threshold proportion is a threshold value based on a limit of detection (LOD). Further details of LOD are described herein.

### Methods for Identifying Universal Cancer Signatures

Methods disclosed herein involve identifying universal cancer signatures, also referred to herein as fully methylated methylation variants. Generally, a universal cancer signature is identified as a signature that is present and detectable across two or more different types of cancer. For example, a universal cancer signature can be a fully methylated string of 5 sequential CpG sites within a range of genomic locations, such as a range of genomic locations identified in Table 1 or Table 2. In various embodiments, the universal cancer signature is identified as a signature that is present and detectable across two, three, four, five, six, seven, eight, nine, ten, eleven, twelve, thirteen, fourteen, fifteen, sixteen, seventeen, eighteen, nineteen, or twenty different types of cancer. Generally, universal cancer signatures may be present in a sufficiently high frequency across cancer samples, but are present at a sufficiently low frequency across non-cancer samples, thereby enabling differentiation between cancer and non-cancer samples.

In various embodiments, a universal cancer signature is present at a frequency of less than 1 in 100,000 across normal plasma samples. In various embodiments, a universal cancer signature is present at a frequency of less than 1 in 50,000, less than 1 in 20,000, less than 1 in 10,000, less than 1 in 5,000, or less than 1 in 1,000 across normal plasma samples. In particular embodiments, a universal cancer signature is present at a frequency of less than 1 in 10,000 across normal plasma samples. In various embodiments, a universal cancer signature are present at a frequency of greater than 1 in 1,000 across cancer samples. In various embodiments, a universal cancer signature is present at a frequency of greater than 1 in 500, greater than 1 in 200, greater than 1 in 100, greater than 1 in 50, greater than 1 in 25, or greater than 1 in 10 across cancer samples. In particular embodiments, a universal cancer signature is present at a frequency of greater than 1 in 10 across cancer samples. In particular embodiments, a universal cancer signature is present at a frequency of less than 1 in 10,000 across normal plasma samples and is present at a frequency of greater than 1 in 10 across cancer samples.

Reference is now made to FIG. 3B, which is an example flow chart for identifying a universal cancer signature, in accordance with an embodiment.

Step 355 involves obtaining cell-free DNA fragments from a first plurality of samples and cell-free DNA fragments from a second plurality of samples. In particular embodiments, the first plurality of samples are known to have a first type of cancer and the second plurality of samples are known to have a second type of cancer. In various embodiments, step 355 can further involve obtaining cell-free DNA fragments from additional pluralities of samples known to have additional types of cancers.

Step 360 involves identifying a first set of universal genomic locations each comprising two or more sequential CpG sites that are methylated from cell-free DNA fragments from the first plurality of samples. In particular embodiments, step 360 involves identifying a first set of universal genomic locations each comprising five sequential CpG sites that are fully methylated from cell-free DNA fragments from the first plurality of samples.

Step 365 involves identifying a second set of universal genomic locations each comprising two or more sequential CpG sites that are methylated from cell-free DNA fragments from the second plurality of samples. In particular embodiments, step 365 involves identifying a second set of universal genomic locations each comprising five sequential CpG sites that are fully methylated from cell-free DNA fragments from the second plurality of samples.

Step 370 involves determining the universal signature as an overlap between the first set of universal genomic locations and the second set of universal genomic locations.

### Longitudinal Tracking Using Methylation Variants

Methods disclosed herein further encompass longitudinal tracking in a subject using methylation variants, such as the methylation variants shown in Table 1 or Table 2. In various embodiments, longitudinal tracking involves predicting cancer scores for multiple samples obtained from the subject at different timepoints. For example, the cancer scores can be indicative of predicted tumor content in the multiple samples obtained from the subject at different points. In various embodiments, the cancers scores are predicted tumor content values in the multiple samples determined using the methylation variants described herein, such as methylation variants of Table 1 or Table 2. Therefore, the determined tumor content across the samples obtained from the subject at different timepoints can be valuable for understanding the prognosis for the subject.

In various embodiments, predicting a cancer score can involve implementing a machine-learned model that analyzes the methylation variants, such as the methylation variants of Table 1 or Table 2. For example, such a machine-learned model can analyze the methylation statuses of two or more sequential CpG sites of genomic locations. In particular embodiments, the machine-learned model analyzes the methylation statuses of five sequential CpG sites in ranges of genomic locations shown in Table 1 or Table 2 and outputs a cancer score.

In various embodiments, methods for longitudinal tracking involve obtaining samples from the subject and predicting cancer scores in the obtained samples across at least two timepoints. In various embodiments, methods for longitudinal tracking involve obtaining samples from the subject and predicting cancer scores for the obtained samples across at least three timepoints. In various embodiments, methods for longitudinal tracking involve obtaining samples from the subject and predicting cancer scores for the obtained samples across at least four timepoints. In various embodiments, methods for longitudinal tracking involve obtaining samples from the subject and predicting cancer scores for the obtained samples across at least five timepoints, at least six timepoints, at least seven timepoints, at least eight timepoints, at least nine timepoints, at least ten timepoints, at least eleven timepoints, at least twelve timepoints, at least thirteen timepoints, at least fourteen timepoints, at least fifteen timepoints, at least sixteen timepoints, at least seventeen timepoints, at least eighteen timepoints, at least nineteen timepoints, or at least twenty timepoints. In various embodiments, the time between any two timepoints can be between 1 day and 12 months, between 5 days and 8 months, between 10 days and 6 months, between 15 days and 4 months, between 20 days and 3 months, between 30 days and 2 months. In various embodiments, the time between any two timepoints can be between 1 days and 10 days, between 10 days and 20 days, between 20 days and 30 days, between 30 days and 40 days, between 40 days and 50 days, or between 50 days and 60 days. In various embodiments, the time between any two timepoints can be between 1 day and 100 days, between 5 day and 80 days, between 10 days and 70 days, between 15 days and 60 days, between 20 days and 50 days, between 25 days and 40 days, or between 30 days and 35 days. In various embodiments, the time between any two timepoints can be between 1 days and 10 days, between 10 days and 20 days, between 20 days and 30 days, between 30 days and 40 days, between 40 days and 50 days, or between 50 days and 60 days. In various embodiments, the time between any two timepoints can be between 1 month and 2 months.

In particular embodiments, methods for longitudinal tracking involve obtaining a sample from the subject at a first timepoint (e.g., an initial timepoint) and predicting cancer score for the sample obtained at the first timepoint. In various embodiments, the first timepoint may refer to a timepoint prior to which the subject receives a therapeutic, such as a cancer therapeutic. Thus, the predicted cancer score for from the sample obtained at the first timepoint may represent a baseline cancer score prior to any therapeutic treatment. In various embodiments, the first timepoint may refer to a timepoint immediately after the subject receives a therapeutic, such as a cancer therapeutic. In this context, “immediately after” the subject receives a therapeutic can refer to a timeframe within 1 day after the subject receives the therapeutic. In various embodiments, “immediately after” refers to a timeframe within 12 hours, within 8 hours, within 6 hours, within 4 hours, within 3 hours, within 2 hours, within 1 hour, within 30 minutes, within 15 minutes, within 10 minutes, within 5 minutes, or within 1 minute of the subject receiving the therapeutic.

In particular embodiments, methods for longitudinal tracking further involve obtaining one or more subsequent samples from the subject after the first timepoint (e.g., at a second timepoint, at a third timepoint, at a fourth timepoint, etc.) and predicting cancer scores for the one or more subsequent samples. The cancer scores from the one or more subsequent samples can be indicative of the progression of the tumor within the subject after the first timepoint. In various embodiments, the one or more subsequent samples are obtained from the subject after the subject has received a therapeutic, such as a cancer therapeutic. Thus, the cancer scores of the one or more subsequent samples can be reflective of the progression of the tumor within the subject in response to the provided therapeutic.

In various embodiments, longitudinal tracking using the universal cancer signatures is useful for predicting a prognosis for a subject. In various embodiments, based on the longitudinal tracking of a subject, the subject can be classified in group associated with a particular outcome. For example, the subject can be classified in one of likely to survive or unlikely to survive. As another example, the subject can be classified in one of a responder to a therapeutic or anon-responder to a therapeutic. As another example, the subject can be classified in one of a full responder to a therapeutic, partial responder to a therapeutic, or non-responder to a therapeutic. In various embodiments, the subject can be classified in one of a favorable outcome (examples of which include likely to survive or responder to a therapeutic) or unfavorable outcome (examples of which include unlikely to survive or non-responder to a therapeutic).

In particular embodiments, methods for longitudinal tracking may be useful for early detection of cancer (e.g., prior to diagnosis of cancer in patients). In various embodiments, methods involve obtaining a sample from the subject at one or more timepoints prior to diagnosis of cancer, and predicting cancer scores for the samples obtained across the one or more timepoints. Here, the subject may be suspected of having cancer or may be at risk of having cancer (e.g., subject is identified as in a high-risk cancer group). Thus, the predicted cancer scores for the subject across the one or more timepoints can be useful for early detection of cancer in the subject.

In various embodiments, based on the longitudinal tracking of a subject, the subject can be classified in group associated with a particular outcome. For example, the subject can be classified in an unfavorable category (e.g., a presence of cancer) or a favorable category (e.g., a lack of cancer).

In various embodiments, predicting the prognosis for the subject based on the longitudinal tracking involves determining whether the cancer scores across the multiple samples obtained from the subject are increasing, decreasing, or unchanging. In various embodiments, the cancer scores are determined to be increasing, decreasing, or unchanging by determining a slope across the cancer scores. For example, a linear regression may be fit across the cancer scores and the slope of the linear regression would indicate whether the cancer scores are increasing (e.g., positive slope), decreasing (e.g., negative slope), or unchanging (e.g., slope between ±0.1).

In some embodiments, if cancer scores, which can be reflective of predicted tumor content, are increasing over the successive timepoints, then the prognosis for the subject can indicate that the cancer in the subject is present or is progressing. Thus, the subject may be classified in an unfavorable category, such as one of having cancer, unlikely to survive, or a non-responder to a therapeutic. As another example, if the cancer scores are decreasing over successive timepoints, then the prognosis for the subject can indicate that the cancer in the subject is not present or is not progressing. Thus, the subject may be classified in a favorable category, such as one of absence of cancer, likely to survive, or a responder to a therapeutic. As another example, if the cancer scores are unchanging over successive timepoints, then the prognosis for the subject can indicate the cancer in the subject is not progressing or is remaining steady. here, the subject may be classified as a partial responder.

In various embodiments, predicting the prognosis for the subject based on the longitudinal tracking involves determining the total number or proportion of cancer scores across the timepoints that are greater than a threshold value, also referred to herein as a longitudinal threshold value. Thus, in such embodiments, the relative progression of the cancer scores across the timepoints (e.g., increase, decrease, or unchanging) is not needed to predict prognosis; rather, the total number or proportion of samples obtained from the subject in which cancer is detected as present is informative of the future prognosis. In various embodiments, the threshold value is empirically determined. In various embodiments, the threshold value is a limit of detection (LOD) threshold. In various embodiments, the threshold value is less than 0.04%, 0.039%, 0.038%, 0.037%, 0.036%, 0.035%, 0.034%, 0.033%, 0.032%, 0.31%, 0.030%, 0.029%, 0.028%, 0.027%, 0.026%, 0.025%, 0.024%, 0.023%, 0.022%, 0.021%, 0.020%, 0.019%, 0.018%, 0.017%, 0.016%, 0.015%, 0.014%, 0.013%, 0.012%, 0.011%, or 0.010%. In particular embodiments, the threshold value is 0.037%. Further details of example threshold values, such as longitudinal threshold values, are described herein.

In various embodiments, predicting the prognosis for the subject based on the longitudinal tracking involves determining the total number or proportion of cancer scores across a set of timepoints that are greater than a threshold value, where all timepoints within the set of timepoints are within a sliding window. Thus, only timepoints within the sliding window are considered for the longitudinal tracking analysis whereas timepoints outside the sliding window are not considered for the longitudinal tracking analysis. In various embodiments, the sliding window is between about 0.5 months and about 12 months, between about 2 months and about 11 months, between about 3 months and about 10 months, between about 4 months and about 9 months, between about 5 months and about 8 months, and between about 6 months and about 7 months. In various embodiments, the sliding window is between about 0.5 months and about 6 months, between about 1 month and about 5 months, between about 2 months and 4 months, and between about 2.5 months and about 3.5 months.

In various embodiments, the subject is classified in an unfavorable category if at least two cancer scores for the subject across two timepoints are greater than a threshold value. In various embodiments, the subject is classified in an unfavorable category if at least three cancer scores for the subject across three timepoints are greater than a threshold value. In various embodiments, the subject is classified in an unfavorable category if at least four cancer scores for the subject across four timepoints are greater than a threshold value. In various embodiments, the subject is classified in an unfavorable category if at least five cancer scores for the subject across five timepoints are greater than a threshold value.

In various embodiments, the subject is classified in a favorable category if fewer than three cancer scores for the subject across three timepoints are greater than a threshold value. In various embodiments, the subject is classified in a favorable category if fewer than two cancer scores for the subject across two timepoints are greater than a threshold value. In various embodiments, the subject is classified in a favorable category if fewer than one cancer score for the subject at a single timepoint is greater than a threshold value.

In various embodiments, the subject is classified in an unfavorable category if greater than a proportion of cancer scores for the subject are greater than a threshold value. In various embodiments, if greater than 50% of cancer scores for the subject are greater than a threshold value, the subject is classified in an unfavorable category. For example, assume samples are obtained from the subject across 4 different timepoints. Therefore, if 2 or more (e.g., greater than 50%) of the samples are determined to have a cancer score that is greater than a threshold value, then the subject is classified in an unfavorable category. In various embodiments, if greater than 60%, greater than 70%, greater than 80%, or greater than 90% of cancer scores for the subject are greater than a threshold value, the subject is classified in an unfavorable category. In particular embodiments, if 100% of the cancer scores are greater than a threshold value (e.g., every obtained sample from the subject is associated with a cancer score greater than a threshold), then the subject is classified in an unfavorable category.

In various embodiments, the subject is classified in a favorable category if less than a proportion of cancer scores for the subject are greater than a threshold value. In various embodiments, if less than 100% of cancer scores for the subject are greater than a threshold value, the subject is classified in a favorable category. In various embodiments, if less than 70% of cancer scores for the subject are greater than a threshold value, the subject is classified in a favorable category. In various embodiments, if less than 50% of cancer scores for the subject are greater than a threshold value, the subject is classified in a favorable category. In various embodiments, if less than 40%, less than 30%, less than 20%, or less than 10% of cancer scores for the subject are greater than a threshold value, the subject is classified in a favorable category.

Reference is now made to FIG. 3C, which shows an exemplary flow process 375 for predicting prognosis for a subject. Generally, FIG. 3C shows steps 380, 382, 384, 386, and 388 which involve analyzing a particular sample obtained from a subject. Here, steps 380, 382, 384, 386, 388 may involve the same methods as described in reference to steps 305, 310, 315, 320, and 325 in FIG. 3A. Specifically, step 380 involves obtaining a sample from a subject at a timepoint Tₙ. If the sample is obtained at an initial timepoint, then n=0 and the sample is obtained at initial timepoint T₀. Step 382 involves obtaining cell-free DNA fragments from the sample. Here, cell-free DNA fragments may derive from a tumor in the subject.

Step 384 involves determining methylation statuses of CpG sites in a plurality of genomic locations comprising two or more sequential CpG sites from the cell-free DNA fragments. In particular embodiments, step 315 involves determining methylation statuses of CpG sites in genomic locations that include five or more sequential CpG sites. Example CpG sites are described in Table 1 or Table 2.

Step 386 involves quantifying a proportion of fully methylated methylation variants in which every CpG site of the sequential CpG sites is methylated. Step 388 involves determining whether the sample is positive for cancer using the predicted tumor content. For example, step 388 can involve determining whether the proportion of genomic locations in which the sequential CpG sites are methylated in relation to the total number of genomic locations in the cell-free DNA fragments is greater than a threshold proportion. If the genomic locations in which the sequential CpG sites are methylated is greater than the threshold proportion, the sample can be deemed to be positive for a cancer. Conversely, if the genomic locations in which the sequential CpG sites are methylated is less than the threshold proportion, the sample can be deemed to be negative for a cancer. In various embodiments, the threshold proportion is a threshold value based on a limit of detection (LOD).

As shown in FIG. 3C, step 380, 382, 384, 386, and 388 may be repeated for a new sample obtained at a subsequent timepoint (e.g., by incrementing “n”). Thus, the new sample can be analyzed and presence of cancer can be determined in the new sample.

Step 390 involves determining the total number of timepoints with samples that are determined to be positive for cancer. For example, step 390 involves determining the total number of timepoints with samples with quantified proportions of methylated methylation variants that are greater than the threshold proportion.

Step 392 involves predicting prognosis of the subject according to the determined total number of timepoints. In various embodiments, the total number of timepoints with samples that are determined to be positive for cancer is directly informative for the predicted prognosis. In various embodiments, the total number of samples that are determined to be positive for cancer is useful for determining a total proportion of samples obtained from the subject that are positive for cancer. For example, if 100% of the samples obtained from the subject are determined to be positive for cancer, the subject can be classified in an unfavorable category (e.g., unlikely to survive or likely non-responder to a therapeutic).

### Compositions for Determining Tumor Content and Uses Thereof

Additionally disclosed herein are compositions useful for determining tumor content in a sample. In various embodiments, compositions include two nucleic acids, in which a first nucleic acid includes a binding site sequence with one or more CpG sites, and a second nucleic acid includes a sequence complementary to the binding site sequence.

In various embodiments, the first nucleic acid includes a range of genomic locations. Example ranges of genomic locations are identified in Table 1 or Table 2. Sequences complementary to the genomic locations identified in Table 1 or Table 2 are also example ranges of genomic locations. In various embodiments, the first nucleic acid includes two or more sequential CpG sites. In various embodiments, a range of genomic locations includes two, three, four, five, six, seven, eight, nine, ten, eleven, twelve, thirteen, fourteen, fifteen, sixteen, seventeen, eighteen, nineteen, or twenty sequential CpG sites. In particular embodiments, such as the embodiments shown in Table 1 or Table 2, a range of genomic locations include five sequential CpG sites (e.g., identified in Table 1 or Table 2 as CpG1, CpG2, CpG3, CpG4, and CpG5). In various embodiments, the first nucleic acid includes from about 2 CpG to about 500 CpG sites. In various embodiments, the first nucleic acid includes from about 3 CpG to about 400 CpG sites, from about 4 CpG to about 300 CpG sites, from about 5 CpG to about 200 CpG sites, from about 10 CpG sites to about 100 CpG sites, from about 20 CpG sites to about 80 CpG sites, from about 30 CpG sites to about 70 CpG sites, from about 40 CpG sites to about 60 CpG sites, or from about 45 CpG sites to about 55 CpG sites. In particular embodiments, the first nucleic acid includes from about 5 CpG to about 200 CpG sites.

The first nucleic acid may be a sequence that has previously undergone a bisulfite conversion, and therefore includes particular nucleotides at the one or more CpG sites that are dependent on the methylation status of the CpG site prior to bisulfite conversion. For example, assuming the nucleic acid is a bisulfite-converted DNA sequence, then if the cytosine of the CpG site was previously methylated, then after undergoing bisulfite conversion, the nucleotide includes a cytosine. If the cytosine of the CpG site was previously unmethylated, then after undergoing bisulfite conversion, the nucleotide includes a uracil (or in the case of a complement of the converted DNA 405 strand, the nucleotide includes a thymine). Altogether, the bisulfite-converted sequence may comprise a sequence in which unmethylated cytosines (e.g., methylated cytosines present prior to conversion) have been converted to uracil/thymine and methylated cytosines (e.g., unmethylated cytosines present prior to conversion) are not converted to uracil/thymine.

The second nucleic acid includes a sequence complementary to the binding site sequence of the first nucleic acid including the CpG site. In such embodiments, the composition including the bisulfite-converted DNA sequence and the second nucleic acid (e.g., a complementary oligonucleotide probe) can be useful for performing nucleic acid enrichment (e.g., via hybrid capture or PCR). Thus, the bisulfite-converted DNA sequence can be enriched using the second nucleic acid, relative to other nucleic acid sequences. In various embodiments, the composition further comprises a third nucleic acid with a sequence that is complementary to a second binding site of the first nucleic acid. Here, the third nucleic acid may be an additional oligonucleotide, such as an additional DNA oligonucleotide. As described above, in various embodiments, the composition including the bisulfite-converted DNA sequence and the second nucleic acid (e.g., a complementary oligonucleotide probe) can be useful for performing nucleic acid enrichment (e.g., via hybrid capture or PCR). Here, the third nucleic acid (e.g., an additional complementary oligonucleotide probe) can also be useful for performing nucleic acid enrichment. For example, the second nucleic acid and the third nucleic acid may be a primer pair, where each primer is complementary to a respective binding site of the first nucleic acid (e.g., bisulfite-converted DNA sequence or complement thereof) for performing nucleic acid enrichment (e.g., via hybrid capture or PCR).

In various embodiments, the second nucleic acid is a RNA oligonucleotide probe, an example of which is a RNA guide sequence. In such embodiments, the composition including the first nucleic acid (e.g., bisulfite-converted DNA sequence or complement thereof) and the second nucleic acid (e.g., a complementary RNA oligonucleotide probe) can be useful for performing nucleic acid enrichment (e.g., via CRISPR-based enrichment methods). For example, the RNA guide sequence is complementary or substantially complementary to a sequence of the binding site of the converted DNA and therefore, may guide a CRISPR-Cas protease (e.g., CRISPR-Cas9 or CRISPR-Cas12) for targeted cleaving of the converted DNA. Addition of adapters can be performed followed by enrichment of the target sequence (e.g., sequence including the CpG sites of the converted DNA). Further details of CRISPR-based enrichment is described in Schultzhaus, Z. et al., “CRISPR-based enrichment strategies for targeted sequencing.” Biotechnology Advances 46 (2021): 107672, which is hereby incorporated by reference in its entirety. Thus, the bisulfite-converted DNA sequence can be enriched using the RNA guide sequence, relative to other nucleic acid sequences.

In various embodiments, compositions disclosed herein comprise: a bisulfite-converted sequence from a range of genomic locations identified in Table 1 or Table 2 or the complement thereof; and an oligonucleotide complementary to a binding site within the range of genomic locations identified in Table 1 or Table 2 or the complement thereof. In various embodiments, the oligonucleotide is a DNA oligonucleotide probe. For example, the DNA oligonucleotide probe hybridizes with a bisulfite converted DNA sequence from the range of genomic locations identified in Table 1 or Table 2. In various embodiments, the oligonucleotide is an RNA oligonucleotide probe, an example of which is a guide RNA. In such embodiments, the RNA oligonucleotide probe is useful for performing nucleic acid enrichment (e.g., via CRISPR-based enrichment).

Reference is now made to FIG. 4A and FIG. 4B show example compositions including a converted nucleic acid sequence and an oligonucleotide complementary to a binding site of the converted nucleic acid sequence, in accordance with two embodiments. Specifically, FIG. 4A shows a composition including an example converted DNA 405 (e.g., bisulfite-converted DNA) and a DNA oligonucleotide 410 with a sequence that is complementary to a binding site 420 of the converted DNA 405. Here, within the binding site 420 of the converted DNA 405, FIG. 4A shows three CPG sites (e.g., CpG site 415A, CpG site 415B, and CpG site 415C). In other embodiments, the binding site 420 can include a different number of CpG sites 415 (e.g., can include five CpG sites). The three CpG sites (e.g., CpG site 415A, CpG site 415B, and CpG site 415C) may be sequential CpG sites, meaning that there are no other CpG sites located between CpG site 415A and CpG site 415B or between CpG site 415B and CpG site 415C.

As shown in FIG. 4A, each of the CpG sites (e.g., CpG site 415A, CpG site 415B, and CpG site 415C) of the converted DNA 405 may include a cytosine. This indicates that prior to conversion, each of the cytosines corresponding the CpG sites were methylated cytosines. In other embodiments, not every single one of the CpG sites (e.g., CpG site 415A, CpG site 415B, and CpG site 415C) were methylated prior to conversion and therefore, such CpG sites would include a uracil or thymine after conversion.

The DNA oligonucleotide 410 may include a sequence that is fully complementary or substantially complementary to the sequence of the binding site 420 of the converted DNA 405. As used herein, fully complementary refers to 100% complementarity. As used herein, substantially complementary refers to at least 90% complementarity. In various embodiments, substantially complementary refers to 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% complementarity.

Although not explicitly shown in FIG. 4A, the composition may include additional oligonucleotides and/or additional elements that may be useful for performing nucleic acid enrichment. For example, the composition may include an additional oligonucleotide that is complementary to a sequence of a different binding site of the converted DNA 405. For example, the DNA oligonucleotide 410 and the additional oligonucleotide may be a primer pair for performing nucleic acid amplification, an example of which includes polymerase chain reaction. Thus, by performing nucleic acid amplification, a sequence of the converted DNA 405 including at least the CpG sites 415 of the binding site 420 can be enriched. As another example, the composition may include additional elements for performing hybrid capture. For example, the DNA oligonucleotide 410 may be biotinylated. Thus, the converted DNA 405—DNA oligonucleotide construct can be pulled down by streptavidin beads, thereby capturing at least the sequence of the binding site 420 of the converted DNA 405. Further examples of hybrid capture probe sets include the KAPA HyperPrep Kit and SeqCAP Epi Enrichment System from Roche Diagnostics (Pleasanton, CA). Hybrid capture probe sets are designed to hybridize with particular sequences of the converted DNA 405, thereby capturing and enriching the particular sequences (e.g., sequences including CpG sites 415) of the converted DNA 405.

FIG. 4B shows a composition including an example converted DNA 455 and a RNA oligonucleotide 460 (such as a RNA guide sequence) with a sequence that is complementary to a binding site 470 of the converted RNA 450. Although not explicitly shown in FIG. 4B, the composition may further include a protein, such as a CRISPR-Cas protein (e.g., CRISPR-Cas9 or CRISPR-Cas12) that is guided to the converted DNA 455 via the RNA oligonucleotide 460. Thus, the composition shown in FIG. 4B is useful for performing enrichment of a sequence (e.g., a sequence including CpG sites 465) of the converted DNA 455 through CRISPR-based enrichment methods.

### Longitudinal Threshold Value

Disclosed herein are methods for predicting tumor content, e.g., tumor content in a sample. In various embodiments, such methods can predict a cancer score relative to a threshold value, such as a longitudinal threshold value. In various embodiments, such methods involve predicting tumor content relative to a designated limit of detection (LOD) value. In various embodiments, the methods disclosed herein predict tumor content at a particular LOD without needing a matched biopsy (e.g., a cancer biopsy matched with a non-cancer biopsy).

In various embodiments, the methods disclosed herein predict a cancer score relative to a longitudinal threshold value of less than 0.10%. In various embodiments, the methods disclosed herein predict a cancer score relative to a longitudinal threshold value of less than 0.05%. In various embodiments, the methods disclosed herein predict a cancer score relative to a longitudinal threshold value of less than 0.04%. In various embodiments, the methods disclosed herein predict a cancer score relative to a longitudinal threshold value of less than 0.03%. In various embodiments, the methods disclosed herein predict a cancer score relative to a longitudinal threshold value of less than 0.02%. In various embodiments, the methods disclosed herein predict a cancer score relative to a longitudinal threshold value of less than 0.01%. In various embodiments, the methods disclosed herein predict a cancer score relative to a longitudinal threshold value of less than 0.005%. In various embodiments, the methods disclosed herein predict a cancer score relative to a longitudinal threshold value of less than 0.001%. In various embodiments, the methods disclosed herein predict a cancer score relative to a longitudinal threshold value of less than 0.1%, 0.09%, 0.08%, 0.07%, 0.06%, 0.05%, 0.04%, 0.03%, 0.02%, or 0.01%. In various embodiments, the methods disclosed herein predict a cancer score relative to a longitudinal threshold value of less than 0.04%, 0.039%, 0.038%, 0.037%, 0.036%, 0.035%, 0.034%, 0.033%, 0.032%, 0.31%, 0.030%, 0.029%, 0.028%, 0.027%, 0.026%, 0.025%, 0.024%, 0.023%, 0.022%, 0.021%, 0.020%, 0.019%, 0.018%, 0.017%, 0.016%, 0.015%, 0.014%, 0.013%, 0.012%, 0.011%, or 0.010%. In particular embodiments, the methods disclosed herein predict a cancer score relative to a longitudinal threshold value of less than 0.037%.

In various embodiments, the longitudinal threshold value is determined according to an accuracy value. For example, the longitudinal threshold value can be a limit of detection (LOD) value in which at least a target percentage of samples are successfully identified as having cancer or not having cancer. In various embodiments, the longitudinal can be a limit of detection (LOD) value in which at least a target percentage of cancer samples are successfully identified as having cancer. In various embodiments, the longitudinal can be a limit of detection (LOD) value in which at least a target percentage of non-cancer samples are successfully identified as not having cancer.

As a specific example, the longitudinal threshold value can be a limit of detection (LOD) value at an accuracy of at least 95% (e.g., at least 95% of samples are successfully identified as having cancer or not having cancer). In various embodiments, the longitudinal threshold value is determined at an accuracy of at least 80% (e.g., at least 80% of samples are successfully identified as having cancer or not having cancer). In various embodiments, the longitudinal threshold value is determined at an accuracy of at least 85% (e.g., at least 85% of samples are successfully identified as having cancer or not having cancer). In various embodiments, the longitudinal threshold value is determined at an accuracy of at least 90% (e.g., at least 90% of samples are successfully identified as having cancer or not having cancer). In various embodiments, the longitudinal threshold value is determined at an accuracy of at least 95% (e.g., at least 95% of samples are successfully identified as having cancer or not having cancer). In various embodiments, the longitudinal threshold value is determined at an accuracy of at least 99% (e.g., at least 99% of samples are successfully identified as having cancer or not having cancer). In various embodiments, the longitudinal threshold value is determined at an accuracy of 100% (e.g., 100% of samples are successfully identified as having cancer or not having cancer). In various embodiments, the longitudinal threshold value is determined at an accuracy of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9%.

In various embodiments, the longitudinal threshold value is determined according to a sensitivity value. Sensitivity can be defined as the number of true positives divided by the sum of true positives and false negatives. For example, the longitudinal threshold value can be a limit of detection (LOD) value at a target sensitivity value. In various embodiments, the longitudinal threshold value is determined at a sensitivity of at least 80%. In various embodiments, the longitudinal threshold value is determined at a sensitivity of at least 85%. In various embodiments, the longitudinal threshold value is determined at a sensitivity of at least 90%. In various embodiments, the longitudinal threshold value is determined at a sensitivity of at least 95%. In various embodiments, the longitudinal threshold value is determined at a sensitivity of at least 99%. In various embodiments, the longitudinal threshold value is determined at a sensitivity of 100%. In various embodiments, the longitudinal threshold value is determined at a sensitivity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9%.

In various embodiments, the longitudinal threshold value is determined according to a specificity value. Specificity can be defined as the number of true negatives divided by the sum of true negatives and false positives. For example, the longitudinal threshold value can be a limit of detection (LOD) value at a target specificity value. In various embodiments, the longitudinal threshold value is determined at a specificity of at least 80%. In various embodiments, the longitudinal threshold value is determined at a specificity of at least 85%. In various embodiments, the longitudinal threshold value is determined at a specificity of at least 90%. In various embodiments, the longitudinal threshold value is determined at a specificity of at least 95%. In various embodiments, the longitudinal threshold value is determined at a specificity of at least 99%. In various embodiments, the longitudinal threshold value is determined at a specificity of 100%. In various embodiments, the longitudinal threshold value is determined at a specificity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9%.

### Estimating Tumor Burden

In some embodiments, a method of estimating tumor burden is presented. In various embodiments, tumor burden may be estimated based on one or more methylation markers, such as one or more universal cancer signatures as described above. In some embodiments, methods involve predicting tumor content in a biological sample obtained from a subject using one or more universal cancer signatures. The predicted tumor content can then be used to predict the tumor burden (e.g., tumor size) that is present in the subject. In some embodiments, as disclosed herein, methods may involve performing longitudinal tracking of tumor content using the one or more universal cancer signatures across two or more samples obtained from a subject at two or more timepoints. In various embodiments, the longitudinal tracking may involve tracking the tumor burden over the two or more timepoints in the subject based on the changing tumor content, otherwise referred to as the changing percent tumor in cfDNA, from the subject. For example, rapidly increasing tumor content across two timepoints can be indicative of a particularly aggressive cancer and therefore, the tumor burden (e.g., tumor size) can be significantly larger at a second (e.g., later) timepoint in comparison to the tumor burden at the first (e.g., earlier) timepoint. As another example, tumor content that is relatively changed or slowly increasing across two timepoints can be indicative of an indolent cancer. Therefore, the tumor burden (e.g., tumor size) can be similar or slightly increasing at a second (e.g., later) timepoint in comparison to the tumor burden at the first (e.g., earlier) timepoint.

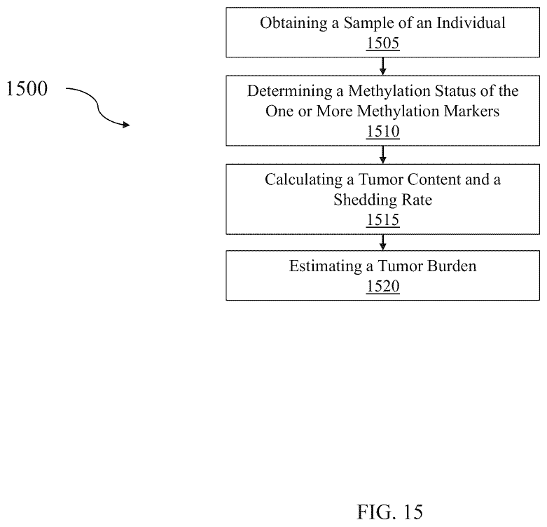

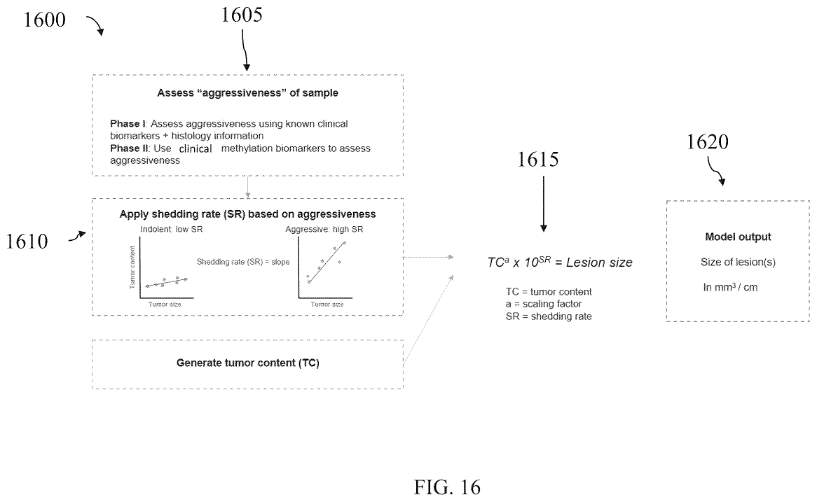

Reference is now made to FIG. 15, which illustrates a flowchart of a method of estimating tumor burden. At Step 1505, method 1500 includes obtaining a sample of an individual. A sample may include any biological sample or tissue described throughout this disclosure, without limitation. A sample may be any of a blood sample, a plasma sample, a stool sample, a urine sample, a semen sample, an intraocular fluid sample, a peritoneal sample, a pleural fluid sample, an amniotic fluid sample, a cerebrospinal fluid sample, a mucous sample, or a saliva sample. In some embodiments, the biological sample may include a liquified biopsy obtained from a solid tissue and processed (e.g., through tissue trituration and/or enzymatic treatment) to produce a liquid sample. In particular embodiments, a sample obtained from may be a blood sample, such as a liquid biopsy. The sample can be obtained by the individual or by a third party, e.g., a medical professional. Examples of medical professionals include physicians, emergency medical technicians, nurses, first responders, psychologists, phlebotomists, medical physics personnel, nurse practitioners, surgeons, dentists, and any other medical professional as would be known to one skilled in the art. In various embodiments, the sample can be obtained from the subject by a reference lab.

In some embodiments, the sample may include nucleic acids that are informative for predicting tumor content in the sample. In various embodiments, the nucleic acids include cell-free DNA (cfDNA). In various embodiments, the nucleic acids include cell-free DNA fragments. In various embodiments, the cfDNA can be derived from tumor cells and is referred to herein as circulating tumor DNA (ctDNA). In particular embodiments, the nucleic acids include cfDNA fragments across a plurality of genomic locations. Genomic locations can include one or more CpG sites whose methylation statuses may be informative for predicting tumor content.

At step 1515, method 1500 includes determining a methylation status of one or more methylation markers. Methylation markers may, in various embodiments, be universal cancer signatures, also referred to as fully methylated variants. In various embodiments, methylation markers can be identified by identifying a first set of universal genomic locations each comprising two or more sequential CpG sites that are methylated from cell-free DNA fragments from the first plurality of samples. In particular embodiments, identifying one or more methylation markers may involve identifying a first set of universal genomic locations, each comprising five sequential CpG sites that are fully methylated from cell-free DNA fragments from the first plurality of samples. In various embodiments, one or more methylation markers may be identified by identifying a second set of universal genomic locations each comprising two or more sequential CpG sites that are methylated from cell-free DNA fragments from the second plurality of samples. Identifying a second set of universal genomic locations may include identifying a second set of universal genomic locations that each comprise five sequential CpG sites that are fully methylated from cell-free DNA fragments from the second plurality of samples. In various embodiments, the one or more methylation markers may include a universal cancer signature that represents an overlap between the first set of universal genomic locations and the second set of universal genomic locations.

A methylation status may include any one of unmethylated, partially methylated, or fully methylated. In some embodiments, determining a methylation status may include determining a methylation status of at least 2 methylation markers identified in a sample, wherein each of the at least 2 methylation markers each comprise a methylation locus comprising at least a portion of a range of genomic locations selected from the group consisting of the range of genomic locations in Table 1 or Table 2.

In various embodiments, methods involve detecting methylation statuses of methylation markers of a subset of the 1043 genomic locations shown in Table 1. In various embodiments, methods involve detecting methylation statuses of methylation markers of each of the 1043 genomic locations shown in Table 1. In various embodiments, methods involve detecting methylation statuses of methylation markers of a subset of the 561 genomic locations shown in Table 2. In various embodiments, methods involve detecting methylation statuses of methylation markers of each of the 561 genomic locations shown in Table 2. In various embodiments, methods involve detecting methylation statuses of methylation markers of at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 of the 1043 genomic locations shown in Table 1. In various embodiments, methods involve detecting methylation statuses of methylation markers of at least 10, at least 20, at least 30, at least 40, at least 50, at least 60, at least 70, at least 80, at least 90, at least 100, at least 200, at least 300, at least 400, or at least 500 of the 561 genomic locations shown in Table 1.

In various embodiments, determining the methylation status of each of at least 2 methylation markers comprises determining methylation status of each of at least 10, at least 20, at least 30, at least 40, at least 50, at least 100, at least 100, at least 500, at least 1000, at least 2000, at least 3000, at least 4000, at least 5000, at least 10,000, at least 20,000, at least 30,000, at least 40,000, or at least 50,000 methylation markers. In various embodiments, each methylation locus comprising at least a portion of a range of genomic locations selected from Table 1 or Table 2 comprises two or more sequential CpG sites. In various embodiments, the two or more sequential CpG sites comprise three, four, or five sequential CpG sites.

In some embodiments, step 1515 may include performing an assay on the sample. Example assays are described in further detail herein. One or more assays may be performed to generate a tumor content prediction. Generally, performing one or more assays involves converting nucleic acids in the sample obtained from the subject. In various embodiments, converting the nucleic acid involves converting unmethylated nucleotides (e.g., cytosines) to another nucleotide (a “converted nucleotide”, as used herein). In various embodiments, methylated cytosines are protected from conversion (e.g., deamination) during the conversion step. This enables subsequent downstream differentiation of methylated cytosines and unmethylated cytosines. Thus, methylation statuses of sequential CpG sites can be determined and used to predict tumor content in a sample. The tumor content prediction refers to a detection of tumor derived nucleic acids in a sample. In various embodiments, the tumor content prediction represents a proportion of tumor derived nucleic acids relative to the total nucleic acids in the sample. In various embodiments, tumor derived nucleic acids are identified as cell-free DNA fragments in which two or more sequential CpG sites of genomic locations are fully methylated. In particular embodiments, tumor derived nucleic acids are identified as cell-free DNA fragments in which five sequential CpG sites of genomic locations (e.g., five sequential CpG sites in ranges of genomic locations shown above in Table 1 or Table 2) are fully methylated. In various embodiments, non-tumor derived nucleic acids are identified as cell-free DNA fragments in which two or more sequential CpG sites of genomic locations are not fully methylated. For example, for a DNA fragment, if a first CpG site is determined to be methylated and a second CpG is determined to be nonmethylated, the DNA fragment can be deemed a non-tumor derived nucleic acid.

At step 1520, method 1500 includes calculating a tumor content and a shedding rate based on the methylation statuses of the one or more methylation markers. Tumor content may be a prediction of an overall amount of detected tumor derived nucleic acids in a sample in relation to non-tumor derived nucleic acids in the sample. Tumor content may be a proportion of tumor derived nucleic acids in a sample to non-tumor derived nucleic acids in the sample. Proportions may include, but are not limited to, ratios, percentages, fractions, or other numerical representations.

A shedding rate refers to an amount of tumor derived nucleic acids released from the tumor over a period of time. For instance and without limitation, a shedding rate may be about 10⁻⁴ tumor fragments released into circulation per tumor volume in cubic millimeters to about 10⁻¹⁵ tumor fragments released into circulation per tumor volume in cubic millimeters. In various embodiments, a shedding rate may be calculated in part based on an amount of tumor-derived DNA in circulation of a subject, which may be estimated as tumor content as described above. In various embodiments, a shedding rate may be calculated in part based on a change in the amount of tumor-derived DNA in circulation of a subject, e.g., a change in the predicted tumor content. A shedding rate may correlate with an aggressiveness or indolence of a tumor. For instance, a more aggressive tumor may have a higher shedding rate which may be a result of a more rapid growing rate of the tumor compared to a less aggressive tumor. An indolent tumor may have a more mild shedding rate, which may be a result of a slower growing rate of a tumor as compared to a more aggressive tumor. A shedding rate may not necessarily correlate with a tumor burden. A shedding rate may be non-linear with a tumor size. For instance, a smaller tumor may have a higher shedding rate than a larger tumor. In some embodiments, a larger tumor may have a higher shedding rate than a smaller tumor. In other embodiments, a smaller tumor may have a higher shedding rate than a larger tumor.

In some embodiments, calculating a shedding rate may involve utilizing training data or “truth” data. Training data may be generated by comparing an estimated tumor content with a calculated tumor size of one or more prior samples. For instance, an estimated tumor content may be calculated as described above and a calculated tumor size may be obtained from one or more of radiology, imaging, pathology reports, or other measurements. Values of an estimated tumor content and a calculated tumor size may be compared to determine a shedding rate, which may map a volume or size of a tumor to the tumor content. A shedding rate may be calculated for separate samples, of which each sample may be marked as aggressive or indolent, in some embodiments. A mapping of a volume or size of a tumor to tumor content from previous samples may be used for a future unknown sample to determine a tumor burden based on tumor content and shedding rate calculated for the future unknown sample.

At step 1525, method 1500 includes estimating a tumor burden. A tumor burden may be estimated based on one or more of a tumor content prediction and a shedding rate. A tumor burden may be calculated as a volumetric measurement. For example, the volumetric measurement can be quantified in cubic millimeters (mm³). In some embodiments, a tumor burden may be calculated as an area or volume. A tumor burden may be calculated based on a linear coefficient which may be derived from a shedding rate. For instance, a linear coefficient may be a numerical value representing a shedding rate of a tumor. A tumor burden may be calculated based in part on a scaling factor. A scaling factor may be representative of a dimension of a tumor. For instance and without limitation, a scaling factor of “2” may be used to estimate a surface area of a tumor and a scaling factor of “3” may be used to estimate a volume of a tumor. A scaling factor may be proportional to an area or volume of a calculated tumor burden.

Referring now to FIG. 16, a flowchart 1600 for calculating a tumor burden is presented. At step 1605, an aggressiveness of a tumor in a sample is assessed. An aggressiveness may be assessed using one or more clinical biomarkers and/or historical information. Biomarkers may include, but are not limited to, triple negative status for breast cancer, Gleason scores for prostate, driver mutation status, histology, and/or other clinical biomarkers/historical information. A triple negative status for breast cancer (TNBC) may be any breast cancer that either lacks or shows low levels of estrogen receptor (ER), progesterone receptor (PR) and human epidermal growth factor receptor 2 (HER2). Gleason scores may be representative of a risk group of individuals with cancer, such as a low/very low risk group, intermediate risk group, and/or high/very high risk group. Gleason scores may be a numerical value out of 10, with scores at or below 6 categorized as low risk, scores at about 7 categorized as intermediate risk, and scores at about 8-10 categorized as high/very high risk. Driver mutation status may include one or more signals relating to a driver mutation, which may be a type of alteration that gives a cancer cell a growth advantage and promotes tumor formation, unlike passenger mutations which do not provide a growth advantage in a cancer cell. In some embodiments, methylation biomarkers may be used to assess an aggressiveness of a tumor in a sample.

At step 1610, a shedding rate is calculated based on the aggressiveness. A shedding rate may be calculated based on training data which may be generated from calculations of one or more prior samples, such as described above with reference to FIG. 15. In some embodiments, an aggressiveness may correlate to a shedding rate and/or size of a tumor. Based on an aggressiveness a shedding rate may be calculated from prior correlations of tumor content, shedding rates, and/or tumor sizes of one or more previous samples. In some embodiments, a shedding rate may be calculated based on a relationship determined between an aggressiveness value and a shedding rate, such as a slope function correlating aggressiveness to shedding rate.

At step 1615, tumor burden is calculated based on tumor content and a shedding rate, using the following Equation 1):

$$Tumor Burden = {TC}^{a} \times {10}^{SR}$$

where “TC” represents tumor content, “a” represents a scaling factor, and “SR” represents a shedding rate. As shown in Equation (1), TC may be raised to a power of “a”, which may account for area or volume of a calculated tumor burden. For instance and without limitation, if “a” is equal to “2”, tumor burden may be calculated in terms of area. Likewise if “a” is equal to “3”, tumor burden may be calculated in terms of volume. “a” may be proportional to an area or volume of a tumor burden. “a” may be used to transform linear measurements of tumor length into volume, in some embodiments. to a shedding rate. Tumor burden may be calculated as a product of “TC” raised to the power of “a” and 10 raised to the power of “SR”. In some embodiments, performing a log-transformation of both sides of Equation 1 may result in Equation 2):

$$a \times log ( TC ) + SR = log ( size )$$

which may also be equivalent to Equation 3):

$$Tumor Burden = {SR}^{′} \times {TC}^{a}$$

where SR′=10^{SR}. Equations 2 and 3 may be straight line approximations of tumor burden. In some embodiments, training data or “truth data” may be used to link tumor content and size (each of which may be known) to shedding rate and a scaling factor (each of which may be unknown).

At step 1620, an output of a calculated and/or estimated tumor burden is made. A tumor burden estimation and/or calculation may be made in cubic millimeters or other volumetric measurements. In some embodiments, a tumor burden calculation and/or estimation may be made in terms of area and/or length.

### Example Cancers

In some embodiments, the cancer is an early stage cancer. In various embodiments, the cancer is a preclinical phase cancer. In some embodiments, the cancer is a stage 0 cancer. In various embodiments, the cancer is a stage 1 cancer. In various embodiments, the cancer is a stage 2 cancer. Thus, the methods disclosed herein enable the screening and diagnosis of an individual for an early stage or preclinical stage cancer. In some embodiments, the cancer is a stage 3 cancer. In some embodiments, the cancer is a stage 4 cancer.

In some embodiment, the cancer is a carcinoma, adenocarcinoma, blastoma, leukemia, seminoma, melanoma, teratoma, lymphoma, neuroblastoma, glioma, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer, lung cancer, and the metastases thereof.

In some embodiments, the cancer is any of an acute lymphoblastic leukemia, acute myeloid leukemia, adrenocortical carcinoma, soft tissue sarcoma, anal cancer, bile duct cancer, bladder cancer, bone cancer, cardiac cancer, central nervous system cancer, chronic lymphocytic leukemia, chronic myelogenous leukemia, chronic myeloproliferative neoplasms, esophageal cancer, head and neck cancer, eye cancer, fallopian tube cancer, gallbladder cancer, gastric cancer, germ cell tumor, gestational trophoblastic cancer, hairy cell leukemia, Hodgkin lymphoma, intraocular melanoma, pancreatic cancer, mesothelioma, metastatic cancer, mouth cancer, multiple endocrine neoplasia syndromes, multiple myeloma neoplasms, myelodysplastic neoplasms, parathyroid cancer, penile cancer, pheochromocytoma, pituitary cancer, plasma cell neoplasm, primary peritoneal cancer, prostate cancer, retinoblastoma, sarcoma, small intestine cancer, testicular cancer, thymoma and thymic carcinoma, urethral cancer, vaginal cancer, and vulvar cancer.

All publications, patents, patent applications and other documents cited in this application are hereby incorporated by reference herein in their entireties for all purposes to the same extent as if each individual publication, patent, patent application or other document were individually indicated to be incorporated by reference for all purposes.

While various specific embodiments have been illustrated and described, the above specification is not restrictive. It will be appreciated that various changes can be made without departing from the spirit and scope of the present disclosure(s). Many variations will become apparent to those skilled in the art upon review of this specification.

### Computer Implementation

The methods of the invention, including the methods of identifying universal cancer signatures and methods of determining tumor content, are, in some embodiments, performed on one or more computers. In various embodiments, the methods of identifying universal cancer signatures and methods of determining tumor content can be implemented in hardware or software, or a combination of both. In one embodiment, a machine-readable storage medium is provided, the medium comprising a data storage material encoded with machine readable data which, when using a machine programmed with instructions for using said data, is capable of displaying data and results. Such data can be used for a variety of purposes, such as for determining whether a sample is positive for cancer. The invention can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the methods of the invention, including the methods of identifying universal cancer signatures and methods of determining tumor content, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### Example Computer

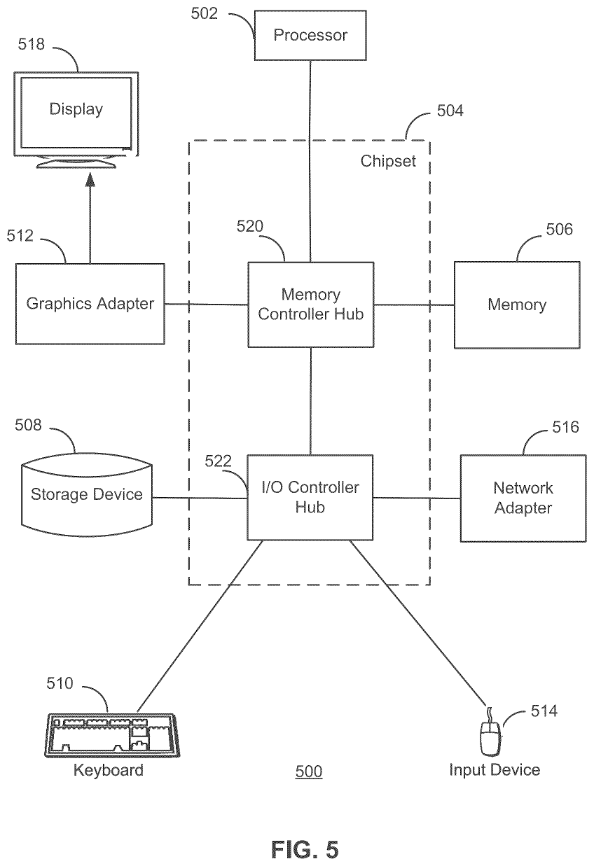

FIG. 5 illustrates an example computer for implementing the entities shown in FIGS. 1A-1B, 2, 3A-3B, and 4A-4B. The computer 500 includes at least one processor 502 coupled to a chipset 504. The chipset 504 includes a memory controller hub 520 and an input/output (I/O) controller hub 522. A memory 506 and a graphics adapter 512 are coupled to the memory controller hub 520, and a display 518 is coupled to the graphics adapter 512. A storage device 508, an input device 514, and network adapter 516 are coupled to the I/O controller hub 522. Other embodiments of the computer 500 have different architectures.

The storage device 508 is a non-transitory computer-readable storage medium such as a hard drive, compact disk read-only memory (CD-ROM), DVD, or a solid-state memory device. The memory 506 holds instructions and data used by the processor 502. The input interface 514 is a touch-screen interface, a mouse, track ball, or other type of pointing device, a keyboard, or some combination thereof, and is used to input data into the computer 500. In some embodiments, the computer 500 may be configured to receive input (e.g., commands) from the input interface 514 via gestures from the user. The graphics adapter 512 displays images and other information on the display 518. The network adapter 516 couples the computer 500 to one or more computer networks.

The computer 500 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 508, loaded into the memory 506, and executed by the processor 502. A module can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

The types of computers 500 can vary depending upon the embodiment and the processing power required by the entity. For example, the methods disclosed herein can run in a single computer 500 or multiple computers 500 communicating with each other through a network such as in a server farm. The computers 500 can lack some of the components described above, such as graphics adapters 512, and displays 518.

### EXAMPLES

### Example 1: Methods for Discovering and Using Methylation Variants

Generally, there are 2 modes for tumor content determination: (1) R&D mode, for discovering CpG sites later used for tumor content estimation; (2) production mode, for computing tumor content for each sample at the discovered CpG sites from R&D mode.

Generally, R&D mode involves identifying informative genomic sites (e.g., CpG sites). The procedure includes the following:
Assemble 2 cohorts: a) a set of cancer tissue comprised of diverse cancers, and b) a set of normal plasma.
Sequence all samples in the 2 cohorts, and align reads to genome, producing a bam file for each.
For each sample's bam file, compute, per-CpG metrics at each computable genomic site.
   CpG sites within the panel were analyzed, which equates to a total of ˜400K sites. The number of 5 sequential CpGs on a sequencing read that are all completely methylated were quantified
   The number of fully methylated sequential CpGs on a read (K5n5), the total number of reads where 5 sequential CpGs can be counted (total K5) and the computed fraction (K5n5/total K5) were calculated for every CpG and every sample. As referred to herein, the term “K5n5” refers to the number of CpG sites in a sequence (e.g., K=5) and the number of CpG sites that are methylated (e.g., n=5); hence, K5n5 refers to the 5 sequential CpG sites that are each methylated.
   These metrics are computed using custom software written in python that takes in a BAM file, and outputs the per-CpG metrics file.
Identify genomic sites that have K5N5>0.1 across cancer samples, while K5N5<0.0001 in normal plasma samples. These genomic sites are saved for use in “production mode”

Generally, production mode involves estimating tumor content for any given sample. The procedure includes the following:
For a given sample, sequence and align to genome, producing a bam file.
Compute K5N5 metrics (as conducted above during R&D mode) at the discovered genomics sites
Compute tumor content as the total observed K5n5 counts divided by the total K5 count

### Example 2: Validation of Methylation Variants

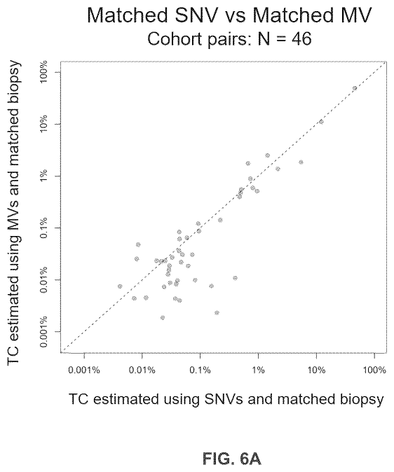

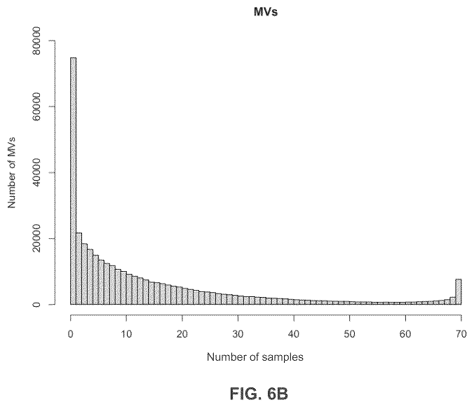

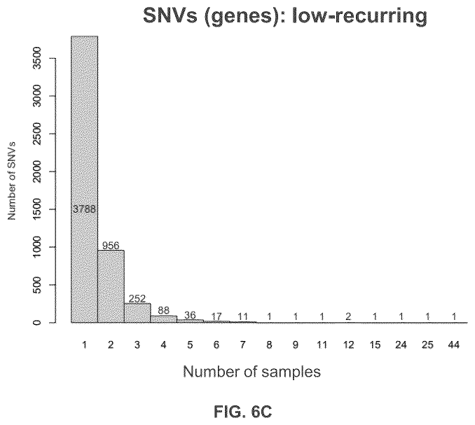

46 cohort pairs were analyzed to evaluate and validate the methylation variants for their ability to predict tumor content. Specifically, methylation variants (MVs) were validated using matched biopsies as reference samples. Reference is made to FIG. 6A, which is a graph showing the correlation between 1) tumor content (TC) estimation using single nucleotide variants (SNVs) and matched biopsy and 2) TC estimation using methylation variants (MVs) (e.g., MVs identified in Table 1). In general, using MVs to estimate TC correlated closely with using SNVs for TC estimation. MVs and SNVs were each compared to reference positive control cancer samples (e.g., matched biopsies). Generally, this experiment revealed that despite the existence of noise at lower estimated TC, MVs can be used to adequately estimate TC.

Methylation variants were further validated for their presence across multiple cancer biopsy samples. Specifically, N=70 cancer biopsy samples were analyzed using whole exome sequencing to identify recurring methylation variants and recurring single nucleotide variants. Reference is made to FIG. 6B, which is a graph showing the number of methylation variants across 70 biopsy samples. Further reference is made to FIG. 6C, which is a graph showing the lack of recurring SNVs across large numbers of samples in contrast to methylation variants.

As can be observed in FIG. 6B, a set of methylation variants were observed across all 70 cancer biopsies, with some number of methylation variants also observed across at least 10, at least 20, at least 30, at least 40, at least 50, and at least 60 of the cancer biopsies. In contrast, low-recurring single nucleotide variants were present across fewer cancer biopsy samples. In particular only 1 SNV was present in 44 cancer biopsies whereas the majority of SNVs were only present in between 1 to 5 cancer biopsies. This indicates that SNVs cannot be used as universal cancer signatures across various cancer biopsies, whereas methylation variants represent more promise as universal cancer signatures.

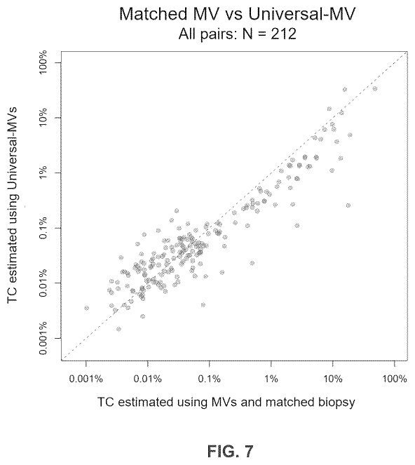

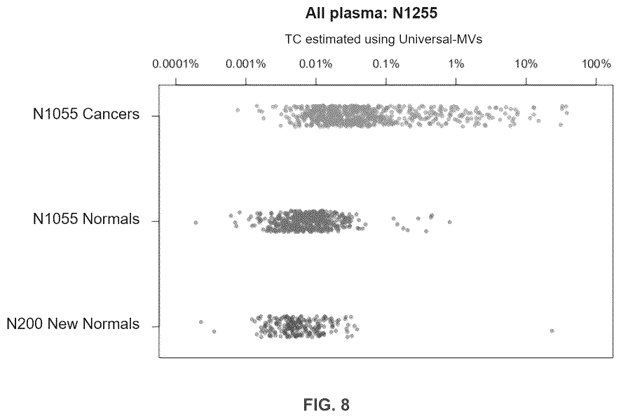

The methylation variants, identified as present across various cancer biopsies, were further analyzed to ensure that they remained predictive when estimating tumor content without using a matched tissue biopsy sample. Reference is made to FIG. 7, which shows a graph depicting the correlation between a TC estimation using MVs versus estimating TC using MVs that use a reference positive control cancer sample (e.g., matched biopsy). Here, the high correlation indicates that the methylation variants, even when used without a matched biopsy, remained predictive of tumor content.

### Example 3: Methylation Variants Predict Tumor Content in Various Cancers and Cancer Stages

The methylation variants identified in Table 1 were used to predict tumor content across various cancers and cancer stages. Here, methylation variants are present in very low quantities (e.g., less than 1:10,000) in normal plasma and are present in higher quantities (e.g., greater than 0.1) across cancer biopsy samples. Using the MVs, tumor content was estimated for plasma samples without matched biopsy. Reference is made to FIG. 8, which is a graph showing the estimated TC using MVs (e.g., without the use of a matched biopsy) in cancer samples and in normal samples. In general, using the MVs, non-cancers (e.g., Normals and New Normals) have lower TC estimation than cancers.

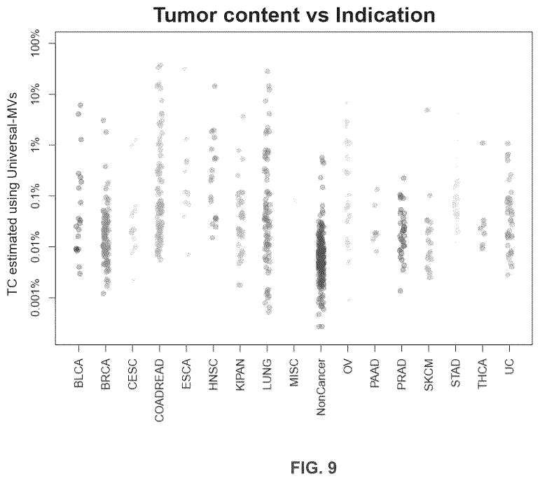

MVs were further used to predict tumor content across a variety of different cancers. For example, FIG. 9 is a graph showing differences in the range of estimated TC across different cancer indications. BLCA refers to bladder cancer. BRCA refers to breast cancer. CESC refers to cervical squamous cancer. COADREAD refers to colorectal cancer. ESCA refers to esophageal carcinoma. HNSC refers to head and neck squamous cell carcinoma. KIPAN refers to kidney cancer. LUNG refers to lung cancer. MISC refers to other cancers. NonCancer refers to a normal sample. OV refers to ovarian cancer. PAAD refers to pancreatic adenocarcinoma. PRAD refers to prostate adenocarcinoma. SKCM refers to skin cutaneous melanoma. STAD refers to stomach adenocarcinoma. THCA refers to thyroid cancer. UC refers to urothelial cancer. Generally, different cancer indications show different ranges of estimated tumor content. However, the estimated tumor content of the various cancer types were higher than the estimated tumor content for non-cancers (labeled as NonCancer in FIG. 9).

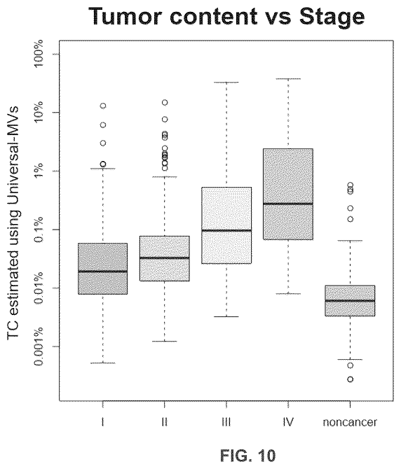

MVs were further used to predict tumor content across cancers at various stages (e.g., Stage 1, Stage 2, Stage 3, and Stage 4). FIG. 10 is a graph showing the relationship between TC determined using MVs and the stage of cancer. In particular, the MVs were able to successfully predict increasing tumor content with corresponding increases in cancer stage. Notably, methylation variants were successful in predicting higher tumor content for early stages of cancer (e.g., Stage 1 and Stage 2) in comparison to non-cancer.

### Example 4: Longitudinal Analysis of Tumor Content

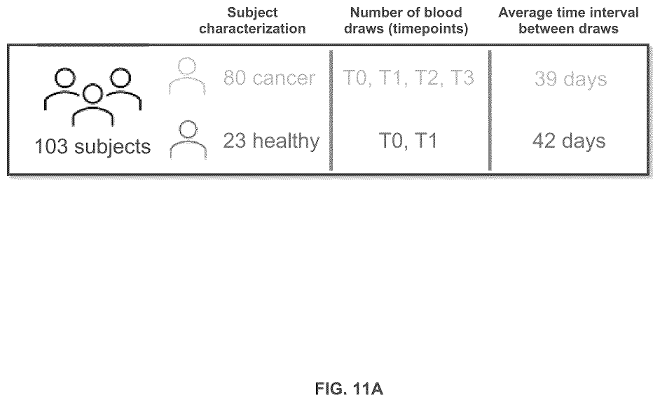

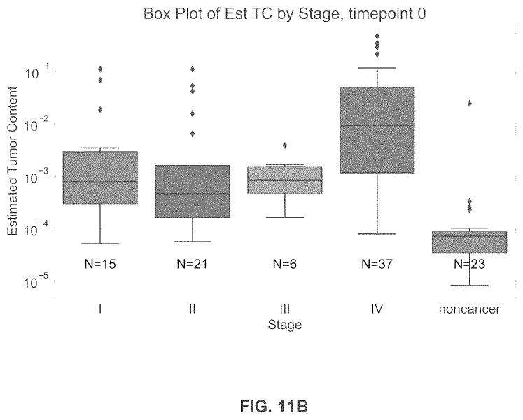

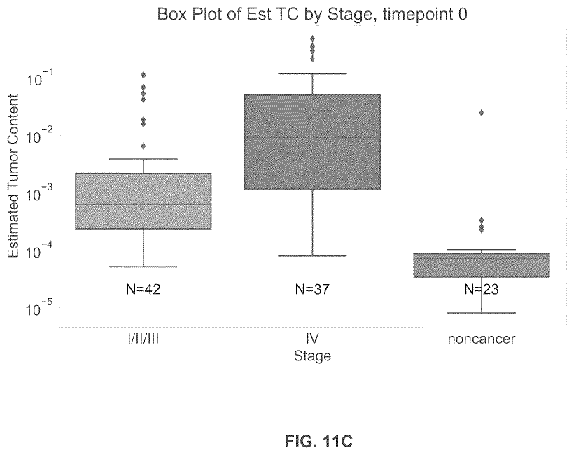

Plasma was sourced from 80 cancer patients across 16 cancer indications over the course of treatment with four timepoints, averaging 39 days apart. Timepoint 0 (TO) was pre-treatment, while T1, T2 and T3 were taken before during the course of treatment. Endpoint RECIST data were available for 31 out of the 80 cancer patients, which categorizes disease progression and response by evaluating the solid tumor lesion. Additionally, 23 healthy, non-cancer subjects were sourced with two timepoints, averaging 42 days apart. FIG. 11A shows the characteristics of the cancer and healthy subjects. All samples across these subject cohorts and timepoints were assessed using methylation variants identified in Table 2.

The longitudinal analysis generally included the following steps:
1) Generate tumor content estimates from methylation variants identified in Table 2.
2) If the tumor content at timepoint TO was above a longitudinal threshold value (e.g., 0.037%) and all 4 timepoints have data, then the longitudinal analysis was further performed for a subject
3) For timepoints after TO, determine the number of samples at subsequent timepoints with a tumor content estimation greater than the longitudinal threshold value (e.g., 0.037%)
4) The number of samples at subsequent timepoints that had a tumor content estimation greater than the longitudinal threshold value was associated with greater likelihood of death

To estimate tumor content, for a cfDNA sample, the fraction of fragments originating from cancer versus healthy cells was estimated, thereby deriving an estimate for cfDNA tumor content. Estimating tumor content involves analyzing methylation variants identified in Table 2, which represent a set of regions that have low methylation within healthy plasma, and high methylation within cancer tissue. This translates to: fully methylated fragments observed within methylation variant sites are most likely derived from cancer, while unmethylated or low methylated fragments are more likely expected to represent the normal/healthy state. The greater the observed fraction of fully methylated fragments, the higher the fraction of reads likely coming from cancer.

To quantify fragments likely deriving from cancer (methylated) vs healthy tissue (unmethylated or not fully methylated), methylation levels for “K5 features” were extracted, where each K5 feature consisted of 5 consecutive CpGs located within the enriched panel. The presence of 5 consecutive CpGs all being fully methylated was assumed to be most likely cancer derived, while all other methylation states are considered “normal”.

Tumor content was estimated for every sample within an independent dataset of cfDNA samples by counting the total fully methylated K5 features observed, divided by the sum of all K5 reads within the 1043 K5 features shown in Table 1. This fraction, e.g. 0.001, corresponds to % tumor content, i.e. 0.10%.

Samples at timepoint T0 shown FIG. 11A were analyzed to determine estimated tumor content. The estimated tumor content was further correlated to cancer stage. FIGS. 11B and 11C show box plots of estimated tumor content by cancer stage at timepoint 0. Tumor content estimated by methylation variants identified in Table 2 showed that cancer patients (T0) ranged from 0.005% to 47% with Stage IV.

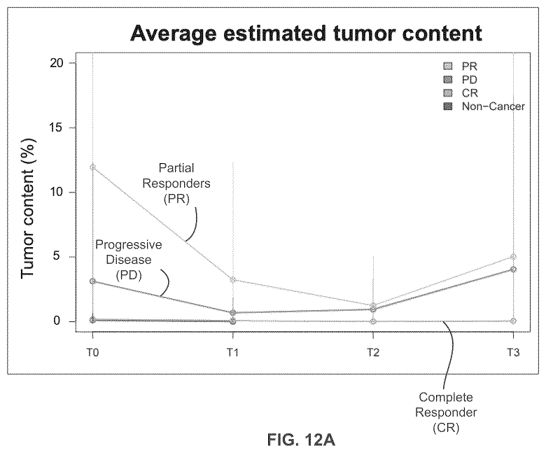

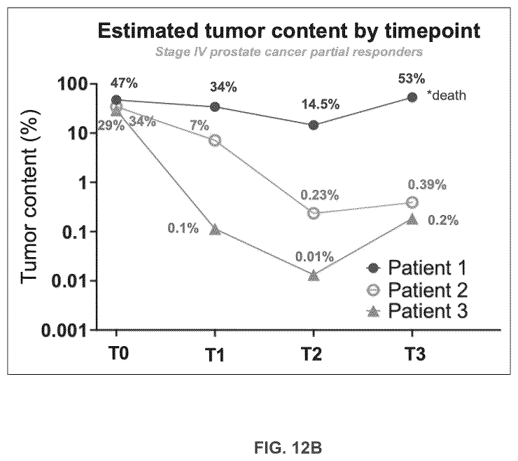

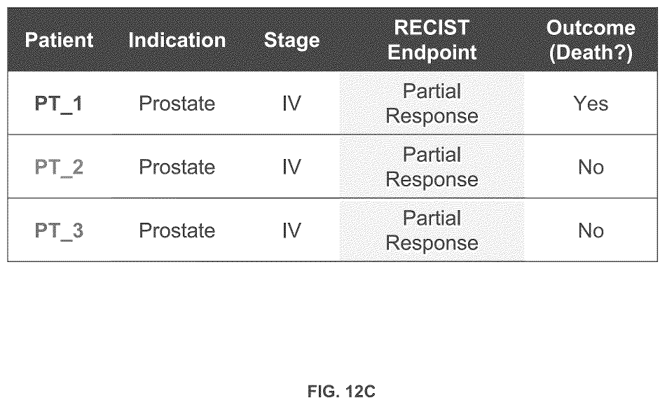

Further analysis was performed to assess longitudinal tracking, predict prognosis and detect disease progression. FIG. 12A shows average estimated tumor content by RECIST response. Error bars are 95% confidence intervals. Partial responders (PR, n=14) had high % TC and an overall decrease from T0 to T3. Patients with progressive disease (PD, n=14) showed an overall increase in % TC between T0 and T3. Complete responders (CR, n=3) had lower initial % TC that sustained throughout treatment. Healthy, non-cancer individuals (n=23) had sustained low tumor content, as expected.

FIGS. 12B-12C show the endpoints of three stage IV prostate cancer partial responders and the corresponding estimated tumor content. In a small subset of three Stage IV prostate cancer patients with high initial % TC, biomarkers were able to identify one patient (e.g., PT_1) that was reported PR but showed sustained high tumor content and an overall increase in % TC. This patient was deceased at last follow-up demonstrating the potential clinical advantage of molecular profiling for identifying disease progression.

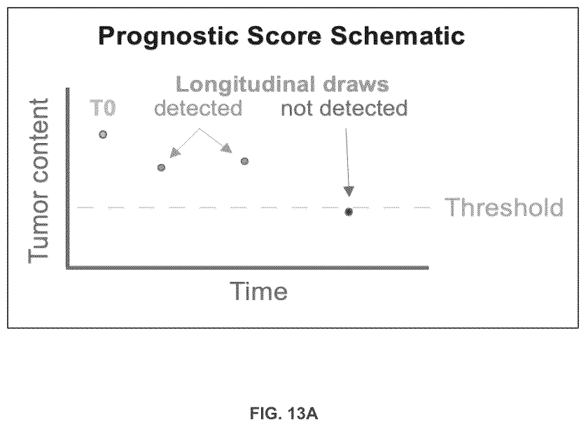

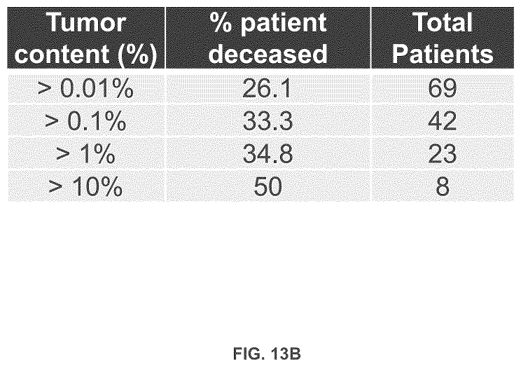

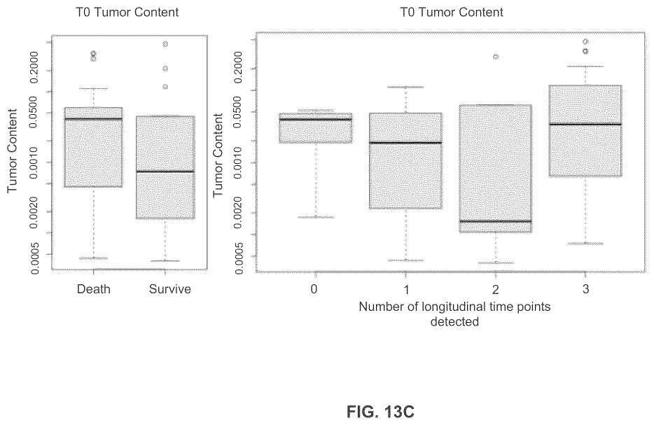

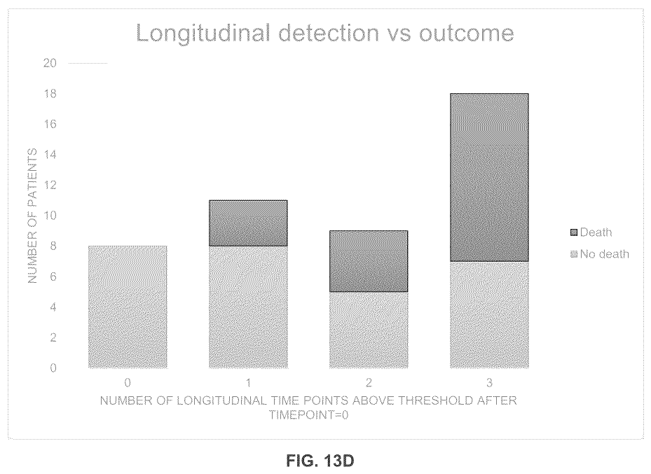

Using a longitudinal threshold (LT) derived from tumor content analysis, prognosis was associated with poor outcome based on the number of serial draws (timepoints) detected that were greater than the LT. Specifically, a longitudinal threshold value of 0.037% based on a limit of detection (LOD) was used. LOD analysis is further described in the subsequent Example 5. Thus, prognosis was associated with outcome according to the number of timepoints in which the tumor content from a patient was determined to be above the longitudinal threshold value of 0.037%. FIG. 13A shows an example prognosis score schematic.

FIG. 13B shows that high initial content values (e.g., >10%) can be associated with prognosis. Specifically, of the 8 patients with greater than a 10% tumor content, 50% of them were deceased upon followup. FIG. 13C shows that lower initial tumor content value is not sufficient to predict prognosis. Specifically, in the left panel of FIG. 13C, no statistical significance was observed in initial tumor content (“T0 tumor content”) between patients that survived or were deceased. In the right panel of FIG. 13C, the initial tumor content (“T0 tumor content”) is shown for patients in which between 0 and 3 predicted tumor content values were above the LT (0.037%). Here, all distributions were non-significant in comparison to each other. For example, the p-value between 0 and 3 predicted tumor content values above the LT was p=0.0915. Thus, the initial tumor content value by itself is not predictive of prognosis.

In contrast, FIG. 13D shows that the number of samples obtained from patients across longitudinal timepoints that are above the LT was predictive of prognosis. Here, a total of 46 patients were identified as having a sample at timepoint 0 with an estimated tumor content above the longitudinal threshold, and further having three additional blood draws at subsequent timepoints. For the three additional blood draws (T1, T2, T3), number of estimated tumor content (e.g., 0, 1, 2 or 3) that were above the longitudinal threshold was quantified. Specifically, using the LT of 0.037%, tumor content was predicted and compared to the threshold to classify whether a sample had a presence of cancer. Notably, patients with 0 additional time points above the threshold had good prognosis (0% died), while patients with all three time points above the threshold had poor prognosis (63% died). This indicated that the number of longitudinal time points for a patient in which the predicted tumor content is above the LT was associated with outcome (e.g., death or survival).

### Example 5: Limit of Blank and Limit of Detection Validation of Methylation Variants

These studies involved 19 subjects with newly diagnosed treatment-naïve cancer (8 cancer types) and 50 with no history, diagnosis, or cancer symptoms. A total of 122 replicate samples were used to assess reproducibility and precision, 8 non-template controls (water) to determine limit of blank (LOB), and cohorts of matched biopsy and cfDNA to determine tumor content limit of detection (LOD).

Precision was assessed within 5 sub-studies, by comparing concordance of predicted binary cancer classification between replicate samples. Each sub-study showed >83% precision.

To determine limit of blank, 8 non-template controls (water) were carried through the entire assay. Table 3 shows the results of the limit of blank study. On average ˜0.02% unique aligned reads of a true sample were detected on the same sequencing run.

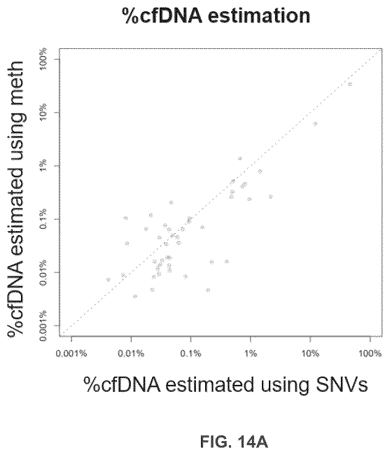

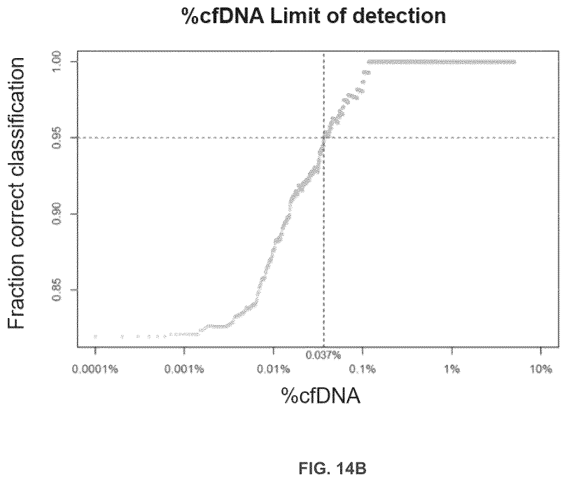

To assess tumor content LOD, the disclosed methodology that analyzes the MVs of Table 1 was implemented to estimate the amount of tumor-derived DNA in each cfDNA sample, without needed matched biopsy. The estimates were further validated using whole exome sequencing, an orthogonal gold-standard approach. FIG. 14A shows the results of whole exome sequencing which was performed on matched biopsy and cfDNA so that SNVs could be used to estimate percentage cfDNA.

Specifically, to determine the LOD threshold, estimated tumor content was first estimated for an independent dataset of 1055 samples (630 cancer and 425 noncancer). This was conducted using a different set of reference data, 200 non-cancer samples. Here, the 1,043 informative K5 features shown in Table 1 were selected as informative. For the same 1055 samples, results (e.g., binary yes/no cancer/no cancer results) were obtained using 95% specificity. Taking only cancers, the amount of tumor content needed whereby 95% cancer samples were correctly predicted as cancer was calculated. This represented the limit of detection value of 0.037% and was further used as the longitudinal threshold value, as described herein. FIG. 14B shows the fraction of correctly classified cancer cfDNA samples by % cfDNA. The horizontal dashed line shows the level of 95% correct predictions. Here, the tumor content LOD whereby 95% true cancer samples were correctly predicted was LOD=0.037%.

### TABLES

TABLE 1
1043 human universal genomic locations and CpG sites mapped to human genome, hg19. The “Start”
and “End” columns indicate the beginning and end, respectively of a range of genomic
locations within a chromosome (“Chrom.”). The SEQ ID NO refers to the nucleotide sequence
of the range of genomic locations (between the “Start” and “End” genomic locations, inclusive)

| | SEQ | | | | | | | |
|---|---|---|---|---|---|---|---|---|
| Chrom. | ID NO: | Start | End | CpG1 | CpG2 | CpG3 | CpG4 | CpG5 |
| chr1 | 1 | 36042792 | 36042826 | 36042792 | 36042799 | 36042816 | 36042822 | 36042826 |
| chr1 | 2 | 36042799 | 36042842 | 36042799 | 36042816 | 36042822 | 36042826 | 36042842 |
| chr1 | 3 | 36042826 | 36042879 | 36042826 | 36042842 | 36042845 | 36042848 | 36042879 |
| chr1 | 4 | 36042842 | 36042901 | 36042842 | 36042845 | 36042848 | 36042879 | 36042901 |
| chr1 | 5 | 36042845 | 36042904 | 36042845 | 36042848 | 36042879 | 36042901 | 36042904 |
| chr1 | 6 | 36042848 | 36042933 | 36042848 | 36042879 | 36042901 | 36042904 | 36042933 |
| chr1 | 7 | 36042879 | 36042935 | 36042879 | 36042901 | 36042904 | 36042933 | 36042935 |
| chr1 | 8 | 36042901 | 36042944 | 36042901 | 36042904 | 36042933 | 36042935 | 36042944 |
| chr1 | 9 | 36042954 | 36043002 | 36042954 | 36042960 | 36042986 | 36042996 | 36043002 |
| chr1 | 10 | 36042960 | 36043011 | 36042960 | 36042986 | 36042996 | 36043002 | 36043011 |
| chr1 | 11 | 36042986 | 36043014 | 36042986 | 36042996 | 36043002 | 36043011 | 36043014 |
| chr1 | 12 | 36042996 | 36043019 | 36042996 | 36043002 | 36043011 | 36043014 | 36043019 |
| chr1 | 13 | 36043002 | 36043050 | 36043002 | 36043011 | 36043014 | 36043019 | 36043050 |
| chr1 | 14 | 36043066 | 36043134 | 36043066 | 36043084 | 36043111 | 36043131 | 36043134 |
| chr1 | 15 | 36043084 | 36043163 | 36043084 | 36043111 | 36043131 | 36043134 | 36043163 |
| chr1 | 16 | 36043111 | 36043169 | 36043111 | 36043131 | 36043134 | 36043163 | 36043169 |
| chr1 | 17 | 36043131 | 36043172 | 36043131 | 36043134 | 36043163 | 36043169 | 36043172 |
| chr1 | 18 | 36043134 | 36043178 | 36043134 | 36043163 | 36043169 | 36043172 | 36043178 |
| chr1 | 19 | 36043172 | 36043245 | 36043172 | 36043178 | 36043181 | 36043205 | 36043245 |
| chr1 | 20 | 36043178 | 36043252 | 36043178 | 36043181 | 36043205 | 36043245 | 36043252 |
| chr1 | 21 | 36043181 | 36043257 | 36043181 | 36043205 | 36043245 | 36043252 | 36043257 |
| chr1 | 22 | 36043205 | 36043262 | 36043205 | 36043245 | 36043252 | 36043257 | 36043262 |
| chr1 | 23 | 36043257 | 36043282 | 36043257 | 36043262 | 36043269 | 36043275 | 36043282 |
| chr1 | 24 | 36043262 | 36043288 | 36043262 | 36043269 | 36043275 | 36043282 | 36043288 |
| chr1 | 25 | 36043269 | 36043294 | 36043269 | 36043275 | 36043282 | 36043288 | 36043294 |
| chr1 | 26 | 36043275 | 36043297 | 36043275 | 36043282 | 36043288 | 36043294 | 36043297 |
| chr1 | 27 | 36043282 | 36043299 | 36043282 | 36043288 | 36043294 | 36043297 | 36043299 |
| chr1 | 28 | 36043288 | 36043321 | 36043288 | 36043294 | 36043297 | 36043299 | 36043321 |
| chr1 | 29 | 36043294 | 36043352 | 36043294 | 36043297 | 36043299 | 36043321 | 36043352 |
| chr1 | 30 | 36043297 | 36043369 | 36043297 | 36043299 | 36043321 | 36043352 | 36043369 |
| chr1 | 31 | 36043299 | 36043373 | 36043299 | 36043321 | 36043352 | 36043369 | 36043373 |
| chr1 | 32 | 36043321 | 36043382 | 36043321 | 36043352 | 36043369 | 36043373 | 36043382 |
| chr1 | 33 | 36043369 | 36043398 | 36043369 | 36043373 | 36043382 | 36043390 | 36043398 |
| chr1 | 34 | 36043373 | 36043401 | 36043373 | 36043382 | 36043390 | 36043398 | 36043401 |
| chr1 | 35 | 36043382 | 36043419 | 36043382 | 36043390 | 36043398 | 36043401 | 36043419 |
| chr1 | 36 | 36043390 | 36043441 | 36043390 | 36043398 | 36043401 | 36043419 | 36043441 |
| chr1 | 37 | 36043398 | 36043443 | 36043398 | 36043401 | 36043419 | 36043441 | 36043443 |
| chr1 | 38 | 41284135 | 41284184 | 41284135 | 41284157 | 41284160 | 41284166 | 41284184 |
| chr1 | 39 | 44883224 | 44883276 | 44883224 | 44883227 | 44883243 | 44883247 | 44883276 |
| chr1 | 40 | 44883227 | 44883283 | 44883227 | 44883243 | 44883247 | 44883276 | 44883283 |
| chr1 | 41 | 46914258 | 46914286 | 46914258 | 46914269 | 46914279 | 46914284 | 46914286 |
| chr1 | 42 | 46914269 | 46914288 | 46914269 | 46914279 | 46914284 | 46914286 | 46914288 |
| chr1 | 43 | 46914288 | 46914334 | 46914288 | 46914296 | 46914300 | 46914312 | 46914334 |
| chr1 | 44 | 47909757 | 47909825 | 47909757 | 47909759 | 47909782 | 47909791 | 47909825 |
| chr1 | 45 | 47910798 | 47910832 | 47910798 | 47910812 | 47910817 | 47910829 | 47910832 |
| chr1 | 46 | 50882791 | 50882910 | 50882791 | 50882824 | 50882826 | 50882854 | 50882910 |
| chr1 | 47 | 50883448 | 50883588 | 50883448 | 50883483 | 50883527 | 50883529 | 50883588 |
| chr1 | 48 | 50883483 | 50883597 | 50883483 | 50883527 | 50883529 | 50883588 | 50883597 |
| chr1 | 49 | 63785392 | 63785429 | 63785392 | 63785397 | 63785408 | 63785414 | 63785429 |
| chr1 | 50 | 63785397 | 63785439 | 63785397 | 63785408 | 63785414 | 63785429 | 63785439 |
| chr1 | 51 | 63785593 | 63785689 | 63785593 | 63785598 | 63785618 | 63785679 | 63785689 |
| chr1 | 52 | 63785598 | 63785701 | 63785598 | 63785618 | 63785679 | 63785689 | 63785701 |
| chr1 | 53 | 63786038 | 63786073 | 63786038 | 63786040 | 63786059 | 63786061 | 63786073 |
| chr1 | 54 | 63786040 | 63786078 | 63786040 | 63786059 | 63786061 | 63786073 | 63786078 |
| chr1 | 55 | 63786061 | 63786150 | 63786061 | 63786073 | 63786078 | 63786083 | 63786150 |
| chr1 | 56 | 63786078 | 63786196 | 63786078 | 63786083 | 63786150 | 63786188 | 63786196 |
| chr1 | 57 | 63795369 | 63795435 | 63795369 | 63795374 | 63795390 | 63795424 | 63795435 |
| chr1 | 58 | 63795424 | 63795464 | 63795424 | 63795435 | 63795446 | 63795449 | 63795464 |
| chr1 | 59 | 63795711 | 63795755 | 63795711 | 63795722 | 63795750 | 63795753 | 63795755 |
| chr1 | 60 | 63796091 | 63796139 | 63796091 | 63796110 | 63796124 | 63796132 | 63796139 |
| chr1 | 61 | 91184633 | 91184770 | 91184633 | 91184636 | 91184658 | 91184765 | 91184770 |
| chr1 | 62 | 91184636 | 91184777 | 91184636 | 91184658 | 91184765 | 91184770 | 91184777 |
| chr1 | 63 | 91184658 | 91184783 | 91184658 | 91184765 | 91184770 | 91184777 | 91184783 |
| chr1 | 64 | 91189207 | 91189310 | 91189207 | 91189226 | 91189264 | 91189289 | 91189310 |
| chr1 | 65 | 91189742 | 91189819 | 91189742 | 91189744 | 91189774 | 91189794 | 91189819 |
| chr1 | 66 | 91190036 | 91190153 | 91190036 | 91190063 | 91190110 | 91190119 | 91190153 |
| chr1 | 67 | 91190119 | 91190205 | 91190119 | 91190153 | 91190173 | 91190183 | 91190205 |
| chr1 | 68 | 110610858 | 110610899 | 110610858 | 110610873 | 110610878 | 110610883 | 110610899 |
| chr1 | 69 | 110610873 | 110610911 | 110610873 | 110610878 | 110610883 | 110610899 | 110610911 |
| chr1 | 70 | 110610878 | 110610924 | 110610878 | 110610883 | 110610899 | 110610911 | 110610924 |
| chr1 | 71 | 110610883 | 110610928 | 110610883 | 110610899 | 110610911 | 110610924 | 110610928 |
| chr1 | 72 | 110610899 | 110610954 | 110610899 | 110610911 | 110610924 | 110610928 | 110610954 |
| chr1 | 73 | 110610911 | 110610995 | 110610911 | 110610924 | 110610928 | 110610954 | 110610995 |
| chr1 | 74 | 110611056 | 110611108 | 110611056 | 110611065 | 110611078 | 110611082 | 110611108 |
| chr1 | 75 | 110611065 | 110611114 | 110611065 | 110611078 | 110611082 | 110611108 | 110611114 |
| chr1 | 76 | 110611078 | 110611129 | 110611078 | 110611082 | 110611108 | 110611114 | 110611129 |
| chr1 | 77 | 110611082 | 110611145 | 110611082 | 110611108 | 110611114 | 110611129 | 110611145 |
| chr1 | 78 | 110611114 | 110611202 | 110611114 | 110611129 | 110611145 | 110611159 | 110611202 |
| chr1 | 79 | 110611129 | 110611218 | 110611129 | 110611145 | 110611159 | 110611202 | 110611218 |
| chr1 | 80 | 110611202 | 110611287 | 110611202 | 110611218 | 110611278 | 110611283 | 110611287 |
| chr1 | 81 | 110611218 | 110611312 | 110611218 | 110611278 | 110611283 | 110611287 | 110611312 |
| chr1 | 82 | 110611384 | 110611457 | 110611384 | 110611391 | 110611431 | 110611435 | 110611457 |
| chr1 | 83 | 110611391 | 110611465 | 110611391 | 110611431 | 110611435 | 110611457 | 110611465 |
| chr1 | 84 | 110611486 | 110611519 | 110611486 | 110611489 | 110611496 | 110611515 | 110611519 |
| chr1 | 85 | 110611489 | 110611523 | 110611489 | 110611496 | 110611515 | 110611519 | 110611523 |
| chr1 | 86 | 110611496 | 110611528 | 110611496 | 110611515 | 110611519 | 110611523 | 110611528 |
| chr1 | 87 | 110611523 | 110611552 | 110611523 | 110611528 | 110611536 | 110611550 | 110611552 |
| chr1 | 88 | 110611570 | 110611598 | 110611570 | 110611578 | 110611586 | 110611592 | 110611598 |
| chr1 | 89 | 110611578 | 110611601 | 110611578 | 110611586 | 110611592 | 110611598 | 110611601 |
| chr1 | 90 | 110611598 | 110611618 | 110611598 | 110611601 | 110611603 | 110611616 | 110611618 |
| chr1 | 91 | 110611601 | 110611643 | 110611601 | 110611603 | 110611616 | 110611618 | 110611643 |
| chr1 | 92 | 110611603 | 110611646 | 110611603 | 110611616 | 110611618 | 110611643 | 110611646 |
| chr1 | 93 | 110611616 | 110611648 | 110611616 | 110611618 | 110611643 | 110611646 | 110611648 |
| chr1 | 94 | 110611643 | 110611666 | 110611643 | 110611646 | 110611648 | 110611653 | 110611666 |
| chr1 | 95 | 110611646 | 110611683 | 110611646 | 110611648 | 110611653 | 110611666 | 110611683 |
| chr1 | 96 | 110611648 | 110611693 | 110611648 | 110611653 | 110611666 | 110611683 | 110611693 |
| chr1 | 97 | 110611653 | 110611700 | 110611653 | 110611666 | 110611683 | 110611693 | 110611700 |
| chr1 | 98 | 110611702 | 110611817 | 110611702 | 110611712 | 110611807 | 110611815 | 110611817 |
| chr1 | 99 | 110612827 | 110612889 | 110612827 | 110612834 | 110612862 | 110612866 | 110612889 |
| chr1 | 100 | 119528095 | 119528190 | 119528095 | 119528098 | 119528141 | 119528164 | 119528190 |
| chr1 | 101 | 119528098 | 119528212 | 119528098 | 119528141 | 119528164 | 119528190 | 119528212 |
| chr1 | 102 | 119528719 | 119528848 | 119528719 | 119528755 | 119528797 | 119528799 | 119528848 |
| chr1 | 103 | 119529044 | 119529151 | 119529044 | 119529080 | 119529087 | 119529104 | 119529151 |
| chr1 | 104 | 119529240 | 119529338 | 119529240 | 119529254 | 119529266 | 119529275 | 119529338 |
| chr1 | 105 | 119529254 | 119529360 | 119529254 | 119529266 | 119529275 | 119529338 | 119529360 |
| chr1 | 106 | 119529686 | 119529793 | 119529686 | 119529692 | 119529728 | 119529756 | 119529793 |
| chr1 | 107 | 119529891 | 119530006 | 119529891 | 119529913 | 119529930 | 119529948 | 119530006 |
| chr1 | 108 | 119549074 | 119549147 | 119549074 | 119549096 | 119549127 | 119549145 | 119549147 |
| chr1 | 109 | 119549205 | 119549261 | 119549205 | 119549230 | 119549236 | 119549240 | 119549261 |
| chr1 | 110 | 119551066 | 119551106 | 119551066 | 119551069 | 119551084 | 119551088 | 119551106 |
| chr1 | 111 | 156863092 | 156863184 | 156863092 | 156863120 | 156863123 | 156863129 | 156863184 |
| chr1 | 112 | 197888750 | 197888839 | 197888750 | 197888753 | 197888790 | 197888796 | 197888839 |
| chr1 | 113 | 197888753 | 197888847 | 197888753 | 197888790 | 197888796 | 197888839 | 197888847 |
| chr1 | 114 | 197888926 | 197889046 | 197888926 | 197888931 | 197888998 | 197889044 | 197889046 |
| chr1 | 115 | 197888931 | 197889049 | 197888931 | 197888998 | 197889044 | 197889046 | 197889049 |
| chr1 | 116 | 197888998 | 197889053 | 197888998 | 197889044 | 197889046 | 197889049 | 197889053 |
| chr1 | 117 | 217311361 | 217311426 | 217311361 | 217311374 | 217311398 | 217311400 | 217311426 |
| chr1 | 118 | 217311374 | 217311468 | 217311374 | 217311398 | 217311400 | 217311426 | 217311468 |
| chr10 | 119 | 16562345 | 16562369 | 16562345 | 16562348 | 16562362 | 16562366 | 16562369 |
| chr10 | 120 | 16562348 | 16562371 | 16562348 | 16562362 | 16562366 | 16562369 | 16562371 |
| chr10 | 121 | 16562468 | 16562526 | 16562468 | 16562470 | 16562515 | 16562524 | 16562526 |
| chr10 | 122 | 16562470 | 16562536 | 16562470 | 16562515 | 16562524 | 16562526 | 16562536 |
| chr10 | 123 | 22765440 | 22765457 | 22765440 | 22765442 | 22765445 | 22765453 | 22765457 |
| chr10 | 124 | 22765442 | 22765459 | 22765442 | 22765445 | 22765453 | 22765457 | 22765459 |
| chr10 | 125 | 22765520 | 22765615 | 22765520 | 22765581 | 22765611 | 22765613 | 22765615 |
| chr10 | 126 | 27548211 | 27548289 | 27548211 | 27548231 | 27548267 | 27548272 | 27548289 |
| chr10 | 127 | 27548231 | 27548312 | 27548231 | 27548267 | 27548272 | 27548289 | 27548312 |
| chr10 | 128 | 100993553 | 100993587 | 100993553 | 100993556 | 100993558 | 100993583 | 100993587 |
| chr10 | 129 | 100993556 | 100993589 | 100993556 | 100993558 | 100993583 | 100993587 | 100993589 |
| chr10 | 130 | 100993558 | 100993591 | 100993558 | 100993583 | 100993587 | 100993589 | 100993591 |
| chr10 | 131 | 100993583 | 100993597 | 100993583 | 100993587 | 100993589 | 100993591 | 100993597 |
| chr10 | 132 | 101290958 | 101291030 | 101290958 | 101290974 | 101290978 | 101291010 | 101291030 |
| chr10 | 133 | 102894441 | 102894571 | 102894441 | 102894490 | 102894547 | 102894562 | 102894571 |
| chr10 | 134 | 102894490 | 102894579 | 102894490 | 102894547 | 102894562 | 102894571 | 102894579 |
| chr10 | 135 | 102894982 | 102895040 | 102894982 | 102895018 | 102895020 | 102895034 | 102895040 |
| chr10 | 136 | 102899886 | 102899949 | 102899886 | 102899888 | 102899913 | 102899934 | 102899949 |
| chr10 | 137 | 102899888 | 102899953 | 102899888 | 102899913 | 102899934 | 102899949 | 102899953 |
| chr10 | 138 | 102899913 | 102899960 | 102899913 | 102899934 | 102899949 | 102899953 | 102899960 |
| chr10 | 139 | 102899934 | 102899976 | 102899934 | 102899949 | 102899953 | 102899960 | 102899976 |
| chr10 | 140 | 102899949 | 102899983 | 102899949 | 102899953 | 102899960 | 102899976 | 102899983 |
| chr10 | 141 | 102900141 | 102900168 | 102900141 | 102900149 | 102900153 | 102900160 | 102900168 |
| chr10 | 142 | 102900354 | 102900409 | 102900354 | 102900365 | 102900392 | 102900396 | 102900409 |
| chr10 | 143 | 102996056 | 102996136 | 102996056 | 102996115 | 102996124 | 102996132 | 102996136 |
| chr10 | 144 | 102996115 | 102996140 | 102996115 | 102996124 | 102996132 | 102996136 | 102996140 |
| chr10 | 145 | 102997052 | 102997095 | 102997052 | 102997059 | 102997073 | 102997084 | 102997095 |
| chr10 | 146 | 103044095 | 103044115 | 103044095 | 103044097 | 103044099 | 103044112 | 103044115 |
| chr10 | 147 | 103044150 | 103044179 | 103044150 | 103044157 | 103044168 | 103044175 | 103044179 |
| chr10 | 148 | 103044211 | 103044231 | 103044211 | 103044219 | 103044226 | 103044229 | 103044231 |
| chr10 | 149 | 103044312 | 103044385 | 103044312 | 103044323 | 103044340 | 103044352 | 103044385 |
| chr10 | 150 | 103044323 | 103044392 | 103044323 | 103044340 | 103044352 | 103044385 | 103044392 |
| chr10 | 151 | 104000829 | 104000870 | 104000829 | 104000831 | 104000859 | 104000865 | 104000870 |
| chr10 | 152 | 118891641 | 118891690 | 118891641 | 118891655 | 118891663 | 118891670 | 118891690 |
| chr10 | 153 | 118899689 | 118899788 | 118899689 | 118899702 | 118899718 | 118899743 | 118899788 |
| chr10 | 154 | 119494463 | 119494525 | 119494463 | 119494494 | 119494502 | 119494516 | 119494525 |
| chr10 | 155 | 119494829 | 119494909 | 119494829 | 119494849 | 119494878 | 119494905 | 119494909 |
| chr10 | 156 | 122708729 | 122708801 | 122708729 | 122708739 | 122708746 | 122708780 | 122708801 |
| chr10 | 157 | 122708739 | 122708812 | 122708739 | 122708746 | 122708780 | 122708801 | 122708812 |
| chr10 | 158 | 123922719 | 123922851 | 123922719 | 123922725 | 123922732 | 123922788 | 123922851 |
| chr10 | 159 | 123922788 | 123922876 | 123922788 | 123922851 | 123922863 | 123922867 | 123922876 |
| chr10 | 160 | 123922851 | 123922904 | 123922851 | 123922863 | 123922867 | 123922876 | 123922904 |
| chr10 | 161 | 123922863 | 123922909 | 123922863 | 123922867 | 123922876 | 123922904 | 123922909 |
| chr10 | 162 | 123922867 | 123922911 | 123922867 | 123922876 | 123922904 | 123922909 | 123922911 |
| chr10 | 163 | 123922876 | 123922916 | 123922876 | 123922904 | 123922909 | 123922911 | 123922916 |
| chr10 | 164 | 123922904 | 123922942 | 123922904 | 123922909 | 123922911 | 123922916 | 123922942 |
| chr10 | 165 | 123922909 | 123922946 | 123922909 | 123922911 | 123922916 | 123922942 | 123922946 |
| chr10 | 166 | 123922911 | 123922952 | 123922911 | 123922916 | 123922942 | 123922946 | 123922952 |
| chr10 | 167 | 123922916 | 123922955 | 123922916 | 123922942 | 123922946 | 123922952 | 123922955 |
| chr10 | 168 | 123922942 | 123922960 | 123922942 | 123922946 | 123922952 | 123922955 | 123922960 |
| chr10 | 169 | 123922946 | 123922970 | 123922946 | 123922952 | 123922955 | 123922960 | 123922970 |
| chr10 | 170 | 123922952 | 123922990 | 123922952 | 123922955 | 123922960 | 123922970 | 123922990 |
| chr10 | 171 | 123922955 | 123923010 | 123922955 | 123922960 | 123922970 | 123922990 | 123923010 |
| chr10 | 172 | 123922960 | 123923016 | 123922960 | 123922970 | 123922990 | 123923010 | 123923016 |
| chr10 | 173 | 123922970 | 123923023 | 123922970 | 123922990 | 123923010 | 123923016 | 123923023 |
| chr10 | 174 | 123922990 | 123923025 | 123922990 | 123923010 | 123923016 | 123923023 | 123923025 |
| chr10 | 175 | 123923066 | 123923131 | 123923066 | 123923081 | 123923099 | 123923106 | 123923131 |
| chr10 | 176 | 123923081 | 123923141 | 123923081 | 123923099 | 123923106 | 123923131 | 123923141 |
| chr10 | 177 | 123923131 | 123923160 | 123923131 | 123923141 | 123923144 | 123923150 | 123923160 |
| chr10 | 178 | 123923141 | 123923164 | 123923141 | 123923144 | 123923150 | 123923160 | 123923164 |
| chr10 | 179 | 123923144 | 123923169 | 123923144 | 123923150 | 123923160 | 123923164 | 123923169 |
| chr10 | 180 | 123923150 | 123923179 | 123923150 | 123923160 | 123923164 | 123923169 | 123923179 |
| chr10 | 181 | 123923160 | 123923181 | 123923160 | 123923164 | 123923169 | 123923179 | 123923181 |
| chr10 | 182 | 123923164 | 123923185 | 123923164 | 123923169 | 123923179 | 123923181 | 123923185 |
| chr10 | 183 | 123923235 | 123923256 | 123923235 | 123923238 | 123923242 | 123923249 | 123923256 |
| chr10 | 184 | 123923238 | 123923261 | 123923238 | 123923242 | 123923249 | 123923256 | 123923261 |
| chr10 | 185 | 123923242 | 123923266 | 123923242 | 123923249 | 123923256 | 123923261 | 123923266 |
| chr10 | 186 | 123923249 | 123923268 | 123923249 | 123923256 | 123923261 | 123923266 | 123923268 |
| chr10 | 187 | 123923256 | 123923273 | 123923256 | 123923261 | 123923266 | 123923268 | 123923273 |
| chr10 | 188 | 123923409 | 123923478 | 123923409 | 123923422 | 123923435 | 123923450 | 123923478 |
| chr10 | 189 | 123923422 | 123923499 | 123923422 | 123923435 | 123923450 | 123923478 | 123923499 |
| chr10 | 190 | 123923435 | 123923518 | 123923435 | 123923450 | 123923478 | 123923499 | 123923518 |
| chr10 | 191 | 123923450 | 123923522 | 123923450 | 123923478 | 123923499 | 123923518 | 123923522 |
| chr10 | 192 | 123923532 | 123923630 | 123923532 | 123923541 | 123923562 | 123923578 | 123923630 |
| chr11 | 193 | 31825712 | 31825792 | 31825712 | 31825744 | 31825751 | 31825756 | 31825792 |
| chr11 | 194 | 31826048 | 31826098 | 31826048 | 31826068 | 31826072 | 31826081 | 31826098 |
| chr11 | 195 | 31826072 | 31826137 | 31826072 | 31826081 | 31826098 | 31826107 | 31826137 |
| chr11 | 196 | 31826081 | 31826143 | 31826081 | 31826098 | 31826107 | 31826137 | 31826143 |
| chr11 | 197 | 31826098 | 31826182 | 31826098 | 31826107 | 31826137 | 31826143 | 31826182 |
| chr11 | 198 | 31826272 | 31826338 | 31826272 | 31826293 | 31826305 | 31826334 | 31826338 |
| chr11 | 199 | 31826293 | 31826340 | 31826293 | 31826305 | 31826334 | 31826338 | 31826340 |
| chr11 | 200 | 31826305 | 31826348 | 31826305 | 31826334 | 31826338 | 31826340 | 31826348 |
| chr11 | 201 | 31826334 | 31826394 | 31826334 | 31826338 | 31826340 | 31826348 | 31826394 |
| chr11 | 202 | 31826338 | 31826409 | 31826338 | 31826340 | 31826348 | 31826394 | 31826409 |
| chr11 | 203 | 31826340 | 31826418 | 31826340 | 31826348 | 31826394 | 31826409 | 31826418 |
| chr11 | 204 | 31826409 | 31826457 | 31826409 | 31826418 | 31826421 | 31826443 | 31826457 |
| chr11 | 205 | 31826418 | 31826472 | 31826418 | 31826421 | 31826443 | 31826457 | 31826472 |
| chr11 | 206 | 31826421 | 31826491 | 31826421 | 31826443 | 31826457 | 31826472 | 31826491 |
| chr11 | 207 | 31826443 | 31826558 | 31826443 | 31826457 | 31826472 | 31826491 | 31826558 |
| chr11 | 208 | 31826472 | 31826574 | 31826472 | 31826491 | 31826558 | 31826564 | 31826574 |
| chr11 | 209 | 31826834 | 31826891 | 31826834 | 31826840 | 31826849 | 31826867 | 31826891 |
| chr11 | 210 | 31826840 | 31826900 | 31826840 | 31826849 | 31826867 | 31826891 | 31826900 |
| chr11 | 211 | 31826849 | 31826928 | 31826849 | 31826867 | 31826891 | 31826900 | 31826928 |
| chr11 | 212 | 31826930 | 31826966 | 31826930 | 31826943 | 31826959 | 31826963 | 31826966 |
| chr11 | 213 | 31827125 | 31827190 | 31827125 | 31827149 | 31827171 | 31827173 | 31827190 |
| chr11 | 214 | 31839934 | 31840012 | 31839934 | 31839947 | 31839980 | 31839987 | 31840012 |
| chr11 | 215 | 31839947 | 31840018 | 31839947 | 31839980 | 31839987 | 31840012 | 31840018 |
| chr11 | 216 | 31841818 | 31841889 | 31841818 | 31841820 | 31841858 | 31841876 | 31841889 |
| chr11 | 217 | 78673053 | 78673104 | 78673053 | 78673073 | 78673085 | 78673100 | 78673104 |
| chr12 | 218 | 6664596 | 6664632 | 6664596 | 6664602 | 6664616 | 6664627 | 6664632 |
| chr12 | 219 | 6664602 | 6664635 | 6664602 | 6664616 | 6664627 | 6664632 | 6664635 |
| chr12 | 220 | 6664616 | 6664668 | 6664616 | 6664627 | 6664632 | 6664635 | 6664668 |
| chr12 | 221 | 6664627 | 6664671 | 6664627 | 6664632 | 6664635 | 6664668 | 6664671 |
| chr12 | 222 | 6664632 | 6664683 | 6664632 | 6664635 | 6664668 | 6664671 | 6664683 |
| chr12 | 223 | 6664635 | 6664685 | 6664635 | 6664668 | 6664671 | 6664683 | 6664685 |
| chr12 | 224 | 6664668 | 6664692 | 6664668 | 6664671 | 6664683 | 6664685 | 6664692 |
| chr12 | 225 | 6664671 | 6664694 | 6664671 | 6664683 | 6664685 | 6664692 | 6664694 |
| chr12 | 226 | 6664683 | 6664703 | 6664683 | 6664685 | 6664692 | 6664694 | 6664703 |
| chr12 | 227 | 6664694 | 6664716 | 6664694 | 6664703 | 6664707 | 6664714 | 6664716 |
| chr12 | 228 | 6665096 | 6665180 | 6665096 | 6665099 | 6665108 | 6665135 | 6665180 |
| chr12 | 229 | 6665099 | 6665187 | 6665099 | 6665108 | 6665135 | 6665180 | 6665187 |
| chr12 | 230 | 52652256 | 52652292 | 52652256 | 52652261 | 52652268 | 52652275 | 52652292 |
| chr12 | 231 | 52652261 | 52652294 | 52652261 | 52652268 | 52652275 | 52652292 | 52652294 |
| chr12 | 232 | 52652268 | 52652301 | 52652268 | 52652275 | 52652292 | 52652294 | 52652301 |
| chr12 | 233 | 52652275 | 52652310 | 52652275 | 52652292 | 52652294 | 52652301 | 52652310 |
| chr12 | 234 | 52652292 | 52652318 | 52652292 | 52652294 | 52652301 | 52652310 | 52652318 |
| chr12 | 235 | 52652301 | 52652348 | 52652301 | 52652310 | 52652318 | 52652342 | 52652348 |
| chr12 | 236 | 54338824 | 54338884 | 54338824 | 54338828 | 54338837 | 54338839 | 54338884 |
| chr12 | 237 | 54338828 | 54338886 | 54338828 | 54338837 | 54338839 | 54338884 | 54338886 |
| chr12 | 238 | 54338837 | 54338915 | 54338837 | 54338839 | 54338884 | 54338886 | 54338915 |
| chr12 | 239 | 54338839 | 54338918 | 54338839 | 54338884 | 54338886 | 54338915 | 54338918 |
| chr12 | 240 | 54338929 | 54338981 | 54338929 | 54338937 | 54338951 | 54338978 | 54338981 |
| chr12 | 241 | 54338937 | 54339012 | 54338937 | 54338951 | 54338978 | 54338981 | 54339012 |
| chr12 | 242 | 54338951 | 54339018 | 54338951 | 54338978 | 54338981 | 54339012 | 54339018 |
| chr12 | 243 | 54339158 | 54339190 | 54339158 | 54339160 | 54339163 | 54339167 | 54339190 |
| chr12 | 244 | 54339160 | 54339258 | 54339160 | 54339163 | 54339167 | 54339190 | 54339258 |
| chr12 | 245 | 54339190 | 54339301 | 54339190 | 54339258 | 54339288 | 54339297 | 54339301 |
| chr12 | 246 | 54339575 | 54339667 | 54339575 | 54339613 | 54339636 | 54339653 | 54339667 |
| chr12 | 247 | 54360480 | 54360585 | 54360480 | 54360494 | 54360507 | 54360511 | 54360585 |
| chr12 | 248 | 54360494 | 54360591 | 54360494 | 54360507 | 54360511 | 54360585 | 54360591 |
| chr12 | 249 | 54377849 | 54377906 | 54377849 | 54377869 | 54377878 | 54377898 | 54377906 |
| chr12 | 250 | 81102252 | 81102324 | 81102252 | 81102258 | 81102280 | 81102313 | 81102324 |
| chr12 | 251 | 113901446 | 113901469 | 113901446 | 113901450 | 113901454 | 113901467 | 113901469 |
| chr12 | 252 | 113901857 | 113901885 | 113901857 | 113901864 | 113901866 | 113901877 | 113901885 |
| chr13 | 253 | 96204718 | 96204762 | 96204718 | 96204739 | 96204746 | 96204758 | 96204762 |
| chr13 | 254 | 96204739 | 96204764 | 96204739 | 96204746 | 96204758 | 96204762 | 96204764 |
| chr13 | 255 | 96204768 | 96204805 | 96204768 | 96204770 | 96204794 | 96204801 | 96204805 |
| chr13 | 256 | 96204770 | 96204807 | 96204770 | 96204794 | 96204801 | 96204805 | 96204807 |
| chr13 | 257 | 96205184 | 96205241 | 96205184 | 96205219 | 96205226 | 96205239 | 96205241 |
| chr13 | 258 | 96205219 | 96205248 | 96205219 | 96205226 | 96205239 | 96205241 | 96205248 |
| chr13 | 259 | 96205226 | 96205280 | 96205226 | 96205239 | 96205241 | 96205248 | 96205280 |
| chr13 | 260 | 100641499 | 100641560 | 100641499 | 100641508 | 100641529 | 100641538 | 100641560 |
| chr13 | 261 | 112712210 | 112712259 | 112712210 | 112712224 | 112712231 | 112712256 | 112712259 |
| chr13 | 262 | 112758939 | 112759004 | 112758939 | 112758986 | 112758996 | 112758998 | 112759004 |
| chr14 | 263 | 24803896 | 24803922 | 24803896 | 24803900 | 24803903 | 24803917 | 24803922 |
| chr14 | 264 | 24803900 | 24803925 | 24803900 | 24803903 | 24803917 | 24803922 | 24803925 |
| chr14 | 265 | 24803903 | 24803951 | 24803903 | 24803917 | 24803922 | 24803925 | 24803951 |
| chr14 | 266 | 24803917 | 24803961 | 24803917 | 24803922 | 24803925 | 24803951 | 24803961 |
| chr14 | 267 | 24803922 | 24803972 | 24803922 | 24803925 | 24803951 | 24803961 | 24803972 |
| chr14 | 268 | 24803925 | 24803974 | 24803925 | 24803951 | 24803961 | 24803972 | 24803974 |
| chr14 | 269 | 24803951 | 24803986 | 24803951 | 24803961 | 24803972 | 24803974 | 24803986 |
| chr14 | 270 | 36991735 | 36991819 | 36991735 | 36991765 | 36991779 | 36991793 | 36991819 |
| chr14 | 271 | 36991883 | 36991952 | 36991883 | 36991900 | 36991908 | 36991922 | 36991952 |
| chr14 | 272 | 36991900 | 36991954 | 36991900 | 36991908 | 36991922 | 36991952 | 36991954 |
| chr14 | 273 | 36993580 | 36993651 | 36993580 | 36993595 | 36993608 | 36993635 | 36993651 |
| chr14 | 274 | 37125008 | 37125090 | 37125008 | 37125033 | 37125041 | 37125057 | 37125090 |
| chr14 | 275 | 37126375 | 37126497 | 37126375 | 37126423 | 37126481 | 37126492 | 37126497 |
| chr14 | 276 | 37127400 | 37127495 | 37127400 | 37127402 | 37127404 | 37127459 | 37127495 |
| chr14 | 277 | 37128425 | 37128556 | 37128425 | 37128481 | 37128546 | 37128549 | 37128556 |
| chr14 | 278 | 37128605 | 37128690 | 37128605 | 37128610 | 37128648 | 37128669 | 37128690 |
| chr14 | 279 | 37130149 | 37130246 | 37130149 | 37130202 | 37130212 | 37130237 | 37130246 |
| chr14 | 280 | 57261256 | 57261325 | 57261256 | 57261293 | 57261313 | 57261323 | 57261325 |
| chr14 | 281 | 57265540 | 57265663 | 57265540 | 57265549 | 57265552 | 57265560 | 57265663 |
| chr14 | 282 | 57265875 | 57265990 | 57265875 | 57265910 | 57265913 | 57265967 | 57265990 |
| chr14 | 283 | 57276527 | 57276605 | 57276527 | 57276529 | 57276545 | 57276548 | 57276605 |
| chr14 | 284 | 57276529 | 57276610 | 57276529 | 57276545 | 57276548 | 57276605 | 57276610 |
| chr14 | 285 | 60973678 | 60973773 | 60973678 | 60973687 | 60973713 | 60973724 | 60973773 |
| chr14 | 286 | 60974106 | 60974193 | 60974106 | 60974115 | 60974122 | 60974165 | 60974193 |
| chr14 | 287 | 60976735 | 60976803 | 60976735 | 60976754 | 60976774 | 60976801 | 60976803 |
| chr14 | 288 | 60977450 | 60977584 | 60977450 | 60977475 | 60977568 | 60977571 | 60977584 |
| chr14 | 289 | 61104329 | 61104420 | 61104329 | 61104371 | 61104386 | 61104408 | 61104420 |
| chr14 | 290 | 61104371 | 61104452 | 61104371 | 61104386 | 61104408 | 61104420 | 61104452 |
| chr14 | 291 | 61104386 | 61104454 | 61104386 | 61104408 | 61104420 | 61104452 | 61104454 |
| chr14 | 292 | 61104503 | 61104558 | 61104503 | 61104511 | 61104520 | 61104539 | 61104558 |
| chr14 | 293 | 61104511 | 61104566 | 61104511 | 61104520 | 61104539 | 61104558 | 61104566 |
| chr14 | 294 | 61104520 | 61104571 | 61104520 | 61104539 | 61104558 | 61104566 | 61104571 |
| chr14 | 295 | 61104539 | 61104574 | 61104539 | 61104558 | 61104566 | 61104571 | 61104574 |
| chr14 | 296 | 61104635 | 61104695 | 61104635 | 61104656 | 61104658 | 61104662 | 61104695 |
| chr14 | 297 | 61104656 | 61104717 | 61104656 | 61104658 | 61104662 | 61104695 | 61104717 |
| chr14 | 298 | 61104658 | 61104719 | 61104658 | 61104662 | 61104695 | 61104717 | 61104719 |
| chr14 | 299 | 61104779 | 61104850 | 61104779 | 61104790 | 61104836 | 61104848 | 61104850 |
| chr14 | 300 | 95239218 | 95239350 | 95239218 | 95239273 | 95239283 | 95239299 | 95239350 |
| chr15 | 301 | 45427519 | 45427594 | 45427519 | 45427548 | 45427577 | 45427592 | 45427594 |
| chr15 | 302 | 45427548 | 45427602 | 45427548 | 45427577 | 45427592 | 45427594 | 45427602 |
| chr15 | 303 | 45427577 | 45427605 | 45427577 | 45427592 | 45427594 | 45427602 | 45427605 |
| chr15 | 304 | 89922315 | 89922359 | 89922315 | 89922324 | 89922328 | 89922345 | 89922359 |
| chr15 | 305 | 89922361 | 89922418 | 89922361 | 89922387 | 89922406 | 89922409 | 89922418 |
| chr15 | 306 | 89922387 | 89922421 | 89922387 | 89922406 | 89922409 | 89922418 | 89922421 |
| chr15 | 307 | 89922423 | 89922450 | 89922423 | 89922431 | 89922438 | 89922444 | 89922450 |
| chr15 | 308 | 89922450 | 89922472 | 89922450 | 89922453 | 89922459 | 89922467 | 89922472 |
| chr15 | 309 | 89922453 | 89922499 | 89922453 | 89922459 | 89922467 | 89922472 | 89922499 |
| chr15 | 310 | 89951787 | 89951870 | 89951787 | 89951796 | 89951810 | 89951856 | 89951870 |
| chr15 | 311 | 89952341 | 89952386 | 89952341 | 89952359 | 89952365 | 89952369 | 89952386 |
| chr15 | 312 | 89952454 | 89952495 | 89952454 | 89952471 | 89952473 | 89952488 | 89952495 |
| chr15 | 313 | 89952517 | 89952556 | 89952517 | 89952528 | 89952534 | 89952540 | 89952556 |
| chr15 | 314 | 89952540 | 89952620 | 89952540 | 89952556 | 89952564 | 89952572 | 89952620 |
| chr16 | 315 | 3221321 | 3221411 | 3221321 | 3221328 | 3221353 | 3221355 | 3221411 |
| chr16 | 316 | 66612967 | 66613072 | 66612967 | 66612973 | 66613033 | 66613053 | 66613072 |
| chr16 | 317 | 66612973 | 66613074 | 66612973 | 66613033 | 66613053 | 66613072 | 66613074 |
| chr17 | 318 | 35299201 | 35299348 | 35299201 | 35299267 | 35299270 | 35299322 | 35299348 |
| chr17 | 319 | 35299621 | 35299672 | 35299621 | 35299625 | 35299658 | 35299670 | 35299672 |
| chr17 | 320 | 35299689 | 35299719 | 35299689 | 35299698 | 35299703 | 35299710 | 35299719 |
| chr17 | 321 | 35300167 | 35300214 | 35300167 | 35300172 | 35300196 | 35300202 | 35300214 |
| chr17 | 322 | 35300172 | 35300217 | 35300172 | 35300196 | 35300202 | 35300214 | 35300217 |
| chr17 | 323 | 37321400 | 37321483 | 37321400 | 37321403 | 37321417 | 37321432 | 37321483 |
| chr17 | 324 | 37321403 | 37321490 | 37321403 | 37321417 | 37321432 | 37321483 | 37321490 |
| chr17 | 325 | 37321417 | 37321514 | 37321417 | 37321432 | 37321483 | 37321490 | 37321514 |
| chr17 | 326 | 37321542 | 37321631 | 37321542 | 37321559 | 37321564 | 37321626 | 37321631 |
| chr17 | 327 | 37321559 | 37321636 | 37321559 | 37321564 | 37321626 | 37321631 | 37321636 |
| chr17 | 328 | 37321564 | 37321653 | 37321564 | 37321626 | 37321631 | 37321636 | 37321653 |
| chr17 | 329 | 37321626 | 37321676 | 37321626 | 37321631 | 37321636 | 37321653 | 37321676 |
| chr17 | 330 | 37321631 | 37321678 | 37321631 | 37321636 | 37321653 | 37321676 | 37321678 |
| chr17 | 331 | 37321636 | 37321690 | 37321636 | 37321653 | 37321676 | 37321678 | 37321690 |
| chr17 | 332 | 37321653 | 37321701 | 37321653 | 37321676 | 37321678 | 37321690 | 37321701 |
| chr17 | 333 | 37321676 | 37321720 | 37321676 | 37321678 | 37321690 | 37321701 | 37321720 |
| chr17 | 334 | 37321678 | 37321723 | 37321678 | 37321690 | 37321701 | 37321720 | 37321723 |
| chr17 | 335 | 37321690 | 37321732 | 37321690 | 37321701 | 37321720 | 37321723 | 37321732 |
| chr17 | 336 | 37321701 | 37321739 | 37321701 | 37321720 | 37321723 | 37321732 | 37321739 |
| chr17 | 337 | 37321720 | 37321756 | 37321720 | 37321723 | 37321732 | 37321739 | 37321756 |
| chr17 | 338 | 37321723 | 37321768 | 37321723 | 37321732 | 37321739 | 37321756 | 37321768 |
| chr17 | 339 | 37321732 | 37321778 | 37321732 | 37321739 | 37321756 | 37321768 | 37321778 |
| chr17 | 340 | 37321739 | 37321783 | 37321739 | 37321756 | 37321768 | 37321778 | 37321783 |
| chr17 | 341 | 37321756 | 37321788 | 37321756 | 37321768 | 37321778 | 37321783 | 37321788 |
| chr17 | 342 | 48042510 | 48042562 | 48042510 | 48042531 | 48042533 | 48042550 | 48042562 |
| chr17 | 343 | 48042531 | 48042564 | 48042531 | 48042533 | 48042550 | 48042562 | 48042564 |
| chr17 | 344 | 48042533 | 48042572 | 48042533 | 48042550 | 48042562 | 48042564 | 48042572 |
| chr17 | 345 | 59529102 | 59529125 | 59529102 | 59529110 | 59529119 | 59529123 | 59529125 |
| chr17 | 346 | 59529123 | 59529169 | 59529123 | 59529125 | 59529138 | 59529152 | 59529169 |
| chr17 | 347 | 59529125 | 59529196 | 59529125 | 59529138 | 59529152 | 59529169 | 59529196 |
| chr17 | 348 | 59529138 | 59529198 | 59529138 | 59529152 | 59529169 | 59529196 | 59529198 |
| chr17 | 349 | 59529152 | 59529220 | 59529152 | 59529169 | 59529196 | 59529198 | 59529220 |
| chr17 | 350 | 59529260 | 59529316 | 59529260 | 59529266 | 59529286 | 59529288 | 59529316 |
| chr17 | 351 | 59529347 | 59529387 | 59529347 | 59529360 | 59529364 | 59529367 | 59529387 |
| chr17 | 352 | 59529360 | 59529391 | 59529360 | 59529364 | 59529367 | 59529387 | 59529391 |
| chr17 | 353 | 59529364 | 59529398 | 59529364 | 59529367 | 59529387 | 59529391 | 59529398 |
| chr17 | 354 | 59529464 | 59529528 | 59529464 | 59529473 | 59529486 | 59529508 | 59529528 |
| chr17 | 355 | 59529528 | 59529568 | 59529528 | 59529533 | 59529535 | 59529542 | 59529568 |
| chr17 | 356 | 59529533 | 59529570 | 59529533 | 59529535 | 59529542 | 59529568 | 59529570 |
| chr17 | 357 | 59529535 | 59529572 | 59529535 | 59529542 | 59529568 | 59529570 | 59529572 |
| chr17 | 358 | 59529542 | 59529584 | 59529542 | 59529568 | 59529570 | 59529572 | 59529584 |
| chr17 | 359 | 59529587 | 59529618 | 59529587 | 59529592 | 59529594 | 59529603 | 59529618 |
| chr17 | 360 | 59529592 | 59529620 | 59529592 | 59529594 | 59529603 | 59529618 | 59529620 |
| chr17 | 361 | 59529967 | 59530021 | 59529967 | 59529976 | 59529980 | 59530015 | 59530021 |
| chr17 | 362 | 59531878 | 59531937 | 59531878 | 59531883 | 59531892 | 59531896 | 59531937 |
| chr17 | 363 | 59531892 | 59531961 | 59531892 | 59531896 | 59531937 | 59531956 | 59531961 |
| chr17 | 364 | 59531896 | 59531993 | 59531896 | 59531937 | 59531956 | 59531961 | 59531993 |
| chr17 | 365 | 59531937 | 59532019 | 59531937 | 59531956 | 59531961 | 59531993 | 59532019 |
| chr17 | 366 | 59531956 | 59532030 | 59531956 | 59531961 | 59531993 | 59532019 | 59532030 |
| chr17 | 367 | 59531961 | 59532037 | 59531961 | 59531993 | 59532019 | 59532030 | 59532037 |
| chr17 | 368 | 59534817 | 59534954 | 59534817 | 59534845 | 59534847 | 59534903 | 59534954 |
| chr17 | 369 | 59534847 | 59534966 | 59534847 | 59534903 | 59534954 | 59534959 | 59534966 |
| chr17 | 370 | 59535016 | 59535112 | 59535016 | 59535025 | 59535070 | 59535076 | 59535112 |
| chr17 | 371 | 59535025 | 59535116 | 59535025 | 59535070 | 59535076 | 59535112 | 59535116 |
| chr17 | 372 | 59535070 | 59535135 | 59535070 | 59535076 | 59535112 | 59535116 | 59535135 |
| chr17 | 373 | 59535425 | 59535499 | 59535425 | 59535436 | 59535463 | 59535466 | 59535499 |
| chr17 | 374 | 59535436 | 59535512 | 59535436 | 59535463 | 59535466 | 59535499 | 59535512 |
| chr17 | 375 | 70113187 | 70113277 | 70113187 | 70113216 | 70113241 | 70113247 | 70113277 |
| chr17 | 376 | 70113216 | 70113295 | 70113216 | 70113241 | 70113247 | 70113277 | 70113295 |
| chr17 | 377 | 70113295 | 70113344 | 70113295 | 70113303 | 70113323 | 70113342 | 70113344 |
| chr17 | 378 | 70113454 | 70113528 | 70113454 | 70113462 | 70113465 | 70113514 | 70113528 |
| chr17 | 379 | 70113462 | 70113537 | 70113462 | 70113465 | 70113514 | 70113528 | 70113537 |
| chr17 | 380 | 70113465 | 70113541 | 70113465 | 70113514 | 70113528 | 70113537 | 70113541 |
| chr17 | 381 | 70113528 | 70113568 | 70113528 | 70113537 | 70113541 | 70113562 | 70113568 |
| chr17 | 382 | 70113641 | 70113763 | 70113641 | 70113643 | 70113648 | 70113696 | 70113763 |
| chr17 | 383 | 70113643 | 70113785 | 70113643 | 70113648 | 70113696 | 70113763 | 70113785 |
| chr17 | 384 | 70113648 | 70113788 | 70113648 | 70113696 | 70113763 | 70113785 | 70113788 |
| chr17 | 385 | 70113828 | 70113908 | 70113828 | 70113837 | 70113843 | 70113856 | 70113908 |
| chr17 | 386 | 70113837 | 70113935 | 70113837 | 70113843 | 70113856 | 70113908 | 70113935 |
| chr17 | 387 | 70113843 | 70113972 | 70113843 | 70113856 | 70113908 | 70113935 | 70113972 |
| chr17 | 388 | 70113856 | 70113981 | 70113856 | 70113908 | 70113935 | 70113972 | 70113981 |
| chr17 | 389 | 70113908 | 70113988 | 70113908 | 70113935 | 70113972 | 70113981 | 70113988 |
| chr17 | 390 | 75369482 | 75369493 | 75369482 | 75369484 | 75369488 | 75369490 | 75369493 |
| chr17 | 391 | 75369484 | 75369505 | 75369484 | 75369488 | 75369490 | 75369493 | 75369505 |
| chr17 | 392 | 75369623 | 75369663 | 75369623 | 75369630 | 75369657 | 75369661 | 75369663 |
| chr17 | 393 | 75369630 | 75369667 | 75369630 | 75369657 | 75369661 | 75369663 | 75369667 |
| chr17 | 394 | 75369667 | 75369706 | 75369667 | 75369686 | 75369699 | 75369701 | 75369706 |
| chr17 | 395 | 75369686 | 75369714 | 75369686 | 75369699 | 75369701 | 75369706 | 75369714 |
| chr17 | 396 | 75369766 | 75369788 | 75369766 | 75369774 | 75369783 | 75369786 | 75369788 |
| chr17 | 397 | 75369783 | 75369796 | 75369783 | 75369786 | 75369788 | 75369790 | 75369796 |
| chr17 | 398 | 75369786 | 75369802 | 75369786 | 75369788 | 75369790 | 75369796 | 75369802 |
| chr17 | 399 | 75369788 | 75369808 | 75369788 | 75369790 | 75369796 | 75369802 | 75369808 |
| chr17 | 400 | 75369808 | 75369844 | 75369808 | 75369810 | 75369813 | 75369819 | 75369844 |
| chr17 | 401 | 75369810 | 75369846 | 75369810 | 75369813 | 75369819 | 75369844 | 75369846 |
| chr17 | 402 | 75369813 | 75369857 | 75369813 | 75369819 | 75369844 | 75369846 | 75369857 |
| chr17 | 403 | 75369819 | 75369859 | 75369819 | 75369844 | 75369846 | 75369857 | 75369859 |
| chr18 | 404 | 44773505 | 44773536 | 44773505 | 44773512 | 44773530 | 44773533 | 44773536 |
| chr18 | 405 | 44773512 | 44773539 | 44773512 | 44773530 | 44773533 | 44773536 | 44773539 |
| chr19 | 406 | 31848008 | 31848124 | 31848008 | 31848049 | 31848085 | 31848122 | 31848124 |
| chr19 | 407 | 58095552 | 58095595 | 58095552 | 58095569 | 58095581 | 58095588 | 58095595 |
| chr19 | 408 | 58095569 | 58095597 | 58095569 | 58095581 | 58095588 | 58095595 | 58095597 |
| chr19 | 409 | 58220190 | 58220229 | 58220190 | 58220194 | 58220208 | 58220221 | 58220229 |
| chr19 | 410 | 58220194 | 58220237 | 58220194 | 58220208 | 58220221 | 58220229 | 58220237 |
| chr19 | 411 | 58220208 | 58220261 | 58220208 | 58220221 | 58220229 | 58220237 | 58220261 |
| chr19 | 412 | 58220221 | 58220287 | 58220221 | 58220229 | 58220237 | 58220261 | 58220287 |
| chr19 | 413 | 58220229 | 58220295 | 58220229 | 58220237 | 58220261 | 58220287 | 58220295 |
| chr19 | 414 | 58220237 | 58220298 | 58220237 | 58220261 | 58220287 | 58220295 | 58220298 |
| chr19 | 415 | 58220261 | 58220316 | 58220261 | 58220287 | 58220295 | 58220298 | 58220316 |
| chr19 | 416 | 58220298 | 58220364 | 58220298 | 58220316 | 58220331 | 58220349 | 58220364 |
| chr19 | 417 | 58220373 | 58220424 | 58220373 | 58220377 | 58220394 | 58220403 | 58220424 |
| chr2 | 418 | 468262 | 468313 | 468262 | 468266 | 468272 | 468303 | 468313 |
| chr2 | 419 | 19561382 | 19561408 | 19561382 | 19561384 | 19561388 | 19561391 | 19561408 |
| chr2 | 420 | 19561384 | 19561418 | 19561384 | 19561388 | 19561391 | 19561408 | 19561418 |
| chr2 | 421 | 19561388 | 19561470 | 19561388 | 19561391 | 19561408 | 19561418 | 19561470 |
| chr2 | 422 | 19561408 | 19561477 | 19561408 | 19561418 | 19561470 | 19561474 | 19561477 |
| chr2 | 423 | 19561541 | 19561596 | 19561541 | 19561549 | 19561561 | 19561578 | 19561596 |
| chr2 | 424 | 19561549 | 19561612 | 19561549 | 19561561 | 19561578 | 19561596 | 19561612 |
| chr2 | 425 | 19561561 | 19561618 | 19561561 | 19561578 | 19561596 | 19561612 | 19561618 |
| chr2 | 426 | 19561596 | 19561632 | 19561596 | 19561612 | 19561618 | 19561627 | 19561632 |
| chr2 | 427 | 19561612 | 19561649 | 19561612 | 19561618 | 19561627 | 19561632 | 19561649 |
| chr2 | 428 | 19561618 | 19561671 | 19561618 | 19561627 | 19561632 | 19561649 | 19561671 |
| chr2 | 429 | 30453730 | 30453775 | 30453730 | 30453758 | 30453768 | 30453770 | 30453775 |
| chr2 | 430 | 30453793 | 30453854 | 30453793 | 30453799 | 30453801 | 30453821 | 30453854 |
| chr2 | 431 | 30453799 | 30453861 | 30453799 | 30453801 | 30453821 | 30453854 | 30453861 |
| chr2 | 432 | 30453801 | 30453871 | 30453801 | 30453821 | 30453854 | 30453861 | 30453871 |
| chr2 | 433 | 38302626 | 38302721 | 38302626 | 38302642 | 38302668 | 38302674 | 38302721 |
| chr2 | 434 | 38302642 | 38302734 | 38302642 | 38302668 | 38302674 | 38302721 | 38302734 |
| chr2 | 435 | 38302668 | 38302747 | 38302668 | 38302674 | 38302721 | 38302734 | 38302747 |
| chr2 | 436 | 38302674 | 38302767 | 38302674 | 38302721 | 38302734 | 38302747 | 38302767 |
| chr2 | 437 | 45155294 | 45155347 | 45155294 | 45155296 | 45155305 | 45155336 | 45155347 |
| chr2 | 438 | 45155296 | 45155356 | 45155296 | 45155305 | 45155336 | 45155347 | 45155356 |
| chr2 | 439 | 45155336 | 45155395 | 45155336 | 45155347 | 45155356 | 45155393 | 45155395 |
| chr2 | 440 | 45156408 | 45156470 | 45156408 | 45156410 | 45156448 | 45156457 | 45156470 |
| chr2 | 441 | 45156965 | 45157023 | 45156965 | 45156992 | 45157003 | 45157005 | 45157023 |
| chr2 | 442 | 45156992 | 45157033 | 45156992 | 45157003 | 45157005 | 45157023 | 45157033 |
| chr2 | 443 | 45157365 | 45157465 | 45157365 | 45157377 | 45157379 | 45157460 | 45157465 |
| chr2 | 444 | 45157377 | 45157472 | 45157377 | 45157379 | 45157460 | 45157465 | 45157472 |
| chr2 | 445 | 63281089 | 63281139 | 63281089 | 63281117 | 63281129 | 63281131 | 63281139 |
| chr2 | 446 | 63281117 | 63281156 | 63281117 | 63281129 | 63281131 | 63281139 | 63281156 |
| chr2 | 447 | 63281129 | 63281158 | 63281129 | 63281131 | 63281139 | 63281156 | 63281158 |
| chr2 | 448 | 63281131 | 63281161 | 63281131 | 63281139 | 63281156 | 63281158 | 63281161 |
| chr2 | 449 | 63281299 | 63281383 | 63281299 | 63281317 | 63281337 | 63281346 | 63281383 |
| chr2 | 450 | 63281337 | 63281466 | 63281337 | 63281346 | 63281383 | 63281460 | 63281466 |
| chr2 | 451 | 63281346 | 63281480 | 63281346 | 63281383 | 63281460 | 63281466 | 63281480 |
| chr2 | 452 | 63281460 | 63281559 | 63281460 | 63281466 | 63281480 | 63281542 | 63281559 |
| chr2 | 453 | 63281466 | 63281565 | 63281466 | 63281480 | 63281542 | 63281559 | 63281565 |
| chr2 | 454 | 63285000 | 63285053 | 63285000 | 63285014 | 63285028 | 63285040 | 63285053 |
| chr2 | 455 | 63285028 | 63285070 | 63285028 | 63285040 | 63285053 | 63285055 | 63285070 |
| chr2 | 456 | 63285040 | 63285097 | 63285040 | 63285053 | 63285055 | 63285070 | 63285097 |
| chr2 | 457 | 63285053 | 63285133 | 63285053 | 63285055 | 63285070 | 63285097 | 63285133 |
| chr2 | 458 | 63285055 | 63285165 | 63285055 | 63285070 | 63285097 | 63285133 | 63285165 |
| chr2 | 459 | 63285070 | 63285173 | 63285070 | 63285097 | 63285133 | 63285165 | 63285173 |
| chr2 | 460 | 63285133 | 63285200 | 63285133 | 63285165 | 63285173 | 63285175 | 63285200 |
| chr2 | 461 | 63285165 | 63285211 | 63285165 | 63285173 | 63285175 | 63285200 | 63285211 |
| chr2 | 462 | 63285173 | 63285223 | 63285173 | 63285175 | 63285200 | 63285211 | 63285223 |
| chr2 | 463 | 63285365 | 63285504 | 63285365 | 63285425 | 63285441 | 63285491 | 63285504 |
| chr2 | 464 | 63285506 | 63285645 | 63285506 | 63285519 | 63285535 | 63285596 | 63285645 |
| chr2 | 465 | 63285519 | 63285654 | 63285519 | 63285535 | 63285596 | 63285645 | 63285654 |
| chr2 | 466 | 63286055 | 63286106 | 63286055 | 63286069 | 63286073 | 63286075 | 63286106 |
| chr2 | 467 | 63286069 | 63286130 | 63286069 | 63286073 | 63286075 | 63286106 | 63286130 |
| chr2 | 468 | 63286073 | 63286136 | 63286073 | 63286075 | 63286106 | 63286130 | 63286136 |
| chr2 | 469 | 63286075 | 63286147 | 63286075 | 63286106 | 63286130 | 63286136 | 63286147 |
| chr2 | 470 | 63286248 | 63286301 | 63286248 | 63286268 | 63286278 | 63286294 | 63286301 |
| chr2 | 471 | 63286268 | 63286305 | 63286268 | 63286278 | 63286294 | 63286301 | 63286305 |
| chr2 | 472 | 63286358 | 63286466 | 63286358 | 63286376 | 63286424 | 63286436 | 63286466 |
| chr2 | 473 | 63286376 | 63286470 | 63286376 | 63286424 | 63286436 | 63286466 | 63286470 |
| chr2 | 474 | 66809280 | 66809347 | 66809280 | 66809282 | 66809303 | 66809327 | 66809347 |
| chr2 | 475 | 73147559 | 73147592 | 73147559 | 73147571 | 73147582 | 73147584 | 73147592 |
| chr2 | 476 | 73147571 | 73147595 | 73147571 | 73147582 | 73147584 | 73147592 | 73147595 |
| chr2 | 477 | 73147584 | 73147607 | 73147584 | 73147592 | 73147595 | 73147598 | 73147607 |
| chr2 | 478 | 73147875 | 73147906 | 73147875 | 73147881 | 73147887 | 73147899 | 73147906 |
| chr2 | 479 | 73147881 | 73147912 | 73147881 | 73147887 | 73147899 | 73147906 | 73147912 |
| chr2 | 480 | 73147887 | 73147916 | 73147887 | 73147899 | 73147906 | 73147912 | 73147916 |
| chr2 | 481 | 73147912 | 73147939 | 73147912 | 73147916 | 73147918 | 73147925 | 73147939 |
| chr2 | 482 | 73147916 | 73147942 | 73147916 | 73147918 | 73147925 | 73147939 | 73147942 |
| chr2 | 483 | 73147918 | 73147960 | 73147918 | 73147925 | 73147939 | 73147942 | 73147960 |
| chr2 | 484 | 73147925 | 73147967 | 73147925 | 73147939 | 73147942 | 73147960 | 73147967 |
| chr2 | 485 | 73147939 | 73147981 | 73147939 | 73147942 | 73147960 | 73147967 | 73147981 |
| chr2 | 486 | 73147942 | 73147998 | 73147942 | 73147960 | 73147967 | 73147981 | 73147998 |
| chr2 | 487 | 114256914 | 114256994 | 114256914 | 114256944 | 114256956 | 114256970 | 114256994 |
| chr2 | 488 | 114256944 | 114257003 | 114256944 | 114256956 | 114256970 | 114256994 | 114257003 |
| chr2 | 489 | 114256956 | 114257020 | 114256956 | 114256970 | 114256994 | 114257003 | 114257020 |
| chr2 | 490 | 114256994 | 114257036 | 114256994 | 114257003 | 114257020 | 114257028 | 114257036 |
| chr2 | 491 | 114257061 | 114257094 | 114257061 | 114257064 | 114257069 | 114257073 | 114257094 |
| chr2 | 492 | 114257069 | 114257120 | 114257069 | 114257073 | 114257094 | 114257108 | 114257120 |
| chr2 | 493 | 114257073 | 114257123 | 114257073 | 114257094 | 114257108 | 114257120 | 114257123 |
| chr2 | 494 | 114257251 | 114257299 | 114257251 | 114257266 | 114257269 | 114257280 | 114257299 |
| chr2 | 495 | 114257266 | 114257312 | 114257266 | 114257269 | 114257280 | 114257299 | 114257312 |
| chr2 | 496 | 114257269 | 114257319 | 114257269 | 114257280 | 114257299 | 114257312 | 114257319 |
| chr2 | 497 | 119599942 | 119600002 | 119599942 | 119599960 | 119599994 | 119599997 | 119600002 |
| chr2 | 498 | 119599960 | 119600007 | 119599960 | 119599994 | 119599997 | 119600002 | 119600007 |
| chr2 | 499 | 119616429 | 119616553 | 119616429 | 119616451 | 119616457 | 119616533 | 119616553 |
| chr2 | 500 | 139536953 | 139536986 | 139536953 | 139536974 | 139536979 | 139536984 | 139536986 |
| chr2 | 501 | 162283659 | 162283698 | 162283659 | 162283663 | 162283671 | 162283679 | 162283698 |
| chr2 | 502 | 176947518 | 176947655 | 176947518 | 176947544 | 176947634 | 176947652 | 176947655 |
| chr2 | 503 | 176947544 | 176947658 | 176947544 | 176947634 | 176947652 | 176947655 | 176947658 |
| chr2 | 504 | 176956377 | 176956509 | 176956377 | 176956396 | 176956452 | 176956505 | 176956509 |
| chr2 | 505 | 176956573 | 176956626 | 176956573 | 176956601 | 176956604 | 176956607 | 176956626 |
| chr2 | 506 | 176956607 | 176956645 | 176956607 | 176956626 | 176956631 | 176956643 | 176956645 |
| chr2 | 507 | 176956626 | 176956666 | 176956626 | 176956631 | 176956643 | 176956645 | 176956666 |
| chr2 | 508 | 176956631 | 176956678 | 176956631 | 176956643 | 176956645 | 176956666 | 176956678 |
| chr2 | 509 | 176956643 | 176956699 | 176956643 | 176956645 | 176956666 | 176956678 | 176956699 |
| chr2 | 510 | 176956645 | 176956706 | 176956645 | 176956666 | 176956678 | 176956699 | 176956706 |
| chr2 | 511 | 176956830 | 176956937 | 176956830 | 176956834 | 176956841 | 176956899 | 176956937 |
| chr2 | 512 | 176959305 | 176959355 | 176959305 | 176959342 | 176959345 | 176959348 | 176959355 |
| chr2 | 513 | 176959348 | 176959497 | 176959348 | 176959355 | 176959364 | 176959405 | 176959497 |
| chr2 | 514 | 176964168 | 176964243 | 176964168 | 176964175 | 176964180 | 176964194 | 176964243 |
| chr2 | 515 | 176964175 | 176964265 | 176964175 | 176964180 | 176964194 | 176964243 | 176964265 |
| chr2 | 516 | 176964180 | 176964279 | 176964180 | 176964194 | 176964243 | 176964265 | 176964279 |
| chr2 | 517 | 176964194 | 176964296 | 176964194 | 176964243 | 176964265 | 176964279 | 176964296 |
| chr2 | 518 | 176964390 | 176964456 | 176964390 | 176964404 | 176964419 | 176964448 | 176964456 |
| chr2 | 519 | 176964570 | 176964640 | 176964570 | 176964588 | 176964621 | 176964628 | 176964640 |
| chr2 | 520 | 176964739 | 176964805 | 176964739 | 176964747 | 176964769 | 176964778 | 176964805 |
| chr2 | 521 | 176964747 | 176964811 | 176964747 | 176964769 | 176964778 | 176964805 | 176964811 |
| chr2 | 522 | 176964769 | 176964834 | 176964769 | 176964778 | 176964805 | 176964811 | 176964834 |
| chr2 | 523 | 176964892 | 176964966 | 176964892 | 176964930 | 176964946 | 176964959 | 176964966 |
| chr2 | 524 | 176965125 | 176965196 | 176965125 | 176965130 | 176965140 | 176965186 | 176965196 |
| chr2 | 525 | 176965214 | 176965271 | 176965214 | 176965227 | 176965256 | 176965265 | 176965271 |
| chr2 | 526 | 176981064 | 176981132 | 176981064 | 176981079 | 176981092 | 176981094 | 176981132 |
| chr2 | 527 | 176981079 | 176981138 | 176981079 | 176981092 | 176981094 | 176981132 | 176981138 |
| chr2 | 528 | 176981380 | 176981422 | 176981380 | 176981384 | 176981402 | 176981409 | 176981422 |
| chr2 | 529 | 200327334 | 200327396 | 200327334 | 200327336 | 200327369 | 200327390 | 200327396 |
| chr2 | 530 | 200327336 | 200327413 | 200327336 | 200327369 | 200327390 | 200327396 | 200327413 |
| chr2 | 531 | 200327369 | 200327416 | 200327369 | 200327390 | 200327396 | 200327413 | 200327416 |
| chr2 | 532 | 219736387 | 219736424 | 219736387 | 219736397 | 219736400 | 219736421 | 219736424 |
| chr2 | 533 | 219736400 | 219736430 | 219736400 | 219736421 | 219736424 | 219736427 | 219736430 |
| chr2 | 534 | 219736421 | 219736439 | 219736421 | 219736424 | 219736427 | 219736430 | 219736439 |
| chr2 | 535 | 219736424 | 219736445 | 219736424 | 219736427 | 219736430 | 219736439 | 219736445 |
| chr2 | 536 | 219736427 | 219736453 | 219736427 | 219736430 | 219736439 | 219736445 | 219736453 |
| chr2 | 537 | 219736430 | 219736469 | 219736430 | 219736439 | 219736445 | 219736453 | 219736469 |
| chr2 | 538 | 219736439 | 219736471 | 219736439 | 219736445 | 219736453 | 219736469 | 219736471 |
| chr2 | 539 | 219736445 | 219736473 | 219736445 | 219736453 | 219736469 | 219736471 | 219736473 |
| chr2 | 540 | 219736490 | 219736526 | 219736490 | 219736500 | 219736502 | 219736504 | 219736526 |
| chr2 | 541 | 219736500 | 219736531 | 219736500 | 219736502 | 219736504 | 219736526 | 219736531 |
| chr2 | 542 | 219736502 | 219736545 | 219736502 | 219736504 | 219736526 | 219736531 | 219736545 |
| chr2 | 543 | 223169342 | 223169470 | 223169342 | 223169352 | 223169441 | 223169457 | 223169470 |
| chr2 | 544 | 223173032 | 223173157 | 223173032 | 223173061 | 223173068 | 223173118 | 223173157 |
| chr20 | 545 | 21491410 | 21491429 | 21491410 | 21491414 | 21491418 | 21491422 | 21491429 |
| chr20 | 546 | 21491414 | 21491436 | 21491414 | 21491418 | 21491422 | 21491429 | 21491436 |
| chr20 | 547 | 21491418 | 21491440 | 21491418 | 21491422 | 21491429 | 21491436 | 21491440 |
| chr20 | 548 | 34188787 | 34188826 | 34188787 | 34188790 | 34188797 | 34188821 | 34188826 |
| chr20 | 549 | 34188790 | 34188845 | 34188790 | 34188797 | 34188821 | 34188826 | 34188845 |
| chr20 | 550 | 34188821 | 34188881 | 34188821 | 34188826 | 34188845 | 34188866 | 34188881 |
| chr20 | 551 | 34188826 | 34188892 | 34188826 | 34188845 | 34188866 | 34188881 | 34188892 |
| chr20 | 552 | 34188971 | 34189010 | 34188971 | 34188981 | 34189004 | 34189008 | 34189010 |
| chr20 | 553 | 34189010 | 34189054 | 34189010 | 34189013 | 34189025 | 34189035 | 34189054 |
| chr20 | 554 | 37356310 | 37356340 | 37356310 | 37356314 | 37356325 | 37356330 | 37356340 |
| chr20 | 555 | 57224841 | 57224925 | 57224841 | 57224858 | 57224919 | 57224923 | 57224925 |
| chr20 | 556 | 57224967 | 57225003 | 57224967 | 57224974 | 57224986 | 57224990 | 57225003 |
| chr20 | 557 | 57224974 | 57225007 | 57224974 | 57224986 | 57224990 | 57225003 | 57225007 |
| chr20 | 558 | 57224986 | 57225009 | 57224986 | 57224990 | 57225003 | 57225007 | 57225009 |
| chr20 | 559 | 57224990 | 57225014 | 57224990 | 57225003 | 57225007 | 57225009 | 57225014 |
| chr20 | 560 | 57225054 | 57225077 | 57225054 | 57225061 | 57225067 | 57225069 | 57225077 |
| chr20 | 561 | 57225083 | 57225122 | 57225083 | 57225089 | 57225100 | 57225111 | 57225122 |
| chr21 | 562 | 36041612 | 36041663 | 36041612 | 36041626 | 36041632 | 36041646 | 36041663 |
| chr21 | 563 | 36041626 | 36041683 | 36041626 | 36041632 | 36041646 | 36041663 | 36041683 |
| chr21 | 564 | 36041632 | 36041693 | 36041632 | 36041646 | 36041663 | 36041683 | 36041693 |
| chr21 | 565 | 36041646 | 36041699 | 36041646 | 36041663 | 36041683 | 36041693 | 36041699 |
| chr21 | 566 | 36041663 | 36041701 | 36041663 | 36041683 | 36041693 | 36041699 | 36041701 |
| chr21 | 567 | 36041693 | 36041722 | 36041693 | 36041699 | 36041701 | 36041707 | 36041722 |
| chr21 | 568 | 36041699 | 36041737 | 36041699 | 36041701 | 36041707 | 36041722 | 36041737 |
| chr21 | 569 | 36041701 | 36041740 | 36041701 | 36041707 | 36041722 | 36041737 | 36041740 |
| chr21 | 570 | 36041707 | 36041750 | 36041707 | 36041722 | 36041737 | 36041740 | 36041750 |
| chr21 | 571 | 36041722 | 36041752 | 36041722 | 36041737 | 36041740 | 36041750 | 36041752 |
| chr21 | 572 | 36041737 | 36041771 | 36041737 | 36041740 | 36041750 | 36041752 | 36041771 |
| chr21 | 573 | 36041740 | 36041786 | 36041740 | 36041750 | 36041752 | 36041771 | 36041786 |
| chr21 | 574 | 36041750 | 36041788 | 36041750 | 36041752 | 36041771 | 36041786 | 36041788 |
| chr21 | 575 | 36041752 | 36041792 | 36041752 | 36041771 | 36041786 | 36041788 | 36041792 |
| chr21 | 576 | 36041771 | 36041809 | 36041771 | 36041786 | 36041788 | 36041792 | 36041809 |
| chr21 | 577 | 36041996 | 36042025 | 36041996 | 36041999 | 36042001 | 36042019 | 36042025 |
| chr21 | 578 | 36041999 | 36042030 | 36041999 | 36042001 | 36042019 | 36042025 | 36042030 |
| chr21 | 579 | 36042001 | 36042032 | 36042001 | 36042019 | 36042025 | 36042030 | 36042032 |
| chr21 | 580 | 36042019 | 36042035 | 36042019 | 36042025 | 36042030 | 36042032 | 36042035 |
| chr21 | 581 | 36042025 | 36042037 | 36042025 | 36042030 | 36042032 | 36042035 | 36042037 |
| chr21 | 582 | 36042030 | 36042044 | 36042030 | 36042032 | 36042035 | 36042037 | 36042044 |
| chr21 | 583 | 36042032 | 36042051 | 36042032 | 36042035 | 36042037 | 36042044 | 36042051 |
| chr21 | 584 | 36042035 | 36042053 | 36042035 | 36042037 | 36042044 | 36042051 | 36042053 |
| chr21 | 585 | 36042037 | 36042074 | 36042037 | 36042044 | 36042051 | 36042053 | 36042074 |
| chr21 | 586 | 36042044 | 36042085 | 36042044 | 36042051 | 36042053 | 36042074 | 36042085 |
| chr21 | 587 | 36042140 | 36042164 | 36042140 | 36042142 | 36042146 | 36042154 | 36042164 |
| chr21 | 588 | 36042142 | 36042170 | 36042142 | 36042146 | 36042154 | 36042164 | 36042170 |
| chr21 | 589 | 36042146 | 36042172 | 36042146 | 36042154 | 36042164 | 36042170 | 36042172 |
| chr21 | 590 | 38065282 | 38065355 | 38065282 | 38065290 | 38065331 | 38065351 | 38065355 |
| chr21 | 591 | 38066993 | 38067129 | 38066993 | 38067014 | 38067076 | 38067101 | 38067129 |
| chr21 | 592 | 38077258 | 38077394 | 38077258 | 38077270 | 38077347 | 38077376 | 38077394 |
| chr21 | 593 | 38077270 | 38077397 | 38077270 | 38077347 | 38077376 | 38077394 | 38077397 |
| chr21 | 594 | 38078767 | 38078871 | 38078767 | 38078776 | 38078781 | 38078851 | 38078871 |
| chr21 | 595 | 38078776 | 38078915 | 38078776 | 38078781 | 38078851 | 38078871 | 38078915 |
| chr21 | 596 | 38080567 | 38080616 | 38080567 | 38080595 | 38080597 | 38080602 | 38080616 |
| chr21 | 597 | 38080595 | 38080644 | 38080595 | 38080597 | 38080602 | 38080616 | 38080644 |
| chr21 | 598 | 38080597 | 38080670 | 38080597 | 38080602 | 38080616 | 38080644 | 38080670 |
| chr21 | 599 | 38080616 | 38080729 | 38080616 | 38080644 | 38080670 | 38080694 | 38080729 |
| chr21 | 600 | 38082794 | 38082870 | 38082794 | 38082799 | 38082836 | 38082850 | 38082870 |
| chr21 | 601 | 38082946 | 38083071 | 38082946 | 38082955 | 38083046 | 38083060 | 38083071 |
| chr21 | 602 | 38082955 | 38083079 | 38082955 | 38083046 | 38083060 | 38083071 | 38083079 |
| chr22 | 603 | 46366887 | 46367000 | 46366887 | 46366907 | 46366909 | 46366975 | 46367000 |
| chr3 | 604 | 44063586 | 44063620 | 44063586 | 44063593 | 44063602 | 44063606 | 44063620 |
| chr3 | 605 | 44063593 | 44063633 | 44063593 | 44063602 | 44063606 | 44063620 | 44063633 |
| chr3 | 606 | 44063602 | 44063638 | 44063602 | 44063606 | 44063620 | 44063633 | 44063638 |
| chr3 | 607 | 44063606 | 44063643 | 44063606 | 44063620 | 44063633 | 44063638 | 44063643 |
| chr3 | 608 | 44063657 | 44063686 | 44063657 | 44063660 | 44063662 | 44063682 | 44063686 |
| chr3 | 609 | 62362921 | 62362968 | 62362921 | 62362924 | 62362939 | 62362965 | 62362968 |
| chr3 | 610 | 62362924 | 62362986 | 62362924 | 62362939 | 62362965 | 62362968 | 62362986 |
| chr3 | 611 | 129693195 | 129693240 | 129693195 | 129693201 | 129693212 | 129693224 | 129693240 |
| chr3 | 612 | 129693201 | 129693244 | 129693201 | 129693212 | 129693224 | 129693240 | 129693244 |
| chr3 | 613 | 129693212 | 129693258 | 129693212 | 129693224 | 129693240 | 129693244 | 129693258 |
| chr3 | 614 | 129693224 | 129693262 | 129693224 | 129693240 | 129693244 | 129693258 | 129693262 |
| chr3 | 615 | 129693240 | 129693303 | 129693240 | 129693244 | 129693258 | 129693262 | 129693303 |
| chr3 | 616 | 129693244 | 129693305 | 129693244 | 129693258 | 129693262 | 129693303 | 129693305 |
| chr3 | 617 | 129693258 | 129693313 | 129693258 | 129693262 | 129693303 | 129693305 | 129693313 |
| chr3 | 618 | 129693262 | 129693344 | 129693262 | 129693303 | 129693305 | 129693313 | 129693344 |
| chr3 | 619 | 129693303 | 129693350 | 129693303 | 129693305 | 129693313 | 129693344 | 129693350 |
| chr3 | 620 | 129693305 | 129693352 | 129693305 | 129693313 | 129693344 | 129693350 | 129693352 |
| chr3 | 621 | 129693313 | 129693355 | 129693313 | 129693344 | 129693350 | 129693352 | 129693355 |
| chr3 | 622 | 129693383 | 129693406 | 129693383 | 129693385 | 129693390 | 129693396 | 129693406 |
| chr3 | 623 | 129693385 | 129693412 | 129693385 | 129693390 | 129693396 | 129693406 | 129693412 |
| chr3 | 624 | 129693390 | 129693425 | 129693390 | 129693396 | 129693406 | 129693412 | 129693425 |
| chr3 | 625 | 129693396 | 129693437 | 129693396 | 129693406 | 129693412 | 129693425 | 129693437 |
| chr3 | 626 | 129693483 | 129693513 | 129693483 | 129693487 | 129693489 | 129693500 | 129693513 |
| chr3 | 627 | 129693487 | 129693518 | 129693487 | 129693489 | 129693500 | 129693513 | 129693518 |
| chr3 | 628 | 129693489 | 129693521 | 129693489 | 129693500 | 129693513 | 129693518 | 129693521 |
| chr3 | 629 | 129693574 | 129693620 | 129693574 | 129693586 | 129693607 | 129693613 | 129693620 |
| chr3 | 630 | 129693586 | 129693628 | 129693586 | 129693607 | 129693613 | 129693620 | 129693628 |
| chr3 | 631 | 129693607 | 129693635 | 129693607 | 129693613 | 129693620 | 129693628 | 129693635 |
| chr3 | 632 | 129693613 | 129693672 | 129693613 | 129693620 | 129693628 | 129693635 | 129693672 |
| chr3 | 633 | 129693620 | 129693692 | 129693620 | 129693628 | 129693635 | 129693672 | 129693692 |
| chr3 | 634 | 129693628 | 129693698 | 129693628 | 129693635 | 129693672 | 129693692 | 129693698 |
| chr3 | 635 | 129693635 | 129693705 | 129693635 | 129693672 | 129693692 | 129693698 | 129693705 |
| chr3 | 636 | 129693672 | 129693731 | 129693672 | 129693692 | 129693698 | 129693705 | 129693731 |
| chr3 | 637 | 138658345 | 138658411 | 138658345 | 138658371 | 138658390 | 138658405 | 138658411 |
| chr3 | 638 | 138658371 | 138658415 | 138658371 | 138658390 | 138658405 | 138658411 | 138658415 |
| chr3 | 639 | 138658597 | 138658622 | 138658597 | 138658608 | 138658614 | 138658617 | 138658622 |
| chr3 | 640 | 138658608 | 138658626 | 138658608 | 138658614 | 138658617 | 138658622 | 138658626 |
| chr3 | 641 | 138658614 | 138658630 | 138658614 | 138658617 | 138658622 | 138658626 | 138658630 |
| chr3 | 642 | 138658617 | 138658646 | 138658617 | 138658622 | 138658626 | 138658630 | 138658646 |
| chr3 | 643 | 138658622 | 138658655 | 138658622 | 138658626 | 138658630 | 138658646 | 138658655 |
| chr3 | 644 | 138658626 | 138658675 | 138658626 | 138658630 | 138658646 | 138658655 | 138658675 |
| chr3 | 645 | 147109203 | 147109284 | 147109203 | 147109209 | 147109226 | 147109241 | 147109284 |
| chr3 | 646 | 147139248 | 147139325 | 147139248 | 147139251 | 147139272 | 147139278 | 147139325 |
| chr3 | 647 | 157812334 | 157812380 | 157812334 | 157812354 | 157812358 | 157812369 | 157812380 |
| chr3 | 648 | 157812654 | 157812721 | 157812654 | 157812677 | 157812688 | 157812718 | 157812721 |
| chr3 | 649 | 157812677 | 157812741 | 157812677 | 157812688 | 157812718 | 157812721 | 157812741 |
| chr3 | 650 | 157812688 | 157812752 | 157812688 | 157812718 | 157812721 | 157812741 | 157812752 |
| chr3 | 651 | 157812718 | 157812763 | 157812718 | 157812721 | 157812741 | 157812752 | 157812763 |
| chr3 | 652 | 157813481 | 157813617 | 157813481 | 157813488 | 157813606 | 157813609 | 157813617 |
| chr3 | 653 | 194208452 | 194208468 | 194208452 | 194208455 | 194208457 | 194208459 | 194208468 |
| chr4 | 654 | 54970235 | 54970346 | 54970235 | 54970254 | 54970309 | 54970327 | 54970346 |
| chr4 | 655 | 85402465 | 85402590 | 85402465 | 85402491 | 85402497 | 85402511 | 85402590 |
| chr4 | 656 | 85418288 | 85418409 | 85418288 | 85418302 | 85418371 | 85418386 | 85418409 |
| chr4 | 657 | 85418302 | 85418413 | 85418302 | 85418371 | 85418386 | 85418409 | 85418413 |
| chr4 | 658 | 94755706 | 94755789 | 94755706 | 94755722 | 94755751 | 94755787 | 94755789 |
| chr4 | 659 | 94755871 | 94755933 | 94755871 | 94755903 | 94755918 | 94755930 | 94755933 |
| chr4 | 660 | 94755903 | 94755975 | 94755903 | 94755918 | 94755930 | 94755933 | 94755975 |
| chr4 | 661 | 111554255 | 111554385 | 111554255 | 111554280 | 111554290 | 111554336 | 111554385 |
| chr4 | 662 | 111554915 | 111555003 | 111554915 | 111554966 | 111554970 | 111554988 | 111555003 |
| chr4 | 663 | 111555070 | 111555152 | 111555070 | 111555083 | 111555100 | 111555102 | 111555152 |
| chr4 | 664 | 111555290 | 111555377 | 111555290 | 111555314 | 111555324 | 111555329 | 111555377 |
| chr4 | 665 | 140200445 | 140200580 | 140200445 | 140200520 | 140200522 | 140200545 | 140200580 |
| chr4 | 666 | 154709597 | 154709633 | 154709597 | 154709607 | 154709612 | 154709617 | 154709633 |
| chr4 | 667 | 154709607 | 154709640 | 154709607 | 154709612 | 154709617 | 154709633 | 154709640 |
| chr4 | 668 | 154713895 | 154713927 | 154713895 | 154713911 | 154713913 | 154713917 | 154713927 |
| chr4 | 669 | 155663225 | 155663311 | 155663225 | 155663234 | 155663254 | 155663263 | 155663311 |
| chr4 | 670 | 155663234 | 155663335 | 155663234 | 155663254 | 155663263 | 155663311 | 155663335 |
| chr4 | 671 | 155663254 | 155663346 | 155663254 | 155663263 | 155663311 | 155663335 | 155663346 |
| chr4 | 672 | 155663263 | 155663396 | 155663263 | 155663311 | 155663335 | 155663346 | 155663396 |
| chr4 | 673 | 155663490 | 155663546 | 155663490 | 155663514 | 155663529 | 155663540 | 155663546 |
| chr4 | 674 | 174430459 | 174430526 | 174430459 | 174430483 | 174430485 | 174430487 | 174430526 |
| chr4 | 675 | 174430487 | 174430555 | 174430487 | 174430526 | 174430528 | 174430542 | 174430555 |
| chr4 | 676 | 174430602 | 174430640 | 174430602 | 174430609 | 174430614 | 174430627 | 174430640 |
| chr4 | 677 | 174430609 | 174430656 | 174430609 | 174430614 | 174430627 | 174430640 | 174430656 |
| chr4 | 678 | 174430614 | 174430662 | 174430614 | 174430627 | 174430640 | 174430656 | 174430662 |
| chr4 | 679 | 174430627 | 174430671 | 174430627 | 174430640 | 174430656 | 174430662 | 174430671 |
| chr4 | 680 | 174430640 | 174430675 | 174430640 | 174430656 | 174430662 | 174430671 | 174430675 |
| chr4 | 681 | 174430656 | 174430698 | 174430656 | 174430662 | 174430671 | 174430675 | 174430698 |
| chr4 | 682 | 174430662 | 174430703 | 174430662 | 174430671 | 174430675 | 174430698 | 174430703 |
| chr4 | 683 | 174430671 | 174430711 | 174430671 | 174430675 | 174430698 | 174430703 | 174430711 |
| chr4 | 684 | 174430675 | 174430718 | 174430675 | 174430698 | 174430703 | 174430711 | 174430718 |
| chr4 | 685 | 174430776 | 174430800 | 174430776 | 174430786 | 174430789 | 174430793 | 174430800 |
| chr4 | 686 | 174430793 | 174430815 | 174430793 | 174430800 | 174430811 | 174430813 | 174430815 |
| chr4 | 687 | 174430957 | 174431009 | 174430957 | 174430981 | 174430984 | 174431004 | 174431009 |
| chr5 | 688 | 1876200 | 1876286 | 1876200 | 1876208 | 1876213 | 1876215 | 1876286 |
| chr5 | 689 | 1876208 | 1876290 | 1876208 | 1876213 | 1876215 | 1876286 | 1876290 |
| chr5 | 690 | 1878228 | 1878310 | 1878228 | 1878241 | 1878256 | 1878265 | 1878310 |
| chr5 | 691 | 1878310 | 1878401 | 1878310 | 1878322 | 1878350 | 1878376 | 1878401 |
| chr5 | 692 | 2738995 | 2739096 | 2738995 | 2739001 | 2739017 | 2739065 | 2739096 |
| chr5 | 693 | 2740584 | 2740718 | 2740584 | 2740642 | 2740652 | 2740666 | 2740718 |
| chr5 | 694 | 2747857 | 2747985 | 2747857 | 2747869 | 2747922 | 2747973 | 2747985 |
| chr5 | 695 | 2747869 | 2747992 | 2747869 | 2747922 | 2747973 | 2747985 | 2747992 |
| chr5 | 696 | 2754908 | 2755002 | 2754908 | 2754939 | 2754943 | 2754971 | 2755002 |
| chr5 | 697 | 2754939 | 2755004 | 2754939 | 2754943 | 2754971 | 2755002 | 2755004 |
| chr5 | 698 | 2754943 | 2755029 | 2754943 | 2754971 | 2755002 | 2755004 | 2755029 |
| chr5 | 699 | 2755469 | 2755550 | 2755469 | 2755475 | 2755499 | 2755540 | 2755550 |
| chr5 | 700 | 2755729 | 2755820 | 2755729 | 2755736 | 2755748 | 2755767 | 2755820 |
| chr5 | 701 | 3596645 | 3596679 | 3596645 | 3596647 | 3596654 | 3596660 | 3596679 |
| chr5 | 702 | 3596647 | 3596683 | 3596647 | 3596654 | 3596660 | 3596679 | 3596683 |
| chr5 | 703 | 3596654 | 3596690 | 3596654 | 3596660 | 3596679 | 3596683 | 3596690 |
| chr5 | 704 | 54518590 | 54518698 | 54518590 | 54518595 | 54518606 | 54518667 | 54518698 |
| chr5 | 705 | 54518595 | 54518704 | 54518595 | 54518606 | 54518667 | 54518698 | 54518704 |
| chr5 | 706 | 54518606 | 54518745 | 54518606 | 54518667 | 54518698 | 54518704 | 54518745 |
| chr5 | 707 | 54518667 | 54518798 | 54518667 | 54518698 | 54518704 | 54518745 | 54518798 |
| chr5 | 708 | 54518698 | 54518834 | 54518698 | 54518704 | 54518745 | 54518798 | 54518834 |
| chr5 | 709 | 54518798 | 54518919 | 54518798 | 54518834 | 54518883 | 54518892 | 54518919 |
| chr5 | 710 | 54518834 | 54518961 | 54518834 | 54518883 | 54518892 | 54518919 | 54518961 |
| chr5 | 711 | 54518883 | 54519017 | 54518883 | 54518892 | 54518919 | 54518961 | 54519017 |
| chr5 | 712 | 54518892 | 54519023 | 54518892 | 54518919 | 54518961 | 54519017 | 54519023 |
| chr5 | 713 | 54518919 | 54519055 | 54518919 | 54518961 | 54519017 | 54519023 | 54519055 |
| chr5 | 714 | 54518961 | 54519061 | 54518961 | 54519017 | 54519023 | 54519055 | 54519061 |
| chr5 | 715 | 54519017 | 54519064 | 54519017 | 54519023 | 54519055 | 54519061 | 54519064 |
| chr5 | 716 | 54519076 | 54519107 | 54519076 | 54519084 | 54519087 | 54519101 | 54519107 |
| chr5 | 717 | 54519084 | 54519126 | 54519084 | 54519087 | 54519101 | 54519107 | 54519126 |
| chr5 | 718 | 54519087 | 54519159 | 54519087 | 54519101 | 54519107 | 54519126 | 54519159 |
| chr5 | 719 | 54519101 | 54519169 | 54519101 | 54519107 | 54519126 | 54519159 | 54519169 |
| chr5 | 720 | 54519107 | 54519174 | 54519107 | 54519126 | 54519159 | 54519169 | 54519174 |
| chr5 | 721 | 54519126 | 54519178 | 54519126 | 54519159 | 54519169 | 54519174 | 54519178 |
| chr5 | 722 | 54519159 | 54519185 | 54519159 | 54519169 | 54519174 | 54519178 | 54519185 |
| chr5 | 723 | 54519169 | 54519216 | 54519169 | 54519174 | 54519178 | 54519185 | 54519216 |
| chr5 | 724 | 54519174 | 54519218 | 54519174 | 54519178 | 54519185 | 54519216 | 54519218 |
| chr5 | 725 | 54519178 | 54519238 | 54519178 | 54519185 | 54519216 | 54519218 | 54519238 |
| chr5 | 726 | 54519185 | 54519243 | 54519185 | 54519216 | 54519218 | 54519238 | 54519243 |
| chr5 | 727 | 54519218 | 54519265 | 54519218 | 54519238 | 54519243 | 54519262 | 54519265 |
| chr5 | 728 | 54519284 | 54519333 | 54519284 | 54519290 | 54519298 | 54519307 | 54519333 |
| chr5 | 729 | 54519290 | 54519348 | 54519290 | 54519298 | 54519307 | 54519333 | 54519348 |
| chr5 | 730 | 54519298 | 54519351 | 54519298 | 54519307 | 54519333 | 54519348 | 54519351 |
| chr5 | 731 | 54519445 | 54519503 | 54519445 | 54519466 | 54519472 | 54519485 | 54519503 |
| chr5 | 732 | 76923741 | 76923806 | 76923741 | 76923745 | 76923753 | 76923794 | 76923806 |
| chr5 | 733 | 76923745 | 76923817 | 76923745 | 76923753 | 76923794 | 76923806 | 76923817 |
| chr5 | 734 | 76923753 | 76923857 | 76923753 | 76923794 | 76923806 | 76923817 | 76923857 |
| chr5 | 735 | 76923806 | 76923893 | 76923806 | 76923817 | 76923857 | 76923888 | 76923893 |
| chr5 | 736 | 76923817 | 76923899 | 76923817 | 76923857 | 76923888 | 76923893 | 76923899 |
| chr5 | 737 | 76923985 | 76924043 | 76923985 | 76923988 | 76924025 | 76924041 | 76924043 |
| chr5 | 738 | 115151479 | 115151534 | 115151479 | 115151505 | 115151517 | 115151526 | 115151534 |
| chr5 | 739 | 115152117 | 115152172 | 115152117 | 115152119 | 115152137 | 115152153 | 115152172 |
| chr5 | 740 | 115152119 | 115152190 | 115152119 | 115152137 | 115152153 | 115152172 | 115152190 |
| chr5 | 741 | 115152137 | 115152198 | 115152137 | 115152153 | 115152172 | 115152190 | 115152198 |
| chr5 | 742 | 115152153 | 115152215 | 115152153 | 115152172 | 115152190 | 115152198 | 115152215 |
| chr5 | 743 | 115152172 | 115152239 | 115152172 | 115152190 | 115152198 | 115152215 | 115152239 |
| chr5 | 744 | 115152190 | 115152244 | 115152190 | 115152198 | 115152215 | 115152239 | 115152244 |
| chr5 | 745 | 115152198 | 115152255 | 115152198 | 115152215 | 115152239 | 115152244 | 115152255 |
| chr5 | 746 | 115152215 | 115152258 | 115152215 | 115152239 | 115152244 | 115152255 | 115152258 |
| chr5 | 747 | 115152431 | 115152479 | 115152431 | 115152436 | 115152460 | 115152466 | 115152479 |
| chr5 | 748 | 115152492 | 115152520 | 115152492 | 115152494 | 115152504 | 115152513 | 115152520 |
| chr5 | 749 | 115152513 | 115152548 | 115152513 | 115152520 | 115152522 | 115152539 | 115152548 |
| chr5 | 750 | 115152548 | 115152574 | 115152548 | 115152551 | 115152563 | 115152565 | 115152574 |
| chr5 | 751 | 115152551 | 115152576 | 115152551 | 115152563 | 115152565 | 115152574 | 115152576 |
| chr5 | 752 | 115152563 | 115152598 | 115152563 | 115152565 | 115152574 | 115152576 | 115152598 |
| chr5 | 753 | 115152565 | 115152601 | 115152565 | 115152574 | 115152576 | 115152598 | 115152601 |
| chr5 | 754 | 115152574 | 115152603 | 115152574 | 115152576 | 115152598 | 115152601 | 115152603 |
| chr5 | 755 | 115152576 | 115152612 | 115152576 | 115152598 | 115152601 | 115152603 | 115152612 |
| chr5 | 756 | 115152598 | 115152616 | 115152598 | 115152601 | 115152603 | 115152612 | 115152616 |
| chr5 | 757 | 115152601 | 115152624 | 115152601 | 115152603 | 115152612 | 115152616 | 115152624 |
| chr5 | 758 | 115152603 | 115152634 | 115152603 | 115152612 | 115152616 | 115152624 | 115152634 |
| chr5 | 759 | 115152660 | 115152702 | 115152660 | 115152663 | 115152671 | 115152687 | 115152702 |
| chr5 | 760 | 122431100 | 122431134 | 122431100 | 122431115 | 122431121 | 122431132 | 122431134 |
| chr5 | 761 | 134366779 | 134366919 | 134366779 | 134366798 | 134366831 | 134366914 | 134366919 |
| chr5 | 762 | 134366798 | 134366932 | 134366798 | 134366831 | 134366914 | 134366919 | 134366932 |
| chr5 | 763 | 134374533 | 134374598 | 134374533 | 134374535 | 134374554 | 134374559 | 134374598 |
| chr5 | 765 | 134374598 | 134374648 | 134374598 | 134374608 | 134374611 | 134374644 | 134374648 |
| chr5 | 766 | 134374611 | 134374656 | 134374611 | 134374644 | 134374648 | 134374653 | 134374656 |
| chr5 | 767 | 134374644 | 134374661 | 134374644 | 134374648 | 134374653 | 134374656 | 134374661 |
| chr5 | 768 | 134375837 | 134375935 | 134375837 | 134375885 | 134375904 | 134375923 | 134375935 |
| chr5 | 769 | 134376361 | 134376489 | 134376361 | 134376381 | 134376442 | 134376475 | 134376489 |
| chr5 | 770 | 134376672 | 134376732 | 134376672 | 134376686 | 134376701 | 134376713 | 134376732 |
| chr5 | 771 | 170740937 | 170741024 | 170740937 | 170740953 | 170740958 | 170740991 | 170741024 |
| chr5 | 772 | 170740953 | 170741026 | 170740953 | 170740958 | 170740991 | 170741024 | 170741026 |
| chr5 | 773 | 170740958 | 170741054 | 170740958 | 170740991 | 170741024 | 170741026 | 170741054 |
| chr5 | 774 | 170740991 | 170741105 | 170740991 | 170741024 | 170741026 | 170741054 | 170741105 |
| chr5 | 775 | 172672550 | 172672672 | 172672550 | 172672567 | 172672614 | 172672649 | 172672672 |
| chr5 | 776 | 172672996 | 172673096 | 172672996 | 172673009 | 172673043 | 172673080 | 172673096 |
| chr5 | 777 | 172673009 | 172673098 | 172673009 | 172673043 | 172673080 | 172673096 | 172673098 |
| chr5 | 778 | 174158836 | 174158912 | 174158836 | 174158853 | 174158859 | 174158866 | 174158912 |
| chr5 | 779 | 174158853 | 174158920 | 174158853 | 174158859 | 174158866 | 174158912 | 174158920 |
| chr5 | 780 | 174158859 | 174158925 | 174158859 | 174158866 | 174158912 | 174158920 | 174158925 |
| chr5 | 781 | 174159021 | 174159088 | 174159021 | 174159034 | 174159039 | 174159058 | 174159088 |
| chr5 | 782 | 174159034 | 174159114 | 174159034 | 174159039 | 174159058 | 174159088 | 174159114 |
| chr5 | 783 | 174159039 | 174159120 | 174159039 | 174159058 | 174159088 | 174159114 | 174159120 |
| chr6 | 784 | 392628 | 392650 | 392628 | 392632 | 392638 | 392648 | 392650 |
| chr6 | 785 | 392648 | 392688 | 392648 | 392650 | 392652 | 392661 | 392688 |
| chr6 | 786 | 1384523 | 1384582 | 1384523 | 1384557 | 1384561 | 1384576 | 1384582 |
| chr6 | 787 | 1384755 | 1384869 | 1384755 | 1384780 | 1384806 | 1384837 | 1384869 |
| chr6 | 788 | 1384780 | 1384894 | 1384780 | 1384806 | 1384837 | 1384869 | 1384894 |
| chr6 | 789 | 1384806 | 1384904 | 1384806 | 1384837 | 1384869 | 1384894 | 1384904 |
| chr6 | 790 | 1385268 | 1385373 | 1385268 | 1385273 | 1385282 | 1385351 | 1385373 |
| chr6 | 791 | 1393591 | 1393675 | 1393591 | 1393607 | 1393611 | 1393626 | 1393675 |
| chr6 | 792 | 1393607 | 1393677 | 1393607 | 1393611 | 1393626 | 1393675 | 1393677 |
| chr6 | 793 | 6003908 | 6003947 | 6003908 | 6003913 | 6003915 | 6003943 | 6003947 |
| chr6 | 794 | 6004240 | 6004274 | 6004240 | 6004249 | 6004252 | 6004268 | 6004274 |
| chr6 | 795 | 10381523 | 10381571 | 10381523 | 10381534 | 10381559 | 10381569 | 10381571 |
| chr6 | 796 | 10381534 | 10381573 | 10381534 | 10381559 | 10381569 | 10381571 | 10381573 |
| chr6 | 797 | 10381571 | 10381629 | 10381571 | 10381573 | 10381591 | 10381594 | 10381629 |
| chr6 | 798 | 10381573 | 10381636 | 10381573 | 10381591 | 10381594 | 10381629 | 10381636 |
| chr6 | 799 | 10381591 | 10381641 | 10381591 | 10381594 | 10381629 | 10381636 | 10381641 |
| chr6 | 800 | 10381594 | 10381667 | 10381594 | 10381629 | 10381636 | 10381641 | 10381667 |
| chr6 | 801 | 10381629 | 10381676 | 10381629 | 10381636 | 10381641 | 10381667 | 10381676 |
| chr6 | 802 | 10381636 | 10381695 | 10381636 | 10381641 | 10381667 | 10381676 | 10381695 |
| chr6 | 803 | 10381641 | 10381698 | 10381641 | 10381667 | 10381676 | 10381695 | 10381698 |
| chr6 | 804 | 10381695 | 10381764 | 10381695 | 10381698 | 10381715 | 10381726 | 10381764 |
| chr6 | 805 | 10381698 | 10381794 | 10381698 | 10381715 | 10381726 | 10381764 | 10381794 |
| chr6 | 806 | 10381867 | 10381943 | 10381867 | 10381875 | 10381882 | 10381907 | 10381943 |
| chr6 | 807 | 10381875 | 10381957 | 10381875 | 10381882 | 10381907 | 10381943 | 10381957 |
| chr6 | 808 | 10382200 | 10382264 | 10382200 | 10382218 | 10382224 | 10382226 | 10382264 |
| chr6 | 809 | 10382218 | 10382281 | 10382218 | 10382224 | 10382226 | 10382264 | 10382281 |
| chr6 | 810 | 10382316 | 10382443 | 10382316 | 10382322 | 10382344 | 10382353 | 10382443 |
| chr6 | 811 | 10382322 | 10382452 | 10382322 | 10382344 | 10382353 | 10382443 | 10382452 |
| chr6 | 812 | 10385515 | 10385630 | 10385515 | 10385548 | 10385554 | 10385601 | 10385630 |
| chr6 | 813 | 10385781 | 10385903 | 10385781 | 10385837 | 10385839 | 10385894 | 10385903 |
| chr6 | 814 | 10385837 | 10385914 | 10385837 | 10385839 | 10385894 | 10385903 | 10385914 |
| chr6 | 815 | 10385839 | 10385925 | 10385839 | 10385894 | 10385903 | 10385914 | 10385925 |
| chr6 | 816 | 10386160 | 10386259 | 10386160 | 10386163 | 10386167 | 10386226 | 10386259 |
| chr6 | 817 | 10390101 | 10390195 | 10390101 | 10390135 | 10390171 | 10390186 | 10390195 |
| chr6 | 818 | 10390135 | 10390204 | 10390135 | 10390171 | 10390186 | 10390195 | 10390204 |
| chr6 | 819 | 10390243 | 10390344 | 10390243 | 10390247 | 10390306 | 10390332 | 10390344 |
| chr6 | 820 | 10390949 | 10391017 | 10390949 | 10390961 | 10390968 | 10391002 | 10391017 |
| chr6 | 821 | 10391341 | 10391433 | 10391341 | 10391353 | 10391382 | 10391412 | 10391433 |
| chr6 | 822 | 10391571 | 10391634 | 10391571 | 10391582 | 10391627 | 10391632 | 10391634 |
| chr6 | 823 | 10391582 | 10391683 | 10391582 | 10391627 | 10391632 | 10391634 | 10391683 |
| chr6 | 824 | 10417577 | 10417631 | 10417577 | 10417583 | 10417611 | 10417625 | 10417631 |
| chr6 | 825 | 10421419 | 10421460 | 10421419 | 10421426 | 10421442 | 10421453 | 10421460 |
| chr6 | 826 | 10421426 | 10421483 | 10421426 | 10421442 | 10421453 | 10421460 | 10421483 |
| chr6 | 827 | 10421442 | 10421486 | 10421442 | 10421453 | 10421460 | 10421483 | 10421486 |
| chr6 | 828 | 10421453 | 10421503 | 10421453 | 10421460 | 10421483 | 10421486 | 10421503 |
| chr6 | 829 | 10421460 | 10421508 | 10421460 | 10421483 | 10421486 | 10421503 | 10421508 |
| chr6 | 830 | 10421513 | 10421559 | 10421513 | 10421521 | 10421543 | 10421549 | 10421559 |
| chr6 | 831 | 10421521 | 10421563 | 10421521 | 10421543 | 10421549 | 10421559 | 10421563 |
| chr6 | 832 | 10421543 | 10421568 | 10421543 | 10421549 | 10421559 | 10421563 | 10421568 |
| chr6 | 833 | 10421549 | 10421573 | 10421549 | 10421559 | 10421563 | 10421568 | 10421573 |
| chr6 | 834 | 10421559 | 10421596 | 10421559 | 10421563 | 10421568 | 10421573 | 10421596 |
| chr6 | 835 | 10421563 | 10421619 | 10421563 | 10421568 | 10421573 | 10421596 | 10421619 |
| chr6 | 836 | 10421573 | 10421631 | 10421573 | 10421596 | 10421619 | 10421628 | 10421631 |
| chr6 | 837 | 10421628 | 10421681 | 10421628 | 10421631 | 10421670 | 10421674 | 10421681 |
| chr6 | 838 | 10422544 | 10422610 | 10422544 | 10422568 | 10422570 | 10422578 | 10422610 |
| chr6 | 839 | 26240855 | 26240881 | 26240855 | 26240860 | 26240863 | 26240874 | 26240881 |
| chr6 | 840 | 26614107 | 26614152 | 26614107 | 26614142 | 26614147 | 26614150 | 26614152 |
| chr6 | 841 | 50791363 | 50791414 | 50791363 | 50791369 | 50791385 | 50791392 | 50791414 |
| chr6 | 842 | 50793351 | 50793410 | 50793351 | 50793354 | 50793380 | 50793390 | 50793410 |
| chr6 | 843 | 50810430 | 50810534 | 50810430 | 50810432 | 50810510 | 50810524 | 50810534 |
| chr6 | 844 | 50810966 | 50811078 | 50810966 | 50810988 | 50810993 | 50811047 | 50811078 |
| chr6 | 845 | 50813698 | 50813801 | 50813698 | 50813740 | 50813771 | 50813785 | 50813801 |
| chr6 | 846 | 50813740 | 50813859 | 50813740 | 50813771 | 50813785 | 50813801 | 50813859 |
| chr6 | 847 | 100915512 | 100915630 | 100915512 | 100915532 | 100915603 | 100915610 | 100915630 |
| chr6 | 848 | 106429019 | 106429122 | 106429019 | 106429065 | 106429112 | 106429118 | 106429122 |
| chr6 | 849 | 106429065 | 106429129 | 106429065 | 106429112 | 106429118 | 106429122 | 106429129 |
| chr6 | 850 | 106429218 | 106429302 | 106429218 | 106429273 | 106429294 | 106429300 | 106429302 |
| chr6 | 851 | 106429408 | 106429443 | 106429408 | 106429414 | 106429428 | 106429436 | 106429443 |
| chr6 | 852 | 106429414 | 106429458 | 106429414 | 106429428 | 106429436 | 106429443 | 106429458 |
| chr6 | 853 | 106429458 | 106429491 | 106429458 | 106429477 | 106429480 | 106429482 | 106429491 |
| chr6 | 854 | 106429477 | 106429494 | 106429477 | 106429480 | 106429482 | 106429491 | 106429494 |
| chr6 | 855 | 108491046 | 108491110 | 108491046 | 108491050 | 108491075 | 108491108 | 108491110 |
| chr6 | 856 | 108491050 | 108491126 | 108491050 | 108491075 | 108491108 | 108491110 | 108491126 |
| chr6 | 857 | 108492406 | 108492535 | 108492406 | 108492409 | 108492437 | 108492527 | 108492535 |
| chr6 | 858 | 108495721 | 108495789 | 108495721 | 108495730 | 108495740 | 108495756 | 108495789 |
| chr6 | 859 | 108495730 | 108495804 | 108495730 | 108495740 | 108495756 | 108495789 | 108495804 |
| chr6 | 860 | 108495740 | 108495819 | 108495740 | 108495756 | 108495789 | 108495804 | 108495819 |
| chr6 | 861 | 108495804 | 108495895 | 108495804 | 108495819 | 108495849 | 108495865 | 108495895 |
| chr6 | 862 | 108495819 | 108495897 | 108495819 | 108495849 | 108495865 | 108495895 | 108495897 |
| chr6 | 863 | 108495960 | 108495985 | 108495960 | 108495968 | 108495972 | 108495979 | 108495985 |
| chr6 | 864 | 108495968 | 108496014 | 108495968 | 108495972 | 108495979 | 108495985 | 108496014 |
| chr6 | 865 | 108495972 | 108496050 | 108495972 | 108495979 | 108495985 | 108496014 | 108496050 |
| chr6 | 866 | 134210812 | 134210919 | 134210812 | 134210868 | 134210894 | 134210906 | 134210919 |
| chr6 | 867 | 134210868 | 134210946 | 134210868 | 134210894 | 134210906 | 134210919 | 134210946 |
| chr6 | 868 | 137244456 | 137244496 | 137244456 | 137244467 | 137244477 | 137244484 | 137244496 |
| chr6 | 869 | 137244477 | 137244527 | 137244477 | 137244484 | 137244496 | 137244511 | 137244527 |
| chr6 | 870 | 137244484 | 137244532 | 137244484 | 137244496 | 137244511 | 137244527 | 137244532 |
| chr6 | 871 | 137244496 | 137244576 | 137244496 | 137244511 | 137244527 | 137244532 | 137244576 |
| chr6 | 872 | 137809343 | 137809390 | 137809343 | 137809348 | 137809367 | 137809380 | 137809390 |
| chr6 | 873 | 137809348 | 137809394 | 137809348 | 137809367 | 137809380 | 137809390 | 137809394 |
| chr6 | 874 | 137809632 | 137809672 | 137809632 | 137809640 | 137809649 | 137809670 | 137809672 |
| chr6 | 875 | 137809771 | 137809830 | 137809771 | 137809790 | 137809809 | 137809828 | 137809830 |
| chr6 | 876 | 137810452 | 137810511 | 137810452 | 137810464 | 137810489 | 137810493 | 137810511 |
| chr6 | 877 | 137810464 | 137810541 | 137810464 | 137810489 | 137810493 | 137810511 | 137810541 |
| chr6 | 878 | 137810964 | 137811039 | 137810964 | 137810997 | 137811000 | 137811003 | 137811039 |
| chr6 | 879 | 137810997 | 137811059 | 137810997 | 137811000 | 137811003 | 137811039 | 137811059 |
| chr6 | 880 | 137811000 | 137811071 | 137811000 | 137811003 | 137811039 | 137811059 | 137811071 |
| chr6 | 881 | 150285860 | 150285901 | 150285860 | 150285887 | 150285890 | 150285892 | 150285901 |
| chr6 | 882 | 150286091 | 150286135 | 150286091 | 150286097 | 150286100 | 150286110 | 150286135 |
| chr6 | 883 | 150286097 | 150286137 | 150286097 | 150286100 | 150286110 | 150286135 | 150286137 |
| chr6 | 884 | 166582670 | 166582714 | 166582670 | 166582692 | 166582694 | 166582709 | 166582714 |
| chr6 | 885 | 166582692 | 166582716 | 166582692 | 166582694 | 166582709 | 166582714 | 166582716 |
| chr7 | 886 | 27204521 | 27204639 | 27204521 | 27204524 | 27204557 | 27204563 | 27204639 |
| chr7 | 887 | 27204524 | 27204663 | 27204524 | 27204557 | 27204563 | 27204639 | 27204663 |
| chr7 | 888 | 27204557 | 27204669 | 27204557 | 27204563 | 27204639 | 27204663 | 27204669 |
| chr7 | 889 | 27205089 | 27205114 | 27205089 | 27205098 | 27205102 | 27205107 | 27205114 |
| chr7 | 890 | 27205760 | 27205810 | 27205760 | 27205784 | 27205791 | 27205800 | 27205810 |
| chr7 | 891 | 27279439 | 27279501 | 27279439 | 27279454 | 27279457 | 27279468 | 27279501 |
| chr7 | 892 | 27279454 | 27279556 | 27279454 | 27279457 | 27279468 | 27279501 | 27279556 |
| chr7 | 893 | 27285041 | 27285164 | 27285041 | 27285043 | 27285059 | 27285136 | 27285164 |
| chr7 | 894 | 27290995 | 27291120 | 27290995 | 27291012 | 27291045 | 27291070 | 27291120 |
| chr7 | 895 | 27291012 | 27291133 | 27291012 | 27291045 | 27291070 | 27291120 | 27291133 |
| chr7 | 896 | 27291045 | 27291137 | 27291045 | 27291070 | 27291120 | 27291133 | 27291137 |
| chr7 | 897 | 27291070 | 27291159 | 27291070 | 27291120 | 27291133 | 27291137 | 27291159 |
| chr7 | 898 | 27291120 | 27291191 | 27291120 | 27291133 | 27291137 | 27291159 | 27291191 |
| chr7 | 899 | 27291133 | 27291201 | 27291133 | 27291137 | 27291159 | 27291191 | 27291201 |
| chr7 | 900 | 27291137 | 27291204 | 27291137 | 27291159 | 27291191 | 27291201 | 27291204 |
| chr7 | 901 | 27291159 | 27291206 | 27291159 | 27291191 | 27291201 | 27291204 | 27291206 |
| chr7 | 902 | 27291486 | 27291578 | 27291486 | 27291541 | 27291547 | 27291561 | 27291578 |
| chr7 | 903 | 27291541 | 27291585 | 27291541 | 27291547 | 27291561 | 27291578 | 27291585 |
| chr7 | 904 | 27291623 | 27291735 | 27291623 | 27291630 | 27291643 | 27291695 | 27291735 |
| chr7 | 905 | 27291826 | 27291924 | 27291826 | 27291837 | 27291855 | 27291917 | 27291924 |
| chr7 | 906 | 35297251 | 35297347 | 35297251 | 35297281 | 35297339 | 35297344 | 35297347 |
| chr7 | 907 | 35297483 | 35297524 | 35297483 | 35297485 | 35297500 | 35297516 | 35297524 |
| chr7 | 908 | 35297485 | 35297532 | 35297485 | 35297500 | 35297516 | 35297524 | 35297532 |
| chr7 | 909 | 35297500 | 35297556 | 35297500 | 35297516 | 35297524 | 35297532 | 35297556 |
| chr7 | 910 | 96651968 | 96652018 | 96651968 | 96651983 | 96651997 | 96652016 | 96652018 |
| chr7 | 911 | 96651983 | 96652024 | 96651983 | 96651997 | 96652016 | 96652018 | 96652024 |
| chr7 | 912 | 121940292 | 121940353 | 121940292 | 121940295 | 121940317 | 121940344 | 121940353 |
| chr7 | 913 | 129418430 | 129418508 | 129418430 | 129418466 | 129418470 | 129418481 | 129418508 |
| chr7 | 914 | 129423045 | 129423096 | 129423045 | 129423053 | 129423062 | 129423085 | 129423096 |
| chr7 | 915 | 129423062 | 129423115 | 129423062 | 129423085 | 129423096 | 129423101 | 129423115 |
| chr7 | 916 | 155258057 | 155258191 | 155258057 | 155258088 | 155258099 | 155258101 | 155258191 |
| chr7 | 917 | 155258088 | 155258197 | 155258088 | 155258099 | 155258101 | 155258191 | 155258197 |
| chr7 | 918 | 155258099 | 155258209 | 155258099 | 155258101 | 155258191 | 155258197 | 155258209 |
| chr7 | 919 | 155259244 | 155259304 | 155259244 | 155259252 | 155259271 | 155259284 | 155259304 |
| chr7 | 920 | 156795394 | 156795467 | 156795394 | 156795402 | 156795427 | 156795437 | 156795467 |
| chr7 | 921 | 156795402 | 156795499 | 156795402 | 156795427 | 156795437 | 156795467 | 156795499 |
| chr7 | 922 | 156795427 | 156795514 | 156795427 | 156795437 | 156795467 | 156795499 | 156795514 |
| chr7 | 923 | 156795437 | 156795527 | 156795437 | 156795467 | 156795499 | 156795514 | 156795527 |
| chr7 | 924 | 156795592 | 156795637 | 156795592 | 156795594 | 156795607 | 156795618 | 156795637 |
| chr7 | 925 | 156795594 | 156795648 | 156795594 | 156795607 | 156795618 | 156795637 | 156795648 |
| chr7 | 926 | 156797298 | 156797346 | 156797298 | 156797307 | 156797314 | 156797338 | 156797346 |
| chr7 | 927 | 156797307 | 156797390 | 156797307 | 156797314 | 156797338 | 156797346 | 156797390 |
| chr7 | 928 | 156797314 | 156797408 | 156797314 | 156797338 | 156797346 | 156797390 | 156797408 |
| chr7 | 929 | 156797338 | 156797410 | 156797338 | 156797346 | 156797390 | 156797408 | 156797410 |
| chr7 | 930 | 156797346 | 156797441 | 156797346 | 156797390 | 156797408 | 156797410 | 156797441 |
| chr7 | 931 | 156797465 | 156797536 | 156797465 | 156797468 | 156797513 | 156797515 | 156797536 |
| chr7 | 932 | 156797468 | 156797542 | 156797468 | 156797513 | 156797515 | 156797536 | 156797542 |
| chr7 | 933 | 156797513 | 156797581 | 156797513 | 156797515 | 156797536 | 156797542 | 156797581 |
| chr7 | 934 | 156797515 | 156797587 | 156797515 | 156797536 | 156797542 | 156797581 | 156797587 |
| chr7 | 935 | 156797536 | 156797623 | 156797536 | 156797542 | 156797581 | 156797587 | 156797623 |
| chr7 | 936 | 156797581 | 156797702 | 156797581 | 156797587 | 156797623 | 156797629 | 156797702 |
| chr7 | 937 | 156797587 | 156797706 | 156797587 | 156797623 | 156797629 | 156797702 | 156797706 |
| chr7 | 938 | 156797629 | 156797734 | 156797629 | 156797702 | 156797706 | 156797722 | 156797734 |
| chr7 | 939 | 156797706 | 156797793 | 156797706 | 156797722 | 156797734 | 156797781 | 156797793 |
| chr7 | 940 | 156797722 | 156797833 | 156797722 | 156797734 | 156797781 | 156797793 | 156797833 |
| chr7 | 941 | 156797734 | 156797841 | 156797734 | 156797781 | 156797793 | 156797833 | 156797841 |
| chr7 | 942 | 156797781 | 156797845 | 156797781 | 156797793 | 156797833 | 156797841 | 156797845 |
| chr7 | 943 | 156797879 | 156797911 | 156797879 | 156797881 | 156797891 | 156797905 | 156797911 |
| chr7 | 944 | 156797881 | 156797913 | 156797881 | 156797891 | 156797905 | 156797911 | 156797913 |
| chr7 | 945 | 156797891 | 156797988 | 156797891 | 156797905 | 156797911 | 156797913 | 156797988 |
| chr7 | 946 | 156798142 | 156798167 | 156798142 | 156798145 | 156798154 | 156798164 | 156798167 |
| chr7 | 947 | 156798145 | 156798171 | 156798145 | 156798154 | 156798164 | 156798167 | 156798171 |
| chr7 | 948 | 156798154 | 156798176 | 156798154 | 156798164 | 156798167 | 156798171 | 156798176 |
| chr7 | 949 | 156798167 | 156798185 | 156798167 | 156798171 | 156798176 | 156798181 | 156798185 |
| chr7 | 950 | 156798336 | 156798380 | 156798336 | 156798342 | 156798351 | 156798374 | 156798380 |
| chr7 | 951 | 156798342 | 156798383 | 156798342 | 156798351 | 156798374 | 156798380 | 156798383 |
| chr7 | 952 | 156798351 | 156798387 | 156798351 | 156798374 | 156798380 | 156798383 | 156798387 |
| chr7 | 953 | 156798387 | 156798429 | 156798387 | 156798402 | 156798406 | 156798412 | 156798429 |
| chr7 | 954 | 156798402 | 156798436 | 156798402 | 156798406 | 156798412 | 156798429 | 156798436 |
| chr7 | 955 | 156798406 | 156798445 | 156798406 | 156798412 | 156798429 | 156798436 | 156798445 |
| chr7 | 956 | 156798412 | 156798447 | 156798412 | 156798429 | 156798436 | 156798445 | 156798447 |
| chr7 | 957 | 156798468 | 156798499 | 156798468 | 156798471 | 156798474 | 156798490 | 156798499 |
| chr7 | 958 | 156798471 | 156798502 | 156798471 | 156798474 | 156798490 | 156798499 | 156798502 |
| chr7 | 959 | 156798474 | 156798527 | 156798474 | 156798490 | 156798499 | 156798502 | 156798527 |
| chr7 | 960 | 156798490 | 156798542 | 156798490 | 156798499 | 156798502 | 156798527 | 156798542 |
| chr7 | 961 | 156798499 | 156798545 | 156798499 | 156798502 | 156798527 | 156798542 | 156798545 |
| chr7 | 962 | 156798542 | 156798585 | 156798542 | 156798545 | 156798571 | 156798581 | 156798585 |
| chr7 | 963 | 156798545 | 156798595 | 156798545 | 156798571 | 156798581 | 156798585 | 156798595 |
| chr7 | 964 | 156798721 | 156798820 | 156798721 | 156798738 | 156798784 | 156798810 | 156798820 |
| chr7 | 965 | 156798738 | 156798836 | 156798738 | 156798784 | 156798810 | 156798820 | 156798836 |
| chr7 | 966 | 156798881 | 156799004 | 156798881 | 156798883 | 156798931 | 156798937 | 156799004 |
| chr7 | 967 | 156798883 | 156799018 | 156798883 | 156798931 | 156798937 | 156799004 | 156799018 |
| chr7 | 968 | 156798931 | 156799021 | 156798931 | 156798937 | 156799004 | 156799018 | 156799021 |
| chr7 | 969 | 156798937 | 156799045 | 156798937 | 156799004 | 156799018 | 156799021 | 156799045 |
| chr7 | 970 | 156799073 | 156799141 | 156799073 | 156799078 | 156799095 | 156799099 | 156799141 |
| chr7 | 971 | 156799078 | 156799148 | 156799078 | 156799095 | 156799099 | 156799141 | 156799148 |
| chr7 | 972 | 156799095 | 156799151 | 156799095 | 156799099 | 156799141 | 156799148 | 156799151 |
| chr7 | 973 | 156799099 | 156799157 | 156799099 | 156799141 | 156799148 | 156799151 | 156799157 |
| chr7 | 974 | 156799141 | 156799166 | 156799141 | 156799148 | 156799151 | 156799157 | 156799166 |
| chr7 | 975 | 156799365 | 156799428 | 156799365 | 156799371 | 156799378 | 156799393 | 156799428 |
| chr7 | 976 | 156799371 | 156799453 | 156799371 | 156799378 | 156799393 | 156799428 | 156799453 |
| chr7 | 977 | 156799378 | 156799496 | 156799378 | 156799393 | 156799428 | 156799453 | 156799496 |
| chr8 | 978 | 54789588 | 54789665 | 54789588 | 54789592 | 54789629 | 54789648 | 54789665 |
| chr8 | 979 | 54789592 | 54789669 | 54789592 | 54789629 | 54789648 | 54789665 | 54789669 |
| chr8 | 980 | 54791885 | 54791937 | 54791885 | 54791898 | 54791923 | 54791926 | 54791937 |
| chr8 | 981 | 54791898 | 54791947 | 54791898 | 54791923 | 54791926 | 54791937 | 54791947 |
| chr8 | 982 | 55367569 | 55367583 | 55367569 | 55367571 | 55367573 | 55367575 | 55367583 |
| chr8 | 983 | 70981994 | 70982027 | 70981994 | 70982003 | 70982012 | 70982014 | 70982027 |
| chr8 | 984 | 70982138 | 70982165 | 70982138 | 70982150 | 70982152 | 70982154 | 70982165 |
| chr8 | 985 | 70982150 | 70982172 | 70982150 | 70982152 | 70982154 | 70982165 | 70982172 |
| chr8 | 986 | 70984598 | 70984650 | 70984598 | 70984604 | 70984621 | 70984626 | 70984650 |
| chr8 | 987 | 97157878 | 97157939 | 97157878 | 97157899 | 97157925 | 97157927 | 97157939 |
| chr8 | 988 | 97157899 | 97157941 | 97157899 | 97157925 | 97157927 | 97157939 | 97157941 |
| chr8 | 989 | 97157925 | 97157949 | 97157925 | 97157927 | 97157939 | 97157941 | 97157949 |
| chr8 | 990 | 97157927 | 97157963 | 97157927 | 97157939 | 97157941 | 97157949 | 97157963 |
| chr8 | 991 | 97157939 | 97157965 | 97157939 | 97157941 | 97157949 | 97157963 | 97157965 |
| chr8 | 992 | 99986148 | 99986225 | 99986148 | 99986161 | 99986175 | 99986200 | 99986225 |
| chr8 | 993 | 99986161 | 99986244 | 99986161 | 99986175 | 99986200 | 99986225 | 99986244 |
| chr8 | 994 | 99986175 | 99986251 | 99986175 | 99986200 | 99986225 | 99986244 | 99986251 |
| chr8 | 995 | 99986292 | 99986389 | 99986292 | 99986321 | 99986344 | 99986368 | 99986389 |
| chr8 | 996 | 99986321 | 99986391 | 99986321 | 99986344 | 99986368 | 99986389 | 99986391 |
| chr8 | 997 | 99986391 | 99986453 | 99986391 | 99986399 | 99986403 | 99986429 | 99986453 |
| chr8 | 998 | 99986399 | 99986461 | 99986399 | 99986403 | 99986429 | 99986453 | 99986461 |
| chr8 | 999 | 99986403 | 99986471 | 99986403 | 99986429 | 99986453 | 99986461 | 99986471 |
| chr8 | 1000 | 99986429 | 99986473 | 99986429 | 99986453 | 99986461 | 99986471 | 99986473 |
| chr8 | 1001 | 99986773 | 99986812 | 99986773 | 99986783 | 99986790 | 99986792 | 99986812 |
| chr8 | 1002 | 99986783 | 99986831 | 99986783 | 99986790 | 99986792 | 99986812 | 99986831 |
| chr8 | 1003 | 99986790 | 99986839 | 99986790 | 99986792 | 99986812 | 99986831 | 99986839 |
| chr8 | 1004 | 99986792 | 99986851 | 99986792 | 99986812 | 99986831 | 99986839 | 99986851 |
| chr8 | 1005 | 99986812 | 99986857 | 99986812 | 99986831 | 99986839 | 99986851 | 99986857 |
| chr8 | 1006 | 99986831 | 99986863 | 99986831 | 99986839 | 99986851 | 99986857 | 99986863 |
| chr8 | 1007 | 99986884 | 99986964 | 99986884 | 99986908 | 99986925 | 99986942 | 99986964 |
| chr8 | 1008 | 99986942 | 99987000 | 99986942 | 99986964 | 99986969 | 99986982 | 99987000 |
| chr9 | 1009 | 37002718 | 37002742 | 37002718 | 37002723 | 37002727 | 37002733 | 37002742 |
| chr9 | 1010 | 71789407 | 71789453 | 71789407 | 71789423 | 71789425 | 71789448 | 71789453 |
| chr9 | 1011 | 71789423 | 71789456 | 71789423 | 71789425 | 71789448 | 71789453 | 71789456 |
| chr9 | 1012 | 79638054 | 79638130 | 79638054 | 79638076 | 79638117 | 79638127 | 79638130 |
| chr9 | 1013 | 96714745 | 96714821 | 96714745 | 96714764 | 96714775 | 96714780 | 96714821 |
| chr9 | 1014 | 96715792 | 96715892 | 96715792 | 96715819 | 96715843 | 96715872 | 96715892 |
| chr9 | 1015 | 100610401 | 100610466 | 100610401 | 100610408 | 100610450 | 100610454 | 100610466 |
| chr9 | 1016 | 100610872 | 100610906 | 100610872 | 100610881 | 100610890 | 100610892 | 100610906 |
| chr9 | 1017 | 100610931 | 100610992 | 100610931 | 100610942 | 100610944 | 100610962 | 100610992 |
| chr9 | 1018 | 100610942 | 100610997 | 100610942 | 100610944 | 100610962 | 100610992 | 100610997 |
| chr9 | 1019 | 100611091 | 100611132 | 100611091 | 100611098 | 100611124 | 100611130 | 100611132 |
| chr9 | 1020 | 100611098 | 100611171 | 100611098 | 100611124 | 100611130 | 100611132 | 100611171 |
| chr9 | 1021 | 100611124 | 100611178 | 100611124 | 100611130 | 100611132 | 100611171 | 100611178 |
| chr9 | 1022 | 100611130 | 100611210 | 100611130 | 100611132 | 100611171 | 100611178 | 100611210 |
| chr9 | 1023 | 100611318 | 100611395 | 100611318 | 100611361 | 100611368 | 100611383 | 100611395 |
| chr9 | 1024 | 100611361 | 100611399 | 100611361 | 100611368 | 100611383 | 100611395 | 100611399 |
| chr9 | 1025 | 100611368 | 100611401 | 100611368 | 100611383 | 100611395 | 100611399 | 100611401 |
| chr9 | 1026 | 100611383 | 100611410 | 100611383 | 100611395 | 100611399 | 100611401 | 100611410 |
| chr9 | 1027 | 100611395 | 100611428 | 100611395 | 100611399 | 100611401 | 100611410 | 100611428 |
| chr9 | 1028 | 100611401 | 100611463 | 100611401 | 100611410 | 100611428 | 100611436 | 100611463 |
| chr9 | 1029 | 100611436 | 100611504 | 100611436 | 100611463 | 100611475 | 100611487 | 100611504 |
| chr9 | 1030 | 100611463 | 100611514 | 100611463 | 100611475 | 100611487 | 100611504 | 100611514 |
| chr9 | 1031 | 100611475 | 100611516 | 100611475 | 100611487 | 100611504 | 100611514 | 100611516 |
| chr9 | 1032 | 100611487 | 100611552 | 100611487 | 100611504 | 100611514 | 100611516 | 100611552 |
| chr9 | 1033 | 100619942 | 100619995 | 100619942 | 100619985 | 100619991 | 100619993 | 100619995 |
| chr9 | 1034 | 100619993 | 100620040 | 100619993 | 100619995 | 100620013 | 100620027 | 100620040 |
| chr9 | 1035 | 100620171 | 100620218 | 100620171 | 100620175 | 100620189 | 100620191 | 100620218 |
| chr9 | 1036 | 100620608 | 100620646 | 100620608 | 100620615 | 100620634 | 100620638 | 100620646 |
| chr9 | 1037 | 100620634 | 100620674 | 100620634 | 100620638 | 100620646 | 100620649 | 100620674 |
| chr9 | 1038 | 100620638 | 100620681 | 100620638 | 100620646 | 100620649 | 100620674 | 100620681 |
| chr9 | 1039 | 100620646 | 100620684 | 100620646 | 100620649 | 100620674 | 100620681 | 100620684 |
| chr9 | 1040 | 100620684 | 100620769 | 100620684 | 100620690 | 100620696 | 100620717 | 100620769 |
| chr9 | 1041 | 126774981 | 126775015 | 126774981 | 126774990 | 126775001 | 126775013 | 126775015 |
| chr9 | 1042 | 135462861 | 135462890 | 135462861 | 135462871 | 135462882 | 135462885 | 135462890 |
| chr9 | 1043 | 135462882 | 135462908 | 135462882 | 135462885 | 135462890 | 135462903 | 135462908 |

TABLE 2
561 human universal genomic locations and CpG sites mapped to human genome, hg19. The “Start”
and “End” columns indicate the beginning and end, respectively of a range of genomic
locations within a chromosome (“Chrom.”). The SEQ ID NO refers to the nucleotide sequence
of the range of genomic locations (between the “Start” and “End” genomic locations, inclusive)

| | SEQ | | | | | | | |
|---|---|---|---|---|---|---|---|---|
| Chrom. | ID NO: | Start | End | CpG1 | CpG2 | CpG3 | CpG4 | CpG5 |
| chr1 | 1044 | 18956306 | 18956421 | 18956306 | 18956319 | 18956327 | 18956364 | 18956421 |
| chr1 | 1045 | 18956716 | 18956827 | 18956716 | 18956739 | 18956762 | 18956769 | 18956827 |
| chr1 | 1046 | 18956739 | 18956834 | 18956739 | 18956762 | 18956769 | 18956827 | 18956834 |
| chr1 | 1047 | 32238359 | 32238384 | 32238359 | 32238364 | 32238376 | 32238382 | 32238384 |
| chr1 | 1048 | 32238424 | 32238452 | 32238424 | 32238426 | 32238447 | 32238449 | 32238452 |
| chr1 | 1049 | 46913982 | 46914005 | 46913982 | 46913987 | 46913993 | 46913998 | 46914005 |
| chr1 | 1050 | 46913987 | 46914007 | 46913987 | 46913993 | 46913998 | 46914005 | 46914007 |
| chr1 | 1051 | 46913998 | 46914019 | 46913998 | 46914005 | 46914007 | 46914014 | 46914019 |
| chr1 | 1052 | 46951589 | 46951645 | 46951589 | 46951591 | 46951602 | 46951628 | 46951645 |
| chr1 | 1053 | 46951591 | 46951649 | 46951591 | 46951602 | 46951628 | 46951645 | 46951649 |
| chr1 | 1054 | 46951602 | 46951658 | 46951602 | 46951628 | 46951645 | 46951649 | 46951658 |
| chr1 | 1055 | 46951628 | 46951671 | 46951628 | 46951645 | 46951649 | 46951658 | 46951671 |
| chr1 | 1056 | 50881007 | 50881077 | 50881007 | 50881019 | 50881038 | 50881071 | 50881077 |
| chr1 | 1057 | 50881019 | 50881107 | 50881019 | 50881038 | 50881071 | 50881077 | 50881107 |
| chr1 | 1058 | 50881690 | 50881810 | 50881690 | 50881701 | 50881719 | 50881781 | 50881810 |
| chr1 | 1059 | 50881701 | 50881820 | 50881701 | 50881719 | 50881781 | 50881810 | 50881820 |
| chr1 | 1060 | 50882826 | 50882932 | 50882826 | 50882854 | 50882910 | 50882913 | 50882932 |
| chr1 | 1061 | 50882854 | 50882951 | 50882854 | 50882910 | 50882913 | 50882932 | 50882951 |
| chr1 | 1062 | 50882910 | 50882998 | 50882910 | 50882913 | 50882932 | 50882951 | 50882998 |
| chr1 | 1063 | 50883029 | 50883098 | 50883029 | 50883031 | 50883056 | 50883091 | 50883098 |
| chr1 | 1064 | 50883031 | 50883109 | 50883031 | 50883056 | 50883091 | 50883098 | 50883109 |
| chr1 | 1065 | 50883091 | 50883125 | 50883091 | 50883098 | 50883109 | 50883117 | 50883125 |
| chr1 | 1066 | 50883098 | 50883136 | 50883098 | 50883109 | 50883117 | 50883125 | 50883136 |
| chr1 | 1067 | 50891296 | 50891380 | 50891296 | 50891302 | 50891341 | 50891362 | 50891380 |
| chr1 | 1068 | 63787318 | 63787380 | 63787318 | 63787328 | 63787342 | 63787369 | 63787380 |
| chr1 | 1069 | 63787328 | 63787401 | 63787328 | 63787342 | 63787369 | 63787380 | 63787401 |
| chr1 | 1070 | 63792798 | 63792870 | 63792798 | 63792823 | 63792863 | 63792867 | 63792870 |
| chr1 | 1071 | 63795424 | 63795464 | 63795424 | 63795435 | 63795446 | 63795449 | 63795464 |
| chr1 | 1072 | 63795435 | 63795471 | 63795435 | 63795446 | 63795449 | 63795464 | 63795471 |
| chr1 | 1073 | 63795446 | 63795474 | 63795446 | 63795449 | 63795464 | 63795471 | 63795474 |
| chr1 | 1074 | 63795449 | 63795496 | 63795449 | 63795464 | 63795471 | 63795474 | 63795496 |
| chr1 | 1075 | 63795522 | 63795576 | 63795522 | 63795537 | 63795549 | 63795556 | 63795576 |
| chr1 | 1076 | 63795537 | 63795581 | 63795537 | 63795549 | 63795556 | 63795576 | 63795581 |
| chr1 | 1077 | 63795549 | 63795588 | 63795549 | 63795556 | 63795576 | 63795581 | 63795588 |
| chr1 | 1078 | 63795662 | 63795753 | 63795662 | 63795711 | 63795722 | 63795750 | 63795753 |
| chr1 | 1079 | 91192551 | 91192592 | 91192551 | 91192557 | 91192578 | 91192581 | 91192592 |
| chr1 | 1080 | 110611129 | 110611218 | 110611129 | 110611145 | 110611159 | 110611202 | 110611218 |
| chr1 | 1081 | 110611391 | 110611465 | 110611391 | 110611431 | 110611435 | 110611457 | 110611465 |
| chr1 | 1082 | 110611431 | 110611486 | 110611431 | 110611435 | 110611457 | 110611465 | 110611486 |
| chr1 | 1083 | 110611435 | 110611489 | 110611435 | 110611457 | 110611465 | 110611486 | 110611489 |
| chr1 | 1084 | 110611457 | 110611496 | 110611457 | 110611465 | 110611486 | 110611489 | 110611496 |
| chr1 | 1085 | 110611465 | 110611515 | 110611465 | 110611486 | 110611489 | 110611496 | 110611515 |
| chr1 | 1086 | 110611486 | 110611519 | 110611486 | 110611489 | 110611496 | 110611515 | 110611519 |
| chr1 | 1087 | 110612827 | 110612889 | 110612827 | 110612834 | 110612862 | 110612866 | 110612889 |
| chr1 | 1088 | 110612834 | 110612899 | 110612834 | 110612862 | 110612866 | 110612889 | 110612899 |
| chr1 | 1089 | 156389969 | 156390057 | 156389969 | 156389997 | 156390001 | 156390055 | 156390057 |
| chr1 | 1090 | 156863129 | 156863253 | 156863129 | 156863184 | 156863206 | 156863213 | 156863253 |
| chr1 | 1091 | 197888790 | 197888880 | 197888790 | 197888796 | 197888839 | 197888847 | 197888880 |
| chr1 | 1092 | 197888796 | 197888888 | 197888796 | 197888839 | 197888847 | 197888880 | 197888888 |
| chr1 | 1093 | 200009988 | 200010012 | 200009988 | 200010001 | 200010005 | 200010008 | 200010012 |
| chr10 | 1094 | 22765160 | 22765188 | 22765160 | 22765175 | 22765182 | 22765184 | 22765188 |
| chr10 | 1095 | 22765211 | 22765235 | 22765211 | 22765213 | 22765222 | 22765233 | 22765235 |
| chr10 | 1096 | 22765241 | 22765282 | 22765241 | 22765248 | 22765259 | 22765278 | 22765282 |
| chr10 | 1097 | 22765459 | 22765483 | 22765459 | 22765463 | 22765470 | 22765476 | 22765483 |
| chr10 | 1098 | 22765476 | 22765611 | 22765476 | 22765483 | 22765520 | 22765581 | 22765611 |
| chr10 | 1099 | 22765483 | 22765613 | 22765483 | 22765520 | 22765581 | 22765611 | 22765613 |
| chr10 | 1100 | 26503791 | 26503897 | 26503791 | 26503817 | 26503881 | 26503883 | 26503897 |
| chr10 | 1101 | 28030628 | 28030747 | 28030628 | 28030694 | 28030732 | 28030734 | 28030747 |
| chr10 | 1102 | 45914688 | 45914716 | 45914688 | 45914692 | 45914698 | 45914702 | 45914716 |
| chr10 | 1103 | 45914692 | 45914718 | 45914692 | 45914698 | 45914702 | 45914716 | 45914718 |
| chr10 | 1104 | 45914698 | 45914728 | 45914698 | 45914702 | 45914716 | 45914718 | 45914728 |
| chr10 | 1105 | 45914702 | 45914740 | 45914702 | 45914716 | 45914718 | 45914728 | 45914740 |
| chr10 | 1106 | 45914716 | 45914749 | 45914716 | 45914718 | 45914728 | 45914740 | 45914749 |
| chr10 | 1107 | 45914718 | 45914753 | 45914718 | 45914728 | 45914740 | 45914749 | 45914753 |
| chr10 | 1108 | 45914775 | 45914834 | 45914775 | 45914784 | 45914799 | 45914829 | 45914834 |
| chr10 | 1109 | 101291046 | 101291107 | 101291046 | 101291092 | 101291095 | 101291100 | 101291107 |
| chr10 | 1110 | 102590202 | 102590272 | 102590202 | 102590209 | 102590225 | 102590237 | 102590272 |
| chr10 | 1111 | 102590209 | 102590274 | 102590209 | 102590225 | 102590237 | 102590272 | 102590274 |
| chr10 | 1112 | 102590225 | 102590290 | 102590225 | 102590237 | 102590272 | 102590274 | 102590290 |
| chr10 | 1113 | 102590237 | 102590314 | 102590237 | 102590272 | 102590274 | 102590290 | 102590314 |
| chr10 | 1114 | 102900239 | 102900328 | 102900239 | 102900248 | 102900262 | 102900287 | 102900328 |
| chr10 | 1115 | 102996124 | 102996197 | 102996124 | 102996132 | 102996136 | 102996140 | 102996197 |
| chr10 | 1116 | 102996132 | 102996215 | 102996132 | 102996136 | 102996140 | 102996197 | 102996215 |
| chr10 | 1117 | 102996136 | 102996217 | 102996136 | 102996140 | 102996197 | 102996215 | 102996217 |
| chr10 | 1118 | 102996140 | 102996219 | 102996140 | 102996197 | 102996215 | 102996217 | 102996219 |
| chr10 | 1119 | 102996257 | 102996326 | 102996257 | 102996269 | 102996271 | 102996310 | 102996326 |
| chr10 | 1120 | 102996485 | 102996554 | 102996485 | 102996490 | 102996510 | 102996514 | 102996554 |
| chr10 | 1121 | 102996514 | 102996574 | 102996514 | 102996554 | 102996565 | 102996571 | 102996574 |
| chr10 | 1122 | 102996841 | 102996942 | 102996841 | 102996849 | 102996856 | 102996900 | 102996942 |
| chr10 | 1123 | 102997102 | 102997229 | 102997102 | 102997134 | 102997143 | 102997208 | 102997229 |
| chr10 | 1124 | 102997237 | 102997326 | 102997237 | 102997253 | 102997263 | 102997282 | 102997326 |
| chr10 | 1125 | 102997253 | 102997332 | 102997253 | 102997263 | 102997282 | 102997326 | 102997332 |
| chr10 | 1126 | 102997263 | 102997345 | 102997263 | 102997282 | 102997326 | 102997332 | 102997345 |
| chr10 | 1127 | 102997282 | 102997359 | 102997282 | 102997326 | 102997332 | 102997345 | 102997359 |
| chr10 | 1128 | 102997872 | 102997993 | 102997872 | 102997889 | 102997913 | 102997940 | 102997993 |
| chr10 | 1129 | 102998294 | 102998403 | 102998294 | 102998372 | 102998379 | 102998388 | 102998403 |
| chr10 | 1130 | 102998372 | 102998412 | 102998372 | 102998379 | 102998388 | 102998403 | 102998412 |
| chr10 | 1131 | 102998379 | 102998421 | 102998379 | 102998388 | 102998403 | 102998412 | 102998421 |
| chr10 | 1132 | 102998388 | 102998426 | 102998388 | 102998403 | 102998412 | 102998421 | 102998426 |
| chr10 | 1133 | 102998403 | 102998437 | 102998403 | 102998412 | 102998421 | 102998426 | 102998437 |
| chr10 | 1134 | 103043997 | 103044078 | 103043997 | 103044008 | 103044017 | 103044033 | 103044078 |
| chr10 | 1135 | 103044112 | 103044139 | 103044112 | 103044115 | 103044124 | 103044126 | 103044139 |
| chr10 | 1136 | 103044139 | 103044168 | 103044139 | 103044144 | 103044150 | 103044157 | 103044168 |
| chr10 | 1137 | 103044231 | 103044281 | 103044231 | 103044236 | 103044244 | 103044246 | 103044281 |
| chr10 | 1138 | 103044236 | 103044301 | 103044236 | 103044244 | 103044246 | 103044281 | 103044301 |
| chr10 | 1139 | 103044244 | 103044312 | 103044244 | 103044246 | 103044281 | 103044301 | 103044312 |
| chr10 | 1140 | 103044246 | 103044323 | 103044246 | 103044281 | 103044301 | 103044312 | 103044323 |
| chr10 | 1141 | 118891193 | 118891247 | 118891193 | 118891199 | 118891214 | 118891224 | 118891247 |
| chr10 | 1142 | 118891199 | 118891257 | 118891199 | 118891214 | 118891224 | 118891247 | 118891257 |
| chr10 | 1143 | 118891214 | 118891266 | 118891214 | 118891224 | 118891247 | 118891257 | 118891266 |
| chr10 | 1144 | 122708522 | 122708554 | 122708522 | 122708529 | 122708532 | 122708552 | 122708554 |
| chr10 | 1145 | 122708529 | 122708560 | 122708529 | 122708532 | 122708552 | 122708554 | 122708560 |
| chr10 | 1146 | 122708552 | 122708573 | 122708552 | 122708554 | 122708560 | 122708571 | 122708573 |
| chr10 | 1147 | 122708554 | 122708579 | 122708554 | 122708560 | 122708571 | 122708573 | 122708579 |
| chr10 | 1148 | 122708560 | 122708590 | 122708560 | 122708571 | 122708573 | 122708579 | 122708590 |
| chr10 | 1149 | 122708571 | 122708594 | 122708571 | 122708573 | 122708579 | 122708590 | 122708594 |
| chr10 | 1150 | 122708573 | 122708596 | 122708573 | 122708579 | 122708590 | 122708594 | 122708596 |
| chr10 | 1151 | 122708579 | 122708609 | 122708579 | 122708590 | 122708594 | 122708596 | 122708609 |
| chr10 | 1152 | 122708590 | 122708613 | 122708590 | 122708594 | 122708596 | 122708609 | 122708613 |
| chr10 | 1153 | 122708637 | 122708677 | 122708637 | 122708644 | 122708657 | 122708670 | 122708677 |
| chr10 | 1154 | 122708644 | 122708684 | 122708644 | 122708657 | 122708670 | 122708677 | 122708684 |
| chr10 | 1155 | 122708657 | 122708690 | 122708657 | 122708670 | 122708677 | 122708684 | 122708690 |
| chr10 | 1156 | 122708677 | 122708700 | 122708677 | 122708684 | 122708690 | 122708698 | 122708700 |
| chr10 | 1157 | 122708684 | 122708729 | 122708684 | 122708690 | 122708698 | 122708700 | 122708729 |
| chr10 | 1158 | 122708690 | 122708739 | 122708690 | 122708698 | 122708700 | 122708729 | 122708739 |
| chr10 | 1159 | 122708698 | 122708746 | 122708698 | 122708700 | 122708729 | 122708739 | 122708746 |
| chr10 | 1160 | 122708700 | 122708780 | 122708700 | 122708729 | 122708739 | 122708746 | 122708780 |
| chr11 | 1161 | 31826182 | 31826305 | 31826182 | 31826241 | 31826272 | 31826293 | 31826305 |
| chr11 | 1162 | 31827365 | 31827454 | 31827365 | 31827377 | 31827379 | 31827392 | 31827454 |
| chr11 | 1163 | 31827518 | 31827598 | 31827518 | 31827521 | 31827572 | 31827593 | 31827598 |
| chr11 | 1164 | 31827572 | 31827638 | 31827572 | 31827593 | 31827598 | 31827625 | 31827638 |
| chr11 | 1165 | 31827905 | 31827979 | 31827905 | 31827907 | 31827914 | 31827920 | 31827979 |
| chr11 | 1166 | 31827907 | 31827981 | 31827907 | 31827914 | 31827920 | 31827979 | 31827981 |
| chr11 | 1167 | 31827914 | 31827991 | 31827914 | 31827920 | 31827979 | 31827981 | 31827991 |
| chr11 | 1168 | 31841647 | 31841706 | 31841647 | 31841663 | 31841691 | 31841703 | 31841706 |
| chr11 | 1169 | 31841663 | 31841720 | 31841663 | 31841691 | 31841703 | 31841706 | 31841720 |
| chr11 | 1170 | 31841985 | 31842090 | 31841985 | 31842002 | 31842044 | 31842050 | 31842090 |
| chr11 | 1171 | 31842002 | 31842115 | 31842002 | 31842044 | 31842050 | 31842090 | 31842115 |
| chr11 | 1172 | 31842345 | 31842444 | 31842345 | 31842366 | 31842376 | 31842418 | 31842444 |
| chr11 | 1173 | 60719012 | 60719046 | 60719012 | 60719015 | 60719017 | 60719032 | 60719046 |
| chr11 | 1174 | 60719015 | 60719048 | 60719015 | 60719017 | 60719032 | 60719046 | 60719048 |
| chr12 | 1175 | 4273900 | 4273917 | 4273900 | 4273906 | 4273908 | 4273910 | 4273917 |
| chr12 | 1176 | 49390978 | 49391019 | 49390978 | 49390983 | 49390996 | 49391003 | 49391019 |
| chr12 | 1177 | 49390983 | 49391028 | 49390983 | 49390996 | 49391003 | 49391019 | 49391028 |
| chr12 | 1178 | 49390996 | 49391051 | 49390996 | 49391003 | 49391019 | 49391028 | 49391051 |
| chr12 | 1179 | 54338682 | 54338779 | 54338682 | 54338720 | 54338743 | 54338762 | 54338779 |
| chr12 | 1180 | 54338720 | 54338785 | 54338720 | 54338743 | 54338762 | 54338779 | 54338785 |
| chr12 | 1181 | 54338743 | 54338803 | 54338743 | 54338762 | 54338779 | 54338785 | 54338803 |
| chr12 | 1182 | 54338762 | 54338819 | 54338762 | 54338779 | 54338785 | 54338803 | 54338819 |
| chr12 | 1183 | 54338779 | 54338822 | 54338779 | 54338785 | 54338803 | 54338819 | 54338822 |
| chr12 | 1184 | 54338785 | 54338824 | 54338785 | 54338803 | 54338819 | 54338822 | 54338824 |
| chr12 | 1185 | 54338937 | 54339012 | 54338937 | 54338951 | 54338978 | 54338981 | 54339012 |
| chr12 | 1186 | 54339160 | 54339258 | 54339160 | 54339163 | 54339167 | 54339190 | 54339258 |
| chr12 | 1187 | 54339163 | 54339288 | 54339163 | 54339167 | 54339190 | 54339258 | 54339288 |
| chr12 | 1188 | 54339167 | 54339297 | 54339167 | 54339190 | 54339258 | 54339288 | 54339297 |
| chr12 | 1189 | 54339190 | 54339301 | 54339190 | 54339258 | 54339288 | 54339297 | 54339301 |
| chr12 | 1190 | 54424964 | 54425042 | 54424964 | 54425019 | 54425032 | 54425039 | 54425042 |
| chr12 | 1191 | 54446944 | 54447038 | 54446944 | 54446976 | 54446996 | 54447035 | 54447038 |
| chr12 | 1192 | 65218762 | 65218789 | 65218762 | 65218777 | 65218782 | 65218784 | 65218789 |
| chr12 | 1193 | 116946335 | 116946349 | 116946335 | 116946341 | 116946344 | 116946346 | 116946349 |
| chr13 | 1194 | 112712261 | 112712290 | 112712261 | 112712267 | 112712271 | 112712277 | 112712290 |
| chr13 | 1195 | 112712267 | 112712292 | 112712267 | 112712271 | 112712277 | 112712290 | 112712292 |
| chr14 | 1196 | 36974377 | 36974436 | 36974377 | 36974389 | 36974416 | 36974428 | 36974436 |
| chr14 | 1197 | 37125912 | 37126040 | 37125912 | 37125914 | 37125926 | 37125997 | 37126040 |
| chr14 | 1198 | 37126715 | 37126787 | 37126715 | 37126723 | 37126737 | 37126751 | 37126787 |
| chr14 | 1199 | 37127716 | 37127813 | 37127716 | 37127758 | 37127774 | 37127781 | 37127813 |
| chr14 | 1200 | 37127813 | 37127860 | 37127813 | 37127815 | 37127820 | 37127850 | 37127860 |
| chr14 | 1201 | 37127815 | 37127874 | 37127815 | 37127820 | 37127850 | 37127860 | 37127874 |
| chr14 | 1202 | 37130149 | 37130246 | 37130149 | 37130202 | 37130212 | 37130237 | 37130246 |
| chr14 | 1203 | 57271167 | 57271252 | 57271167 | 57271188 | 57271214 | 57271235 | 57271252 |
| chr14 | 1204 | 57277950 | 57278069 | 57277950 | 57277995 | 57278014 | 57278021 | 57278069 |
| chr14 | 1205 | 57277995 | 57278076 | 57277995 | 57278014 | 57278021 | 57278069 | 57278076 |
| chr14 | 1206 | 57278854 | 57278909 | 57278854 | 57278863 | 57278870 | 57278885 | 57278909 |
| chr14 | 1207 | 57278863 | 57278917 | 57278863 | 57278870 | 57278885 | 57278909 | 57278917 |
| chr14 | 1208 | 57278870 | 57278927 | 57278870 | 57278885 | 57278909 | 57278917 | 57278927 |
| chr14 | 1209 | 57279027 | 57279088 | 57279027 | 57279031 | 57279037 | 57279084 | 57279088 |
| chr14 | 1210 | 57284132 | 57284177 | 57284132 | 57284154 | 57284162 | 57284167 | 57284177 |
| chr14 | 1211 | 60975447 | 60975524 | 60975447 | 60975464 | 60975472 | 60975520 | 60975524 |
| chr14 | 1212 | 60975464 | 60975532 | 60975464 | 60975472 | 60975520 | 60975524 | 60975532 |
| chr14 | 1213 | 60978163 | 60978252 | 60978163 | 60978169 | 60978179 | 60978202 | 60978252 |
| chr14 | 1214 | 61104656 | 61104717 | 61104656 | 61104658 | 61104662 | 61104695 | 61104717 |
| chr14 | 1215 | 61104658 | 61104719 | 61104658 | 61104662 | 61104695 | 61104717 | 61104719 |
| chr14 | 1216 | 61104662 | 61104754 | 61104662 | 61104695 | 61104717 | 61104719 | 61104754 |
| chr14 | 1217 | 61104695 | 61104779 | 61104695 | 61104717 | 61104719 | 61104754 | 61104779 |
| chr14 | 1218 | 61104717 | 61104790 | 61104717 | 61104719 | 61104754 | 61104779 | 61104790 |
| chr14 | 1219 | 61104719 | 61104836 | 61104719 | 61104754 | 61104779 | 61104790 | 61104836 |
| chr14 | 1220 | 61104754 | 61104848 | 61104754 | 61104779 | 61104790 | 61104836 | 61104848 |
| chr15 | 1221 | 45427548 | 45427602 | 45427548 | 45427577 | 45427592 | 45427594 | 45427602 |
| chr15 | 1222 | 45427833 | 45427861 | 45427833 | 45427839 | 45427845 | 45427854 | 45427861 |
| chr15 | 1223 | 45427845 | 45427889 | 45427845 | 45427854 | 45427861 | 45427872 | 45427889 |
| chr15 | 1224 | 45427854 | 45427894 | 45427854 | 45427861 | 45427872 | 45427889 | 45427894 |
| chr15 | 1225 | 45427861 | 45427900 | 45427861 | 45427872 | 45427889 | 45427894 | 45427900 |
| chr15 | 1226 | 76630441 | 76630471 | 76630441 | 76630449 | 76630455 | 76630465 | 76630471 |
| chr15 | 1227 | 76630455 | 76630512 | 76630455 | 76630465 | 76630471 | 76630479 | 76630512 |
| chr15 | 1228 | 76632202 | 76632283 | 76632202 | 76632205 | 76632263 | 76632273 | 76632283 |
| chr15 | 1229 | 76632205 | 76632287 | 76632205 | 76632263 | 76632273 | 76632283 | 76632287 |
| chr15 | 1230 | 76632414 | 76632461 | 76632414 | 76632420 | 76632432 | 76632452 | 76632461 |
| chr15 | 1231 | 76632432 | 76632493 | 76632432 | 76632452 | 76632461 | 76632481 | 76632493 |
| chr15 | 1232 | 76632802 | 76632891 | 76632802 | 76632811 | 76632813 | 76632832 | 76632891 |
| chr15 | 1233 | 76632811 | 76632899 | 76632811 | 76632813 | 76632832 | 76632891 | 76632899 |
| chr15 | 1234 | 76632813 | 76632910 | 76632813 | 76632832 | 76632891 | 76632899 | 76632910 |
| chr15 | 1235 | 76632955 | 76633086 | 76632955 | 76632964 | 76632980 | 76633010 | 76633086 |
| chr15 | 1236 | 89949661 | 89949776 | 89949661 | 89949672 | 89949694 | 89949745 | 89949776 |
| chr15 | 1237 | 89949672 | 89949780 | 89949672 | 89949694 | 89949745 | 89949776 | 89949780 |
| chr15 | 1238 | 89951499 | 89951623 | 89951499 | 89951535 | 89951541 | 89951592 | 89951623 |
| chr15 | 1239 | 89952135 | 89952204 | 89952135 | 89952161 | 89952165 | 89952169 | 89952204 |
| chr15 | 1240 | 89952161 | 89952247 | 89952161 | 89952165 | 89952169 | 89952204 | 89952247 |
| chr15 | 1241 | 89952165 | 89952272 | 89952165 | 89952169 | 89952204 | 89952247 | 89952272 |
| chr15 | 1242 | 89952690 | 89952824 | 89952690 | 89952700 | 89952761 | 89952773 | 89952824 |
| chr15 | 1243 | 89952938 | 89952984 | 89952938 | 89952947 | 89952965 | 89952973 | 89952984 |
| chr16 | 1244 | 3220982 | 3221039 | 3220982 | 3220992 | 3221003 | 3221033 | 3221039 |
| chr16 | 1245 | 3221003 | 3221087 | 3221003 | 3221033 | 3221039 | 3221082 | 3221087 |
| chr16 | 1246 | 3221033 | 3221089 | 3221033 | 3221039 | 3221082 | 3221087 | 3221089 |
| chr16 | 1247 | 3221176 | 3221222 | 3221176 | 3221180 | 3221182 | 3221188 | 3221222 |
| chr16 | 1248 | 3221180 | 3221235 | 3221180 | 3221182 | 3221188 | 3221222 | 3221235 |
| chr16 | 1249 | 3221182 | 3221276 | 3221182 | 3221188 | 3221222 | 3221235 | 3221276 |
| chr16 | 1250 | 3221188 | 3221280 | 3221188 | 3221222 | 3221235 | 3221276 | 3221280 |
| chr16 | 1251 | 3221961 | 3222030 | 3221961 | 3221980 | 3221990 | 3222000 | 3222030 |
| chr16 | 1252 | 3221980 | 3222040 | 3221980 | 3221990 | 3222000 | 3222030 | 3222040 |
| chr17 | 1253 | 27038809 | 27038848 | 27038809 | 27038822 | 27038831 | 27038834 | 27038848 |
| chr17 | 1254 | 35296813 | 35296874 | 35296813 | 35296836 | 35296838 | 35296847 | 35296874 |
| chr17 | 1255 | 35297848 | 35297937 | 35297848 | 35297870 | 35297881 | 35297913 | 35297937 |
| chr17 | 1256 | 35297870 | 35297952 | 35297870 | 35297881 | 35297913 | 35297937 | 35297952 |
| chr17 | 1257 | 35300874 | 35300981 | 35300874 | 35300942 | 35300970 | 35300973 | 35300981 |
| chr17 | 1258 | 46711207 | 46711264 | 46711207 | 46711224 | 46711237 | 46711239 | 46711264 |
| chr17 | 1259 | 46711224 | 46711278 | 46711224 | 46711237 | 46711239 | 46711264 | 46711278 |
| chr17 | 1260 | 46711237 | 46711281 | 46711237 | 46711239 | 46711264 | 46711278 | 46711281 |
| chr17 | 1261 | 46711239 | 46711286 | 46711239 | 46711264 | 46711278 | 46711281 | 46711286 |
| chr17 | 1262 | 46795981 | 46796042 | 46795981 | 46795990 | 46796019 | 46796036 | 46796042 |
| chr17 | 1263 | 46796104 | 46796199 | 46796104 | 46796119 | 46796141 | 46796171 | 46796199 |
| chr17 | 1264 | 46799858 | 46799986 | 46799858 | 46799882 | 46799926 | 46799948 | 46799986 |
| chr17 | 1265 | 46824922 | 46824993 | 46824922 | 46824950 | 46824953 | 46824991 | 46824993 |
| chr17 | 1266 | 46827026 | 46827113 | 46827026 | 46827033 | 46827046 | 46827080 | 46827113 |
| chr17 | 1267 | 48042572 | 48042649 | 48042572 | 48042590 | 48042605 | 48042612 | 48042649 |
| chr19 | 1268 | 46916854 | 46916949 | 46916854 | 46916861 | 46916886 | 46916944 | 46916949 |
| chr19 | 1269 | 46996668 | 46996690 | 46996668 | 46996679 | 46996681 | 46996686 | 46996690 |
| chr19 | 1270 | 58951728 | 58951790 | 58951728 | 58951756 | 58951774 | 58951778 | 58951790 |
| chr19 | 1271 | 58951778 | 58951826 | 58951778 | 58951790 | 58951814 | 58951823 | 58951826 |
| chr2 | 1272 | 45157379 | 45157503 | 45157379 | 45157460 | 45157465 | 45157472 | 45157503 |
| chr2 | 1273 | 45157472 | 45157536 | 45157472 | 45157503 | 45157512 | 45157529 | 45157536 |
| chr2 | 1274 | 45157503 | 45157578 | 45157503 | 45157512 | 45157529 | 45157536 | 45157578 |
| chr2 | 1275 | 45169679 | 45169758 | 45169679 | 45169681 | 45169729 | 45169753 | 45169758 |
| chr2 | 1276 | 45169681 | 45169774 | 45169681 | 45169729 | 45169753 | 45169758 | 45169774 |
| chr2 | 1277 | 45171565 | 45171639 | 45171565 | 45171583 | 45171621 | 45171627 | 45171639 |
| chr2 | 1278 | 63285358 | 63285491 | 63285358 | 63285365 | 63285425 | 63285441 | 63285491 |
| chr2 | 1279 | 63285365 | 63285504 | 63285365 | 63285425 | 63285441 | 63285491 | 63285504 |
| chr2 | 1280 | 63285519 | 63285654 | 63285519 | 63285535 | 63285596 | 63285645 | 63285654 |
| chr2 | 1281 | 66809107 | 66809255 | 66809107 | 66809119 | 66809126 | 66809241 | 66809255 |
| chr2 | 1282 | 66809119 | 66809257 | 66809119 | 66809126 | 66809241 | 66809255 | 66809257 |
| chr2 | 1283 | 73147529 | 73147582 | 73147529 | 73147537 | 73147559 | 73147571 | 73147582 |
| chr2 | 1284 | 73147673 | 73147693 | 73147673 | 73147678 | 73147687 | 73147690 | 73147693 |
| chr2 | 1285 | 73147678 | 73147695 | 73147678 | 73147687 | 73147690 | 73147693 | 73147695 |
| chr2 | 1286 | 73147738 | 73147789 | 73147738 | 73147755 | 73147767 | 73147771 | 73147789 |
| chr2 | 1287 | 73147939 | 73147981 | 73147939 | 73147942 | 73147960 | 73147967 | 73147981 |
| chr2 | 1288 | 73147942 | 73147998 | 73147942 | 73147960 | 73147967 | 73147981 | 73147998 |
| chr2 | 1289 | 73147960 | 73148013 | 73147960 | 73147967 | 73147981 | 73147998 | 73148013 |
| chr2 | 1290 | 73147967 | 73148045 | 73147967 | 73147981 | 73147998 | 73148013 | 73148045 |
| chr2 | 1291 | 74782066 | 74782141 | 74782066 | 74782089 | 74782096 | 74782130 | 74782141 |
| chr2 | 1292 | 74782089 | 74782157 | 74782089 | 74782096 | 74782130 | 74782141 | 74782157 |
| chr2 | 1293 | 74782096 | 74782169 | 74782096 | 74782130 | 74782141 | 74782157 | 74782169 |
| chr2 | 1294 | 74782141 | 74782201 | 74782141 | 74782157 | 74782169 | 74782194 | 74782201 |
| chr2 | 1295 | 74782157 | 74782204 | 74782157 | 74782169 | 74782194 | 74782201 | 74782204 |
| chr2 | 1296 | 74782169 | 74782219 | 74782169 | 74782194 | 74782201 | 74782204 | 74782219 |
| chr2 | 1297 | 74782194 | 74782222 | 74782194 | 74782201 | 74782204 | 74782219 | 74782222 |
| chr2 | 1298 | 114256914 | 114256994 | 114256914 | 114256944 | 114256956 | 114256970 | 114256994 |
| chr2 | 1299 | 114256944 | 114257003 | 114256944 | 114256956 | 114256970 | 114256994 | 114257003 |
| chr2 | 1300 | 114257064 | 114257108 | 114257064 | 114257069 | 114257073 | 114257094 | 114257108 |
| chr2 | 1301 | 114257069 | 114257120 | 114257069 | 114257073 | 114257094 | 114257108 | 114257120 |
| chr2 | 1302 | 114257073 | 114257123 | 114257073 | 114257094 | 114257108 | 114257120 | 114257123 |
| chr2 | 1303 | 118981920 | 118981946 | 118981920 | 118981934 | 118981939 | 118981944 | 118981946 |
| chr2 | 1304 | 118981954 | 118981984 | 118981954 | 118981957 | 118981965 | 118981973 | 118981984 |
| chr2 | 1305 | 118981957 | 118982004 | 118981957 | 118981965 | 118981973 | 118981984 | 118982004 |
| chr2 | 1306 | 118981965 | 118982007 | 118981965 | 118981973 | 118981984 | 118982004 | 118982007 |
| chr2 | 1307 | 118981973 | 118982012 | 118981973 | 118981984 | 118982004 | 118982007 | 118982012 |
| chr2 | 1308 | 118982004 | 118982060 | 118982004 | 118982007 | 118982012 | 118982056 | 118982060 |
| chr2 | 1309 | 118982007 | 118982063 | 118982007 | 118982012 | 118982056 | 118982060 | 118982063 |
| chr2 | 1310 | 118982012 | 118982067 | 118982012 | 118982056 | 118982060 | 118982063 | 118982067 |
| chr2 | 1311 | 118982056 | 118982076 | 118982056 | 118982060 | 118982063 | 118982067 | 118982076 |
| chr2 | 1312 | 118982060 | 118982081 | 118982060 | 118982063 | 118982067 | 118982076 | 118982081 |
| chr2 | 1313 | 118982076 | 118982098 | 118982076 | 118982081 | 118982086 | 118982088 | 118982098 |
| chr2 | 1314 | 118982115 | 118982149 | 118982115 | 118982120 | 118982123 | 118982127 | 118982149 |
| chr2 | 1315 | 118982120 | 118982155 | 118982120 | 118982123 | 118982127 | 118982149 | 118982155 |
| chr2 | 1316 | 118982123 | 118982157 | 118982123 | 118982127 | 118982149 | 118982155 | 118982157 |
| chr2 | 1317 | 118982127 | 118982161 | 118982127 | 118982149 | 118982155 | 118982157 | 118982161 |
| chr2 | 1318 | 118982149 | 118982174 | 118982149 | 118982155 | 118982157 | 118982161 | 118982174 |
| chr2 | 1319 | 118982155 | 118982189 | 118982155 | 118982157 | 118982161 | 118982174 | 118982189 |
| chr2 | 1320 | 118982157 | 118982191 | 118982157 | 118982161 | 118982174 | 118982189 | 118982191 |
| chr2 | 1321 | 118982189 | 118982247 | 118982189 | 118982191 | 118982200 | 118982214 | 118982247 |
| chr2 | 1322 | 119599942 | 119600002 | 119599942 | 119599960 | 119599994 | 119599997 | 119600002 |
| chr2 | 1323 | 119600477 | 119600495 | 119600477 | 119600487 | 119600489 | 119600493 | 119600495 |
| chr2 | 1324 | 119600487 | 119600501 | 119600487 | 119600489 | 119600493 | 119600495 | 119600501 |
| chr2 | 1325 | 119607675 | 119607730 | 119607675 | 119607678 | 119607719 | 119607727 | 119607730 |
| chr2 | 1326 | 119615120 | 119615222 | 119615120 | 119615140 | 119615167 | 119615196 | 119615222 |
| chr2 | 1327 | 119615167 | 119615270 | 119615167 | 119615196 | 119615222 | 119615225 | 119615270 |
| chr2 | 1328 | 119616331 | 119616405 | 119616331 | 119616334 | 119616341 | 119616368 | 119616405 |
| chr2 | 1329 | 162283663 | 162283705 | 162283663 | 162283671 | 162283679 | 162283698 | 162283705 |
| chr2 | 1330 | 162283671 | 162283711 | 162283671 | 162283679 | 162283698 | 162283705 | 162283711 |
| chr2 | 1331 | 162283679 | 162283713 | 162283679 | 162283698 | 162283705 | 162283711 | 162283713 |
| chr2 | 1332 | 162283698 | 162283719 | 162283698 | 162283705 | 162283711 | 162283713 | 162283719 |
| chr2 | 1333 | 162283705 | 162283721 | 162283705 | 162283711 | 162283713 | 162283719 | 162283721 |
| chr2 | 1334 | 175193461 | 175193533 | 175193461 | 175193470 | 175193478 | 175193517 | 175193533 |
| chr2 | 1335 | 175193670 | 175193735 | 175193670 | 175193673 | 175193680 | 175193716 | 175193735 |
| chr2 | 1336 | 175193673 | 175193744 | 175193673 | 175193680 | 175193716 | 175193735 | 175193744 |
| chr2 | 1337 | 175193680 | 175193754 | 175193680 | 175193716 | 175193735 | 175193744 | 175193754 |
| chr2 | 1338 | 175205564 | 175205633 | 175205564 | 175205577 | 175205588 | 175205607 | 175205633 |
| chr2 | 1339 | 176946500 | 176946626 | 176946500 | 176946509 | 176946594 | 176946614 | 176946626 |
| chr2 | 1340 | 176946742 | 176946869 | 176946742 | 176946749 | 176946788 | 176946790 | 176946869 |
| chr2 | 1341 | 176956631 | 176956678 | 176956631 | 176956643 | 176956645 | 176956666 | 176956678 |
| chr2 | 1342 | 176956643 | 176956699 | 176956643 | 176956645 | 176956666 | 176956678 | 176956699 |
| chr2 | 1343 | 176956830 | 176956937 | 176956830 | 176956834 | 176956841 | 176956899 | 176956937 |
| chr2 | 1344 | 176956834 | 176956957 | 176956834 | 176956841 | 176956899 | 176956937 | 176956957 |
| chr2 | 1345 | 176964175 | 176964265 | 176964175 | 176964180 | 176964194 | 176964243 | 176964265 |
| chr2 | 1346 | 176964180 | 176964279 | 176964180 | 176964194 | 176964243 | 176964265 | 176964279 |
| chr2 | 1347 | 176964194 | 176964296 | 176964194 | 176964243 | 176964265 | 176964279 | 176964296 |
| chr2 | 1348 | 176964390 | 176964456 | 176964390 | 176964404 | 176964419 | 176964448 | 176964456 |
| chr2 | 1349 | 176981384 | 176981469 | 176981384 | 176981402 | 176981409 | 176981422 | 176981469 |
| chr2 | 1350 | 176981402 | 176981492 | 176981402 | 176981409 | 176981422 | 176981469 | 176981492 |
| chr2 | 1351 | 176993588 | 176993632 | 176993588 | 176993597 | 176993600 | 176993628 | 176993632 |
| chr2 | 1352 | 182321418 | 182321541 | 182321418 | 182321489 | 182321496 | 182321530 | 182321541 |
| chr2 | 1353 | 200329056 | 200329147 | 200329056 | 200329061 | 200329084 | 200329145 | 200329147 |
| chr2 | 1354 | 200329061 | 200329149 | 200329061 | 200329084 | 200329145 | 200329147 | 200329149 |
| chr2 | 1355 | 200329084 | 200329151 | 200329084 | 200329145 | 200329147 | 200329149 | 200329151 |
| chr2 | 1356 | 200329420 | 200329506 | 200329420 | 200329422 | 200329433 | 200329486 | 200329506 |
| chr2 | 1357 | 200329422 | 200329508 | 200329422 | 200329433 | 200329486 | 200329506 | 200329508 |
| chr2 | 1358 | 200335659 | 200335752 | 200335659 | 200335663 | 200335681 | 200335683 | 200335752 |
| chr2 | 1359 | 200335663 | 200335787 | 200335663 | 200335681 | 200335683 | 200335752 | 200335787 |
| chr2 | 1360 | 200335681 | 200335790 | 200335681 | 200335683 | 200335752 | 200335787 | 200335790 |
| chr2 | 1361 | 200335683 | 200335807 | 200335683 | 200335752 | 200335787 | 200335790 | 200335807 |
| chr2 | 1362 | 219736376 | 219736400 | 219736376 | 219736378 | 219736387 | 219736397 | 219736400 |
| chr2 | 1363 | 219736378 | 219736421 | 219736378 | 219736387 | 219736397 | 219736400 | 219736421 |
| chr2 | 1364 | 223159005 | 223159127 | 223159005 | 223159028 | 223159053 | 223159082 | 223159127 |
| chr2 | 1365 | 223159028 | 223159144 | 223159028 | 223159053 | 223159082 | 223159127 | 223159144 |
| chr2 | 1366 | 223166015 | 223166094 | 223166015 | 223166036 | 223166055 | 223166059 | 223166094 |
| chr2 | 1367 | 223166363 | 223166500 | 223166363 | 223166394 | 223166435 | 223166465 | 223166500 |
| chr2 | 1368 | 223166394 | 223166506 | 223166394 | 223166435 | 223166465 | 223166500 | 223166506 |
| chr2 | 1369 | 223169441 | 223169501 | 223169441 | 223169457 | 223169470 | 223169477 | 223169501 |
| chr2 | 1370 | 223172940 | 223173068 | 223172940 | 223172965 | 223173032 | 223173061 | 223173068 |
| chr2 | 1371 | 223177068 | 223177172 | 223177068 | 223177072 | 223177093 | 223177159 | 223177172 |
| chr2 | 1372 | 223177072 | 223177198 | 223177072 | 223177093 | 223177159 | 223177172 | 223177198 |
| chr2 | 1373 | 223177093 | 223177205 | 223177093 | 223177159 | 223177172 | 223177198 | 223177205 |
| chr2 | 1374 | 237078197 | 237078248 | 237078197 | 237078208 | 237078223 | 237078238 | 237078248 |
| chr2 | 1375 | 237078208 | 237078262 | 237078208 | 237078223 | 237078238 | 237078248 | 237078262 |
| chr20 | 1376 | 61886205 | 61886255 | 61886205 | 61886229 | 61886244 | 61886249 | 61886255 |
| chr3 | 1377 | 62353328 | 62353466 | 62353328 | 62353387 | 62353431 | 62353452 | 62353466 |
| chr3 | 1378 | 62353387 | 62353504 | 62353387 | 62353431 | 62353452 | 62353466 | 62353504 |
| chr3 | 1379 | 62353431 | 62353512 | 62353431 | 62353452 | 62353466 | 62353504 | 62353512 |
| chr3 | 1380 | 62353452 | 62353557 | 62353452 | 62353466 | 62353504 | 62353512 | 62353557 |
| chr3 | 1381 | 62353466 | 62353573 | 62353466 | 62353504 | 62353512 | 62353557 | 62353573 |
| chr3 | 1382 | 62353854 | 62353939 | 62353854 | 62353894 | 62353904 | 62353917 | 62353939 |
| chr3 | 1383 | 62353894 | 62353959 | 62353894 | 62353904 | 62353917 | 62353939 | 62353959 |
| chr3 | 1384 | 127794876 | 127794956 | 127794876 | 127794903 | 127794917 | 127794941 | 127794956 |
| chr3 | 1385 | 138658470 | 138658534 | 138658470 | 138658506 | 138658508 | 138658531 | 138658534 |
| chr3 | 1386 | 138658506 | 138658546 | 138658506 | 138658508 | 138658531 | 138658534 | 138658546 |
| chr3 | 1387 | 138658531 | 138658559 | 138658531 | 138658534 | 138658546 | 138658554 | 138658559 |
| chr3 | 1388 | 138658646 | 138658735 | 138658646 | 138658655 | 138658675 | 138658704 | 138658735 |
| chr3 | 1389 | 138658888 | 138658964 | 138658888 | 138658916 | 138658927 | 138658941 | 138658964 |
| chr3 | 1390 | 138658916 | 138658968 | 138658916 | 138658927 | 138658941 | 138658964 | 138658968 |
| chr3 | 1391 | 138658927 | 138658979 | 138658927 | 138658941 | 138658964 | 138658968 | 138658979 |
| chr3 | 1392 | 138658941 | 138659013 | 138658941 | 138658964 | 138658968 | 138658979 | 138659013 |
| chr3 | 1393 | 138658964 | 138659021 | 138658964 | 138658968 | 138658979 | 138659013 | 138659021 |
| chr3 | 1394 | 147110581 | 147110711 | 147110581 | 147110595 | 147110667 | 147110669 | 147110711 |
| chr3 | 1395 | 157821132 | 157821233 | 157821132 | 157821155 | 157821179 | 157821197 | 157821233 |
| chr3 | 1396 | 157821297 | 157821369 | 157821297 | 157821304 | 157821319 | 157821364 | 157821369 |
| chr3 | 1397 | 157821304 | 157821377 | 157821304 | 157821319 | 157821364 | 157821369 | 157821377 |
| chr3 | 1398 | 157821319 | 157821381 | 157821319 | 157821364 | 157821369 | 157821377 | 157821381 |
| chr3 | 1399 | 157821364 | 157821387 | 157821364 | 157821369 | 157821377 | 157821381 | 157821387 |
| chr3 | 1400 | 157821528 | 157821585 | 157821528 | 157821536 | 157821544 | 157821554 | 157821585 |
| chr3 | 1401 | 157821536 | 157821594 | 157821536 | 157821544 | 157821554 | 157821585 | 157821594 |
| chr3 | 1402 | 157821544 | 157821596 | 157821544 | 157821554 | 157821585 | 157821594 | 157821596 |
| chr3 | 1403 | 181413405 | 181413472 | 181413405 | 181413421 | 181413434 | 181413440 | 181413472 |
| chr4 | 1404 | 10462906 | 10462956 | 10462906 | 10462936 | 10462949 | 10462953 | 10462956 |
| chr4 | 1405 | 10463167 | 10463188 | 10463167 | 10463173 | 10463179 | 10463183 | 10463188 |
| chr4 | 1406 | 10463173 | 10463190 | 10463173 | 10463179 | 10463183 | 10463188 | 10463190 |
| chr4 | 1407 | 10463179 | 10463196 | 10463179 | 10463183 | 10463188 | 10463190 | 10463196 |
| chr4 | 1408 | 57521506 | 57521581 | 57521506 | 57521518 | 57521537 | 57521545 | 57521581 |
| chr4 | 1409 | 57521680 | 57521725 | 57521680 | 57521687 | 57521701 | 57521716 | 57521725 |
| chr4 | 1410 | 57521687 | 57521733 | 57521687 | 57521701 | 57521716 | 57521725 | 57521733 |
| chr4 | 1411 | 85403097 | 85403144 | 85403097 | 85403100 | 85403104 | 85403109 | 85403144 |
| chr4 | 1412 | 85403100 | 85403160 | 85403100 | 85403104 | 85403109 | 85403144 | 85403160 |
| chr4 | 1413 | 85403144 | 85403223 | 85403144 | 85403160 | 85403167 | 85403174 | 85403223 |
| chr4 | 1414 | 140200545 | 140200615 | 140200545 | 140200580 | 140200583 | 140200608 | 140200615 |
| chr4 | 1415 | 140200580 | 140200652 | 140200580 | 140200583 | 140200608 | 140200615 | 140200652 |
| chr4 | 1416 | 140200615 | 140200708 | 140200615 | 140200652 | 140200656 | 140200658 | 140200708 |
| chr4 | 1417 | 140200652 | 140200716 | 140200652 | 140200656 | 140200658 | 140200708 | 140200716 |
| chr4 | 1418 | 140200656 | 140200723 | 140200656 | 140200658 | 140200708 | 140200716 | 140200723 |
| chr4 | 1419 | 140200658 | 140200734 | 140200658 | 140200708 | 140200716 | 140200723 | 140200734 |
| chr4 | 1420 | 155663155 | 155663234 | 155663155 | 155663164 | 155663210 | 155663225 | 155663234 |
| chr4 | 1421 | 155663164 | 155663254 | 155663164 | 155663210 | 155663225 | 155663234 | 155663254 |
| chr4 | 1422 | 155663210 | 155663263 | 155663210 | 155663225 | 155663234 | 155663254 | 155663263 |
| chr4 | 1423 | 155663311 | 155663434 | 155663311 | 155663335 | 155663346 | 155663396 | 155663434 |
| chr4 | 1424 | 155663546 | 155663633 | 155663546 | 155663574 | 155663598 | 155663610 | 155663633 |
| chr4 | 1425 | 174439990 | 174440058 | 174439990 | 174439998 | 174440012 | 174440019 | 174440058 |
| chr4 | 1426 | 174440019 | 174440079 | 174440019 | 174440058 | 174440061 | 174440071 | 174440079 |
| chr4 | 1427 | 185937206 | 185937269 | 185937206 | 185937224 | 185937243 | 185937248 | 185937269 |
| chr5 | 1428 | 1885333 | 1885444 | 1885333 | 1885350 | 1885367 | 1885430 | 1885444 |
| chr5 | 1429 | 1885594 | 1885639 | 1885594 | 1885597 | 1885607 | 1885623 | 1885639 |
| chr5 | 1430 | 2756026 | 2756111 | 2756026 | 2756062 | 2756083 | 2756101 | 2756111 |
| chr5 | 1431 | 2757398 | 2757457 | 2757398 | 2757413 | 2757444 | 2757447 | 2757457 |
| chr5 | 1432 | 76923753 | 76923857 | 76923753 | 76923794 | 76923806 | 76923817 | 76923857 |
| chr5 | 1433 | 76923794 | 76923888 | 76923794 | 76923806 | 76923817 | 76923857 | 76923888 |
| chr5 | 1434 | 76923918 | 76923985 | 76923918 | 76923943 | 76923956 | 76923963 | 76923985 |
| chr5 | 1435 | 76923956 | 76924025 | 76923956 | 76923963 | 76923985 | 76923988 | 76924025 |
| chr5 | 1436 | 76923963 | 76924041 | 76923963 | 76923985 | 76923988 | 76924025 | 76924041 |
| chr5 | 1437 | 77268417 | 77268455 | 77268417 | 77268435 | 77268446 | 77268452 | 77268455 |
| chr5 | 1438 | 77268671 | 77268694 | 77268671 | 77268673 | 77268687 | 77268690 | 77268694 |
| chr5 | 1439 | 77268673 | 77268698 | 77268673 | 77268687 | 77268690 | 77268694 | 77268698 |
| chr5 | 1440 | 77268687 | 77268705 | 77268687 | 77268690 | 77268694 | 77268698 | 77268705 |
| chr5 | 1441 | 77268855 | 77268948 | 77268855 | 77268888 | 77268898 | 77268942 | 77268948 |
| chr5 | 1442 | 87970750 | 87970769 | 87970750 | 87970755 | 87970757 | 87970763 | 87970769 |
| chr5 | 1443 | 87970755 | 87970772 | 87970755 | 87970757 | 87970763 | 87970769 | 87970772 |
| chr5 | 1444 | 87970757 | 87970775 | 87970757 | 87970763 | 87970769 | 87970772 | 87970775 |
| chr5 | 1445 | 172660946 | 172661033 | 172660946 | 172660996 | 172661002 | 172661015 | 172661033 |
| chr6 | 1446 | 1378219 | 1378346 | 1378219 | 1378238 | 1378268 | 1378276 | 1378346 |
| chr6 | 1447 | 1383830 | 1383859 | 1383830 | 1383837 | 1383841 | 1383853 | 1383859 |
| chr6 | 1448 | 1389022 | 1389156 | 1389022 | 1389140 | 1389146 | 1389152 | 1389156 |
| chr6 | 1449 | 6004286 | 6004328 | 6004286 | 6004292 | 6004298 | 6004323 | 6004328 |
| chr6 | 1450 | 6004298 | 6004349 | 6004298 | 6004323 | 6004328 | 6004337 | 6004349 |
| chr6 | 1451 | 6004409 | 6004484 | 6004409 | 6004429 | 6004445 | 6004464 | 6004484 |
| chr6 | 1452 | 6004429 | 6004494 | 6004429 | 6004445 | 6004464 | 6004484 | 6004494 |
| chr6 | 1453 | 6004445 | 6004514 | 6004445 | 6004464 | 6004484 | 6004494 | 6004514 |
| chr6 | 1454 | 6004638 | 6004739 | 6004638 | 6004644 | 6004686 | 6004736 | 6004739 |
| chr6 | 1455 | 6004644 | 6004745 | 6004644 | 6004686 | 6004736 | 6004739 | 6004745 |
| chr6 | 1456 | 10381594 | 10381667 | 10381594 | 10381629 | 10381636 | 10381641 | 10381667 |
| chr6 | 1457 | 10384844 | 10384975 | 10384844 | 10384864 | 10384966 | 10384971 | 10384975 |
| chr6 | 1458 | 10384864 | 10384980 | 10384864 | 10384966 | 10384971 | 10384975 | 10384980 |
| chr6 | 1459 | 10385781 | 10385903 | 10385781 | 10385837 | 10385839 | 10385894 | 10385903 |
| chr6 | 1460 | 10385837 | 10385914 | 10385837 | 10385839 | 10385894 | 10385903 | 10385914 |
| chr6 | 1461 | 10385839 | 10385925 | 10385839 | 10385894 | 10385903 | 10385914 | 10385925 |
| chr6 | 1462 | 10385903 | 10386007 | 10385903 | 10385914 | 10385925 | 10385965 | 10386007 |
| chr6 | 1463 | 10386160 | 10386259 | 10386160 | 10386163 | 10386167 | 10386226 | 10386259 |
| chr6 | 1464 | 26614046 | 26614107 | 26614046 | 26614067 | 26614091 | 26614100 | 26614107 |
| chr6 | 1465 | 26614067 | 26614142 | 26614067 | 26614091 | 26614100 | 26614107 | 26614142 |
| chr6 | 1466 | 26614091 | 26614147 | 26614091 | 26614100 | 26614107 | 26614142 | 26614147 |
| chr6 | 1467 | 26614218 | 26614334 | 26614218 | 26614231 | 26614268 | 26614300 | 26614334 |
| chr6 | 1468 | 26614420 | 26614495 | 26614420 | 26614433 | 26614461 | 26614483 | 26614495 |
| chr6 | 1469 | 26614495 | 26614553 | 26614495 | 26614500 | 26614535 | 26614545 | 26614553 |
| chr6 | 1470 | 26614535 | 26614589 | 26614535 | 26614545 | 26614553 | 26614560 | 26614589 |
| chr6 | 1471 | 26614553 | 26614596 | 26614553 | 26614560 | 26614589 | 26614593 | 26614596 |
| chr6 | 1472 | 26614619 | 26614660 | 26614619 | 26614646 | 26614649 | 26614651 | 26614660 |
| chr6 | 1473 | 26614734 | 26614797 | 26614734 | 26614749 | 26614753 | 26614794 | 26614797 |
| chr6 | 1474 | 50791385 | 50791436 | 50791385 | 50791392 | 50791414 | 50791419 | 50791436 |
| chr6 | 1475 | 50791392 | 50791450 | 50791392 | 50791414 | 50791419 | 50791436 | 50791450 |
| chr6 | 1476 | 50791414 | 50791466 | 50791414 | 50791419 | 50791436 | 50791450 | 50791466 |
| chr6 | 1477 | 50793354 | 50793468 | 50793354 | 50793380 | 50793390 | 50793410 | 50793468 |
| chr6 | 1478 | 50810618 | 50810682 | 50810618 | 50810643 | 50810656 | 50810676 | 50810682 |
| chr6 | 1479 | 50810643 | 50810697 | 50810643 | 50810656 | 50810676 | 50810682 | 50810697 |
| chr6 | 1480 | 50810712 | 50810788 | 50810712 | 50810715 | 50810735 | 50810761 | 50810788 |
| chr6 | 1481 | 50810715 | 50810825 | 50810715 | 50810735 | 50810761 | 50810788 | 50810825 |
| chr6 | 1482 | 50810735 | 50810837 | 50810735 | 50810761 | 50810788 | 50810825 | 50810837 |
| chr6 | 1483 | 50810761 | 50810845 | 50810761 | 50810788 | 50810825 | 50810837 | 50810845 |
| chr6 | 1484 | 108489506 | 108489556 | 108489506 | 108489510 | 108489515 | 108489528 | 108489556 |
| chr6 | 1485 | 108489510 | 108489566 | 108489510 | 108489515 | 108489528 | 108489556 | 108489566 |
| chr6 | 1486 | 108489515 | 108489575 | 108489515 | 108489528 | 108489556 | 108489566 | 108489575 |
| chr6 | 1487 | 108489656 | 108489722 | 108489656 | 108489662 | 108489668 | 108489698 | 108489722 |
| chr6 | 1488 | 108489662 | 108489733 | 108489662 | 108489668 | 108489698 | 108489722 | 108489733 |
| chr6 | 1489 | 108489668 | 108489746 | 108489668 | 108489698 | 108489722 | 108489733 | 108489746 |
| chr6 | 1490 | 108489861 | 108489907 | 108489861 | 108489864 | 108489889 | 108489895 | 108489907 |
| chr6 | 1491 | 108489864 | 108489916 | 108489864 | 108489889 | 108489895 | 108489907 | 108489916 |
| chr6 | 1492 | 108489889 | 108489923 | 108489889 | 108489895 | 108489907 | 108489916 | 108489923 |
| chr6 | 1493 | 108489895 | 108489929 | 108489895 | 108489907 | 108489916 | 108489923 | 108489929 |
| chr6 | 1494 | 108489929 | 108489956 | 108489929 | 108489946 | 108489949 | 108489951 | 108489956 |
| chr6 | 1495 | 108489949 | 108489977 | 108489949 | 108489951 | 108489956 | 108489961 | 108489977 |
| chr6 | 1496 | 108489951 | 108490000 | 108489951 | 108489956 | 108489961 | 108489977 | 108490000 |
| chr6 | 1497 | 108490257 | 108490317 | 108490257 | 108490264 | 108490268 | 108490297 | 108490317 |
| chr6 | 1498 | 108490455 | 108490528 | 108490455 | 108490485 | 108490516 | 108490524 | 108490528 |
| chr6 | 1499 | 108490485 | 108490538 | 108490485 | 108490516 | 108490524 | 108490528 | 108490538 |
| chr6 | 1500 | 108490516 | 108490583 | 108490516 | 108490524 | 108490528 | 108490538 | 108490583 |
| chr6 | 1501 | 108490861 | 108490976 | 108490861 | 108490881 | 108490895 | 108490917 | 108490976 |
| chr6 | 1502 | 108490881 | 108490994 | 108490881 | 108490895 | 108490917 | 108490976 | 108490994 |
| chr6 | 1503 | 108491050 | 108491126 | 108491050 | 108491075 | 108491108 | 108491110 | 108491126 |
| chr6 | 1504 | 137810404 | 137810464 | 137810404 | 137810417 | 137810432 | 137810452 | 137810464 |
| chr6 | 1505 | 137810432 | 137810493 | 137810432 | 137810452 | 137810464 | 137810489 | 137810493 |
| chr6 | 1506 | 137810712 | 137810865 | 137810712 | 137810736 | 137810767 | 137810810 | 137810865 |
| chr6 | 1507 | 137811147 | 137811242 | 137811147 | 137811173 | 137811176 | 137811220 | 137811242 |
| chr6 | 1508 | 137814027 | 137814153 | 137814027 | 137814078 | 137814093 | 137814134 | 137814153 |
| chr6 | 1509 | 150285568 | 150285641 | 150285568 | 150285577 | 150285620 | 150285634 | 150285641 |
| chr6 | 1510 | 150285641 | 150285670 | 150285641 | 150285652 | 150285659 | 150285661 | 150285670 |
| chr7 | 1511 | 27196449 | 27196489 | 27196449 | 27196470 | 27196472 | 27196478 | 27196489 |
| chr7 | 1512 | 27196624 | 27196712 | 27196624 | 27196658 | 27196673 | 27196675 | 27196712 |
| chr7 | 1513 | 27196658 | 27196751 | 27196658 | 27196673 | 27196675 | 27196712 | 27196751 |
| chr7 | 1514 | 27205514 | 27205658 | 27205514 | 27205521 | 27205577 | 27205585 | 27205658 |
| chr7 | 1515 | 27205685 | 27205760 | 27205685 | 27205694 | 27205697 | 27205722 | 27205760 |
| chr7 | 1516 | 27205694 | 27205784 | 27205694 | 27205697 | 27205722 | 27205760 | 27205784 |
| chr7 | 1517 | 27205697 | 27205791 | 27205697 | 27205722 | 27205760 | 27205784 | 27205791 |
| chr7 | 1518 | 27205722 | 27205800 | 27205722 | 27205760 | 27205784 | 27205791 | 27205800 |
| chr7 | 1519 | 27205760 | 27205810 | 27205760 | 27205784 | 27205791 | 27205800 | 27205810 |
| chr7 | 1520 | 27279173 | 27279202 | 27279173 | 27279175 | 27279177 | 27279195 | 27279202 |
| chr7 | 1521 | 27279175 | 27279217 | 27279175 | 27279177 | 27279195 | 27279202 | 27279217 |
| chr7 | 1522 | 27279177 | 27279222 | 27279177 | 27279195 | 27279202 | 27279217 | 27279222 |
| chr7 | 1523 | 27279195 | 27279224 | 27279195 | 27279202 | 27279217 | 27279222 | 27279224 |
| chr7 | 1524 | 27279217 | 27279240 | 27279217 | 27279222 | 27279224 | 27279237 | 27279240 |
| chr7 | 1525 | 27279501 | 27279585 | 27279501 | 27279556 | 27279575 | 27279580 | 27279585 |
| chr7 | 1526 | 27279587 | 27279717 | 27279587 | 27279600 | 27279614 | 27279645 | 27279717 |
| chr7 | 1527 | 27283217 | 27283303 | 27283217 | 27283225 | 27283255 | 27283287 | 27283303 |
| chr7 | 1528 | 113723041 | 113723098 | 113723041 | 113723043 | 113723046 | 113723093 | 113723098 |
| chr7 | 1529 | 121939899 | 121940019 | 121939899 | 121939919 | 121939982 | 121940007 | 121940019 |
| chr7 | 1530 | 121940081 | 121940169 | 121940081 | 121940083 | 121940128 | 121940157 | 121940169 |
| chr7 | 1531 | 121940083 | 121940184 | 121940083 | 121940128 | 121940157 | 121940169 | 121940184 |
| chr7 | 1532 | 121940128 | 121940202 | 121940128 | 121940157 | 121940169 | 121940184 | 121940202 |
| chr7 | 1533 | 121946825 | 121946919 | 121946825 | 121946828 | 121946859 | 121946871 | 121946919 |
| chr7 | 1534 | 121946828 | 121946923 | 121946828 | 121946859 | 121946871 | 121946919 | 121946923 |
| chr7 | 1535 | 121946859 | 121946944 | 121946859 | 121946871 | 121946919 | 121946923 | 121946944 |
| chr7 | 1536 | 121947005 | 121947091 | 121947005 | 121947045 | 121947072 | 121947080 | 121947091 |
| chr7 | 1537 | 121951977 | 121952001 | 121951977 | 121951981 | 121951983 | 121951993 | 121952001 |
| chr7 | 1538 | 129422847 | 129422965 | 129422847 | 129422853 | 129422915 | 129422952 | 129422965 |
| chr7 | 1539 | 129422853 | 129422970 | 129422853 | 129422915 | 129422952 | 129422965 | 129422970 |
| chr7 | 1540 | 129423101 | 129423179 | 129423101 | 129423115 | 129423126 | 129423151 | 129423179 |
| chr7 | 1541 | 129424571 | 129424671 | 129424571 | 129424575 | 129424588 | 129424607 | 129424671 |
| chr7 | 1542 | 129424766 | 129424871 | 129424766 | 129424778 | 129424791 | 129424800 | 129424871 |
| chr7 | 1543 | 129424778 | 129424873 | 129424778 | 129424791 | 129424800 | 129424871 | 129424873 |
| chr7 | 1544 | 129424791 | 129424888 | 129424791 | 129424800 | 129424871 | 129424873 | 129424888 |
| chr7 | 1545 | 129424871 | 129424929 | 129424871 | 129424873 | 129424888 | 129424922 | 129424929 |
| chr7 | 1546 | 129424873 | 129424932 | 129424873 | 129424888 | 129424922 | 129424929 | 129424932 |
| chr7 | 1547 | 129424888 | 129424943 | 129424888 | 129424922 | 129424929 | 129424932 | 129424943 |
| chr7 | 1548 | 129424929 | 129424999 | 129424929 | 129424932 | 129424943 | 129424952 | 129424999 |
| chr7 | 1549 | 129424932 | 129425010 | 129424932 | 129424943 | 129424952 | 129424999 | 129425010 |
| chr7 | 1550 | 129424943 | 129425025 | 129424943 | 129424952 | 129424999 | 129425010 | 129425025 |
| chr7 | 1551 | 155165141 | 155165186 | 155165141 | 155165144 | 155165165 | 155165168 | 155165186 |
| chr7 | 1552 | 155302657 | 155302687 | 155302657 | 155302667 | 155302675 | 155302684 | 155302687 |
| chr7 | 1553 | 155302667 | 155302692 | 155302667 | 155302675 | 155302684 | 155302687 | 155302692 |
| chr7 | 1554 | 155600583 | 155600664 | 155600583 | 155600624 | 155600645 | 155600658 | 155600664 |
| chr7 | 1555 | 155600645 | 155600684 | 155600645 | 155600658 | 155600664 | 155600681 | 155600684 |
| chr8 | 1556 | 70947141 | 70947218 | 70947141 | 70947164 | 70947171 | 70947175 | 70947218 |
| chr8 | 1557 | 70981921 | 70982003 | 70981921 | 70981935 | 70981960 | 70981994 | 70982003 |
| chr8 | 1558 | 70981935 | 70982012 | 70981935 | 70981960 | 70981994 | 70982003 | 70982012 |
| chr8 | 1559 | 70981960 | 70982014 | 70981960 | 70981994 | 70982003 | 70982012 | 70982014 |
| chr8 | 1560 | 70984476 | 70984538 | 70984476 | 70984487 | 70984524 | 70984535 | 70984538 |
| chr9 | 1561 | 970299 | 970433 | 970299 | 970307 | 970321 | 970379 | 970433 |
| chr9 | 1562 | 974840 | 974981 | 974840 | 974854 | 974961 | 974979 | 974981 |
| chr9 | 1563 | 79631424 | 79631490 | 79631424 | 79631432 | 79631442 | 79631488 | 79631490 |
| chr9 | 1564 | 79631432 | 79631497 | 79631432 | 79631442 | 79631488 | 79631490 | 79631497 |
| chr9 | 1565 | 79636823 | 79636893 | 79636823 | 79636846 | 79636863 | 79636875 | 79636893 |
| chr9 | 1566 | 79637009 | 79637125 | 79637009 | 79637026 | 79637050 | 79637077 | 79637125 |
| chr9 | 1567 | 79637673 | 79637790 | 79637673 | 79637730 | 79637740 | 79637762 | 79637790 |
| chr9 | 1568 | 79637740 | 79637824 | 79637740 | 79637762 | 79637790 | 79637815 | 79637824 |
| chr9 | 1569 | 79637762 | 79637837 | 79637762 | 79637790 | 79637815 | 79637824 | 79637837 |
| chr9 | 1570 | 79637790 | 79637840 | 79637790 | 79637815 | 79637824 | 79637837 | 79637840 |
| chr9 | 1571 | 79637815 | 79637843 | 79637815 | 79637824 | 79637837 | 79637840 | 79637843 |
| chr9 | 1572 | 79637824 | 79637859 | 79637824 | 79637837 | 79637840 | 79637843 | 79637859 |
| chr9 | 1573 | 79637978 | 79638047 | 79637978 | 79638006 | 79638031 | 79638034 | 79638047 |
| chr9 | 1574 | 79638006 | 79638054 | 79638006 | 79638031 | 79638034 | 79638047 | 79638054 |
| chr9 | 1575 | 91792498 | 91792563 | 91792498 | 91792514 | 91792538 | 91792540 | 91792563 |
| chr9 | 1576 | 96715047 | 96715183 | 96715047 | 96715111 | 96715150 | 96715170 | 96715183 |
| chr9 | 1577 | 96716987 | 96717004 | 96716987 | 96716991 | 96716996 | 96716998 | 96717004 |
| chr9 | 1578 | 96716991 | 96717043 | 96716991 | 96716996 | 96716998 | 96717004 | 96717043 |
| chr9 | 1579 | 96716996 | 96717050 | 96716996 | 96716998 | 96717004 | 96717043 | 96717050 |
| chr9 | 1580 | 98112032 | 98112109 | 98112032 | 98112065 | 98112082 | 98112106 | 98112109 |
| chr9 | 1581 | 100609955 | 100610074 | 100609955 | 100609973 | 100610007 | 100610055 | 100610074 |
| chr9 | 1582 | 100610007 | 100610121 | 100610007 | 100610055 | 100610074 | 100610111 | 100610121 |
| chr9 | 1583 | 100610055 | 100610123 | 100610055 | 100610074 | 100610111 | 100610121 | 100610123 |
| chr9 | 1584 | 100610074 | 100610136 | 100610074 | 100610111 | 100610121 | 100610123 | 100610136 |
| chr9 | 1585 | 100610597 | 100610741 | 100610597 | 100610697 | 100610699 | 100610710 | 100610741 |
| chr9 | 1586 | 100610710 | 100610793 | 100610710 | 100610741 | 100610755 | 100610763 | 100610793 |
| chr9 | 1587 | 100610741 | 100610799 | 100610741 | 100610755 | 100610763 | 100610793 | 100610799 |
| chr9 | 1588 | 100611132 | 100611219 | 100611132 | 100611171 | 100611178 | 100611210 | 100611219 |
| chr9 | 1589 | 100611171 | 100611228 | 100611171 | 100611178 | 100611210 | 100611219 | 100611228 |
| chr9 | 1590 | 100611178 | 100611243 | 100611178 | 100611210 | 100611219 | 100611228 | 100611243 |
| chr9 | 1591 | 100611210 | 100611318 | 100611210 | 100611219 | 100611228 | 100611243 | 100611318 |
| chr9 | 1592 | 100619738 | 100619858 | 100619738 | 100619774 | 100619818 | 100619848 | 100619858 |
| chr9 | 1593 | 100619774 | 100619884 | 100619774 | 100619818 | 100619848 | 100619858 | 100619884 |
| chr9 | 1594 | 100619848 | 100619937 | 100619848 | 100619858 | 100619884 | 100619910 | 100619937 |
| chr9 | 1595 | 100619858 | 100619942 | 100619858 | 100619884 | 100619910 | 100619937 | 100619942 |
| chr9 | 1596 | 100619884 | 100619985 | 100619884 | 100619910 | 100619937 | 100619942 | 100619985 |
| chr9 | 1597 | 126779686 | 126779761 | 126779686 | 126779716 | 126779720 | 126779738 | 126779761 |
| chr9 | 1598 | 126779716 | 126779773 | 126779716 | 126779720 | 126779738 | 126779761 | 126779773 |
| chr9 | 1599 | 126779851 | 126779883 | 126779851 | 126779857 | 126779869 | 126779875 | 126779883 |
| chr9 | 1600 | 126780098 | 126780130 | 126780098 | 126780104 | 126780109 | 126780120 | 126780130 |
| chr9 | 1601 | 129377432 | 129377464 | 129377432 | 129377444 | 129377451 | 129377453 | 129377464 |
| chr9 | 1602 | 129377563 | 129377597 | 129377563 | 129377571 | 129377577 | 129377592 | 129377597 |
| chr9 | 1603 | 129377571 | 129377601 | 129377571 | 129377577 | 129377592 | 129377597 | 129377601 |
| chr9 | 1604 | 129377577 | 129377606 | 129377577 | 129377592 | 129377597 | 129377601 | 129377606 |

TABLE 3
Limit of blank study results

| Sample | Mean Total Reads | Mean unique aligned reads |
|---|---|---|
| True | 254,246,578 | 34,263,591 |
| Blank | 227,666 | 8,013 |
| Blank % of True | 0.09% | 0.02% |

What is claimed is:

1. A method of determining tumor DNA content in a biological sample comprising:
a) obtaining cell-free DNA fragments from the sample;
b) determining methylation statuses of methylation variants each representing two or more sequential CpG sites in a genomic location from the cell-free DNA fragments; and
c) quantifying a proportion of fully methylated methylation variants,
wherein the quantification in step c) is performed without requiring a matched tissue sample.

2. (canceled)

3. The method of claim 1, wherein the methylation variants are conserved across two or more, three or more, four or more, or five or more cancers.

4. The method of claim 1, wherein each methylation variant comprises 3 or more sequential CpG sites, 4 or more sequential CpG sites, or 5 or more sequential CpG sites.

5. The method of claim 1, wherein each methylation variant comprises 5 sequential CpG sites.

6. The method of claim 1, wherein each methylation variant refers to a range of genomic locations and corresponding CpG sites identified in Table 1 or Table 2.

7. The method of claim 1, wherein determining methylation statuses of methylation variants comprises performing one or more assays on the cell-free DNA fragments.

8. The method of claim 7, wherein the one or more assays comprises bisulfite conversion, nucleic acid amplification, polymerase chain reaction (PCR), methylation-specific PCR, bisulfite pyrosequencing, single-strand conformation polymorphism (SSCP) analysis, methylation-sensitive single-strand conformation analysis, high resolution melting analysis, methylation-sensitive single-nucleotide primer extension, restriction analysis, microarray technology, next generation methylation sequencing, nanopore sequencing, endonuclease digestion, affinity enrichment, target enrichment, hybrid capture, or enzymatic conversion.

9. The method of claim 1, wherein determining methylation statuses of methylation variants comprises determining methylation statuses of about 5 to about 5000 methylation variants.

10. The method of claim 1, wherein determining methylation statuses of methylation variants comprises determining methylation statuses of 1043 methylation variants.

11. The method of claim 1, wherein the proportion of fully methylated methylation variants relative to a total number of methylation variants is from about 0.00001 to about 0.9, optionally from about 0.0001 to about 0.001.

12. The method of claim 1, further comprising determining whether the sample is positive for cancer if the proportion of fully methylated methylation variants relative to a total number of methylation variants is greater than a threshold proportion.

13. The method of claim 12, wherein the threshold proportion is from about 0.0001 to about 0.001.

14-35. (canceled)

36. A method of determining tumor DNA content in a biological sample comprising:
a) obtaining cell-free DNA fragments from the sample;
b) determining methylation statuses of methylation variants each representing two or more sequential CpG sites in a genomic location from the cell-free DNA fragments; and
c) quantifying a proportion of fully methylated methylation variants,
wherein the cell-free DNA fragments are detected with a sensitivity of at least 85% when the proportion of the cell-free DNA fragments in which the sequential CpG sites of the genomic locations are methylated is greater than 0.0001.

37. The method of claim 36, wherein the methylation variants are conserved across two or more, three or more, four or more, or five or more cancers.

38. The method of claim 37, wherein each methylation variant comprises 3 or more sequential CpG sites, 4 or more sequential CpG sites, or 5 or more sequential CpG sites.

39. The method of claim 36, wherein each methylation variant comprises 5 sequential CpG sites.

40. The method of claim 36, wherein each methylation variant refers to a range of genomic locations and corresponding CpG sites identified in Table 1 or Table 2.

41. The method of claim 36, wherein determining methylation statuses of methylation variants comprises performing one or more assays on the cell-free DNA fragments.

42. The method of claim 41, wherein the genomic assay comprises bisulfite conversion, nucleic acid amplification, polymerase chain reaction (PCR), methylation-specific PCR, bisulfite pyrosequencing, single-strand conformation polymorphism (SSCP) analysis, methylation-sensitive single-strand conformation analysis, high resolution melting analysis, methylation-sensitive single-nucleotide primer extension, restriction analysis, microarray technology, next generation methylation sequencing, nanopore sequencing, endonuclease digestion, affinity enrichment, target enrichment, hybrid capture, or enzymatic conversion.

43-135. (canceled)

136. A method of identifying a subject as a candidate subject for treatment, the method comprising:
a) performing longitudinal tracking of tumor content across two or more samples obtained from the subject, wherein the performing comprises:
i) obtaining cell-free DNA fragments from a sample obtained from a subject at a timepoint;
ii) determining methylation statuses of methylation variants each representing two or more sequential CpG sites in a genomic location from the cell-free DNA fragments;
iii) quantifying a proportion of fully methylated methylation variants without requiring a matched tissue sample;
iv) determining whether the sample is positive for cancer if the proportion of fully methylated methylation variants relative to a total number of methylation variants is greater than a threshold proportion;
v) repeating steps (i)-(iv) for an additional sample of the two or more samples obtained from the subject at a different timepoint; and
f) determining a total number or proportion of samples of the two or more samples that are positive for cancer; and
g) responsive to determining that at least a threshold of the two or more samples are positive for cancer, identifying the subject as a candidate subject for treatment.

137-159. (canceled)


SEQUENCE LISTING (1604 sequences)

SEQ ID NO: 1 - DNA, 36 bp, Homo sapiens
  source 1..36: mol_type = genomic DNA
cggaggtcgtgacccaggggaaagcgtgggcggtcg

SEQ ID NO: 2 - DNA, 45 bp, Homo sapiens
  source 1..45: mol_type = genomic DNA
cgtgacccaggggaaagcgtgggcggtcgacccagggcagctgcg

SEQ ID NO: 3 - DNA, 55 bp, Homo sapiens
  source 1..55: mol_type = genomic DNA
cgacccagggcagctgcggcggcgaggcaggtgggctccttgctccctggagccg

SEQ ID NO: 4 - DNA, 61 bp, Homo sapiens
  source 1..61: mol_type = genomic DNA
cggcggcgaggcaggtgggctccttgctccctggagccgcccctccccacacctgccctcg

SEQ ID NO: 5 - DNA, 61 bp, Homo sapiens
  source 1..61: mol_type = genomic DNA
cggcgaggcaggtgggctccttgctccctggagccgcccctccccacacctgccctcggcg

SEQ ID NO: 6 - DNA, 87 bp, Homo sapiens
  source 1..87: mol_type = genomic DNA
cgaggcaggtgggctccttgctccctggagccgcccctccccacacctgccctcggcgcccccagcagttttcaccttggccctccg

SEQ ID NO: 7 - DNA, 58 bp, Homo sapiens
  source 1..58: mol_type = genomic DNA
cgcccctccccacacctgccctcggcgcccccagcagttttcaccttggccctccgcg

SEQ ID NO: 8 - DNA, 45 bp, Homo sapiens
  source 1..45: mol_type = genomic DNA
cggcgcccccagcagttttcaccttggccctccgcggtcactgcg

SEQ ID NO: 9 - DNA, 50 bp, Homo sapiens
  source 1..50: mol_type = genomic DNA
cgttgccgccagcccagtggggagtgaattagcgccctccttcgtcctcg

SEQ ID NO: 10 - DNA, 53 bp, Homo sapiens
  source 1..53: mol_type = genomic DNA
cgccagcccagtggggagtgaattagcgccctccttcgtcctcggcccttccg

SEQ ID NO: 11 - DNA, 30 bp, Homo sapiens
  source 1..30: mol_type = genomic DNA
cgccctccttcgtcctcggcccttccgacg

SEQ ID NO: 12 - DNA, 25 bp, Homo sapiens
  source 1..25: mol_type = genomic DNA
cgtcctcggcccttccgacggcacg

SEQ ID NO: 13 - DNA, 50 bp, Homo sapiens
  source 1..50: mol_type = genomic DNA
cggcccttccgacggcacgaggaactcctgtcctgccccacagaccttcg

SEQ ID NO: 14 - DNA, 70 bp, Homo sapiens
  source 1..70: mol_type = genomic DNA
cggtactggagcctgccccgccagggccctggaatcagagaaagtcgctctttggccacctgaagcgtcg

SEQ ID NO: 15 - DNA, 81 bp, Homo sapiens
  source 1..81: mol_type = genomic DNA
cgccagggccctggaatcagagaaagtcgctctttggccacctgaagcgtcggatccctacagtgcctcccagcctgggcg

SEQ ID NO: 16 - DNA, 60 bp, Homo sapiens
  source 1..60: mol_type = genomic DNA
cgctctttggccacctgaagcgtcggatccctacagtgcctcccagcctgggcgggagcg

SEQ ID NO: 17 - DNA, 43 bp, Homo sapiens
  source 1..43: mol_type = genomic DNA
cgtcggatccctacagtgcctcccagcctgggcgggagcggcg

SEQ ID NO: 18 - DNA, 46 bp, Homo sapiens
  source 1..46: mol_type = genomic DNA
cggatccctacagtgcctcccagcctgggcgggagcggcggctgcg

SEQ ID NO: 19 - DNA, 75 bp, Homo sapiens
  source 1..75: mol_type = genomic DNA
cggctgcgtcgctgaaggttggggtccttggtgcgaaagggaggcagctgcagcctcagccccaccccagaagcg

SEQ ID NO: 20 - DNA, 76 bp, Homo sapiens
  source 1..76: mol_type = genomic DNA
cgtcgctgaaggttggggtccttggtgcgaaagggaggcagctgcagcctcagccccaccccagaagcggccttcg

SEQ ID NO: 21 - DNA, 78 bp, Homo sapiens
  source 1..78: mol_type = genomic DNA
cgctgaaggttggggtccttggtgcgaaagggaggcagctgcagcctcagccccaccccagaagcggccttcgcatcg

SEQ ID NO: 22 - DNA, 59 bp, Homo sapiens
  source 1..59: mol_type = genomic DNA
cgaaagggaggcagctgcagcctcagccccaccccagaagcggccttcgcatcgctgcg

SEQ ID NO: 23 - DNA, 27 bp, Homo sapiens
  source 1..27: mol_type = genomic DNA
cgctgcggtgggcgttctcgggcttcg

SEQ ID NO: 24 - DNA, 28 bp, Homo sapiens
  source 1..28: mol_type = genomic DNA
cggtgggcgttctcgggcttcgacttcg

SEQ ID NO: 25 - DNA, 27 bp, Homo sapiens
  source 1..27: mol_type = genomic DNA
cgttctcgggcttcgacttcgccagcg

SEQ ID NO: 26 - DNA, 24 bp, Homo sapiens
  source 1..24: mol_type = genomic DNA
cgggcttcgacttcgccagcgccg

SEQ ID NO: 27 - DNA, 19 bp, Homo sapiens
  source 1..19: mol_type = genomic DNA
cgacttcgccagcgccgcg

SEQ ID NO: 28 - DNA, 35 bp, Homo sapiens
  source 1..35: mol_type = genomic DNA
cgccagcgccgcggggcagaggcacctggagctcg

SEQ ID NO: 29 - DNA, 60 bp, Homo sapiens
  source 1..60: mol_type = genomic DNA
cgccgcggggcagaggcacctggagctcgcagggcccagacctgggttggaaaagcttcg

SEQ ID NO: 30 - DNA, 74 bp, Homo sapiens
  source 1..74: mol_type = genomic DNA
cgcggggcagaggcacctggagctcgcagggcccagacctgggttggaaaagcttcgctgactgcaggcaagcg

SEQ ID NO: 31 - DNA, 76 bp, Homo sapiens
  source 1..76: mol_type = genomic DNA
cggggcagaggcacctggagctcgcagggcccagacctgggttggaaaagcttcgctgactgcaggcaagcgtccg

SEQ ID NO: 32 - DNA, 63 bp, Homo sapiens
  source 1..63: mol_type = genomic DNA
cgcagggcccagacctgggttggaaaagcttcgctgactgcaggcaagcgtccgggaggggcg

SEQ ID NO: 33 - DNA, 31 bp, Homo sapiens
  source 1..31: mol_type = genomic DNA
cgtccgggaggggcggccaggcgaagccccg

SEQ ID NO: 34 - DNA, 30 bp, Homo sapiens
  source 1..30: mol_type = genomic DNA
cgggaggggcggccaggcgaagccccggcg

SEQ ID NO: 35 - DNA, 39 bp, Homo sapiens
  source 1..39: mol_type = genomic DNA
cggccaggcgaagccccggcgctttaccacacacttccg

SEQ ID NO: 36 - DNA, 53 bp, Homo sapiens
  source 1..53: mol_type = genomic DNA
cgaagccccggcgctttaccacacacttccgggtcccatgccagttgcatccg

SEQ ID NO: 37 - DNA, 47 bp, Homo sapiens
  source 1..47: mol_type = genomic DNA
cggcgctttaccacacacttccgggtcccatgccagttgcatccgcg

SEQ ID NO: 38 - DNA, 51 bp, Homo sapiens
  source 1..51: mol_type = genomic DNA
cggggtaggctggctgtgatctcgccgcccccgcccctgcagacttcatcg

SEQ ID NO: 39 - DNA, 54 bp, Homo sapiens
  source 1..54: mol_type = genomic DNA
cggcggctgcctcctcttacggccggaatgcctaatcaccatggtgagaaaacg

SEQ ID NO: 40 - DNA, 58 bp, Homo sapiens
  source 1..58: mol_type = genomic DNA
cggctgcctcctcttacggccggaatgcctaatcaccatggtgagaaaacggacaccg

SEQ ID NO: 41 - DNA, 30 bp, Homo sapiens
  source 1..30: mol_type = genomic DNA
cggggagagaccgagattcagcgagacgcg

SEQ ID NO: 42 - DNA, 21 bp, Homo sapiens
  source 1..21: mol_type = genomic DNA
cgagattcagcgagacgcgcg

SEQ ID NO: 43 - DNA, 48 bp, Homo sapiens
  source 1..48: mol_type = genomic DNA
cggagggccggacgggagccaaggcggctcagttcctcttctcccccg

SEQ ID NO: 44 - DNA, 70 bp, Homo sapiens
  source 1..70: mol_type = genomic DNA
cgcgcacttaatgaagttgaaggctcggtgagcccgggtgaagagggtttggaatctgttgaaaggggcg

SEQ ID NO: 45 - DNA, 36 bp, Homo sapiens
  source 1..36: mol_type = genomic DNA
cggtgccttcctcccggcacgctagcctcctcgccg

SEQ ID NO: 46 - DNA, 121 bp, Homo sapiens
  source 1..121: mol_type = genomic DNA
cgttgcctgtggttccaaatctgagatggggagcgcgcagtgtgtacatccaaatctacacctcggaaagagcagggactgggcccccagccttgcaagatccctgtgaagactggggccg

SEQ ID NO: 47 - DNA, 142 bp, Homo sapiens
  source 1..142: mol_type = genomic DNA
cgcaggcaagaggggcaggtgggcaggattcatcccgagtttccaatatatttaatctctaaacctgataagtacaatgcgcgcatagtctaatttttttccattttgtccctttttttgcttgttttgaatgtttttcacg

SEQ ID NO: 48 - DNA, 116 bp, Homo sapiens
  source 1..116: mol_type = genomic DNA
cgagtttccaatatatttaatctctaaacctgataagtacaatgcgcgcatagtctaatttttttccattttgtccctttttttgcttgttttgaatgtttttcacgttaataccg

SEQ ID NO: 49 - DNA, 39 bp, Homo sapiens
  source 1..39: mol_type = genomic DNA
cgcttcgcctccttcccgcccccggaggttgtgcaggcg

SEQ ID NO: 50 - DNA, 44 bp, Homo sapiens
  source 1..44: mol_type = genomic DNA
cgcctccttcccgcccccggaggttgtgcaggcgctatggtccg

SEQ ID NO: 51 - DNA, 98 bp, Homo sapiens
  source 1..98: mol_type = genomic DNA
cgagtcgaaaagtttctgtaagggccggacccagcatcagatggtaacactgattgaacaagagattagcacaatagatctctaaccgaggggaagcg

SEQ ID NO: 52 - DNA, 105 bp, Homo sapiens
  source 1..105: mol_type = genomic DNA
cgaaaagtttctgtaagggccggacccagcatcagatggtaacactgattgaacaagagattagcacaatagatctctaaccgaggggaagcgttgcttttcacg

SEQ ID NO: 53 - DNA, 37 bp, Homo sapiens
  source 1..37: mol_type = genomic DNA
cgcgggcccagccaccaaggccgcggcagggccagcg

SEQ ID NO: 54 - DNA, 40 bp, Homo sapiens
  source 1..40: mol_type = genomic DNA
cgggcccagccaccaaggccgcggcagggccagcgctgcg

SEQ ID NO: 55 - DNA, 91 bp, Homo sapiens
  source 1..91: mol_type = genomic DNA
cggcagggccagcgctgcgccccgggcccctgcctttagggctgggcaacccaagcagagcaaaggaggttcctgaatgtgtaaatttccg

SEQ ID NO: 56 - DNA, 120 bp, Homo sapiens
  source 1..120: mol_type = genomic DNA
cgccccgggcccctgcctttagggctgggcaacccaagcagagcaaaggaggttcctgaatgtgtaaatttccgctttttagctttttttttttttttttttggaccttccgacacttcg

SEQ ID NO: 57 - DNA, 68 bp, Homo sapiens
  source 1..68: mol_type = genomic DNA
cgggccggtggaactcagctccgggaaggctgtggacctggaccctggggctccccgctgcctgggcg

SEQ ID NO: 58 - DNA, 42 bp, Homo sapiens
  source 1..42: mol_type = genomic DNA
cgctgcctgggcgcctctagcccggcgatgagtggttctgcg

SEQ ID NO: 59 - DNA, 46 bp, Homo sapiens
  source 1..46: mol_type = genomic DNA
cgaattaattacgaaggaaacccaggtgtgaaaggcacccgccgcg

SEQ ID NO: 60 - DNA, 50 bp, Homo sapiens
  source 1..50: mol_type = genomic DNA
cgtttgccatgctccttcccggggtccagggaccggagctgcggtgcacg

SEQ ID NO: 61 - DNA, 139 bp, Homo sapiens
  source 1..139: mol_type = genomic DNA
cggcgtattactcctgattaagtgccgtgtcaacctaattagcagagtaattataaggtgctaatgaatgattcaaagttctttgagttacattttactccaaattaccattttaagaaccctatgaatctgcgaggcg

SEQ ID NO: 62 - DNA, 143 bp, Homo sapiens
  source 1..143: mol_type = genomic DNA
cgtattactcctgattaagtgccgtgtcaacctaattagcagagtaattataaggtgctaatgaatgattcaaagttctttgagttacattttactccaaattaccattttaagaaccctatgaatctgcgaggcgggcatcg

SEQ ID NO: 63 - DNA, 127 bp, Homo sapiens
  source 1..127: mol_type = genomic DNA
cgtgtcaacctaattagcagagtaattataaggtgctaatgaatgattcaaagttctttgagttacattttactccaaattaccattttaagaaccctatgaatctgcgaggcgggcatcgcttccg

SEQ ID NO: 64 - DNA, 105 bp, Homo sapiens
  source 1..105: mol_type = genomic DNA
cggaggttgggggcagcctcggtcagctttctcagtctctcccaggtctacagaatacgccactggacaagtgcctaagcagcgacttctggtccagacacaccg

SEQ ID NO: 65 - DNA, 79 bp, Homo sapiens
  source 1..79: mol_type = genomic DNA
cgcgcttccccacccccctggccctccaaaatcggtaagagaattaagatttcgaatccctattttgaggagccttccg

SEQ ID NO: 66 - DNA, 119 bp, Homo sapiens
  source 1..119: mol_type = genomic DNA
cggactcctgtctggtttgctttttaccgactcttacagaaaaaaagagaatgccattggaagaagctcttttgcgtggtgggcgatgtgtgggtgggggacttgtggcatggcccacg

SEQ ID NO: 67 - DNA, 88 bp, Homo sapiens
  source 1..88: mol_type = genomic DNA
cgatgtgtgggtgggggacttgtggcatggcccacggtgttgtttctgtgcctgcgatgacacacgtatgtcttgagctgtgggctcg

SEQ ID NO: 68 - DNA, 43 bp, Homo sapiens
  source 1..43: mol_type = genomic DNA
cgtgtggactggaaacgctccgagccggttattttaaaaaccg

SEQ ID NO: 69 - DNA, 40 bp, Homo sapiens
  source 1..40: mol_type = genomic DNA
cgctccgagccggttattttaaaaaccgggaaataaggcg

SEQ ID NO: 70 - DNA, 48 bp, Homo sapiens
  source 1..48: mol_type = genomic DNA
cgagccggttattttaaaaaccgggaaataaggcgggttccctcttcg

SEQ ID NO: 71 - DNA, 47 bp, Homo sapiens
  source 1..47: mol_type = genomic DNA
cggttattttaaaaaccgggaaataaggcgggttccctcttcgcccg

SEQ ID NO: 72 - DNA, 57 bp, Homo sapiens
  source 1..57: mol_type = genomic DNA
cgggaaataaggcgggttccctcttcgcccgccacttcccaccaagtaggctgtgcg

SEQ ID NO: 73 - DNA, 86 bp, Homo sapiens
  source 1..86: mol_type = genomic DNA
cgggttccctcttcgcccgccacttcccaccaagtaggctgtgcggccctgggggctgactgtcctcaagcagccaggctccaccg

SEQ ID NO: 74 - DNA, 54 bp, Homo sapiens
  source 1..54: mol_type = genomic DNA
cggcaagagcgccagaggctagcggccgccaggatctctaccaggctctgctcg

SEQ ID NO: 75 - DNA, 51 bp, Homo sapiens
  source 1..51: mol_type = genomic DNA
cgccagaggctagcggccgccaggatctctaccaggctctgctcgcacccg

SEQ ID NO: 76 - DNA, 53 bp, Homo sapiens
  source 1..53: mol_type = genomic DNA
cggccgccaggatctctaccaggctctgctcgcacccgcctgcctccctttcg

SEQ ID NO: 77 - DNA, 65 bp, Homo sapiens
  source 1..65: mol_type = genomic DNA
cgccaggatctctaccaggctctgctcgcacccgcctgcctccctttcgtttggcctgtcctccg

SEQ ID NO: 78 - DNA, 90 bp, Homo sapiens
  source 1..90: mol_type = genomic DNA
cgcctgcctccctttcgtttggcctgtcctccgttcaactgaaatcgttaattttcttacccccttgttctcattttgatatattctacg

SEQ ID NO: 79 - DNA, 91 bp, Homo sapiens
  source 1..91: mol_type = genomic DNA
cgtttggcctgtcctccgttcaactgaaatcgttaattttcttacccccttgttctcattttgatatattctacgctttaaacatgctccg

SEQ ID NO: 80 - DNA, 87 bp, Homo sapiens
  source 1..87: mol_type = genomic DNA
cgctttaaacatgctccgttttcttttgtttagtctgctccctccctctttgtcctttcccccttctctagttatccgtttcgttcg

SEQ ID NO: 81 - DNA, 96 bp, Homo sapiens
  source 1..96: mol_type = genomic DNA
cgttttcttttgtttagtctgctccctccctctttgtcctttcccccttctctagttatccgtttcgttcgatcttgctcctgctttttttattcg

SEQ ID NO: 82 - DNA, 75 bp, Homo sapiens
  source 1..75: mol_type = genomic DNA
cgctctccgttgttttgttcaatttcccttccccttttcttggttgtcgctcgctttctttggttttctttctcg

SEQ ID NO: 83 - DNA, 76 bp, Homo sapiens
  source 1..76: mol_type = genomic DNA
cgttgttttgttcaatttcccttccccttttcttggttgtcgctcgctttctttggttttctttctcggtatttcg

SEQ ID NO: 84 - DNA, 35 bp, Homo sapiens
  source 1..35: mol_type = genomic DNA
cgtcggatcccggggtgctgggtttctcccggccg

SEQ ID NO: 85 - DNA, 36 bp, Homo sapiens
  source 1..36: mol_type = genomic DNA
cggatcccggggtgctgggtttctcccggccgctcg

SEQ ID NO: 86 - DNA, 34 bp, Homo sapiens
  source 1..34: mol_type = genomic DNA
cggggtgctgggtttctcccggccgctcgttccg

SEQ ID NO: 87 - DNA, 31 bp, Homo sapiens
  source 1..31: mol_type = genomic DNA
cgttccgcaccagcgctctctgcagttcgcg

SEQ ID NO: 88 - DNA, 30 bp, Homo sapiens
  source 1..30: mol_type = genomic DNA
cgggggcccgagctgtcggtgccggatgcg

SEQ ID NO: 89 - DNA, 25 bp, Homo sapiens
  source 1..25: mol_type = genomic DNA
cgagctgtcggtgccggatgcggcg

SEQ ID NO: 90 - DNA, 22 bp, Homo sapiens
  source 1..22: mol_type = genomic DNA
cggcgcgcctagcagggacgcg

SEQ ID NO: 91 - DNA, 44 bp, Homo sapiens
  source 1..44: mol_type = genomic DNA
cgcgcctagcagggacgcgggcctgggggggtggctcctgcccg

SEQ ID NO: 92 - DNA, 45 bp, Homo sapiens
  source 1..45: mol_type = genomic DNA
cgcctagcagggacgcgggcctgggggggtggctcctgcccgacg

SEQ ID NO: 93 - DNA, 34 bp, Homo sapiens
  source 1..34: mol_type = genomic DNA
cgcgggcctgggggggtggctcctgcccgacgcg

SEQ ID NO: 94 - DNA, 25 bp, Homo sapiens
  source 1..25: mol_type = genomic DNA
cgacgcggagcgctgagccaggccg

SEQ ID NO: 95 - DNA, 39 bp, Homo sapiens
  source 1..39: mol_type = genomic DNA
cgcggagcgctgagccaggccgggtacctgtctctggcg

SEQ ID NO: 96 - DNA, 47 bp, Homo sapiens
  source 1..47: mol_type = genomic DNA
cggagcgctgagccaggccgggtacctgtctctggcggtgctcaccg

SEQ ID NO: 97 - DNA, 49 bp, Homo sapiens
  source 1..49: mol_type = genomic DNA
cgctgagccaggccgggtacctgtctctggcggtgctcaccgcactgcg

SEQ ID NO: 98 - DNA, 117 bp, Homo sapiens
  source 1..117: mol_type = genomic DNA
cggcctctgccgtctggctgggatcagaggagccaggccaactgcttctcattaagtcccaactgtggtttttatcaggaaagcctctttcaaagggcacagacacgaagctccgcg

SEQ ID NO: 99 - DNA, 64 bp, Homo sapiens
  source 1..64: mol_type = genomic DNA
cgaaaggcgagaggggcaaaaagttgagaaataggcgagcgggagagataagcaggaaggcccg

SEQ ID NO: 100 - DNA, 97 bp, Homo sapiens
  source 1..97: mol_type = genomic DNA
cggcgccctttttactaaaagatcttttctcatcctatcagcaaatcgttaagaaaggcttagccattgcgggggctccaacttaaggattcccccg

SEQ ID NO: 101 - DNA, 116 bp, Homo sapiens
  source 1..116: mol_type = genomic DNA
cgccctttttactaaaagatcttttctcatcctatcagcaaatcgttaagaaaggcttagccattgcgggggctccaacttaaggattcccccggcccactaaaaggctaggcccg

SEQ ID NO: 102 - DNA, 131 bp, Homo sapiens
  source 1..131: mol_type = genomic DNA
cgtggggaaaataaaagaaccttacaaattctaaggcgtcataacccctgcaagaacttctaactgtatgaaggcccacgcgagattttgacaatagataaatgagctgaggaaatagggtctggccagcg

SEQ ID NO: 103 - DNA, 109 bp, Homo sapiens
  source 1..109: mol_type = genomic DNA
cggagaattcataaaactctatctctgcaggtcacccgctaatcgcattattattagcctcgggagcatggaaattgaactgtcactgcctaaagagaaaatgtaagcg

SEQ ID NO: 104 - DNA, 100 bp, Homo sapiens
  source 1..100: mol_type = genomic DNA
cgctgccccctccccgggcttgtcagcgtgtgaagcggggtctactccccagctctggctgttcagcaggagcaccttccagcatggttttagaagtccg

SEQ ID NO: 105 - DNA, 108 bp, Homo sapiens
  source 1..108: mol_type = genomic DNA
cgggcttgtcagcgtgtgaagcggggtctactccccagctctggctgttcagcaggagcaccttccagcatggttttagaagtccgggctggaaagcttctgaaaccg

SEQ ID NO: 106 - DNA, 109 bp, Homo sapiens
  source 1..109: mol_type = genomic DNA
cgtaaacgccaaaggagtagaaatataattatgttccaccatcgaaaagttggtgggagaaaggcaaggacgaggttgaaggctaaatgagacaactggtcattccccg

SEQ ID NO: 107 - DNA, 117 bp, Homo sapiens
  source 1..117: mol_type = genomic DNA
cgcccctcccaatcccagggaccggcagcccttcatccacggacacccagaaagctgcgctctccacccaaaagaaaaagaaatgcaaacaaacaggactccttgcttaggctggcg

SEQ ID NO: 108 - DNA, 75 bp, Homo sapiens
  source 1..75: mol_type = genomic DNA
cgggttttccctttactaacatcgctcttgtttttccatttagtctttgggaacgtagcctcaatcttaaccgcg

SEQ ID NO: 109 - DNA, 58 bp, Homo sapiens
  source 1..58: mol_type = genomic DNA
cggatctctaaattatctaatctggcggctgcgtacgactcagggaaagccctggccg

SEQ ID NO: 110 - DNA, 42 bp, Homo sapiens
  source 1..42: mol_type = genomic DNA
cggcgggccaggaactcccgggcgtgtgtgtgcagtggtgcg

SEQ ID NO: 111 - DNA, 94 bp, Homo sapiens
  source 1..94: mol_type = genomic DNA
cggttgctgggcagcactggacagtgcccgacgtaggcgatctcattgattccttcccacaaccccactttgcagataggaaagctgaggcacg

SEQ ID NO: 112 - DNA, 91 bp, Homo sapiens
  source 1..91: mol_type = genomic DNA
cgtcgaaaaccacaatttttagcttgtaattatttgaattcggaaacgatcaccaaactgtagtcaatattttcctagtcctttttaagcg

SEQ ID NO: 113 - DNA, 96 bp, Homo sapiens
  source 1..96: mol_type = genomic DNA
cgaaaaccacaatttttagcttgtaattatttgaattcggaaacgatcaccaaactgtagtcaatattttcctagtcctttttaagcgaggctccg

SEQ ID NO: 114 - DNA, 122 bp, Homo sapiens
  source 1..122: mol_type = genomic DNA
cgggccgcaaagagagaaatcacaaatgtttcccctgacttttgcctttgtgcagtggaccctggcccctgccgctctccctgcacaccctgggcttggtctgcctgggggagaaccccgcg

SEQ ID NO: 115 - DNA, 120 bp, Homo sapiens
  source 1..120: mol_type = genomic DNA
cgcaaagagagaaatcacaaatgtttcccctgacttttgcctttgtgcagtggaccctggcccctgccgctctccctgcacaccctgggcttggtctgcctgggggagaaccccgcgtcg

SEQ ID NO: 116 - DNA, 57 bp, Homo sapiens
  source 1..57: mol_type = genomic DNA
cgctctccctgcacaccctgggcttggtctgcctgggggagaaccccgcgtcgtgcg

SEQ ID NO: 117 - DNA, 67 bp, Homo sapiens
  source 1..67: mol_type = genomic DNA
cgaatgtggaggtcgggtgggtgggtgggtgggtactcgcggcatttctcaaggttctgggtgttcg

SEQ ID NO: 118 - DNA, 96 bp, Homo sapiens
  source 1..96: mol_type = genomic DNA
cgggtgggtgggtgggtgggtactcgcggcatttctcaaggttctgggtgttcgtaactaggggaacaggccctatgggggcagaaatgacagacg

SEQ ID NO: 119 - DNA, 26 bp, Homo sapiens
  source 1..26: mol_type = genomic DNA
cgtcgctccagggagcccggccgccg

SEQ ID NO: 120 - DNA, 25 bp, Homo sapiens
  source 1..25: mol_type = genomic DNA
cgctccagggagcccggccgccgcg

SEQ ID NO: 121 - DNA, 60 bp, Homo sapiens
  source 1..60: mol_type = genomic DNA
cgcgctcacctggttgtttttgcagagatcagcccacatgctggtgccgtcccctccgcg

SEQ ID NO: 122 - DNA, 68 bp, Homo sapiens
  source 1..68: mol_type = genomic DNA
cgctcacctggttgtttttgcagagatcagcccacatgctggtgccgtcccctccgcgcatcaggacg

SEQ ID NO: 123 - DNA, 19 bp, Homo sapiens
  source 1..19: mol_type = genomic DNA
cgcggcgcctggccgaccg

SEQ ID NO: 124 - DNA, 19 bp, Homo sapiens
  source 1..19: mol_type = genomic DNA
cggcgcctggccgaccgcg

SEQ ID NO: 125 - DNA, 97 bp, Homo sapiens
  source 1..97: mol_type = genomic DNA
cgcatgtacctactgtctcctttgccccacatgctccaagaaaataagacacattctaccccgagtcctaattattgggccatttccttaacgcgcg

SEQ ID NO: 126 - DNA, 80 bp, Homo sapiens
  source 1..80: mol_type = genomic DNA
cgtctgctcctcagtccctgcgcccctctctgcactaccctggacccatcacctcccggatcggttctccacttaccccg

SEQ ID NO: 127 - DNA, 83 bp, Homo sapiens
  source 1..83: mol_type = genomic DNA
cgcccctctctgcactaccctggacccatcacctcccggatcggttctccacttaccccgctgtttccctgtctctccttccg

SEQ ID NO: 128 - DNA, 36 bp, Homo sapiens
  source 1..36: mol_type = genomic DNA
cgtcgcgtgggcctactgtggcaaaggagacgtccg

SEQ ID NO: 129 - DNA, 35 bp, Homo sapiens
  source 1..35: mol_type = genomic DNA
cgcgtgggcctactgtggcaaaggagacgtccgcg

SEQ ID NO: 130 - DNA, 35 bp, Homo sapiens
  source 1..35: mol_type = genomic DNA
cgtgggcctactgtggcaaaggagacgtccgcgcg

SEQ ID NO: 131 - DNA, 16 bp, Homo sapiens
  source 1..16: mol_type = genomic DNA
cgtccgcgcgaatccg

SEQ ID NO: 132 - DNA, 74 bp, Homo sapiens
  source 1..74: mol_type = genomic DNA
cggcatcccagctccccgggcggacccaggggctgcctctccacaacagttccgctcccttctgcagagccccg

SEQ ID NO: 133 - DNA, 132 bp, Homo sapiens
  source 1..132: mol_type = genomic DNA
cgcagtccaggctccagggatctgtgagtcctggggagctttttgtttgcgcaaactcttgcttatggaatcctgctctgtcctggagactggatgcagaacagcccgcaccttgttgcagcgctccagccg

SEQ ID NO: 134 - DNA, 91 bp, Homo sapiens
  source 1..91: mol_type = genomic DNA
cgcaaactcttgcttatggaatcctgctctgtcctggagactggatgcagaacagcccgcaccttgttgcagcgctccagccgggctcccg

SEQ ID NO: 135 - DNA, 60 bp, Homo sapiens
  source 1..60: mol_type = genomic DNA
cggagtttctcccctagctcaggccccatggggtagcgcgggagagtctgggcgaagtcg

SEQ ID NO: 136 - DNA, 65 bp, Homo sapiens
  source 1..65: mol_type = genomic DNA
cgcggactcatgcagaagaggacattccgcaggtaggtacaatcccagcgctggggcctggggcg

SEQ ID NO: 137 - DNA, 67 bp, Homo sapiens
  source 1..67: mol_type = genomic DNA
cggactcatgcagaagaggacattccgcaggtaggtacaatcccagcgctggggcctggggcgtccg

SEQ ID NO: 138 - DNA, 49 bp, Homo sapiens
  source 1..49: mol_type = genomic DNA
cgcaggtaggtacaatcccagcgctggggcctggggcgtccggggggcg

SEQ ID NO: 139 - DNA, 44 bp, Homo sapiens
  source 1..44: mol_type = genomic DNA
cgctggggcctggggcgtccggggggcggcctttgagcttcccg

SEQ ID NO: 140 - DNA, 36 bp, Homo sapiens
  source 1..36: mol_type = genomic DNA
cgtccggggggcggcctttgagcttcccggataccg

SEQ ID NO: 141 - DNA, 29 bp, Homo sapiens
  source 1..29: mol_type = genomic DNA
cgcctctccgcccggaattcgttgagacg

SEQ ID NO: 142 - DNA, 57 bp, Homo sapiens
  source 1..57: mol_type = genomic DNA
cggaaatgacccgtctctgttcaaaaatagaaaaagaacgatcggaataattgttcg

SEQ ID NO: 143 - DNA, 82 bp, Homo sapiens
  source 1..82: mol_type = genomic DNA
cgggcatggggcctgagcttaggccaggtggaaaggtggaaggacctagtggggacagacgaagagaccgaggagccgcccg

SEQ ID NO: 144 - DNA, 27 bp, Homo sapiens
  source 1..27: mol_type = genomic DNA
cgaagagaccgaggagccgcccgcccg

SEQ ID NO: 145 - DNA, 45 bp, Homo sapiens
  source 1..45: mol_type = genomic DNA
cgagcctcgggccaagacctccgccctggctccgccagtcctgcg

SEQ ID NO: 146 - DNA, 22 bp, Homo sapiens
  source 1..22: mol_type = genomic DNA
cgcgcgcagctggagcccggcg

SEQ ID NO: 147 - DNA, 31 bp, Homo sapiens
  source 1..31: mol_type = genomic DNA
cggaccccggggaggagccgggaggcgagcg

SEQ ID NO: 148 - DNA, 22 bp, Homo sapiens
  source 1..22: mol_type = genomic DNA
cggggtttcgcactgcgccgcg

SEQ ID NO: 149 - DNA, 75 bp, Homo sapiens
  source 1..75: mol_type = genomic DNA
cgcctccagcccggggtaaggagatccacggaggcctctgcgcctaaactcaggtggccagacagagttggggcg

SEQ ID NO: 150 - DNA, 71 bp, Homo sapiens
  source 1..71: mol_type = genomic DNA
cggggtaaggagatccacggaggcctctgcgcctaaactcaggtggccagacagagttggggcgggaggcg

SEQ ID NO: 151 - DNA, 43 bp, Homo sapiens
  source 1..43: mol_type = genomic DNA
cgcgaagcccctatcctggtttcaagtctccggcttcgttccg

SEQ ID NO: 152 - DNA, 51 bp, Homo sapiens
  source 1..51: mol_type = genomic DNA
cgtctctgcctctccggaaacccgaaatccgcccaaacctcatcccaggcg

SEQ ID NO: 153 - DNA, 101 bp, Homo sapiens
  source 1..101: mol_type = genomic DNA
cgggttggtgaggcgggtgggagcagagacgggtaagaggggtaagggactttgcggccaccctgttctgaagatgtagagtgggattgtgttaaaatccg

SEQ ID NO: 154 - DNA, 64 bp, Homo sapiens
  source 1..64: mol_type = genomic DNA
cgctcagagccagggtctctgctggggtcagcgtctgtacgctaaagccagggcgcaaagtgcg

SEQ ID NO: 155 - DNA, 82 bp, Homo sapiens
  source 1..82: mol_type = genomic DNA
cgccagttctctttcagaagcgggagaagttcaggtgggacaggagacacgggggtgaggagatctttgagagggccggccg

SEQ ID NO: 156 - DNA, 74 bp, Homo sapiens
  source 1..74: mol_type = genomic DNA
cgggcctcctcgggcctcgctgcagcccagctaggttaatcaatcagcagccgctcagtcacccatcccttccg

SEQ ID NO: 157 - DNA, 75 bp, Homo sapiens
  source 1..75: mol_type = genomic DNA
cgggcctcgctgcagcccagctaggttaatcaatcagcagccgctcagtcacccatcccttccgcatcagttgcg

SEQ ID NO: 158 - DNA, 134 bp, Homo sapiens
  source 1..134: mol_type = genomic DNA
cgctcccgctgcccgctgcccactgcagcctatttacctgttcctccagccatccccttagtcctgggccgggagagaccatttcctccttcctggagggatgagccaacagggctgagccccctactctctcg

SEQ ID NO: 159 - DNA, 90 bp, Homo sapiens
  source 1..90: mol_type = genomic DNA
cgggagagaccatttcctccttcctggagggatgagccaacagggctgagccccctactctctcggtggtcagcccgcccgaccaccacg

SEQ ID NO: 160 - DNA, 55 bp, Homo sapiens
  source 1..55: mol_type = genomic DNA
cggtggtcagcccgcccgaccaccacgcccctcctttctagggccccagctcccg

SEQ ID NO: 161 - DNA, 48 bp, Homo sapiens
  source 1..48: mol_type = genomic DNA
cgcccgaccaccacgcccctcctttctagggccccagctcccgggccg

SEQ ID NO: 162 - DNA, 46 bp, Homo sapiens
  source 1..46: mol_type = genomic DNA
cgaccaccacgcccctcctttctagggccccagctcccgggccgcg

SEQ ID NO: 163 - DNA, 42 bp, Homo sapiens
  source 1..42: mol_type = genomic DNA
cgcccctcctttctagggccccagctcccgggccgcggctcg

SEQ ID NO: 164 - DNA, 40 bp, Homo sapiens
  source 1..40: mol_type = genomic DNA
cgggccgcggctcgtttgcatttcaaagctacacaatgcg

SEQ ID NO: 165 - DNA, 39 bp, Homo sapiens
  source 1..39: mol_type = genomic DNA
cgcggctcgtttgcatttcaaagctacacaatgcgggcg

SEQ ID NO: 166 - DNA, 43 bp, Homo sapiens
  source 1..43: mol_type = genomic DNA
cggctcgtttgcatttcaaagctacacaatgcgggcggtcccg

SEQ ID NO: 167 - DNA, 41 bp, Homo sapiens
  source 1..41: mol_type = genomic DNA
cgtttgcatttcaaagctacacaatgcgggcggtcccggcg

SEQ ID NO: 168 - DNA, 20 bp, Homo sapiens
  source 1..20: mol_type = genomic DNA
cgggcggtcccggcgaggcg

SEQ ID NO: 169 - DNA, 26 bp, Homo sapiens
  source 1..26: mol_type = genomic DNA
cggtcccggcgaggcgggaggggccg

SEQ ID NO: 170 - DNA, 40 bp, Homo sapiens
  source 1..40: mol_type = genomic DNA
cggcgaggcgggaggggccggggcagagggagggtcttcg

SEQ ID NO: 171 - DNA, 57 bp, Homo sapiens
  source 1..57: mol_type = genomic DNA
cgaggcgggaggggccggggcagagggagggtcttcgcctccccacccccagccccg

SEQ ID NO: 172 - DNA, 58 bp, Homo sapiens
  source 1..58: mol_type = genomic DNA
cgggaggggccggggcagagggagggtcttcgcctccccacccccagccccgtttccg

SEQ ID NO: 173 - DNA, 55 bp, Homo sapiens
  source 1..55: mol_type = genomic DNA
cggggcagagggagggtcttcgcctccccacccccagccccgtttccggagcccg

SEQ ID NO: 174 - DNA, 37 bp, Homo sapiens
  source 1..37: mol_type = genomic DNA
cgcctccccacccccagccccgtttccggagcccgcg

SEQ ID NO: 175 - DNA, 67 bp, Homo sapiens
  source 1..67: mol_type = genomic DNA
cgatgatgacatcatcgtgtcacacacaggagccggctgccggggggtttaagagaagagcctttcg

SEQ ID NO: 176 - DNA, 62 bp, Homo sapiens
  source 1..62: mol_type = genomic DNA
cgtgtcacacacaggagccggctgccggggggtttaagagaagagcctttcggaccacaccg

SEQ ID NO: 177 - DNA, 31 bp, Homo sapiens
  source 1..31: mol_type = genomic DNA
cggaccacaccggcgctcacgctcatacccg

SEQ ID NO: 178 - DNA, 25 bp, Homo sapiens
  source 1..25: mol_type = genomic DNA
cggcgctcacgctcatacccgcacg

SEQ ID NO: 179 - DNA, 27 bp, Homo sapiens
  source 1..27: mol_type = genomic DNA
cgctcacgctcatacccgcacgccccg

SEQ ID NO: 180 - DNA, 31 bp, Homo sapiens
  source 1..31: mol_type = genomic DNA
cgctcatacccgcacgccccgggcagagccg

SEQ ID NO: 181 - DNA, 23 bp, Homo sapiens
  source 1..23: mol_type = genomic DNA
cgcacgccccgggcagagccgcg

SEQ ID NO: 182 - DNA, 23 bp, Homo sapiens
  source 1..23: mol_type = genomic DNA
cgccccgggcagagccgcgcacg

SEQ ID NO: 183 - DNA, 23 bp, Homo sapiens
  source 1..23: mol_type = genomic DNA
cggcgctcgccccccggcccccg

SEQ ID NO: 184 - DNA, 25 bp, Homo sapiens
  source 1..25: mol_type = genomic DNA
cgctcgccccccggcccccggctcg

SEQ ID NO: 185 - DNA, 26 bp, Homo sapiens
  source 1..26: mol_type = genomic DNA
cgccccccggcccccggctcgggccg

SEQ ID NO: 186 - DNA, 21 bp, Homo sapiens
  source 1..21: mol_type = genomic DNA
cggcccccggctcgggccgcg

SEQ ID NO: 187 - DNA, 19 bp, Homo sapiens
  source 1..19: mol_type = genomic DNA
cggctcgggccgcgagtcg

SEQ ID NO: 188 - DNA, 71 bp, Homo sapiens
  source 1..71: mol_type = genomic DNA
cggctagcttgcacgccagggcacagcgaggatgggagggtcgcagtccctgcagccagccccagccagcg

SEQ ID NO: 189 - DNA, 79 bp, Homo sapiens
  source 1..79: mol_type = genomic DNA
cgccagggcacagcgaggatgggagggtcgcagtccctgcagccagccccagccagcgacctgaacctggaggcttccg

SEQ ID NO: 190 - DNA, 85 bp, Homo sapiens
  source 1..85: mol_type = genomic DNA
cgaggatgggagggtcgcagtccctgcagccagccccagccagcgacctgaacctggaggcttccgaggcaatgtaagtgctccg

SEQ ID NO: 191 - DNA, 74 bp, Homo sapiens
  source 1..74: mol_type = genomic DNA
cgcagtccctgcagccagccccagccagcgacctgaacctggaggcttccgaggcaatgtaagtgctccgcccg

SEQ ID NO: 192 - DNA, 100 bp, Homo sapiens
  source 1..100: mol_type = genomic DNA
cgagtctcccggggaggaggagaggatgcccgggctttgttagtgtcgcctttcctaatgcctgtgcaacctggagcccaggctggcctggggttcttcg

SEQ ID NO: 193 - DNA, 82 bp, Homo sapiens
  source 1..82: mol_type = genomic DNA
cgtcaagtttgagcttaatttctggaaataggcggaagtcgctccggatcatgcatggaaggctaattgaaaagatcagtcg

SEQ ID NO: 194 - DNA, 52 bp, Homo sapiens
  source 1..52: mol_type = genomic DNA
cgtttgggggatctgggctccggccgggaccagcgcaagcagggactttgcg

SEQ ID NO: 195 - DNA, 67 bp, Homo sapiens
  source 1..67: mol_type = genomic DNA
cgggaccagcgcaagcagggactttgcggggacaccgcttctccaacagagcaaggcctggcccacg

SEQ ID NO: 196 - DNA, 64 bp, Homo sapiens
  source 1..64: mol_type = genomic DNA
cgcaagcagggactttgcggggacaccgcttctccaacagagcaaggcctggcccacgtttccg

SEQ ID NO: 197 - DNA, 86 bp, Homo sapiens
  source 1..86: mol_type = genomic DNA
cggggacaccgcttctccaacagagcaaggcctggcccacgtttccggtttctcctaacttccttttattgccttcctttgcttcg

SEQ ID NO: 198 - DNA, 68 bp, Homo sapiens
  source 1..68: mol_type = genomic DNA
cgagagagactttccaaacttcgggcatagccacggcaattcccagtctgctaatgccaaggcgggcg

SEQ ID NO: 199 - DNA, 49 bp, Homo sapiens
  source 1..49: mol_type = genomic DNA
cgggcatagccacggcaattcccagtctgctaatgccaaggcgggcgcg

SEQ ID NO: 200 - DNA, 45 bp, Homo sapiens
  source 1..45: mol_type = genomic DNA
cggcaattcccagtctgctaatgccaaggcgggcgcgtaaggccg

SEQ ID NO: 201 - DNA, 62 bp, Homo sapiens
  source 1..62: mol_type = genomic DNA
cgggcgcgtaaggccgcctaaatctagacctccctcctcactcatttcaaaaaataacaacg

SEQ ID NO: 202 - DNA, 73 bp, Homo sapiens
  source 1..73: mol_type = genomic DNA
cgcgtaaggccgcctaaatctagacctccctcctcactcatttcaaaaaataacaacgtgccagccacctccg

SEQ ID NO: 203 - DNA, 80 bp, Homo sapiens
  source 1..80: mol_type = genomic DNA
cgtaaggccgcctaaatctagacctccctcctcactcatttcaaaaaataacaacgtgccagccacctccgcagataccg

SEQ ID NO: 204 - DNA, 50 bp, Homo sapiens
  source 1..50: mol_type = genomic DNA
cgcagataccgccggctggtgcttgcccaggagacgccagggccagagcg

SEQ ID NO: 205 - DNA, 56 bp, Homo sapiens
  source 1..56: mol_type = genomic DNA
cgccggctggtgcttgcccaggagacgccagggccagagcgccactcccagcatcg

SEQ ID NO: 206 - DNA, 72 bp, Homo sapiens
  source 1..72: mol_type = genomic DNA
cggctggtgcttgcccaggagacgccagggccagagcgccactcccagcatcgaaatggcagagagaaagcg

SEQ ID NO: 207 - DNA, 117 bp, Homo sapiens
  source 1..117: mol_type = genomic DNA
cgccagggccagagcgccactcccagcatcgaaatggcagagagaaagcgcagctccaaattccccttcagaggttaagcctcaatcattgtgtcccttccctagggactgctggcg

SEQ ID NO: 208 - DNA, 104 bp, Homo sapiens
  source 1..104: mol_type = genomic DNA
cgaaatggcagagagaaagcgcagctccaaattccccttcagaggttaagcctcaatcattgtgtcccttccctagggactgctggcgctctcgcccactggcg

SEQ ID NO: 209 - DNA, 59 bp, Homo sapiens
  source 1..59: mol_type = genomic DNA
cgaaggcgagcctaccggccaagccctggggtccggcaggttctgcacaactactcccg

SEQ ID NO: 210 - DNA, 62 bp, Homo sapiens
  source 1..62: mol_type = genomic DNA
cgagcctaccggccaagccctggggtccggcaggttctgcacaactactcccgcaaagctcg

SEQ ID NO: 211 - DNA, 81 bp, Homo sapiens
  source 1..81: mol_type = genomic DNA
cggccaagccctggggtccggcaggttctgcacaactactcccgcaaagctcgccacctttgtgccctttcctcagctacg

SEQ ID NO: 212 - DNA, 38 bp, Homo sapiens
  source 1..38: mol_type = genomic DNA
cgcttaccagccccggaagcaccagggggcgaccggcg

SEQ ID NO: 213 - DNA, 67 bp, Homo sapiens
  source 1..67: mol_type = genomic DNA
cgctgggcctccaggctcccaggtcggagctctagactagccctgccgcgtaccagcacccaccacg

SEQ ID NO: 214 - DNA, 80 bp, Homo sapiens
  source 1..80: mol_type = genomic DNA
cgggatggagaggcgcttttggcaggaatcatttttaggaggagttcgaggctcgatgagtaccctcaacctggccctcg

SEQ ID NO: 215 - DNA, 73 bp, Homo sapiens
  source 1..73: mol_type = genomic DNA
cgcttttggcaggaatcatttttaggaggagttcgaggctcgatgagtaccctcaacctggccctcgagcccg

SEQ ID NO: 216 - DNA, 73 bp, Homo sapiens
  source 1..73: mol_type = genomic DNA
cgcgcctgccaccagccttggaggaagctgggcagggccccgtgtggtgcccagcctacgaggtcccacctcg

SEQ ID NO: 217 - DNA, 53 bp, Homo sapiens
  source 1..53: mol_type = genomic DNA
cgtcaggcccccagctcccccggatcccaccacgcaccaggcccctccgcccg

SEQ ID NO: 218 - DNA, 38 bp, Homo sapiens
  source 1..38: mol_type = genomic DNA
cgagaacgaccagatggtgccgggcatgaagcgggccg

SEQ ID NO: 219 - DNA, 35 bp, Homo sapiens
  source 1..35: mol_type = genomic DNA
cgaccagatggtgccgggcatgaagcgggccgacg

SEQ ID NO: 220 - DNA, 54 bp, Homo sapiens
  source 1..54: mol_type = genomic DNA
cgggcatgaagcgggccgacgaggaataggtggtggaggtggaggtggaggacg

SEQ ID NO: 221 - DNA, 46 bp, Homo sapiens
  source 1..46: mol_type = genomic DNA
cgggccgacgaggaataggtggtggaggtggaggtggaggacgacg

SEQ ID NO: 222 - DNA, 53 bp, Homo sapiens
  source 1..53: mol_type = genomic DNA
cgacgaggaataggtggtggaggtggaggtggaggacgacgagaggctggccg

SEQ ID NO: 223 - DNA, 52 bp, Homo sapiens
  source 1..52: mol_type = genomic DNA
cgaggaataggtggtggaggtggaggtggaggacgacgagaggctggccgcg

SEQ ID NO: 224 - DNA, 26 bp, Homo sapiens
  source 1..26: mol_type = genomic DNA
cgacgagaggctggccgcggagggcg

SEQ ID NO: 225 - DNA, 25 bp, Homo sapiens
  source 1..25: mol_type = genomic DNA
cgagaggctggccgcggagggcgcg

SEQ ID NO: 226 - DNA, 22 bp, Homo sapiens
  source 1..22: mol_type = genomic DNA
cgcggagggcgcgagggggccg

SEQ ID NO: 227 - DNA, 24 bp, Homo sapiens
  source 1..24: mol_type = genomic DNA
cgagggggccggccggggagcgcg

SEQ ID NO: 228 - DNA, 86 bp, Homo sapiens
  source 1..86: mol_type = genomic DNA
cggcgaagtggtcgcctcccagtgagtcccccagtggcccggccaggccctgctgctcctgctgcaggaggaagaggttggggccg

SEQ ID NO: 229 - DNA, 90 bp, Homo sapiens
  source 1..90: mol_type = genomic DNA
cgaagtggtcgcctcccagtgagtcccccagtggcccggccaggccctgctgctcctgctgcaggaggaagaggttggggccgaataacg

SEQ ID NO: 230 - DNA, 38 bp, Homo sapiens
  source 1..38: mol_type = genomic DNA
cgggccggggcccgcaggccgagatgcagctgaaggcg

SEQ ID NO: 231 - DNA, 35 bp, Homo sapiens
  source 1..35: mol_type = genomic DNA
cggggcccgcaggccgagatgcagctgaaggcgcg

SEQ ID NO: 232 - DNA, 35 bp, Homo sapiens
  source 1..35: mol_type = genomic DNA
cgcaggccgagatgcagctgaaggcgcggaccccg

SEQ ID NO: 233 - DNA, 37 bp, Homo sapiens
  source 1..37: mol_type = genomic DNA
cgagatgcagctgaaggcgcggaccccgcagggggcg

SEQ ID NO: 234 - DNA, 28 bp, Homo sapiens
  source 1..28: mol_type = genomic DNA
cgcggaccccgcagggggcgctgaggcg

SEQ ID NO: 235 - DNA, 49 bp, Homo sapiens
  source 1..49: mol_type = genomic DNA
cgcagggggcgctgaggcgggagtagaggcaggacatggtgcggtggcg

SEQ ID NO: 236 - DNA, 62 bp, Homo sapiens
  source 1..62: mol_type = genomic DNA
cgggcgcaagaaacgcgtgccctacactaaggtgcagctgaaggagctagagaaggaatacg

SEQ ID NO: 237 - DNA, 60 bp, Homo sapiens
  source 1..60: mol_type = genomic DNA
cgcaagaaacgcgtgccctacactaaggtgcagctgaaggagctagagaaggaatacgcg

SEQ ID NO: 238 - DNA, 80 bp, Homo sapiens
  source 1..80: mol_type = genomic DNA
cgcgtgccctacactaaggtgcagctgaaggagctagagaaggaatacgcggctagcaagttcatcaccaaagagaagcg

SEQ ID NO: 239 - DNA, 81 bp, Homo sapiens
  source 1..81: mol_type = genomic DNA
cgtgccctacactaaggtgcagctgaaggagctagagaaggaatacgcggctagcaagttcatcaccaaagagaagcgccg

SEQ ID NO: 240 - DNA, 54 bp, Homo sapiens
  source 1..54: mol_type = genomic DNA
cgccaccacgaacctctctgagcgccaggtaaccatctggttccagaaccggcg

SEQ ID NO: 241 - DNA, 77 bp, Homo sapiens
  source 1..77: mol_type = genomic DNA
cgaacctctctgagcgccaggtaaccatctggttccagaaccggcgggtcaaagagaagaaggtggtcagcaaatcg

SEQ ID NO: 242 - DNA, 69 bp, Homo sapiens
  source 1..69: mol_type = genomic DNA
cgccaggtaaccatctggttccagaaccggcgggtcaaagagaagaaggtggtcagcaaatcgaaagcg

SEQ ID NO: 243 - DNA, 34 bp, Homo sapiens
  source 1..34: mol_type = genomic DNA
cgcgccgcccggaggcagagaggctccatggccg

SEQ ID NO: 244 - DNA, 100 bp, Homo sapiens
  source 1..100: mol_type = genomic DNA
cgccgcccggaggcagagaggctccatggccgtgctgctgggccatccccaactccctatcccatccccagcctccacccccatccagatgggactcacg

SEQ ID NO: 245 - DNA, 113 bp, Homo sapiens
  source 1..113: mol_type = genomic DNA
cgtgctgctgggccatccccaactccctatcccatccccagcctccacccccatccagatgggactcacgtggcttcaacagctttggaaatgggtcccgagtgggccgtgcg

SEQ ID NO: 246 - DNA, 94 bp, Homo sapiens
  source 1..94: mol_type = genomic DNA
cgtgttaatactcatgataattattagggacctggcagcgtgattggagtatggatgtttccgtaaaagctggaattccgtaaaagcattgacg

SEQ ID NO: 247 - DNA, 107 bp, Homo sapiens
  source 1..107: mol_type = genomic DNA
cgctgagctggctgcgctgaggcctggcgtgcggagaaacaaatatattggatatttgctggtctaggggaggggtgaggcaagaatggagggagacagggagaccg

SEQ ID NO: 248 - DNA, 99 bp, Homo sapiens
  source 1..99: mol_type = genomic DNA
cgctgaggcctggcgtgcggagaaacaaatatattggatatttgctggtctaggggaggggtgaggcaagaatggagggagacagggagaccgggagcg

SEQ ID NO: 249 - DNA, 59 bp, Homo sapiens
  source 1..59: mol_type = genomic DNA
cgtcttgtgtgctggtttcccgtgtgatacgtgactcctccaaactgaccgaggctccg

SEQ ID NO: 250 - DNA, 74 bp, Homo sapiens
  source 1..74: mol_type = genomic DNA
cggaaccgatgtttctttcctaatctgccgctgccttggttttccctccagcttgagggtgcggatttcctgcg

SEQ ID NO: 251 - DNA, 25 bp, Homo sapiens
  source 1..25: mol_type = genomic DNA
cgggcgggcgagagaggggagcgcg

SEQ ID NO: 252 - DNA, 30 bp, Homo sapiens
  source 1..30: mol_type = genomic DNA
cggagtccgcggctgagacccgaaagcccg

SEQ ID NO: 253 - DNA, 46 bp, Homo sapiens
  source 1..46: mol_type = genomic DNA
cggccctttggctcaggggagcgcaggccggccttcctcccgcacg

SEQ ID NO: 254 - DNA, 27 bp, Homo sapiens
  source 1..27: mol_type = genomic DNA
cgcaggccggccttcctcccgcacgcg

SEQ ID NO: 255 - DNA, 39 bp, Homo sapiens
  source 1..39: mol_type = genomic DNA
cgcgggccagctgcagatggagaacccggggggcgaccg

SEQ ID NO: 256 - DNA, 39 bp, Homo sapiens
  source 1..39: mol_type = genomic DNA
cgggccagctgcagatggagaacccggggggcgaccgcg

SEQ ID NO: 257 - DNA, 59 bp, Homo sapiens
  source 1..59: mol_type = genomic DNA
cgtctccaactgcaaggacttcccctccatgctggcgctggacggtctgcatccccgcg

SEQ ID NO: 258 - DNA, 31 bp, Homo sapiens
  source 1..31: mol_type = genomic DNA
cgctggacggtctgcatccccgcggcccccg

SEQ ID NO: 259 - DNA, 56 bp, Homo sapiens
  source 1..56: mol_type = genomic DNA
cggtctgcatccccgcggcccccgccctcagccctccttccttgatggccagcccg

SEQ ID NO: 260 - DNA, 63 bp, Homo sapiens
  source 1..63: mol_type = genomic DNA
cgctttatccggaggcagctggagccccctcgggaggcccgtcattattattattaattatcg

SEQ ID NO: 261 - DNA, 51 bp, Homo sapiens
  source 1..51: mol_type = genomic DNA
cgtgcctaggttctcggtctgcgcagctgggagtctcccccaccagcgccg

SEQ ID NO: 262 - DNA, 67 bp, Homo sapiens
  source 1..67: mol_type = genomic DNA
cgccctttctccccagcccacctttcccattggtcccctctcctgtccgagtggcagcgcggccccg

SEQ ID NO: 263 - DNA, 28 bp, Homo sapiens
  source 1..28: mol_type = genomic DNA
cgggcgccgggttacctccttcggcccg

SEQ ID NO: 264 - DNA, 27 bp, Homo sapiens
  source 1..27: mol_type = genomic DNA
cgccgggttacctccttcggcccggcg

SEQ ID NO: 265 - DNA, 50 bp, Homo sapiens
  source 1..50: mol_type = genomic DNA
cgggttacctccttcggcccggcgggccccacctgagcttttctcacccg

SEQ ID NO: 266 - DNA, 46 bp, Homo sapiens
  source 1..46: mol_type = genomic DNA
cggcccggcgggccccacctgagcttttctcacccgctcaaagccg

SEQ ID NO: 267 - DNA, 52 bp, Homo sapiens
  source 1..52: mol_type = genomic DNA
cggcgggccccacctgagcttttctcacccgctcaaagccgctaccaccccg

SEQ ID NO: 268 - DNA, 51 bp, Homo sapiens
  source 1..51: mol_type = genomic DNA
cgggccccacctgagcttttctcacccgctcaaagccgctaccaccccgcg

SEQ ID NO: 269 - DNA, 37 bp, Homo sapiens
  source 1..37: mol_type = genomic DNA
cgctcaaagccgctaccaccccgcgcccccaacctcg

SEQ ID NO: 270 - DNA, 86 bp, Homo sapiens
  source 1..86: mol_type = genomic DNA
cgactctgggagctgaactggagtggagctcgaagtggctggggcgggcaaagactcccgcttcactctgggacacaactgggacg

SEQ ID NO: 271 - DNA, 71 bp, Homo sapiens
  source 1..71: mol_type = genomic DNA
cgcctgggcctttgcagcgccctgccgcccaggctccagcgcctctactaccctagccccttctcaacacg

SEQ ID NO: 272 - DNA, 56 bp, Homo sapiens
  source 1..56: mol_type = genomic DNA
cgccctgccgcccaggctccagcgcctctactaccctagccccttctcaacacgcg

SEQ ID NO: 273 - DNA, 73 bp, Homo sapiens
  source 1..73: mol_type = genomic DNA
cgcaaccccaagatccgaggaatgcctccgggggatgatttctctggaggggtctcgcagggccccctctgcg

SEQ ID NO: 274 - DNA, 84 bp, Homo sapiens
  source 1..84: mol_type = genomic DNA
cggtagggcatagaagatgggttttcggtgaatcgtgaaaagacatgatcgggtttcccttggagcatggtggagaatgatgcg

SEQ ID NO: 275 - DNA, 124 bp, Homo sapiens
  source 1..124: mol_type = genomic DNA
cgtaaccatatcacatggagctccacactgtccttctgaaagcaattccgctgttgaaaactgaaaaatgggtgtaatttgctttcagaaagtaatgttagggtcacggcaatttctcgggccg

SEQ ID NO: 276 - DNA, 97 bp, Homo sapiens
  source 1..97: mol_type = genomic DNA
cgcgcgcaatgccccaactgaccctgaagggtcttgcctttactgcatttgcccccagacggctagaagtcaatgcactatgaaaatcccagtcccg

SEQ ID NO: 277 - DNA, 133 bp, Homo sapiens
  source 1..133: mol_type = genomic DNA
cggcagactccagctgacccttgttcttgttctaggtcctgcatccttacattcatcgggactgaccagaccttagtgttctctcttttccttccttcatcctctttctctgcctcagatccgtcgtctctcg

SEQ ID NO: 278 - DNA, 87 bp, Homo sapiens
  source 1..87: mol_type = genomic DNA
cgccccgggaaggcttcagcatcaccctcttctgctgggatcccgctgaccctgtctgcagcttcggcttctcttgctcccagagcg

SEQ ID NO: 279 - DNA, 99 bp, Homo sapiens
  source 1..99: mol_type = genomic DNA
cggtgtccactgtcttgggactaagtctctttcatatttaaacaaaactctttcgagagggctcgctggtttggagaaccctcacaaacgctgccaccg

SEQ ID NO: 280 - DNA, 71 bp, Homo sapiens
  source 1..71: mol_type = genomic DNA
cgcaaagaccacagaggagtaggaagagggctgccctcgtactccccaagccacccccgcctctgagcgcg

SEQ ID NO: 281 - DNA, 125 bp, Homo sapiens
  source 1..125: mol_type = genomic DNA
cgcctgtggcggcggcagtacgggcctggggggtcacccacaggaaaggtgagattagcagcaccatgtgggagaggggttgaggaaacccaccattatctaaaacttggaggtttccaaagccg

SEQ ID NO: 282 - DNA, 117 bp, Homo sapiens
  source 1..117: mol_type = genomic DNA
cgcagctgaagtagctccctgacctctttggaggacggcgtcatttcagtaccaagtatcaccattattgggcttattaacttcattaaaaccggtcttgactaggagtggagatcg

SEQ ID NO: 283 - DNA, 80 bp, Homo sapiens
  source 1..80: mol_type = genomic DNA
cgcgggagaagcccagaacggcgttgacataaaaacaaaaccacaaacaaaaccttccaaaacaccccagattacattcg

SEQ ID NO: 284 - DNA, 83 bp, Homo sapiens
  source 1..83: mol_type = genomic DNA
cgggagaagcccagaacggcgttgacataaaaacaaaaccacaaacaaaaccttccaaaacaccccagattacattcgcagcg

SEQ ID NO: 285 - DNA, 97 bp, Homo sapiens
  source 1..97: mol_type = genomic DNA
cgaatgtgccgcttccaaactgtccagtaaaagggcgagagggagacgtgtggtctcctggggcatggacaacctctcccacctcagcagctaggcg

SEQ ID NO: 286 - DNA, 89 bp, Homo sapiens
  source 1..89: mol_type = genomic DNA
cggctgattcggaagtcgctccaagggaaagaaggtttatgctttgtcattatcttcatcgccctctttattttgcccctgccttgtcg

SEQ ID NO: 287 - DNA, 70 bp, Homo sapiens
  source 1..70: mol_type = genomic DNA
cgggtggaggcacctctggcgcccttacccagtccctggcgactccaattcagcaggagttgggagcgcg

SEQ ID NO: 288 - DNA, 136 bp, Homo sapiens
  source 1..136: mol_type = genomic DNA
cgatctgtccttgtcacccacacttcgtttatttcctgacagtcctgtaaatctcccaaaagtgcacaacaaacagggaggacactgcaagcccagtatataaaagacctgggagctgcggcgctgagaaagggcg

SEQ ID NO: 289 - DNA, 93 bp, Homo sapiens
  source 1..93: mol_type = genomic DNA
cgcctctcctaagagctggcctggagtcctggagcctccaagcgggcaagtaggggccgcaaaggctgggctgggcctccgctgaggccctcg

SEQ ID NO: 290 - DNA, 83 bp, Homo sapiens
  source 1..83: mol_type = genomic DNA
cgggcaagtaggggccgcaaaggctgggctgggcctccgctgaggccctcgggggcctctgggccctcatggcctggaggccg

SEQ ID NO: 291 - DNA, 70 bp, Homo sapiens
  source 1..70: mol_type = genomic DNA
cgcaaaggctgggctgggcctccgctgaggccctcgggggcctctgggccctcatggcctggaggccgcg

SEQ ID NO: 292 - DNA, 57 bp, Homo sapiens
  source 1..57: mol_type = genomic DNA
cgcagggccggaggcagcggcttttccttcccactccgggttgaccctaaagacacg

SEQ ID NO: 293 - DNA, 57 bp, Homo sapiens
  source 1..57: mol_type = genomic DNA
cggaggcagcggcttttccttcccactccgggttgaccctaaagacacgatttaacg

SEQ ID NO: 294 - DNA, 53 bp, Homo sapiens
  source 1..53: mol_type = genomic DNA
cggcttttccttcccactccgggttgaccctaaagacacgatttaacgtggcg

SEQ ID NO: 295 - DNA, 37 bp, Homo sapiens
  source 1..37: mol_type = genomic DNA
cgggttgaccctaaagacacgatttaacgtggcggcg

SEQ ID NO: 296 - DNA, 62 bp, Homo sapiens
  source 1..62: mol_type = genomic DNA
cggaggggggcccaggcctcacgcgcccggctctctcccagctctcctcctcctccttctcg

SEQ ID NO: 297 - DNA, 63 bp, Homo sapiens
  source 1..63: mol_type = genomic DNA
cgcgcccggctctctcccagctctcctcctcctccttctcgggctcagaaagccttccagccg

SEQ ID NO: 298 - DNA, 63 bp, Homo sapiens
  source 1..63: mol_type = genomic DNA
cgcccggctctctcccagctctcctcctcctccttctcgggctcagaaagccttccagccgcg

SEQ ID NO: 299 - DNA, 73 bp, Homo sapiens
  source 1..73: mol_type = genomic DNA
cgaattcaatccgagaaggctcctttgagcttttgtgtttgctgggggagatgtgggcgcaggagggatcgcg

SEQ ID NO: 300 - DNA, 134 bp, Homo sapiens
  source 1..134: mol_type = genomic DNA
cgcccccagacaaggacagggttcctacatcagctgggtctaaaataaggaaaagcgagtgctgccgcagaggggactttccgggaagggggtcacaggaaccccaaggccacacagctccagacactctgccg

SEQ ID NO: 301 - DNA, 77 bp, Homo sapiens
  source 1..77: mol_type = genomic DNA
cgcaccccagccaggtgggacctgggcttcgggcctggcagggcctggaggggagaggcgcccactccccagccgcg

SEQ ID NO: 302 - DNA, 56 bp, Homo sapiens
  source 1..56: mol_type = genomic DNA
cgggcctggcagggcctggaggggagaggcgcccactccccagccgcggacacccg

SEQ ID NO: 303 - DNA, 30 bp, Homo sapiens
  source 1..30: mol_type = genomic DNA
cgcccactccccagccgcggacacccgccg

SEQ ID NO: 304 - DNA, 46 bp, Homo sapiens
  source 1..46: mol_type = genomic DNA
cgccctgtccgctcgccccagacagatgcccggttattcattaccg

SEQ ID NO: 305 - DNA, 59 bp, Homo sapiens
  source 1..59: mol_type = genomic DNA
cgaggcctagaggaaagagtggctgccgtcttcctgcccacagcccgccggaccctccg

SEQ ID NO: 306 - DNA, 36 bp, Homo sapiens
  source 1..36: mol_type = genomic DNA
cgtcttcctgcccacagcccgccggaccctccgtcg

SEQ ID NO: 307 - DNA, 29 bp, Homo sapiens
  source 1..29: mol_type = genomic DNA
cggctgcccggtccccggagccgcagccg

SEQ ID NO: 308 - DNA, 24 bp, Homo sapiens
  source 1..24: mol_type = genomic DNA
cgccgagcccggctgtgcgtgtcg

SEQ ID NO: 309 - DNA, 48 bp, Homo sapiens
  source 1..48: mol_type = genomic DNA
cgagcccggctgtgcgtgtcgtggctgctggggagaaagaggcttccg

SEQ ID NO: 310 - DNA, 85 bp, Homo sapiens
  source 1..85: mol_type = genomic DNA
cggatgggccgaacagcagcttccggcagaggccctcaggtttcagagttctccaagtggggagaagggcggcctgctcagcccg

SEQ ID NO: 311 - DNA, 47 bp, Homo sapiens
  source 1..47: mol_type = genomic DNA
cgttccatttatactgaacgcaggcggccgctgaaaatccagcctcg

SEQ ID NO: 312 - DNA, 43 bp, Homo sapiens
  source 1..43: mol_type = genomic DNA
cgatgtggggaggcctgcgcggggactaggttctcgagaggcg

SEQ ID NO: 313 - DNA, 41 bp, Homo sapiens
  source 1..41: mol_type = genomic DNA
cgagactgctgcggggccggatgcgggatccctgggctgcg

SEQ ID NO: 314 - DNA, 82 bp, Homo sapiens
  source 1..82: mol_type = genomic DNA
cgggatccctgggctgcggttctacgcagaaacgccaatggccatgcctccccagctcctcccagccccagtcactaggccg

SEQ ID NO: 315 - DNA, 92 bp, Homo sapiens
  source 1..92: mol_type = genomic DNA
cggaactcgctggatcctctccacatccaggccgcgtccactggctttccaacctgctcaggtccttaaagaaggattcaaaaggtgttgcg

SEQ ID NO: 316 - DNA, 107 bp, Homo sapiens
  source 1..107: mol_type = genomic DNA
cgagctcgcatgctgtcacccattccagcctaaatgtgaccataaaattagggctgctgcttttatcgagaacacctgcttcctctcgttgccttgtgtgaaagtcg

SEQ ID NO: 317 - DNA, 103 bp, Homo sapiens
  source 1..103: mol_type = genomic DNA
cgcatgctgtcacccattccagcctaaatgtgaccataaaattagggctgctgcttttatcgagaacacctgcttcctctcgttgccttgtgtgaaagtcgcg

SEQ ID NO: 318 - DNA, 149 bp, Homo sapiens
  source 1..149: mol_type = genomic DNA
cggtaactgtgatagtgtgactgttagaaaaactccagctataccacaactctccccctccagacacggcggcctgtccaggtcctcctgtgggattcctgctcaccattggctcagggtccgccctgagtggtcctagtcagagatcg

SEQ ID NO: 319 - DNA, 53 bp, Homo sapiens
  source 1..53: mol_type = genomic DNA
cgggcgagctcatccccaatggtcccttctccttctacggaggtgggtgcgcg

SEQ ID NO: 320 - DNA, 32 bp, Homo sapiens
  source 1..32: mol_type = genomic DNA
cggccaggtcggggcgggcttcgttggaagcg

SEQ ID NO: 321 - DNA, 49 bp, Homo sapiens
  source 1..49: mol_type = genomic DNA
cgggacgcccctgggtggcctggagcacccgctgccgggccaccacccg

SEQ ID NO: 322 - DNA, 47 bp, Homo sapiens
  source 1..47: mol_type = genomic DNA
cgcccctgggtggcctggagcacccgctgccgggccaccacccgtcg

SEQ ID NO: 323 - DNA, 85 bp, Homo sapiens
  source 1..85: mol_type = genomic DNA
cgtcggaggaagggcagcgtcagggccaaacccgaagaccaagggactgtgtgcccccaccagacctcccaggaaggagatagcg

SEQ ID NO: 324 - DNA, 89 bp, Homo sapiens
  source 1..89: mol_type = genomic DNA
cggaggaagggcagcgtcagggccaaacccgaagaccaagggactgtgtgcccccaccagacctcccaggaaggagatagcgctccccg

SEQ ID NO: 325 - DNA, 99 bp, Homo sapiens
  source 1..99: mol_type = genomic DNA
cgtcagggccaaacccgaagaccaagggactgtgtgcccccaccagacctcccaggaaggagatagcgctccccgcaggctggggacctctgcccaccg

SEQ ID NO: 326 - DNA, 91 bp, Homo sapiens
  source 1..91: mol_type = genomic DNA
cgacacctgccccactccgggacggtggaaaagacagctgagcccccacctcccttcacattccagaaaagtgtctgaaaggcccggggcg

SEQ ID NO: 327 - DNA, 79 bp, Homo sapiens
  source 1..79: mol_type = genomic DNA
cgggacggtggaaaagacagctgagcccccacctcccttcacattccagaaaagtgtctgaaaggcccggggcgcttcg

SEQ ID NO: 328 - DNA, 91 bp, Homo sapiens
  source 1..91: mol_type = genomic DNA
cggtggaaaagacagctgagcccccacctcccttcacattccagaaaagtgtctgaaaggcccggggcgcttcggggcttgccaagagacg

SEQ ID NO: 329 - DNA, 52 bp, Homo sapiens
  source 1..52: mol_type = genomic DNA
cggggcgcttcggggcttgccaagagacggtgtttagagaaagagcataacg

SEQ ID NO: 330 - DNA, 49 bp, Homo sapiens
  source 1..49: mol_type = genomic DNA
cgcttcggggcttgccaagagacggtgtttagagaaagagcataacgcg

SEQ ID NO: 331 - DNA, 56 bp, Homo sapiens
  source 1..56: mol_type = genomic DNA
cggggcttgccaagagacggtgtttagagaaagagcataacgcgaagtcacaatcg

SEQ ID NO: 332 - DNA, 50 bp, Homo sapiens
  source 1..50: mol_type = genomic DNA
cggtgtttagagaaagagcataacgcgaagtcacaatcgcaggaaactcg

SEQ ID NO: 333 - DNA, 46 bp, Homo sapiens
  source 1..46: mol_type = genomic DNA
cgcgaagtcacaatcgcaggaaactcgcagcagccccccatccccg

SEQ ID NO: 334 - DNA, 47 bp, Homo sapiens
  source 1..47: mol_type = genomic DNA
cgaagtcacaatcgcaggaaactcgcagcagccccccatccccgccg

SEQ ID NO: 335 - DNA, 44 bp, Homo sapiens
  source 1..44: mol_type = genomic DNA
cgcaggaaactcgcagcagccccccatccccgccgctggctccg

SEQ ID NO: 336 - DNA, 40 bp, Homo sapiens
  source 1..40: mol_type = genomic DNA
cgcagcagccccccatccccgccgctggctccgtttagcg

SEQ ID NO: 337 - DNA, 38 bp, Homo sapiens
  source 1..38: mol_type = genomic DNA
cgccgctggctccgtttagcggggagaaaggagggtcg

SEQ ID NO: 338 - DNA, 47 bp, Homo sapiens
  source 1..47: mol_type = genomic DNA
cgctggctccgtttagcggggagaaaggagggtcgcccagctttgcg

SEQ ID NO: 339 - DNA, 48 bp, Homo sapiens
  source 1..48: mol_type = genomic DNA
cgtttagcggggagaaaggagggtcgcccagctttgcgtcctggggcg

SEQ ID NO: 340 - DNA, 46 bp, Homo sapiens
  source 1..46: mol_type = genomic DNA
cggggagaaaggagggtcgcccagctttgcgtcctggggcgcaccg

SEQ ID NO: 341 - DNA, 34 bp, Homo sapiens
  source 1..34: mol_type = genomic DNA
cgcccagctttgcgtcctggggcgcaccgaagcg

SEQ ID NO: 342 - DNA, 54 bp, Homo sapiens
  source 1..54: mol_type = genomic DNA
cggagtgaggaggactatggtcgcggtggaatctctgttccgctggcacatccg

SEQ ID NO: 343 - DNA, 35 bp, Homo sapiens
  source 1..35: mol_type = genomic DNA
cgcggtggaatctctgttccgctggcacatccgcg

SEQ ID NO: 344 - DNA, 41 bp, Homo sapiens
  source 1..41: mol_type = genomic DNA
cggtggaatctctgttccgctggcacatccgcgcaggtgcg

SEQ ID NO: 345 - DNA, 25 bp, Homo sapiens
  source 1..25: mol_type = genomic DNA
cgccaggccgggtccagcgcccgcg

SEQ ID NO: 346 - DNA, 48 bp, Homo sapiens
  source 1..48: mol_type = genomic DNA
cgcggccccctgggccgggacaaggagggcgggtgagcagaagggccg

SEQ ID NO: 347 - DNA, 73 bp, Homo sapiens
  source 1..73: mol_type = genomic DNA
cggccccctgggccgggacaaggagggcgggtgagcagaagggccgtgcccagggcctggaagtgcaaggccg

SEQ ID NO: 348 - DNA, 62 bp, Homo sapiens
  source 1..62: mol_type = genomic DNA
cgggacaaggagggcgggtgagcagaagggccgtgcccagggcctggaagtgcaaggccgcg

SEQ ID NO: 349 - DNA, 70 bp, Homo sapiens
  source 1..70: mol_type = genomic DNA
cgggtgagcagaagggccgtgcccagggcctggaagtgcaaggccgcgtggtgggcatggtagggaagcg

SEQ ID NO: 350 - DNA, 58 bp, Homo sapiens
  source 1..58: mol_type = genomic DNA
cgggaccggggctcccaaatgaacagcgcgcacagctgggagcagggcttggggagcg

SEQ ID NO: 351 - DNA, 42 bp, Homo sapiens
  source 1..42: mol_type = genomic DNA
cgctgcctggaagcgggcgccggcagggggtggccagacccg

SEQ ID NO: 352 - DNA, 33 bp, Homo sapiens
  source 1..33: mol_type = genomic DNA
cgggcgccggcagggggtggccagacccggtcg

SEQ ID NO: 353 - DNA, 36 bp, Homo sapiens
  source 1..36: mol_type = genomic DNA
cgccggcagggggtggccagacccggtcgtggagcg

SEQ ID NO: 354 - DNA, 66 bp, Homo sapiens
  source 1..66: mol_type = genomic DNA
cgggctgctcgcccttgcttcccgcctctctaatctaaaccatgcggggctcccagcccctacccg

SEQ ID NO: 355 - DNA, 42 bp, Homo sapiens
  source 1..42: mol_type = genomic DNA
cggctcgcggggcccgagggccaagaggagggcactgggccg

SEQ ID NO: 356 - DNA, 39 bp, Homo sapiens
  source 1..39: mol_type = genomic DNA
cgcggggcccgagggccaagaggagggcactgggccgcg

SEQ ID NO: 357 - DNA, 39 bp, Homo sapiens
  source 1..39: mol_type = genomic DNA
cggggcccgagggccaagaggagggcactgggccgcgcg

SEQ ID NO: 358 - DNA, 44 bp, Homo sapiens
  source 1..44: mol_type = genomic DNA
cgagggccaagaggagggcactgggccgcgcggtggaggagccg

SEQ ID NO: 359 - DNA, 33 bp, Homo sapiens
  source 1..33: mol_type = genomic DNA
cggctcgcgtggggctcgggatcttggggaccg

SEQ ID NO: 360 - DNA, 30 bp, Homo sapiens
  source 1..30: mol_type = genomic DNA
cgcgtggggctcgggatcttggggaccgcg

SEQ ID NO: 361 - DNA, 56 bp, Homo sapiens
  source 1..56: mol_type = genomic DNA
cgagctgggcgtgcggggctggggccacaggggctcagggttaggctgcggggacg

SEQ ID NO: 362 - DNA, 61 bp, Homo sapiens
  source 1..61: mol_type = genomic DNA
cgccccgggccagccgggcgtggaaaaaaggaaattgctgggaagggggcaagctgtcccg

SEQ ID NO: 363 - DNA, 71 bp, Homo sapiens
  source 1..71: mol_type = genomic DNA
cgggcgtggaaaaaaggaaattgctgggaagggggcaagctgtcccgaggtctggatgtggcttcgtggcg

SEQ ID NO: 364 - DNA, 99 bp, Homo sapiens
  source 1..99: mol_type = genomic DNA
cgtggaaaaaaggaaattgctgggaagggggcaagctgtcccgaggtctggatgtggcttcgtggcggagagaaaaggaagaggcaaaccctgagcccg

SEQ ID NO: 365 - DNA, 84 bp, Homo sapiens
  source 1..84: mol_type = genomic DNA
cgaggtctggatgtggcttcgtggcggagagaaaaggaagaggcaaaccctgagcccgggagggagagcactgagcaggcctcg

SEQ ID NO: 366 - DNA, 76 bp, Homo sapiens
  source 1..76: mol_type = genomic DNA
cgtggcggagagaaaaggaagaggcaaaccctgagcccgggagggagagcactgagcaggcctcgtccccagtccg

SEQ ID NO: 367 - DNA, 78 bp, Homo sapiens
  source 1..78: mol_type = genomic DNA
cggagagaaaaggaagaggcaaaccctgagcccgggagggagagcactgagcaggcctcgtccccagtccgagctgcg

SEQ ID NO: 368 - DNA, 139 bp, Homo sapiens
  source 1..139: mol_type = genomic DNA
cgggtctggttcttctttcctcaggctccgcgtggagccctgggcctggcagacacaagtttctctccctcccatccccagaccatcgagaacatcaaggtggggctgcatgagaaggagctctggaagaagttccacg

SEQ ID NO: 369 - DNA, 121 bp, Homo sapiens
  source 1..121: mol_type = genomic DNA
cgtggagccctgggcctggcagacacaagtttctctccctcccatccccagaccatcgagaacatcaaggtggggctgcatgagaaggagctctggaagaagttccacgaggcgggcaccg

SEQ ID NO: 370 - DNA, 98 bp, Homo sapiens
  source 1..98: mol_type = genomic DNA
cggggatggcggtggggagcaaggggggccagggaggtcccttagaagtcctctcggccccggcctggtggcctgtgggaggcctctctgcctcctcg

SEQ ID NO: 371 - DNA, 93 bp, Homo sapiens
  source 1..93: mol_type = genomic DNA
cggtggggagcaaggggggccagggaggtcccttagaagtcctctcggccccggcctggtggcctgtgggaggcctctctgcctcctcgggcg

SEQ ID NO: 372 - DNA, 67 bp, Homo sapiens
  source 1..67: mol_type = genomic DNA
cggccccggcctggtggcctgtgggaggcctctctgcctcctcgggcgtcagtgccacctcccttcg

SEQ ID NO: 373 - DNA, 76 bp, Homo sapiens
  source 1..76: mol_type = genomic DNA
cgtgagtgactcgaaccccaagtttttcatcccaggggcgccgctcttctgtttccatccccatctctggccaacg

SEQ ID NO: 374 - DNA, 78 bp, Homo sapiens
  source 1..78: mol_type = genomic DNA
cgaaccccaagtttttcatcccaggggcgccgctcttctgtttccatccccatctctggccaacgggaaagcactacg

SEQ ID NO: 375 - DNA, 92 bp, Homo sapiens
  source 1..92: mol_type = genomic DNA
cgaaaacaggaaagggaagaaaactgaaacgggcctcttggtctgcagttttagcggagtcgggattccacagccctgagtggcacatgccg

SEQ ID NO: 376 - DNA, 81 bp, Homo sapiens
  source 1..81: mol_type = genomic DNA
cgggcctcttggtctgcagttttagcggagtcgggattccacagccctgagtggcacatgccggtcaacttcccaaagtcg

SEQ ID NO: 377 - DNA, 51 bp, Homo sapiens
  source 1..51: mol_type = genomic DNA
cgggctcccgtgtggggagaaatacacacgcaggaatgcacaagcatcgcg

SEQ ID NO: 378 - DNA, 76 bp, Homo sapiens
  source 1..76: mol_type = genomic DNA
cgcacctacgacgctatctggtactttccttttccccttagctgttgctgtccagcccagcgccagtccagagacg

SEQ ID NO: 379 - DNA, 77 bp, Homo sapiens
  source 1..77: mol_type = genomic DNA
cgacgctatctggtactttccttttccccttagctgttgctgtccagcccagcgccagtccagagacgggcttaccg

SEQ ID NO: 380 - DNA, 78 bp, Homo sapiens
  source 1..78: mol_type = genomic DNA
cgctatctggtactttccttttccccttagctgttgctgtccagcccagcgccagtccagagacgggcttaccggccg

SEQ ID NO: 381 - DNA, 42 bp, Homo sapiens
  source 1..42: mol_type = genomic DNA
cgggcttaccggccgctcccttccacaagaaactcgtgtccg

SEQ ID NO: 382 - DNA, 124 bp, Homo sapiens
  source 1..124: mol_type = genomic DNA
cgcgacacggggacctgtccctccagggataggagtcttctacctgcaagggacccggcctggggaactggaatagacaaggggtaaccccttctcccatcacctctaatgagaacattgtccg

SEQ ID NO: 383 - DNA, 144 bp, Homo sapiens
  source 1..144: mol_type = genomic DNA
cgacacggggacctgtccctccagggataggagtcttctacctgcaagggacccggcctggggaactggaatagacaaggggtaaccccttctcccatcacctctaatgagaacattgtccggggctgagatatttgggggacg

SEQ ID NO: 384 - DNA, 142 bp, Homo sapiens
  source 1..142: mol_type = genomic DNA
cggggacctgtccctccagggataggagtcttctacctgcaagggacccggcctggggaactggaatagacaaggggtaaccccttctcccatcacctctaatgagaacattgtccggggctgagatatttgggggacgccg

SEQ ID NO: 385 - DNA, 82 bp, Homo sapiens
  source 1..82: mol_type = genomic DNA
cgtccctaccgaaaacggatttaaacaccgtccccacttttgctaaggaagtttggatagggaggttgtggtccttaattcg

SEQ ID NO: 386 - DNA, 100 bp, Homo sapiens
  source 1..100: mol_type = genomic DNA
cgaaaacggatttaaacaccgtccccacttttgctaaggaagtttggatagggaggttgtggtccttaattcgccaggtaaaaggaaccccatcagctcg

SEQ ID NO: 387 - DNA, 131 bp, Homo sapiens
  source 1..131: mol_type = genomic DNA
cggatttaaacaccgtccccacttttgctaaggaagtttggatagggaggttgtggtccttaattcgccaggtaaaaggaaccccatcagctcgcatccctcacccccatcctgagcactgagtgtttgcg

SEQ ID NO: 388 - DNA, 127 bp, Homo sapiens
  source 1..127: mol_type = genomic DNA
cgtccccacttttgctaaggaagtttggatagggaggttgtggtccttaattcgccaggtaaaaggaaccccatcagctcgcatccctcacccccatcctgagcactgagtgtttgcgagaaactcg

SEQ ID NO: 389 - DNA, 82 bp, Homo sapiens
  source 1..82: mol_type = genomic DNA
cgccaggtaaaaggaaccccatcagctcgcatccctcacccccatcctgagcactgagtgtttgcgagaaactcgccctgcg

SEQ ID NO: 390 - DNA, 13 bp, Homo sapiens
  source 1..13: mol_type = genomic DNA
cgcgggcgcggcg

SEQ ID NO: 391 - DNA, 23 bp, Homo sapiens
  source 1..23: mol_type = genomic DNA
cgggcgcggcgccccagccagcg

SEQ ID NO: 392 - DNA, 42 bp, Homo sapiens
  source 1..42: mol_type = genomic DNA
cggacttcgaaggtgggtgctgggctggctgctgcggccgcg

SEQ ID NO: 393 - DNA, 39 bp, Homo sapiens
  source 1..39: mol_type = genomic DNA
cgaaggtgggtgctgggctggctgctgcggccgcggacg

SEQ ID NO: 394 - DNA, 41 bp, Homo sapiens
  source 1..41: mol_type = genomic DNA
cgtgctggagaggaccctgcgggtgggcctggcgcgggacg

SEQ ID NO: 395 - DNA, 30 bp, Homo sapiens
  source 1..30: mol_type = genomic DNA
cgggtgggcctggcgcgggacgggggtgcg

SEQ ID NO: 396 - DNA, 24 bp, Homo sapiens
  source 1..24: mol_type = genomic DNA
cgccctcccgctgagagcgccgcg

SEQ ID NO: 397 - DNA, 15 bp, Homo sapiens
  source 1..15: mol_type = genomic DNA
cgccgcgcgcccccg

SEQ ID NO: 398 - DNA, 18 bp, Homo sapiens
  source 1..18: mol_type = genomic DNA
cgcgcgcccccggccccg

SEQ ID NO: 399 - DNA, 22 bp, Homo sapiens
  source 1..22: mol_type = genomic DNA
cgcgcccccggccccgtgcccg

SEQ ID NO: 400 - DNA, 38 bp, Homo sapiens
  source 1..38: mol_type = genomic DNA
cgcgccgcctacgtgggggaccctgttaggggcacccg

SEQ ID NO: 401 - DNA, 38 bp, Homo sapiens
  source 1..38: mol_type = genomic DNA
cgccgcctacgtgggggaccctgttaggggcacccgcg

SEQ ID NO: 402 - DNA, 46 bp, Homo sapiens
  source 1..46: mol_type = genomic DNA
cgcctacgtgggggaccctgttaggggcacccgcgtagaccctgcg

SEQ ID NO: 403 - DNA, 42 bp, Homo sapiens
  source 1..42: mol_type = genomic DNA
cgtgggggaccctgttaggggcacccgcgtagaccctgcgcg

SEQ ID NO: 404 - DNA, 33 bp, Homo sapiens
  source 1..33: mol_type = genomic DNA
cgggctgcgggggcagcagcagaagcggcgacg

SEQ ID NO: 405 - DNA, 29 bp, Homo sapiens
  source 1..29: mol_type = genomic DNA
cgggggcagcagcagaagcggcgacggcg

SEQ ID NO: 406 - DNA, 118 bp, Homo sapiens
  source 1..118: mol_type = genomic DNA
cggagggcacccacatttgcatgataaattgaccagactggcggcagccaatgaaatattcaaattaggctgggtggcggtagtaggggtggagtccctggggctgccccttggcgcg

SEQ ID NO: 407 - DNA, 45 bp, Homo sapiens
  source 1..45: mol_type = genomic DNA
cgtcagagctttgggagcgctttgtttggcgacagtcggaaggcg

SEQ ID NO: 408 - DNA, 30 bp, Homo sapiens
  source 1..30: mol_type = genomic DNA
cgctttgtttggcgacagtcggaaggcgcg

SEQ ID NO: 409 - DNA, 41 bp, Homo sapiens
  source 1..41: mol_type = genomic DNA
cgaacgcctcagtgtccccgaccctgggcagcggggactcg

SEQ ID NO: 410 - DNA, 45 bp, Homo sapiens
  source 1..45: mol_type = genomic DNA
cgcctcagtgtccccgaccctgggcagcggggactcgagcaggcg

SEQ ID NO: 411 - DNA, 55 bp, Homo sapiens
  source 1..55: mol_type = genomic DNA
cgaccctgggcagcggggactcgagcaggcgcccctcactgatggctttagaacg

SEQ ID NO: 412 - DNA, 68 bp, Homo sapiens
  source 1..68: mol_type = genomic DNA
cggggactcgagcaggcgcccctcactgatggctttagaacgtgggtgggggaaggtgtgtgaggacg

SEQ ID NO: 413 - DNA, 68 bp, Homo sapiens
  source 1..68: mol_type = genomic DNA
cgagcaggcgcccctcactgatggctttagaacgtgggtgggggaaggtgtgtgaggacgggaagacg

SEQ ID NO: 414 - DNA, 63 bp, Homo sapiens
  source 1..63: mol_type = genomic DNA
cgcccctcactgatggctttagaacgtgggtgggggaaggtgtgtgaggacgggaagacgccg

SEQ ID NO: 415 - DNA, 57 bp, Homo sapiens
  source 1..57: mol_type = genomic DNA
cgtgggtgggggaaggtgtgtgaggacgggaagacgccgcactcacctgagttggcg

SEQ ID NO: 416 - DNA, 68 bp, Homo sapiens
  source 1..68: mol_type = genomic DNA
cgcactcacctgagttggcgtcctcagagtggccgctgccatcagactctgcgggtagagctgggccg

SEQ ID NO: 417 - DNA, 53 bp, Homo sapiens
  source 1..53: mol_type = genomic DNA
cgggcgacattggtagggacccggggacagcggtccctatcccaggcctgacg

SEQ ID NO: 418 - DNA, 53 bp, Homo sapiens
  source 1..53: mol_type = genomic DNA
cgggcgaggccgtccagggagccctggtccctcagctgcaccggactgagccg

SEQ ID NO: 419 - DNA, 28 bp, Homo sapiens
  source 1..28: mol_type = genomic DNA
cgcgggcgccggagtggtcccagactcg

SEQ ID NO: 420 - DNA, 36 bp, Homo sapiens
  source 1..36: mol_type = genomic DNA
cgggcgccggagtggtcccagactcggctgggaccg

SEQ ID NO: 421 - DNA, 84 bp, Homo sapiens
  source 1..84: mol_type = genomic DNA
cgccggagtggtcccagactcggctgggaccgggcagcctagaagaagggtcccctcagtagagaccaggcctccagctctccg

SEQ ID NO: 422 - DNA, 71 bp, Homo sapiens
  source 1..71: mol_type = genomic DNA
cggctgggaccgggcagcctagaagaagggtcccctcagtagagaccaggcctccagctctccgtccggcg

SEQ ID NO: 423 - DNA, 57 bp, Homo sapiens
  source 1..57: mol_type = genomic DNA
cgggcttccgggctgtggaccgcactgaacccagtcccgcagcaagccctgctgccg

SEQ ID NO: 424 - DNA, 65 bp, Homo sapiens
  source 1..65: mol_type = genomic DNA
cgggctgtggaccgcactgaacccagtcccgcagcaagccctgctgccgccttttcctgggcacg

SEQ ID NO: 425 - DNA, 59 bp, Homo sapiens
  source 1..59: mol_type = genomic DNA
cgcactgaacccagtcccgcagcaagccctgctgccgccttttcctgggcacgcctgcg

SEQ ID NO: 426 - DNA, 38 bp, Homo sapiens
  source 1..38: mol_type = genomic DNA
cgccttttcctgggcacgcctgcggtggtgacggcccg

SEQ ID NO: 427 - DNA, 39 bp, Homo sapiens
  source 1..39: mol_type = genomic DNA
cgcctgcggtggtgacggcccgctgctcatgtctccacg

SEQ ID NO: 428 - DNA, 55 bp, Homo sapiens
  source 1..55: mol_type = genomic DNA
cggtggtgacggcccgctgctcatgtctccacgccaaaccccagcccctctcccg

SEQ ID NO: 429 - DNA, 47 bp, Homo sapiens
  source 1..47: mol_type = genomic DNA
cggaactcagggctgcagaccagcccttcgcaagccaacgcgccccg

SEQ ID NO: 430 - DNA, 63 bp, Homo sapiens
  source 1..63: mol_type = genomic DNA
cggctccgcgtctctgccaccttcccaccgcttcttctttaaccatgctcttgtttcccctcg

SEQ ID NO: 431 - DNA, 64 bp, Homo sapiens
  source 1..64: mol_type = genomic DNA
cgcgtctctgccaccttcccaccgcttcttctttaaccatgctcttgtttcccctcgctgatcg

SEQ ID NO: 432 - DNA, 72 bp, Homo sapiens
  source 1..72: mol_type = genomic DNA
cgtctctgccaccttcccaccgcttcttctttaaccatgctcttgtttcccctcgctgatcgcaaggctgcg

SEQ ID NO: 433 - DNA, 97 bp, Homo sapiens
  source 1..97: mol_type = genomic DNA
cgttgggtggagaggtcggagctgactctctggagaaatggccgaagacgccccttcccatccccaacccactctcccttggagaagagaaggggcg

SEQ ID NO: 434 - DNA, 94 bp, Homo sapiens
  source 1..94: mol_type = genomic DNA
cggagctgactctctggagaaatggccgaagacgccccttcccatccccaacccactctcccttggagaagagaaggggcgtgtttccacctcg

SEQ ID NO: 435 - DNA, 81 bp, Homo sapiens
  source 1..81: mol_type = genomic DNA
cgaagacgccccttcccatccccaacccactctcccttggagaagagaaggggcgtgtttccacctcgctgtaacccagcg

SEQ ID NO: 436 - DNA, 95 bp, Homo sapiens
  source 1..95: mol_type = genomic DNA
cgccccttcccatccccaacccactctcccttggagaagagaaggggcgtgtttccacctcgctgtaacccagcgccaaacccttcccctccccg

SEQ ID NO: 437 - DNA, 55 bp, Homo sapiens
  source 1..55: mol_type = genomic DNA
cgcgcagggcccggctgaatgctaatttgggatacacaggagcgcaagttcaccg

SEQ ID NO: 438 - DNA, 62 bp, Homo sapiens
  source 1..62: mol_type = genomic DNA
cgcagggcccggctgaatgctaatttgggatacacaggagcgcaagttcaccgagttttccg

SEQ ID NO: 439 - DNA, 61 bp, Homo sapiens
  source 1..61: mol_type = genomic DNA
cgcaagttcaccgagttttccgcagttttgcaaatccttttccctctccccatgcctcgcg

SEQ ID NO: 440 - DNA, 64 bp, Homo sapiens
  source 1..64: mol_type = genomic DNA
cgcgccaggaaggccctggagctccctctccccctcacaccgcttcctgcggggccagggctcg

SEQ ID NO: 441 - DNA, 60 bp, Homo sapiens
  source 1..60: mol_type = genomic DNA
cgctggagccaccattctgccagcagccggggaggggccgcgagggctcccaagtggacg

SEQ ID NO: 442 - DNA, 43 bp, Homo sapiens
  source 1..43: mol_type = genomic DNA
cggggaggggccgcgagggctcccaagtggacggaagtaggcg

SEQ ID NO: 443 - DNA, 102 bp, Homo sapiens
  source 1..102: mol_type = genomic DNA
cggcctctctagcgcgttgacactgttgatggaagggattaaaggggtatcaggttattttaatttattgttgaagatgatgactgccccctccccgatccg

SEQ ID NO: 444 - DNA, 97 bp, Homo sapiens
  source 1..97: mol_type = genomic DNA
cgcgttgacactgttgatggaagggattaaaggggtatcaggttattttaatttattgttgaagatgatgactgccccctccccgatccgcataccg

SEQ ID NO: 445 - DNA, 52 bp, Homo sapiens
  source 1..52: mol_type = genomic DNA
cgagatctgggcctgccaggggcctgcccgagtcctctatcgcgggtccacg

SEQ ID NO: 446 - DNA, 41 bp, Homo sapiens
  source 1..41: mol_type = genomic DNA
cgagtcctctatcgcgggtccacgtggccaccaatgacccg

SEQ ID NO: 447 - DNA, 31 bp, Homo sapiens
  source 1..31: mol_type = genomic DNA
cgcgggtccacgtggccaccaatgacccgcg

SEQ ID NO: 448 - DNA, 32 bp, Homo sapiens
  source 1..32: mol_type = genomic DNA
cgggtccacgtggccaccaatgacccgcggcg

SEQ ID NO: 449 - DNA, 86 bp, Homo sapiens
  source 1..86: mol_type = genomic DNA
cgctcaagatcaacctgccggagtctagagtccaggtgcgcactccccgggctccagggtctgggtaggggagctgaggctgctcg

SEQ ID NO: 450 - DNA, 131 bp, Homo sapiens
  source 1..131: mol_type = genomic DNA
cgcactccccgggctccagggtctgggtaggggagctgaggctgctcggggaaggtctaggaaggctcttgagtagttcttgaggagaccatcctgtgggggtgggcttacagactgggcagccgctctcg

SEQ ID NO: 451 - DNA, 136 bp, Homo sapiens
  source 1..136: mol_type = genomic DNA
cgggctccagggtctgggtaggggagctgaggctgctcggggaaggtctaggaaggctcttgagtagttcttgaggagaccatcctgtgggggtgggcttacagactgggcagccgctctcggactccccctagcg

SEQ ID NO: 452 - DNA, 101 bp, Homo sapiens
  source 1..101: mol_type = genomic DNA
cgctctcggactccccctagcgctgggccccagccaggaaagggggcaaaagttaaaggaaatggcttagagccctggattccgcctttagccctagggcg

SEQ ID NO: 453 - DNA, 101 bp, Homo sapiens
  source 1..101: mol_type = genomic DNA
cggactccccctagcgctgggccccagccaggaaagggggcaaaagttaaaggaaatggcttagagccctggattccgcctttagccctagggcgccttcg

SEQ ID NO: 454 - DNA, 55 bp, Homo sapiens
  source 1..55: mol_type = genomic DNA
cgaggtcctggtctcggggggctaaaagcgagggaaactccgtgttaataacccg

SEQ ID NO: 455 - DNA, 44 bp, Homo sapiens
  source 1..44: mol_type = genomic DNA
cgagggaaactccgtgttaataacccgcgtgttaatgacctgcg

SEQ ID NO: 456 - DNA, 59 bp, Homo sapiens
  source 1..59: mol_type = genomic DNA
cgtgttaataacccgcgtgttaatgacctgcgtgaagctggaggccaaaggatgaggcg

SEQ ID NO: 457 - DNA, 82 bp, Homo sapiens
  source 1..82: mol_type = genomic DNA
cgcgtgttaatgacctgcgtgaagctggaggccaaaggatgaggcgggaggacttggcaagagaagtccccagaaactttcg

SEQ ID NO: 458 - DNA, 112 bp, Homo sapiens
  source 1..112: mol_type = genomic DNA
cgtgttaatgacctgcgtgaagctggaggccaaaggatgaggcgggaggacttggcaagagaagtccccagaaactttcgcttagggagtgtctccccccacccccaccacg

SEQ ID NO: 459 - DNA, 105 bp, Homo sapiens
  source 1..105: mol_type = genomic DNA
cgtgaagctggaggccaaaggatgaggcgggaggacttggcaagagaagtccccagaaactttcgcttagggagtgtctccccccacccccaccacgcccctccg

SEQ ID NO: 460 - DNA, 69 bp, Homo sapiens
  source 1..69: mol_type = genomic DNA
cgcttagggagtgtctccccccacccccaccacgcccctccgcgtcagcctggcctcttcccccaggcg

SEQ ID NO: 461 - DNA, 48 bp, Homo sapiens
  source 1..48: mol_type = genomic DNA
cgcccctccgcgtcagcctggcctcttcccccaggcgttttgaccccg

SEQ ID NO: 462 - DNA, 52 bp, Homo sapiens
  source 1..52: mol_type = genomic DNA
cgcgtcagcctggcctcttcccccaggcgttttgaccccgcatcccccctcg

SEQ ID NO: 463 - DNA, 141 bp, Homo sapiens
  source 1..141: mol_type = genomic DNA
cgggaaaggatagctctgtaatgctctcagagctgagccaaaaaggaaaagaaagcaaaacgacaaaaaactgatccgtgtcctgcatgttggcagcagacaaccttccttgctgctgagctgtcccgggtggcttcaccg

SEQ ID NO: 464 - DNA, 141 bp, Homo sapiens
  source 1..141: mol_type = genomic DNA
cggctggggaatccgagccattccttccacgtgctaagatgtccctctaagccctcagctcctgccagaagtagccaaattacctttcttcgtactgtaaacttcctttaaaacatacaaactcataacctggaaaagtcg

SEQ ID NO: 465 - DNA, 137 bp, Homo sapiens
  source 1..137: mol_type = genomic DNA
cgagccattccttccacgtgctaagatgtccctctaagccctcagctcctgccagaagtagccaaattacctttcttcgtactgtaaacttcctttaaaacatacaaactcataacctggaaaagtcgacaaggacg

SEQ ID NO: 466 - DNA, 53 bp, Homo sapiens
  source 1..53: mol_type = genomic DNA
cggtaggccaggctcgggcgcgcccttcccacaccaacaaaccatctttcccg

SEQ ID NO: 467 - DNA, 63 bp, Homo sapiens
  source 1..63: mol_type = genomic DNA
cgggcgcgcccttcccacaccaacaaaccatctttcccgactcagcagaggcccacaggggcg

SEQ ID NO: 468 - DNA, 65 bp, Homo sapiens
  source 1..65: mol_type = genomic DNA
cgcgcccttcccacaccaacaaaccatctttcccgactcagcagaggcccacaggggcgcagccg

SEQ ID NO: 469 - DNA, 74 bp, Homo sapiens
  source 1..74: mol_type = genomic DNA
cgcccttcccacaccaacaaaccatctttcccgactcagcagaggcccacaggggcgcagccgctgtccctccg

SEQ ID NO: 470 - DNA, 55 bp, Homo sapiens
  source 1..55: mol_type = genomic DNA
cggggccaagcctgacctgccggggtgccccgtggcatctctggtgcggaccccg

SEQ ID NO: 471 - DNA, 39 bp, Homo sapiens
  source 1..39: mol_type = genomic DNA
cggggtgccccgtggcatctctggtgcggaccccgcacg

SEQ ID NO: 472 - DNA, 110 bp, Homo sapiens
  source 1..110: mol_type = genomic DNA
cgcactggcctgggcacccgggctgcagggccttttgggccctcccctcagtgaccctgcctgtagcggccccagggccgctttggctcaggcagcagctgccaatgccg

SEQ ID NO: 473 - DNA, 96 bp, Homo sapiens
  source 1..96: mol_type = genomic DNA
cgggctgcagggccttttgggccctcccctcagtgaccctgcctgtagcggccccagggccgctttggctcaggcagcagctgccaatgccgctcg

SEQ ID NO: 474 - DNA, 69 bp, Homo sapiens
  source 1..69: mol_type = genomic DNA
cgcggaagcacagatcatcttcccgggactgggtctcctggaccctgcgttgctccctttttcctagcg

SEQ ID NO: 475 - DNA, 35 bp, Homo sapiens
  source 1..35: mol_type = genomic DNA
cgaaccctgtagcgctgttgcttcgcggtccatcg

SEQ ID NO: 476 - DNA, 26 bp, Homo sapiens
  source 1..26: mol_type = genomic DNA
cgctgttgcttcgcggtccatcgtcg

SEQ ID NO: 477 - DNA, 25 bp, Homo sapiens
  source 1..25: mol_type = genomic DNA
cggtccatcgtcgccgctgcagacg

SEQ ID NO: 478 - DNA, 33 bp, Homo sapiens
  source 1..33: mol_type = genomic DNA
cgaatgcggaatcgtccaggactccgaaggtcg

SEQ ID NO: 479 - DNA, 33 bp, Homo sapiens
  source 1..33: mol_type = genomic DNA
cggaatcgtccaggactccgaaggtcggggccg

SEQ ID NO: 480 - DNA, 31 bp, Homo sapiens
  source 1..31: mol_type = genomic DNA
cgtccaggactccgaaggtcggggccgctcg

SEQ ID NO: 481 - DNA, 29 bp, Homo sapiens
  source 1..29: mol_type = genomic DNA
cgctcgcgagcaccgaaggggaggagccg

SEQ ID NO: 482 - DNA, 28 bp, Homo sapiens
  source 1..28: mol_type = genomic DNA
cgcgagcaccgaaggggaggagccgacg

SEQ ID NO: 483 - DNA, 44 bp, Homo sapiens
  source 1..44: mol_type = genomic DNA
cgagcaccgaaggggaggagccgacgaagaccaggagtgggccg

SEQ ID NO: 484 - DNA, 44 bp, Homo sapiens
  source 1..44: mol_type = genomic DNA
cgaaggggaggagccgacgaagaccaggagtgggccgcatttcg

SEQ ID NO: 485 - DNA, 44 bp, Homo sapiens
  source 1..44: mol_type = genomic DNA
cgacgaagaccaggagtgggccgcatttcggtactgtttccccg

SEQ ID NO: 486 - DNA, 58 bp, Homo sapiens
  source 1..58: mol_type = genomic DNA
cgaagaccaggagtgggccgcatttcggtactgtttccccgagatcaggaactttccg

SEQ ID NO: 487 - DNA, 82 bp, Homo sapiens
  source 1..82: mol_type = genomic DNA
cgatgtcctgggagaggaggaagatgaagacgaggtggaagacgaggaggaggaggcgagccagaagttcctagagcagtcg

SEQ ID NO: 488 - DNA, 61 bp, Homo sapiens
  source 1..61: mol_type = genomic DNA
cgaggtggaagacgaggaggaggaggcgagccagaagttcctagagcagtcgctccagccg

SEQ ID NO: 489 - DNA, 66 bp, Homo sapiens
  source 1..66: mol_type = genomic DNA
cgaggaggaggaggcgagccagaagttcctagagcagtcgctccagccggggctgcaggtggcccg

SEQ ID NO: 490 - DNA, 44 bp, Homo sapiens
  source 1..44: mol_type = genomic DNA
cgctccagccggggctgcaggtggcccggtggggcggggttgcg

SEQ ID NO: 491 - DNA, 35 bp, Homo sapiens
  source 1..35: mol_type = genomic DNA
cggcggcccgagcgacccctcagagtttggcaccg

SEQ ID NO: 492 - DNA, 53 bp, Homo sapiens
  source 1..53: mol_type = genomic DNA
cgagcgacccctcagagtttggcaccgagttcagggcaccgccaaggtctgcg

SEQ ID NO: 493 - DNA, 52 bp, Homo sapiens
  source 1..52: mol_type = genomic DNA
cgacccctcagagtttggcaccgagttcagggcaccgccaaggtctgcggcg

SEQ ID NO: 494 - DNA, 50 bp, Homo sapiens
  source 1..50: mol_type = genomic DNA
cgcttcccctactaccgccgcaagttccccgcctggcagaacagcatccg

SEQ ID NO: 495 - DNA, 48 bp, Homo sapiens
  source 1..48: mol_type = genomic DNA
cgccgcaagttccccgcctggcagaacagcatccgccacaacctctcg

SEQ ID NO: 496 - DNA, 52 bp, Homo sapiens
  source 1..52: mol_type = genomic DNA
cgcaagttccccgcctggcagaacagcatccgccacaacctctcgctgaacg

SEQ ID NO: 497 - DNA, 62 bp, Homo sapiens
  source 1..62: mol_type = genomic DNA
cgaaaggaaacacactctcgcacacaaagaaaagtcccagagaaaccagggccggcgatgcg

SEQ ID NO: 498 - DNA, 49 bp, Homo sapiens
  source 1..49: mol_type = genomic DNA
cgcacacaaagaaaagtcccagagaaaccagggccggcgatgcgggtcg

SEQ ID NO: 499 - DNA, 126 bp, Homo sapiens
  source 1..126: mol_type = genomic DNA
cggcatgctggttggctgcccccggaggcggaggtagggggccagaaatgctgacctgggcaggcccccagccctgagctcctggggtggacatctcagggtcccgggcctccaagctcatggccg

SEQ ID NO: 500 - DNA, 35 bp, Homo sapiens
  source 1..35: mol_type = genomic DNA
cgtatatcaggaggaggagagcgtggcgcctcgcg

SEQ ID NO: 501 - DNA, 41 bp, Homo sapiens
  source 1..41: mol_type = genomic DNA
cggtcggtggggcgcagggccggggagccaggggggtgccg

SEQ ID NO: 502 - DNA, 139 bp, Homo sapiens
  source 1..139: mol_type = genomic DNA
cggcctctaacctttatctgagtcttcggggtttcaaatattttaagagttccaacacagcagtagctcaaacccaagccaatagggggtgaaatatacttttcaactctttctcccgctgacctaaacagagacgccg

SEQ ID NO: 503 - DNA, 116 bp, Homo sapiens
  source 1..116: mol_type = genomic DNA
cggggtttcaaatattttaagagttccaacacagcagtagctcaaacccaagccaatagggggtgaaatatacttttcaactctttctcccgctgacctaaacagagacgccggcg

SEQ ID NO: 504 - DNA, 134 bp, Homo sapiens
  source 1..134: mol_type = genomic DNA
cgaatttgcttggaacaagcgccctcttaatttgcccctctcagttctctgcagttgacagcttggaaaaggaagcgaaacaaaggtccctgggaaagtgaagttttcaattaatttggtgtgagaaacgggcg

SEQ ID NO: 505 - DNA, 55 bp, Homo sapiens
  source 1..55: mol_type = genomic DNA
cggtgggaggctgagtggctttctcccccggcgccgttctcagggtctttctgcg

SEQ ID NO: 506 - DNA, 40 bp, Homo sapiens
  source 1..40: mol_type = genomic DNA
cgttctcagggtctttctgcgggtcgaagaaggacccgcg

SEQ ID NO: 507 - DNA, 42 bp, Homo sapiens
  source 1..42: mol_type = genomic DNA
cgggtcgaagaaggacccgcgggagctgagaggcccaggtcg

SEQ ID NO: 508 - DNA, 49 bp, Homo sapiens
  source 1..49: mol_type = genomic DNA
cgaagaaggacccgcgggagctgagaggcccaggtcggaagcactcccg

SEQ ID NO: 509 - DNA, 58 bp, Homo sapiens
  source 1..58: mol_type = genomic DNA
cgcgggagctgagaggcccaggtcggaagcactcccggctggcccaagagtagaggcg

SEQ ID NO: 510 - DNA, 63 bp, Homo sapiens
  source 1..63: mol_type = genomic DNA
cgggagctgagaggcccaggtcggaagcactcccggctggcccaagagtagaggcgaagagcg

SEQ ID NO: 511 - DNA, 109 bp, Homo sapiens
  source 1..109: mol_type = genomic DNA
cgaacggacagcgtgtgtgtataggaagggtctgctccaatgcctcttacctgtgtgaaatgcctttgccgggtaccagtgcacaaggtagggcaaattagctcactcg

SEQ ID NO: 512 - DNA, 52 bp, Homo sapiens
  source 1..52: mol_type = genomic DNA
cgagtatgccattaacaaattcattaacaaggacaagcggcggcgtatctcg

SEQ ID NO: 513 - DNA, 151 bp, Homo sapiens
  source 1..151: mol_type = genomic DNA
cgtatctcggctgctacgaacctatctgagagacaagtgaccatttggtttcagaaccgaagagtgaaggacaagaaaattgtctccaagctcaaagatactgtctcctgatgtggtccaggttggccacagacagcttagaagccattcg

SEQ ID NO: 514 - DNA, 77 bp, Homo sapiens
  source 1..77: mol_type = genomic DNA
cggaaaacgaatcgcaaagccaatcacgaccaagaagagtcccaggggacacttgggcagagtcaccctcttgcccg

SEQ ID NO: 515 - DNA, 92 bp, Homo sapiens
  source 1..92: mol_type = genomic DNA
cgaatcgcaaagccaatcacgaccaagaagagtcccaggggacacttgggcagagtcaccctcttgcccgatgtccccagctgctgaagccg

SEQ ID NO: 516 - DNA, 101 bp, Homo sapiens
  source 1..101: mol_type = genomic DNA
cgcaaagccaatcacgaccaagaagagtcccaggggacacttgggcagagtcaccctcttgcccgatgtccccagctgctgaagccgggcctggaaacccg

SEQ ID NO: 517 - DNA, 104 bp, Homo sapiens
  source 1..104: mol_type = genomic DNA
cgaccaagaagagtcccaggggacacttgggcagagtcaccctcttgcccgatgtccccagctgctgaagccgggcctggaaacccgcagacagttagtcttcg

SEQ ID NO: 518 - DNA, 68 bp, Homo sapiens
  source 1..68: mol_type = genomic DNA
cggggggatgtaggcggtgctgaaatgaccggctttgaagaacctgcaggcaaagtttcgtccaatcg

SEQ ID NO: 519 - DNA, 72 bp, Homo sapiens
  source 1..72: mol_type = genomic DNA
cgcttctgaatctgcagtcgccagactctttctacttctccaacctgaggccgaatggcggccagttggccg

SEQ ID NO: 520 - DNA, 68 bp, Homo sapiens
  source 1..68: mol_type = genomic DNA
cggcttctcgcagccctacctggctggctccgggcctctcggcctgcagcccccaacagccaaagacg

SEQ ID NO: 521 - DNA, 66 bp, Homo sapiens
  source 1..66: mol_type = genomic DNA
cgcagccctacctggctggctccgggcctctcggcctgcagcccccaacagccaaagacggacccg

SEQ ID NO: 522 - DNA, 67 bp, Homo sapiens
  source 1..67: mol_type = genomic DNA
cgggcctctcggcctgcagcccccaacagccaaagacggacccgaagagcaggctaagttctatgcg

SEQ ID NO: 523 - DNA, 76 bp, Homo sapiens
  source 1..76: mol_type = genomic DNA
cgagtctagcctggctcctgcagtggctgctctcaaagcggccaagtatgactacgctggtgtgggtcgtgccacg

SEQ ID NO: 524 - DNA, 73 bp, Homo sapiens
  source 1..73: mol_type = genomic DNA
cgggccggtttgggccgggatgggaggtggggttcaagggagagtgtaaggggaggtgaaccgcctgggggcg

SEQ ID NO: 525 - DNA, 59 bp, Homo sapiens
  source 1..59: mol_type = genomic DNA
cgggctgggttgacgtgggttggggctgtgttgcaggcctgccgtggggggcggccccg

SEQ ID NO: 526 - DNA, 70 bp, Homo sapiens
  source 1..70: mol_type = genomic DNA
cgcttgtcctggacccggccctcttaagcgcgttctgcctggttgtgtgtggggggaatgcttctgtgcg

SEQ ID NO: 527 - DNA, 61 bp, Homo sapiens
  source 1..61: mol_type = genomic DNA
cggccctcttaagcgcgttctgcctggttgtgtgtggggggaatgcttctgtgcgctggcg

SEQ ID NO: 528 - DNA, 44 bp, Homo sapiens
  source 1..44: mol_type = genomic DNA
cggccggccaggctctggagcacgtgacccgagaggaggctgcg

SEQ ID NO: 529 - DNA, 64 bp, Homo sapiens
  source 1..64: mol_type = genomic DNA
cgcgccaggcaaagataaatgactcctggctcccccggcagctcagggggtctgggcgggggcg

SEQ ID NO: 530 - DNA, 79 bp, Homo sapiens
  source 1..79: mol_type = genomic DNA
cgccaggcaaagataaatgactcctggctcccccggcagctcagggggtctgggcgggggcggagagggaaggaagccg

SEQ ID NO: 531 - DNA, 49 bp, Homo sapiens
  source 1..49: mol_type = genomic DNA
cggcagctcagggggtctgggcgggggcggagagggaaggaagccgtcg

SEQ ID NO: 532 - DNA, 39 bp, Homo sapiens
  source 1..39: mol_type = genomic DNA
cggaaggcagcgccgcctgggagtggggaggctgcggcg

SEQ ID NO: 533 - DNA, 32 bp, Homo sapiens
  source 1..32: mol_type = genomic DNA
cgcctgggagtggggaggctgcggcgacgacg

SEQ ID NO: 534 - DNA, 20 bp, Homo sapiens
  source 1..20: mol_type = genomic DNA
cggcgacgacgtggacttcg

SEQ ID NO: 535 - DNA, 23 bp, Homo sapiens
  source 1..23: mol_type = genomic DNA
cgacgacgtggacttcggggacg

SEQ ID NO: 536 - DNA, 28 bp, Homo sapiens
  source 1..28: mol_type = genomic DNA
cgacgtggacttcggggacgagaagtcg

SEQ ID NO: 537 - DNA, 41 bp, Homo sapiens
  source 1..41: mol_type = genomic DNA
cgtggacttcggggacgagaagtcgaggctctttatggacg

SEQ ID NO: 538 - DNA, 34 bp, Homo sapiens
  source 1..34: mol_type = genomic DNA
cggggacgagaagtcgaggctctttatggacgcg

SEQ ID NO: 539 - DNA, 30 bp, Homo sapiens
  source 1..30: mol_type = genomic DNA
cgagaagtcgaggctctttatggacgcgcg

SEQ ID NO: 540 - DNA, 38 bp, Homo sapiens
  source 1..38: mol_type = genomic DNA
cggagacatccgcgcgttggtgcaactgcacaacaacg

SEQ ID NO: 541 - DNA, 33 bp, Homo sapiens
  source 1..33: mol_type = genomic DNA
cgcgcgttggtgcaactgcacaacaacgaggcg

SEQ ID NO: 542 - DNA, 45 bp, Homo sapiens
  source 1..45: mol_type = genomic DNA
cgcgttggtgcaactgcacaacaacgaggcgggcaggctggtgcg

SEQ ID NO: 543 - DNA, 130 bp, Homo sapiens
  source 1..130: mol_type = genomic DNA
cgtttgggagcgagggacaggagaataattttgatccagaaccttggagattggagaagcaggtggaaaaattctaaaaagatgaaaactgaccatctccgacacagaaacccatcgaaaggggccaccg

SEQ ID NO: 544 - DNA, 127 bp, Homo sapiens
  source 1..127: mol_type = genomic DNA
cgtgccagaggtatgagcctcctcccttccggtgatcgtggctcaaaaatccaggatggacctgggcctcttcatctagcccccatcggctgaaaagtgcatttttatttgacagaaaatgacagcg

SEQ ID NO: 545 - DNA, 21 bp, Homo sapiens
  source 1..21: mol_type = genomic DNA
cggccgaccgctcgcaggccg

SEQ ID NO: 546 - DNA, 24 bp, Homo sapiens
  source 1..24: mol_type = genomic DNA
cgaccgctcgcaggccggaggccg

SEQ ID NO: 547 - DNA, 24 bp, Homo sapiens
  source 1..24: mol_type = genomic DNA
cgctcgcaggccggaggccgagcg

SEQ ID NO: 548 - DNA, 41 bp, Homo sapiens
  source 1..41: mol_type = genomic DNA
cgccggggggcgagccatagaggggcacccaggccgggccg

SEQ ID NO: 549 - DNA, 57 bp, Homo sapiens
  source 1..57: mol_type = genomic DNA
cggggggcgagccatagaggggcacccaggccgggccgaaggtagggttaaaccccg

SEQ ID NO: 550 - DNA, 62 bp, Homo sapiens
  source 1..62: mol_type = genomic DNA
cgggccgaaggtagggttaaaccccgctgtggggaacaagagaagcgtgttggaggggagcg

SEQ ID NO: 551 - DNA, 68 bp, Homo sapiens
  source 1..68: mol_type = genomic DNA
cgaaggtagggttaaaccccgctgtggggaacaagagaagcgtgttggaggggagcgtggggagggcg

SEQ ID NO: 552 - DNA, 41 bp, Homo sapiens
  source 1..41: mol_type = genomic DNA
cgctctgaaacggaagaagttggccagcagctgcggacgcg

SEQ ID NO: 553 - DNA, 46 bp, Homo sapiens
  source 1..46: mol_type = genomic DNA
cgccgctgtggaacacgacctggggcgcctccaaaacagctcagcg

SEQ ID NO: 554 - DNA, 32 bp, Homo sapiens
  source 1..32: mol_type = genomic DNA
cggccgagtggtgaacgtagcgcagatcatcg

SEQ ID NO: 555 - DNA, 86 bp, Homo sapiens
  source 1..86: mol_type = genomic DNA
cgattttatgtgggtttcgttggatgcattttgttatttgcctcaatggatgtttcacattaatttttagttgcacaacgaacgcg

SEQ ID NO: 556 - DNA, 38 bp, Homo sapiens
  source 1..38: mol_type = genomic DNA
cggttaccggtgcttcccccggtcgcctgggggtcccg

SEQ ID NO: 557 - DNA, 35 bp, Homo sapiens
  source 1..35: mol_type = genomic DNA
cggtgcttcccccggtcgcctgggggtcccgcacg

SEQ ID NO: 558 - DNA, 25 bp, Homo sapiens
  source 1..25: mol_type = genomic DNA
cggtcgcctgggggtcccgcacgcg

SEQ ID NO: 559 - DNA, 26 bp, Homo sapiens
  source 1..26: mol_type = genomic DNA
cgcctgggggtcccgcacgcgccccg

SEQ ID NO: 560 - DNA, 25 bp, Homo sapiens
  source 1..25: mol_type = genomic DNA
cgggcaccggcttcgcgtctccccg

SEQ ID NO: 561 - DNA, 41 bp, Homo sapiens
  source 1..41: mol_type = genomic DNA
cgactccgctttgcaagcgctcagcacacgggaaaccctcg

SEQ ID NO: 562 - DNA, 53 bp, Homo sapiens
  source 1..53: mol_type = genomic DNA
cgccccttgcccagcgccaccgacccttaagcagcgtcaaggaaggagtcccg

SEQ ID NO: 563 - DNA, 59 bp, Homo sapiens
  source 1..59: mol_type = genomic DNA
cgccaccgacccttaagcagcgtcaaggaaggagtcccgatcaaggacagggatctgcg

SEQ ID NO: 564 - DNA, 63 bp, Homo sapiens
  source 1..63: mol_type = genomic DNA
cgacccttaagcagcgtcaaggaaggagtcccgatcaaggacagggatctgcggccatggccg

SEQ ID NO: 565 - DNA, 55 bp, Homo sapiens
  source 1..55: mol_type = genomic DNA
cgtcaaggaaggagtcccgatcaaggacagggatctgcggccatggccgaggccg

SEQ ID NO: 566 - DNA, 40 bp, Homo sapiens
  source 1..40: mol_type = genomic DNA
cgatcaaggacagggatctgcggccatggccgaggccgcg

SEQ ID NO: 567 - DNA, 31 bp, Homo sapiens
  source 1..31: mol_type = genomic DNA
cgaggccgcggagccggagggggttgccccg

SEQ ID NO: 568 - DNA, 40 bp, Homo sapiens
  source 1..40: mol_type = genomic DNA
cgcggagccggagggggttgccccgggtccccaggggccg

SEQ ID NO: 569 - DNA, 41 bp, Homo sapiens
  source 1..41: mol_type = genomic DNA
cggagccggagggggttgccccgggtccccaggggccgccg

SEQ ID NO: 570 - DNA, 45 bp, Homo sapiens
  source 1..45: mol_type = genomic DNA
cggagggggttgccccgggtccccaggggccgccggaggtccccg

SEQ ID NO: 571 - DNA, 32 bp, Homo sapiens
  source 1..32: mol_type = genomic DNA
cgggtccccaggggccgccggaggtccccgcg

SEQ ID NO: 572 - DNA, 36 bp, Homo sapiens
  source 1..36: mol_type = genomic DNA
cgccggaggtccccgcgcctctggctgagagacccg

SEQ ID NO: 573 - DNA, 48 bp, Homo sapiens
  source 1..48: mol_type = genomic DNA
cggaggtccccgcgcctctggctgagagacccggagagccaggagccg

SEQ ID NO: 574 - DNA, 40 bp, Homo sapiens
  source 1..40: mol_type = genomic DNA
cgcgcctctggctgagagacccggagagccaggagccgcg

SEQ ID NO: 575 - DNA, 42 bp, Homo sapiens
  source 1..42: mol_type = genomic DNA
cgcctctggctgagagacccggagagccaggagccgcgggcg

SEQ ID NO: 576 - DNA, 40 bp, Homo sapiens
  source 1..40: mol_type = genomic DNA
cggagagccaggagccgcgggcggggaggcagaagggccg

SEQ ID NO: 577 - DNA, 31 bp, Homo sapiens
  source 1..31: mol_type = genomic DNA
cggcgcgcagcaggtggagggggcgagcccg

SEQ ID NO: 578 - DNA, 33 bp, Homo sapiens
  source 1..33: mol_type = genomic DNA
cgcgcagcaggtggagggggcgagcccgggacg

SEQ ID NO: 579 - DNA, 33 bp, Homo sapiens
  source 1..33: mol_type = genomic DNA
cgcagcaggtggagggggcgagcccgggacgcg

SEQ ID NO: 580 - DNA, 18 bp, Homo sapiens
  source 1..18: mol_type = genomic DNA
cgagcccgggacgcggcg

SEQ ID NO: 581 - DNA, 14 bp, Homo sapiens
  source 1..14: mol_type = genomic DNA
cgggacgcggcgcg

SEQ ID NO: 582 - DNA, 16 bp, Homo sapiens
  source 1..16: mol_type = genomic DNA
cgcggcgcgcagggcg

SEQ ID NO: 583 - DNA, 21 bp, Homo sapiens
  source 1..21: mol_type = genomic DNA
cggcgcgcagggcgagccccg

SEQ ID NO: 584 - DNA, 20 bp, Homo sapiens
  source 1..20: mol_type = genomic DNA
cgcgcagggcgagccccgcg

SEQ ID NO: 585 - DNA, 39 bp, Homo sapiens
  source 1..39: mol_type = genomic DNA
cgcagggcgagccccgcggggaggctcagagggagcccg

SEQ ID NO: 586 - DNA, 43 bp, Homo sapiens
  source 1..43: mol_type = genomic DNA
cgagccccgcggggaggctcagagggagcccgaggactctgcg

SEQ ID NO: 587 - DNA, 26 bp, Homo sapiens
  source 1..26: mol_type = genomic DNA
cgcgtccggggaggcgggggacagcg

SEQ ID NO: 588 - DNA, 30 bp, Homo sapiens
  source 1..30: mol_type = genomic DNA
cgtccggggaggcgggggacagcgtagacg

SEQ ID NO: 589 - DNA, 28 bp, Homo sapiens
  source 1..28: mol_type = genomic DNA
cggggaggcgggggacagcgtagacgcg

SEQ ID NO: 590 - DNA, 75 bp, Homo sapiens
  source 1..75: mol_type = genomic DNA
cggctgatcggagagtgggagggaggatgggagatgggcagaggctgcccgcatcagggccaggacagacgtccg

SEQ ID NO: 591 - DNA, 138 bp, Homo sapiens
  source 1..138: mol_type = genomic DNA
cgaagccttctcagggaccagcgagaaagaggagcaggcttgggagacagggaaggaaaatgcaggggaaagggctcacccctcgaccccaggtaaaattagaaggaacgtgtggcaacccaggtgcagctttggtcg

SEQ ID NO: 592 - DNA, 138 bp, Homo sapiens
  source 1..138: mol_type = genomic DNA
cgccccaaattgcgacaatttactgggatccttttgtggggaaaggaggcttagaggctcaagctataggctgtcctagagcaactaggcgagaacctggccccaaactccctccttacgccctggcacaggttcccg

SEQ ID NO: 593 - DNA, 129 bp, Homo sapiens
  source 1..129: mol_type = genomic DNA
cgacaatttactgggatccttttgtggggaaaggaggcttagaggctcaagctataggctgtcctagagcaactaggcgagaacctggccccaaactccctccttacgccctggcacaggttcccggcg

SEQ ID NO: 594 - DNA, 106 bp, Homo sapiens
  source 1..106: mol_type = genomic DNA
cgctaaccccgaggcgcctagcacagactcttctcaccccttatttctgaaataaagcccttccttaggtccagatgaggaccacgtgctcagtgcctcactttcg

SEQ ID NO: 595 - DNA, 141 bp, Homo sapiens
  source 1..141: mol_type = genomic DNA
cgaggcgcctagcacagactcttctcaccccttatttctgaaataaagcccttccttaggtccagatgaggaccacgtgctcagtgcctcactttcgtgggagtgtatatcactttacagtatcaagacaattttctttcg

SEQ ID NO: 596 - DNA, 51 bp, Homo sapiens
  source 1..51: mol_type = genomic DNA
cggactgtgcaggggaagcctggggtctcgcggggcgcagcagtcaggtcg

SEQ ID NO: 597 - DNA, 51 bp, Homo sapiens
  source 1..51: mol_type = genomic DNA
cgcggggcgcagcagtcaggtcgagggtgcagcaggaggggagtcctgacg

SEQ ID NO: 598 - DNA, 75 bp, Homo sapiens
  source 1..75: mol_type = genomic DNA
cggggcgcagcagtcaggtcgagggtgcagcaggaggggagtcctgacgggcaggtccctctttcccctggtgcg

SEQ ID NO: 599 - DNA, 115 bp, Homo sapiens
  source 1..115: mol_type = genomic DNA
cgagggtgcagcaggaggggagtcctgacgggcaggtccctctttcccctggtgcgcaacactggttggtagcttttgcggaggtggtgaagaagggcaggaggcctgttgagcg

SEQ ID NO: 600 - DNA, 78 bp, Homo sapiens
  source 1..78: mol_type = genomic DNA
cgacccgattcattcccccctcctgtcatttgtctctgccatcgaaaaatgcctaccgagagctgctctgcatttccg

SEQ ID NO: 601 - DNA, 127 bp, Homo sapiens
  source 1..127: mol_type = genomic DNA
cgagtcagccgctagctctgcatttccaaaaagcagatcttttcacaactctcttgccccaagtgccctggtgtggtttattttttaaaatgcatgcctgcggaagagaagacccggggaatattcg

SEQ ID NO: 602 - DNA, 126 bp, Homo sapiens
  source 1..126: mol_type = genomic DNA
cgctagctctgcatttccaaaaagcagatcttttcacaactctcttgccccaagtgccctggtgtggtttattttttaaaatgcatgcctgcggaagagaagacccggggaatattcgaaaccccg

SEQ ID NO: 603 - DNA, 115 bp, Homo sapiens
  source 1..115: mol_type = genomic DNA
cgaacttttgtttcaagatgcgcgactatttatagaaggaaaactggtaggatagtaatttcctaggagcaagaggagaaattggctgcgtctttattaaatgggggtgggggcg

SEQ ID NO: 604 - DNA, 36 bp, Homo sapiens
  source 1..36: mol_type = genomic DNA
cgggctgcgtggtcctcggccggcctcctgcatccg

SEQ ID NO: 605 - DNA, 42 bp, Homo sapiens
  source 1..42: mol_type = genomic DNA
cgtggtcctcggccggcctcctgcatccgctgcccctgtgcg

SEQ ID NO: 606 - DNA, 38 bp, Homo sapiens
  source 1..38: mol_type = genomic DNA
cggccggcctcctgcatccgctgcccctgtgcgctccg

SEQ ID NO: 607 - DNA, 39 bp, Homo sapiens
  source 1..39: mol_type = genomic DNA
cggcctcctgcatccgctgcccctgtgcgctccgggccg

SEQ ID NO: 608 - DNA, 31 bp, Homo sapiens
  source 1..31: mol_type = genomic DNA
cggcgcggggaccaagcctggctgccggccg

SEQ ID NO: 609 - DNA, 49 bp, Homo sapiens
  source 1..49: mol_type = genomic DNA
cggcggtagggaaacagacgatcacagttaggcctgaggcccctcggcg

SEQ ID NO: 610 - DNA, 64 bp, Homo sapiens
  source 1..64: mol_type = genomic DNA
cggtagggaaacagacgatcacagttaggcctgaggcccctcggcggcctgtgtccttcccacg

SEQ ID NO: 611 - DNA, 47 bp, Homo sapiens
  source 1..47: mol_type = genomic DNA
cgtctccgtgtggatctcggtctttgtctcgccctctagctctctcg

SEQ ID NO: 612 - DNA, 45 bp, Homo sapiens
  source 1..45: mol_type = genomic DNA
cgtgtggatctcggtctttgtctcgccctctagctctctcgtgcg

SEQ ID NO: 613 - DNA, 48 bp, Homo sapiens
  source 1..48: mol_type = genomic DNA
cggtctttgtctcgccctctagctctctcgtgcgcctgtgtttaatcg

SEQ ID NO: 614 - DNA, 40 bp, Homo sapiens
  source 1..40: mol_type = genomic DNA
cgccctctagctctctcgtgcgcctgtgtttaatcgtgcg

SEQ ID NO: 615 - DNA, 65 bp, Homo sapiens
  source 1..65: mol_type = genomic DNA
cgtgcgcctgtgtttaatcgtgcgcttctctcaatctctctgtccctccctttgccccctacacg

SEQ ID NO: 616 - DNA, 63 bp, Homo sapiens
  source 1..63: mol_type = genomic DNA
cgcctgtgtttaatcgtgcgcttctctcaatctctctgtccctccctttgccccctacacgcg

SEQ ID NO: 617 - DNA, 57 bp, Homo sapiens
  source 1..57: mol_type = genomic DNA
cgtgcgcttctctcaatctctctgtccctccctttgccccctacacgcgtgttctcg

SEQ ID NO: 618 - DNA, 84 bp, Homo sapiens
  source 1..84: mol_type = genomic DNA
cgcttctctcaatctctctgtccctccctttgccccctacacgcgtgttctcgtcaacagatctccagaggtggtgcagaaacg

SEQ ID NO: 619 - DNA, 49 bp, Homo sapiens
  source 1..49: mol_type = genomic DNA
cgcgtgttctcgtcaacagatctccagaggtggtgcagaaacgaccccg

SEQ ID NO: 620 - DNA, 49 bp, Homo sapiens
  source 1..49: mol_type = genomic DNA
cgtgttctcgtcaacagatctccagaggtggtgcagaaacgaccccgcg

SEQ ID NO: 621 - DNA, 44 bp, Homo sapiens
  source 1..44: mol_type = genomic DNA
cgtcaacagatctccagaggtggtgcagaaacgaccccgcgccg

SEQ ID NO: 622 - DNA, 25 bp, Homo sapiens
  source 1..25: mol_type = genomic DNA
cgcgccccggctccggtccccaccg

SEQ ID NO: 623 - DNA, 29 bp, Homo sapiens
  source 1..29: mol_type = genomic DNA
cgccccggctccggtccccaccgtccccg

SEQ ID NO: 624 - DNA, 37 bp, Homo sapiens
  source 1..37: mol_type = genomic DNA
cggctccggtccccaccgtccccgccccagatttccg

SEQ ID NO: 625 - DNA, 43 bp, Homo sapiens
  source 1..43: mol_type = genomic DNA
cggtccccaccgtccccgccccagatttccggaggagcaggcg

SEQ ID NO: 626 - DNA, 32 bp, Homo sapiens
  source 1..32: mol_type = genomic DNA
cggccgcgacccctccccgctgacctcactcg

SEQ ID NO: 627 - DNA, 33 bp, Homo sapiens
  source 1..33: mol_type = genomic DNA
cgcgacccctccccgctgacctcactcgagccg

SEQ ID NO: 628 - DNA, 34 bp, Homo sapiens
  source 1..34: mol_type = genomic DNA
cgacccctccccgctgacctcactcgagccgccg

SEQ ID NO: 629 - DNA, 48 bp, Homo sapiens
  source 1..48: mol_type = genomic DNA
cggggtcctcagcgctgcagactcctgacctgccgactgcggatcccg

SEQ ID NO: 630 - DNA, 44 bp, Homo sapiens
  source 1..44: mol_type = genomic DNA
cgctgcagactcctgacctgccgactgcggatcccgagtccccg

SEQ ID NO: 631 - DNA, 30 bp, Homo sapiens
  source 1..30: mol_type = genomic DNA
cgactgcggatcccgagtccccggatcccg

SEQ ID NO: 632 - DNA, 61 bp, Homo sapiens
  source 1..61: mol_type = genomic DNA
cggatcccgagtccccggatcccggacccatcctgtggagcccactcctggcaggtaaccg

SEQ ID NO: 633 - DNA, 74 bp, Homo sapiens
  source 1..74: mol_type = genomic DNA
cgagtccccggatcccggacccatcctgtggagcccactcctggcaggtaaccgccccaacccctctccttccg

SEQ ID NO: 634 - DNA, 72 bp, Homo sapiens
  source 1..72: mol_type = genomic DNA
cggatcccggacccatcctgtggagcccactcctggcaggtaaccgccccaacccctctccttccgcagacg

SEQ ID NO: 635 - DNA, 72 bp, Homo sapiens
  source 1..72: mol_type = genomic DNA
cggacccatcctgtggagcccactcctggcaggtaaccgccccaacccctctccttccgcagacggtgtccg

SEQ ID NO: 636 - DNA, 61 bp, Homo sapiens
  source 1..61: mol_type = genomic DNA
cgccccaacccctctccttccgcagacggtgtccgggagcactggaaagggagccctctcg

SEQ ID NO: 637 - DNA, 68 bp, Homo sapiens
  source 1..68: mol_type = genomic DNA
cggggctctgcagccactgccctgtgcgctcagcacctctgggggcgatcagggcccctgcgcttccg

SEQ ID NO: 638 - DNA, 46 bp, Homo sapiens
  source 1..46: mol_type = genomic DNA
cgctcagcacctctgggggcgatcagggcccctgcgcttccgcccg

SEQ ID NO: 639 - DNA, 27 bp, Homo sapiens
  source 1..27: mol_type = genomic DNA
cgctccccacccgctggcggcgcctcg

SEQ ID NO: 640 - DNA, 20 bp, Homo sapiens
  source 1..20: mol_type = genomic DNA
cgctggcggcgcctcggtcg

SEQ ID NO: 641 - DNA, 18 bp, Homo sapiens
  source 1..18: mol_type = genomic DNA
cggcgcctcggtcgcccg

SEQ ID NO: 642 - DNA, 31 bp, Homo sapiens
  source 1..31: mol_type = genomic DNA
cgcctcggtcgcccgcaattgacccaacccg

SEQ ID NO: 643 - DNA, 35 bp, Homo sapiens
  source 1..35: mol_type = genomic DNA
cggtcgcccgcaattgacccaacccgcttcctgcg

SEQ ID NO: 644 - DNA, 51 bp, Homo sapiens
  source 1..51: mol_type = genomic DNA
cgcccgcaattgacccaacccgcttcctgcgtttgcccctcaggtttcccg

SEQ ID NO: 645 - DNA, 83 bp, Homo sapiens
  source 1..83: mol_type = genomic DNA
cgcccccggtgccctctcttgaacgcctccatccctcccgccttcctcctctgggctcatggggagggtatggaggaggagcg

SEQ ID NO: 646 - DNA, 79 bp, Homo sapiens
  source 1..79: mol_type = genomic DNA
cgccgtagacctccccaaattgcccgtctccggtatgactgaatctgtctgctggcctctcagctaccaactctgcccg

SEQ ID NO: 647 - DNA, 48 bp, Homo sapiens
  source 1..48: mol_type = genomic DNA
cgaacccacctggctcctatcgcacgggacattcccgacccacccacg

SEQ ID NO: 648 - DNA, 69 bp, Homo sapiens
  source 1..69: mol_type = genomic DNA
cggatggcctggctgcttcagggcgtaccatgcccgcaggcagatgtttattattaaaaactaccgacg

SEQ ID NO: 649 - DNA, 66 bp, Homo sapiens
  source 1..66: mol_type = genomic DNA
cgtaccatgcccgcaggcagatgtttattattaaaaactaccgacgttcatcaaccaggagacccg

SEQ ID NO: 650 - DNA, 66 bp, Homo sapiens
  source 1..66: mol_type = genomic DNA
cgcaggcagatgtttattattaaaaactaccgacgttcatcaaccaggagacccgcaaggcctgcg

SEQ ID NO: 651 - DNA, 47 bp, Homo sapiens
  source 1..47: mol_type = genomic DNA
cgacgttcatcaaccaggagacccgcaaggcctgcgcactgaatacg

SEQ ID NO: 652 - DNA, 138 bp, Homo sapiens
  source 1..138: mol_type = genomic DNA
cgaataacgagagtccatcttggggaaggaagttactctatcctatgttattttacttcttatctgttctttcattaatttggtataaccctgtttctatgctgggtggataaaatggagggggccggcggaaatgcg

SEQ ID NO: 653 - DNA, 18 bp, Homo sapiens
  source 1..18: mol_type = genomic DNA
cgacgcgcgcagggagcg

SEQ ID NO: 654 - DNA, 113 bp, Homo sapiens
  source 1..113: mol_type = genomic DNA
cgcctgctgcagctctttgcgctcttgcccaaagctaagcttcctctgggctgtggggagactcctctggtccccgttccatctacccagcccggcccctctggggctcagcg

SEQ ID NO: 655 - DNA, 127 bp, Homo sapiens
  source 1..127: mol_type = genomic DNA
cgatgagaaaccaccaagtcagaaatcgtcagcgatgtggatgactcgggaaacttctctgtctgggtctgggagtatgaagatctataagaacctctctgcacacagaaatccaagagctcacacg

SEQ ID NO: 656 - DNA, 123 bp, Homo sapiens
  source 1..123: mol_type = genomic DNA
cgacagggaggagccgggaaagcagagatgcagtggcctctcctcccctctcctccccatctttctaatccaggccaggcctgcggtcctggccaggccgtttcaggaacagccagggcctcg

SEQ ID NO: 657 - DNA, 113 bp, Homo sapiens
  source 1..113: mol_type = genomic DNA
cgggaaagcagagatgcagtggcctctcctcccctctcctccccatctttctaatccaggccaggcctgcggtcctggccaggccgtttcaggaacagccagggcctcggacg

SEQ ID NO: 658 - DNA, 85 bp, Homo sapiens
  source 1..85: mol_type = genomic DNA
cgtgggggtggtttgccgtaatgtgagtgtttcttaattagagaacggttgacaatagagggtctggcagaggctcctggccgcg

SEQ ID NO: 659 - DNA, 64 bp, Homo sapiens
  source 1..64: mol_type = genomic DNA
cggccacatttaacaccatcatcacccctccccggcctcctcaacctcggcctcctcctcgtcg

SEQ ID NO: 660 - DNA, 74 bp, Homo sapiens
  source 1..74: mol_type = genomic DNA
cggcctcctcaacctcggcctcctcctcgtcgacagccttccttggccccccaccagcagagctcacagtagcg

SEQ ID NO: 661 - DNA, 132 bp, Homo sapiens
  source 1..132: mol_type = genomic DNA
cgcctactgtgtgctaggcttcaggcgtgtttttccgacctgacagcctctggctgtgcagcagcatctccagcctagcctcgggcccaggacatttatttatcaagaggggatctccctccagagttgccg

SEQ ID NO: 662 - DNA, 90 bp, Homo sapiens
  source 1..90: mol_type = genomic DNA
cggagaaatagctctggtataactgctacccagttctccccctttctctcccgggcgagccaaatctttttcacgttttcaaccacaacg

SEQ ID NO: 663 - DNA, 84 bp, Homo sapiens
  source 1..84: mol_type = genomic DNA
cgaggggctccaccgggtgggtagaagagccgcggttgctttaaagacaagaaaagaaggtccagggctccctaggccccttcg

SEQ ID NO: 664 - DNA, 89 bp, Homo sapiens
  source 1..89: mol_type = genomic DNA
cgggcctgcctctggcctgagtaccgtttccctgcgtctcggcctctcctgaagggagctgggccctggggagcctctggccaaggtcg

SEQ ID NO: 665 - DNA, 137 bp, Homo sapiens
  source 1..137: mol_type = genomic DNA
cgtttttatttaataagcctccagttcacactgcaactccccctttagttaattgctgagtattctgctttttctcgcgtatcctcagaaccaaaggtgtcgatatttggactacagagccaagcagttgcaggacg

SEQ ID NO: 666 - DNA, 38 bp, Homo sapiens
  source 1..38: mol_type = genomic DNA
cggacatgaccggggcgcagcggtccttcacctgcacg

SEQ ID NO: 667 - DNA, 35 bp, Homo sapiens
  source 1..35: mol_type = genomic DNA
cggggcgcagcggtccttcacctgcacgcagagcg

SEQ ID NO: 668 - DNA, 34 bp, Homo sapiens
  source 1..34: mol_type = genomic DNA
cggggggacctgtgcccgcggccgtgcacctccg

SEQ ID NO: 669 - DNA, 88 bp, Homo sapiens
  source 1..88: mol_type = genomic DNA
cgtacacaccgaggtctatcagactcccccgattttagcgaagggtgctgactgctgtgctgctagaggctagcaagctccctgtgcg

SEQ ID NO: 670 - DNA, 103 bp, Homo sapiens
  source 1..103: mol_type = genomic DNA
cgaggtctatcagactcccccgattttagcgaagggtgctgactgctgtgctgctagaggctagcaagctccctgtgcgcagctgatgagtttcagcaactcg

SEQ ID NO: 671 - DNA, 94 bp, Homo sapiens
  source 1..94: mol_type = genomic DNA
cgattttagcgaagggtgctgactgctgtgctgctagaggctagcaagctccctgtgcgcagctgatgagtttcagcaactcgccacctgggcg

SEQ ID NO: 672 - DNA, 135 bp, Homo sapiens
  source 1..135: mol_type = genomic DNA
cgaagggtgctgactgctgtgctgctagaggctagcaagctccctgtgcgcagctgatgagtttcagcaactcgccacctgggcgcttttctttaaattttgggagtaaactgggaaaataaaaaaatctccacg

SEQ ID NO: 673 - DNA, 58 bp, Homo sapiens
  source 1..58: mol_type = genomic DNA
cgtttgtgaatgaacaaaagtcaccggcaagcagggagacggggacagatcgctgacg

SEQ ID NO: 674 - DNA, 69 bp, Homo sapiens
  source 1..69: mol_type = genomic DNA
cggcatctgcctgaataaagccctcgcgcgctctgagcctcccactctgtcctggaggcctgaacctcg

SEQ ID NO: 675 - DNA, 70 bp, Homo sapiens
  source 1..70: mol_type = genomic DNA
cgctctgagcctcccactctgtcctggaggcctgaacctcgcgagtagagcctggcgcttttcctccccg

SEQ ID NO: 676 - DNA, 40 bp, Homo sapiens
  source 1..40: mol_type = genomic DNA
cgggggccgcagcgctttgcaaagccggcctctgcccccg

SEQ ID NO: 677 - DNA, 49 bp, Homo sapiens
  source 1..49: mol_type = genomic DNA
cgcagcgctttgcaaagccggcctctgcccccggcctccttggagggcg

SEQ ID NO: 678 - DNA, 50 bp, Homo sapiens
  source 1..50: mol_type = genomic DNA
cgctttgcaaagccggcctctgcccccggcctccttggagggcggtagcg

SEQ ID NO: 679 - DNA, 46 bp, Homo sapiens
  source 1..46: mol_type = genomic DNA
cggcctctgcccccggcctccttggagggcggtagcgggacctgcg

SEQ ID NO: 680 - DNA, 37 bp, Homo sapiens
  source 1..37: mol_type = genomic DNA
cggcctccttggagggcggtagcgggacctgcgctcg

SEQ ID NO: 681 - DNA, 44 bp, Homo sapiens
  source 1..44: mol_type = genomic DNA
cggtagcgggacctgcgctcgcagcctctccccagggctgtgcg

SEQ ID NO: 682 - DNA, 43 bp, Homo sapiens
  source 1..43: mol_type = genomic DNA
cgggacctgcgctcgcagcctctccccagggctgtgcgcttcg

SEQ ID NO: 683 - DNA, 42 bp, Homo sapiens
  source 1..42: mol_type = genomic DNA
cgctcgcagcctctccccagggctgtgcgcttcggcctggcg

SEQ ID NO: 684 - DNA, 45 bp, Homo sapiens
  source 1..45: mol_type = genomic DNA
cgcagcctctccccagggctgtgcgcttcggcctggcggctgccg

SEQ ID NO: 685 - DNA, 26 bp, Homo sapiens
  source 1..26: mol_type = genomic DNA
cgctcccttccgtcggccggaacccg

SEQ ID NO: 686 - DNA, 24 bp, Homo sapiens
  source 1..24: mol_type = genomic DNA
cggaacccgctcctgggccgcgcg

SEQ ID NO: 687 - DNA, 54 bp, Homo sapiens
  source 1..54: mol_type = genomic DNA
cgactcttttgcaagaaaactgagcgccggctggaaggcaaggaagccgaggcg

SEQ ID NO: 688 - DNA, 88 bp, Homo sapiens
  source 1..88: mol_type = genomic DNA
cgagctctcggagcgcggagcctttagaaattgaggggtttactgtcaaaatgaaaatttcacttcaaattaccttggctgatgctcg

SEQ ID NO: 689 - DNA, 84 bp, Homo sapiens
  source 1..84: mol_type = genomic DNA
cggagcgcggagcctttagaaattgaggggtttactgtcaaaatgaaaatttcacttcaaattaccttggctgatgctcgctcg

SEQ ID NO: 690 - DNA, 84 bp, Homo sapiens
  source 1..84: mol_type = genomic DNA
cgtcccagaggcccggggtccaggagggcgcccttggcggtggcccaggcctggttcaaagtgctgtgcctgaggatggggtcg

SEQ ID NO: 691 - DNA, 93 bp, Homo sapiens
  source 1..93: mol_type = genomic DNA
cgtggaagaccccgtccacccagtttctgagactggttaccggggagtcctggtgcctgtccagggcgccagagtggggaagggccacagacg

SEQ ID NO: 692 - DNA, 103 bp, Homo sapiens
  source 1..103: mol_type = genomic DNA
cgaggccggggagaggaaggagcgaggtaatggctgtggctggagaccctcttctgactccccaggctgccgggtaggcctactgtgggatgggactcaggcg

SEQ ID NO: 693 - DNA, 136 bp, Homo sapiens
  source 1..136: mol_type = genomic DNA
cgcttggggagactgcagggcagaaatgggcctagaggtgaaggagcccttcccaaatcggacagtcccggggctggagagccgggagcaacccagttccctgaagaaatgattgcagctgctcaggcaaaacacg

SEQ ID NO: 694 - DNA, 130 bp, Homo sapiens
  source 1..130: mol_type = genomic DNA
cgctatctgagacgacttttggtgatggtccctagttcttaacttccccccttgaactcctctttcgggtagcctgacttaggagcaacacaggcaacatagcacaggggaaaaaacggttttctttccg

SEQ ID NO: 695 - DNA, 125 bp, Homo sapiens
  source 1..125: mol_type = genomic DNA
cgacttttggtgatggtccctagttcttaacttccccccttgaactcctctttcgggtagcctgacttaggagcaacacaggcaacatagcacaggggaaaaaacggttttctttccgaacaacg

SEQ ID NO: 696 - DNA, 96 bp, Homo sapiens
  source 1..96: mol_type = genomic DNA
cggcacttggcttctgaggcttttcagttgccgtccgagcaggagggagggaatgagcaggcacgcagggaaaacccagtaactgctttgcaaacg

SEQ ID NO: 697 - DNA, 67 bp, Homo sapiens
  source 1..67: mol_type = genomic DNA
cgtccgagcaggagggagggaatgagcaggcacgcagggaaaacccagtaactgctttgcaaacgcg

SEQ ID NO: 698 - DNA, 88 bp, Homo sapiens
  source 1..88: mol_type = genomic DNA
cgagcaggagggagggaatgagcaggcacgcagggaaaacccagtaactgctttgcaaacgcgacccctggtcacaataacccaggcg

SEQ ID NO: 699 - DNA, 83 bp, Homo sapiens
  source 1..83: mol_type = genomic DNA
cggcatcgaggcatttcaataaaacctaagcgactcctgatcctttctcattgtggttccttatgagctttcggtgtctggcg

SEQ ID NO: 700 - DNA, 93 bp, Homo sapiens
  source 1..93: mol_type = genomic DNA
cgtttgtcgagggagagagcgaggcaggaacagggggccggggacatgagaagtttggctaagaaatttttcttttctttgaaaacaaaagcg

SEQ ID NO: 701 - DNA, 36 bp, Homo sapiens
  source 1..36: mol_type = genomic DNA
cgcggcccccggggacgcaagagggctgggaggccg

SEQ ID NO: 702 - DNA, 38 bp, Homo sapiens
  source 1..38: mol_type = genomic DNA
cggcccccggggacgcaagagggctgggaggccgggcg

SEQ ID NO: 703 - DNA, 38 bp, Homo sapiens
  source 1..38: mol_type = genomic DNA
cggggacgcaagagggctgggaggccgggcgggtgacg

SEQ ID NO: 704 - DNA, 110 bp, Homo sapiens
  source 1..110: mol_type = genomic DNA
cggttcgacttctccacgggggagagaaatgtcctaaatatggaaaaggctgactctttgatgagttgctggagagtcgttgacagttggctgggtttaccaccttcacg

SEQ ID NO: 705 - DNA, 111 bp, Homo sapiens
  source 1..111: mol_type = genomic DNA
cgacttctccacgggggagagaaatgtcctaaatatggaaaaggctgactctttgatgagttgctggagagtcgttgacagttggctgggtttaccaccttcacgaccacg

SEQ ID NO: 706 - DNA, 141 bp, Homo sapiens
  source 1..141: mol_type = genomic DNA
cgggggagagaaatgtcctaaatatggaaaaggctgactctttgatgagttgctggagagtcgttgacagttggctgggtttaccaccttcacgaccacgaaatctgaactagtctgatttgggagtgggggacacccccg

SEQ ID NO: 707 - DNA, 133 bp, Homo sapiens
  source 1..133: mol_type = genomic DNA
cgttgacagttggctgggtttaccaccttcacgaccacgaaatctgaactagtctgatttgggagtgggggacacccccgctgttatttaactgccaggagactgcacttgcctgaaatgagatcatccaccg

SEQ ID NO: 708 - DNA, 138 bp, Homo sapiens
  source 1..138: mol_type = genomic DNA
cgaccacgaaatctgaactagtctgatttgggagtgggggacacccccgctgttatttaactgccaggagactgcacttgcctgaaatgagatcatccaccgtgtctctgaaatcttgcagattgaagtctgcttccg

SEQ ID NO: 709 - DNA, 123 bp, Homo sapiens
  source 1..123: mol_type = genomic DNA
cgtgtctctgaaatcttgcagattgaagtctgcttccgtttggtgggaatggttctgaaaaaaaccagaggtgtacactggttaacgagtctggcgttagaaagccttcaataaatattggcg

SEQ ID NO: 710 - DNA, 129 bp, Homo sapiens
  source 1..129: mol_type = genomic DNA
cgtttggtgggaatggttctgaaaaaaaccagaggtgtacactggttaacgagtctggcgttagaaagccttcaataaatattggcgtccctgttaaaaatctggcaggcactatgcaatatatgcacg

SEQ ID NO: 711 - DNA, 136 bp, Homo sapiens
  source 1..136: mol_type = genomic DNA
cgagtctggcgttagaaagccttcaataaatattggcgtccctgttaaaaatctggcaggcactatgcaatatatgcacgtaacacaactgcacttgtacccctaagtccccccaaataaaacaatttttgtggcg

SEQ ID NO: 712 - DNA, 133 bp, Homo sapiens
  source 1..133: mol_type = genomic DNA
cgttagaaagccttcaataaatattggcgtccctgttaaaaatctggcaggcactatgcaatatatgcacgtaacacaactgcacttgtacccctaagtccccccaaataaaacaatttttgtggcgggtccg

SEQ ID NO: 713 - DNA, 138 bp, Homo sapiens
  source 1..138: mol_type = genomic DNA
cgtccctgttaaaaatctggcaggcactatgcaatatatgcacgtaacacaactgcacttgtacccctaagtccccccaaataaaacaatttttgtggcgggtccggaaccctttctagaccaacagacctcctcccg

SEQ ID NO: 714 - DNA, 102 bp, Homo sapiens
  source 1..102: mol_type = genomic DNA
cgtaacacaactgcacttgtacccctaagtccccccaaataaaacaatttttgtggcgggtccggaaccctttctagaccaacagacctcctcccgggctcg

SEQ ID NO: 715 - DNA, 49 bp, Homo sapiens
  source 1..49: mol_type = genomic DNA
cgggtccggaaccctttctagaccaacagacctcctcccgggctcggcg

SEQ ID NO: 716 - DNA, 33 bp, Homo sapiens
  source 1..33: mol_type = genomic DNA
cgcacttacgtcgccagcccagtctcgtacccg

SEQ ID NO: 717 - DNA, 44 bp, Homo sapiens
  source 1..44: mol_type = genomic DNA
cgtcgccagcccagtctcgtacccgaaaattcaagccccatccg

SEQ ID NO: 718 - DNA, 74 bp, Homo sapiens
  source 1..74: mol_type = genomic DNA
cgccagcccagtctcgtacccgaaaattcaagccccatccgagacagggaacccagcaggcttgcactgccacg

SEQ ID NO: 719 - DNA, 70 bp, Homo sapiens
  source 1..70: mol_type = genomic DNA
cgtacccgaaaattcaagccccatccgagacagggaacccagcaggcttgcactgccacggtggggagcg

SEQ ID NO: 720 - DNA, 69 bp, Homo sapiens
  source 1..69: mol_type = genomic DNA
cgaaaattcaagccccatccgagacagggaacccagcaggcttgcactgccacggtggggagcgggacg

SEQ ID NO: 721 - DNA, 54 bp, Homo sapiens
  source 1..54: mol_type = genomic DNA
cgagacagggaacccagcaggcttgcactgccacggtggggagcgggacgcacg

SEQ ID NO: 722 - DNA, 28 bp, Homo sapiens
  source 1..28: mol_type = genomic DNA
cggtggggagcgggacgcacggagcacg

SEQ ID NO: 723 - DNA, 49 bp, Homo sapiens
  source 1..49: mol_type = genomic DNA
cgggacgcacggagcacgacactgactgggggaagggggcagcagttcg

SEQ ID NO: 724 - DNA, 46 bp, Homo sapiens
  source 1..46: mol_type = genomic DNA
cgcacggagcacgacactgactgggggaagggggcagcagttcgcg

SEQ ID NO: 725 - DNA, 62 bp, Homo sapiens
  source 1..62: mol_type = genomic DNA
cggagcacgacactgactgggggaagggggcagcagttcgcggctcctgcagagcagctgcg

SEQ ID NO: 726 - DNA, 60 bp, Homo sapiens
  source 1..60: mol_type = genomic DNA
cgacactgactgggggaagggggcagcagttcgcggctcctgcagagcagctgcgtggcg

SEQ ID NO: 727 - DNA, 49 bp, Homo sapiens
  source 1..49: mol_type = genomic DNA
cggctcctgcagagcagctgcgtggcgggaatgggtccttccaccggcg

SEQ ID NO: 728 - DNA, 51 bp, Homo sapiens
  source 1..51: mol_type = genomic DNA
cggctccgggcagccgagtagcccgccacccaccaactagctaagcagccg

SEQ ID NO: 729 - DNA, 60 bp, Homo sapiens
  source 1..60: mol_type = genomic DNA
cgggcagccgagtagcccgccacccaccaactagctaagcagccgcctctgtgaagctcg

SEQ ID NO: 730 - DNA, 55 bp, Homo sapiens
  source 1..55: mol_type = genomic DNA
cgagtagcccgccacccaccaactagctaagcagccgcctctgtgaagctcggcg

SEQ ID NO: 731 - DNA, 60 bp, Homo sapiens
  source 1..60: mol_type = genomic DNA
cggatctttccacccaagacccgacagcgtgcaggggcctcgagcagtaatttgaggccg

SEQ ID NO: 732 - DNA, 67 bp, Homo sapiens
  source 1..67: mol_type = genomic DNA
cgaccgaagaaacggcatttttttcctactcctttggggcagtcccctgagcacgtgcctgaagtcg

SEQ ID NO: 733 - DNA, 74 bp, Homo sapiens
  source 1..74: mol_type = genomic DNA
cgaagaaacggcatttttttcctactcctttggggcagtcccctgagcacgtgcctgaagtcgaggttagtacg

SEQ ID NO: 734 - DNA, 106 bp, Homo sapiens
  source 1..106: mol_type = genomic DNA
cggcatttttttcctactcctttggggcagtcccctgagcacgtgcctgaagtcgaggttagtacgcagccagcagggaccctgacaggaaaccagagcagccccg

SEQ ID NO: 735 - DNA, 89 bp, Homo sapiens
  source 1..89: mol_type = genomic DNA
cgaggttagtacgcagccagcagggaccctgacaggaaaccagagcagccccggggttgggggagccctggtaggtgctggtcgtcccg

SEQ ID NO: 736 - DNA, 84 bp, Homo sapiens
  source 1..84: mol_type = genomic DNA
cgcagccagcagggaccctgacaggaaaccagagcagccccggggttgggggagccctggtaggtgctggtcgtcccggacacg

SEQ ID NO: 737 - DNA, 60 bp, Homo sapiens
  source 1..60: mol_type = genomic DNA
cgccgtggccttagggaaggccccaaagaggcctaggccccggaggaggcaagagccgcg

SEQ ID NO: 738 - DNA, 57 bp, Homo sapiens
  source 1..57: mol_type = genomic DNA
cggttccttaaggtgtcaggagggcccgaacctgagtgcggtctctccgcatctccg

SEQ ID NO: 739 - DNA, 57 bp, Homo sapiens
  source 1..57: mol_type = genomic DNA
cgcgtctcactgctgggctgcggtggaggagctgagcgagccaaggagctgggggcg

SEQ ID NO: 740 - DNA, 73 bp, Homo sapiens
  source 1..73: mol_type = genomic DNA
cgtctcactgctgggctgcggtggaggagctgagcgagccaaggagctgggggcgagggagcctaacagcccg

SEQ ID NO: 741 - DNA, 63 bp, Homo sapiens
  source 1..63: mol_type = genomic DNA
cggtggaggagctgagcgagccaaggagctgggggcgagggagcctaacagcccgctagaccg

SEQ ID NO: 742 - DNA, 64 bp, Homo sapiens
  source 1..64: mol_type = genomic DNA
cgagccaaggagctgggggcgagggagcctaacagcccgctagaccgctaagcagacacacacg

SEQ ID NO: 743 - DNA, 69 bp, Homo sapiens
  source 1..69: mol_type = genomic DNA
cgagggagcctaacagcccgctagaccgctaagcagacacacacgcacaaacccagcattagagtgccg

SEQ ID NO: 744 - DNA, 56 bp, Homo sapiens
  source 1..56: mol_type = genomic DNA
cgctagaccgctaagcagacacacacgcacaaacccagcattagagtgccgaaacg

SEQ ID NO: 745 - DNA, 59 bp, Homo sapiens
  source 1..59: mol_type = genomic DNA
cgctaagcagacacacacgcacaaacccagcattagagtgccgaaacgtaaggatgtcg

SEQ ID NO: 746 - DNA, 45 bp, Homo sapiens
  source 1..45: mol_type = genomic DNA
cgcacaaacccagcattagagtgccgaaacgtaaggatgtcgtcg

SEQ ID NO: 747 - DNA, 50 bp, Homo sapiens
  source 1..50: mol_type = genomic DNA
cgaggcggagagccacccaagaaaggtggcggaggcggggagaccctgcg

SEQ ID NO: 748 - DNA, 30 bp, Homo sapiens
  source 1..30: mol_type = genomic DNA
cgcgcacatccccggcttccccgggctccg

SEQ ID NO: 749 - DNA, 37 bp, Homo sapiens
  source 1..37: mol_type = genomic DNA
cgggctccgcgccttcccaagagccccgttgtctccg

SEQ ID NO: 750 - DNA, 28 bp, Homo sapiens
  source 1..28: mol_type = genomic DNA
cggcgtcccagggatcgcgtgggctccg

SEQ ID NO: 751 - DNA, 27 bp, Homo sapiens
  source 1..27: mol_type = genomic DNA
cgtcccagggatcgcgtgggctccgcg

SEQ ID NO: 752 - DNA, 37 bp, Homo sapiens
  source 1..37: mol_type = genomic DNA
cgcgtgggctccgcgcaatctctcccccactttaacg

SEQ ID NO: 753 - DNA, 38 bp, Homo sapiens
  source 1..38: mol_type = genomic DNA
cgtgggctccgcgcaatctctcccccactttaacggcg

SEQ ID NO: 754 - DNA, 31 bp, Homo sapiens
  source 1..31: mol_type = genomic DNA
cgcgcaatctctcccccactttaacggcgcg

SEQ ID NO: 755 - DNA, 38 bp, Homo sapiens
  source 1..38: mol_type = genomic DNA
cgcaatctctcccccactttaacggcgcgttttagccg

SEQ ID NO: 756 - DNA, 20 bp, Homo sapiens
  source 1..20: mol_type = genomic DNA
cggcgcgttttagccgcccg

SEQ ID NO: 757 - DNA, 25 bp, Homo sapiens
  source 1..25: mol_type = genomic DNA
cgcgttttagccgcccggcctaacg

SEQ ID NO: 758 - DNA, 33 bp, Homo sapiens
  source 1..33: mol_type = genomic DNA
cgttttagccgcccggcctaacgcctctccccg

SEQ ID NO: 759 - DNA, 44 bp, Homo sapiens
  source 1..44: mol_type = genomic DNA
cgacgctgggacgtagacaaaaagggtcggaggagatggctgcg

SEQ ID NO: 760 - DNA, 36 bp, Homo sapiens
  source 1..36: mol_type = genomic DNA
cgcaggctggagccccgggggcgtgaaccccacgcg

SEQ ID NO: 761 - DNA, 142 bp, Homo sapiens
  source 1..142: mol_type = genomic DNA
cgcctggaggcctggcagacgctagtgggccagggcctaaggtggggatctccggtggagggagggagcaggtctgaatggtgggagagtgaagttctgcttgtgggtctaagctttggagggctgcagcagaggcggcccg

SEQ ID NO: 762 - DNA, 136 bp, Homo sapiens
  source 1..136: mol_type = genomic DNA
cgctagtgggccagggcctaaggtggggatctccggtggagggagggagcaggtctgaatggtgggagagtgaagttctgcttgtgggtctaagctttggagggctgcagcagaggcggcccgggagccctctgcg

SEQ ID NO: 763 - DNA, 67 bp, Homo sapiens
  source 1..67: mol_type = genomic DNA
cgcgggaatgtctttattggacgttacgaagggaattggggtttctctcctgggaagaaaaagcccg

SEQ ID NO: 764 - DNA, 75 bp, Homo sapiens
  source 1..75: mol_type = genomic DNA
cgggaatgtctttattggacgttacgaagggaattggggtttctctcctgggaagaaaaagcccgacctgtgccg

SEQ ID NO: 765 - DNA, 52 bp, Homo sapiens
  source 1..52: mol_type = genomic DNA
cgacctgtgccggcgagagcaggctgccttgggctgggggcacctccgagcg

SEQ ID NO: 766 - DNA, 47 bp, Homo sapiens
  source 1..47: mol_type = genomic DNA
cgagagcaggctgccttgggctgggggcacctccgagcgctgcggcg

SEQ ID NO: 767 - DNA, 19 bp, Homo sapiens
  source 1..19: mol_type = genomic DNA
cgagcgctgcggcgggacg

SEQ ID NO: 768 - DNA, 100 bp, Homo sapiens
  source 1..100: mol_type = genomic DNA
cgagtttggggttttgagattggacctttcttctttctgctcagcctgcgggagggagctggtcttgcgtgttggcagggagagtccgctgcccagagcg

SEQ ID NO: 769 - DNA, 130 bp, Homo sapiens
  source 1..130: mol_type = genomic DNA
cgaacctactcctggtaccccgaagtgcccagcctgagcctgcttgctgttgggattatgggatgggaccacagggcaggtcgggctgtggcctgcctttctcagggactggagcggctaagcctgcccg

SEQ ID NO: 770 - DNA, 62 bp, Homo sapiens
  source 1..62: mol_type = genomic DNA
cgtgcctccagggccgcttggcctgggaacgcagagacccacgaccccctccccaacctccg

SEQ ID NO: 771 - DNA, 89 bp, Homo sapiens
  source 1..89: mol_type = genomic DNA
cggtccagctcccttccgttccgttttattctgttgctccataataaggcctagcggggttttttttcttaattgtttttatttcaacg

SEQ ID NO: 772 - DNA, 75 bp, Homo sapiens
  source 1..75: mol_type = genomic DNA
cgttccgttttattctgttgctccataataaggcctagcggggttttttttcttaattgtttttatttcaacgcg

SEQ ID NO: 773 - DNA, 98 bp, Homo sapiens
  source 1..98: mol_type = genomic DNA
cgttttattctgttgctccataataaggcctagcggggttttttttcttaattgtttttatttcaacgcgcttttggggaacagatggtcaggtggcg

SEQ ID NO: 774 - DNA, 116 bp, Homo sapiens
  source 1..116: mol_type = genomic DNA
cggggttttttttcttaattgtttttatttcaacgcgcttttggggaacagatggtcaggtggcgtttggttacatgaataagttctttagtggtgatttctgagattttggggcg

SEQ ID NO: 775 - DNA, 124 bp, Homo sapiens
  source 1..124: mol_type = genomic DNA
cgcctgtgcagaccctccgttgtgtggccttgtgtctttaggcagggaacatcttttgtctctccgcaagaagctttcctccaggaaagataaagtaatcgatagggtcttttaaatagctccg

SEQ ID NO: 776 - DNA, 102 bp, Homo sapiens
  source 1..102: mol_type = genomic DNA
cgcatgtctctcccggagggcttccttttttttttttttcctaaccacgaaaatagggaatctttgttacaggctagttgtgagcgaatcctcctttgtccg

SEQ ID NO: 777 - DNA, 91 bp, Homo sapiens
  source 1..91: mol_type = genomic DNA
cggagggcttccttttttttttttttcctaaccacgaaaatagggaatctttgttacaggctagttgtgagcgaatcctcctttgtccgcg

SEQ ID NO: 778 - DNA, 78 bp, Homo sapiens
  source 1..78: mol_type = genomic DNA
cgtttttcttatggggccggggtcgggcaacgtggttcagaggtttcacaagctccagcacagaagtgatccaaaccg

SEQ ID NO: 779 - DNA, 69 bp, Homo sapiens
  source 1..69: mol_type = genomic DNA
cggggtcgggcaacgtggttcagaggtttcacaagctccagcacagaagtgatccaaaccgaggggtcg

SEQ ID NO: 780 - DNA, 68 bp, Homo sapiens
  source 1..68: mol_type = genomic DNA
cgggcaacgtggttcagaggtttcacaagctccagcacagaagtgatccaaaccgaggggtcggctcg

SEQ ID NO: 781 - DNA, 69 bp, Homo sapiens
  source 1..69: mol_type = genomic DNA
cgctggacttctccggctcgaactcctaatctcagcccggaatccagccatattcatttaaggattgcg

SEQ ID NO: 782 - DNA, 82 bp, Homo sapiens
  source 1..82: mol_type = genomic DNA
cggctcgaactcctaatctcagcccggaatccagccatattcatttaaggattgcgttttccaaaggccaaagcctttcccg

SEQ ID NO: 783 - DNA, 83 bp, Homo sapiens
  source 1..83: mol_type = genomic DNA
cgaactcctaatctcagcccggaatccagccatattcatttaaggattgcgttttccaaaggccaaagcctttcccggtttcg

SEQ ID NO: 784 - DNA, 24 bp, Homo sapiens
  source 1..24: mol_type = genomic DNA
cggccgcaggcgaggtcctccgcg

SEQ ID NO: 785 - DNA, 42 bp, Homo sapiens
  source 1..42: mol_type = genomic DNA
cgcgcgtggaggccgccaggggagtggaaactgacagagtcg

SEQ ID NO: 786 - DNA, 61 bp, Homo sapiens
  source 1..61: mol_type = genomic DNA
cggcctagccatctttctccccagcctgggaaggcgggcgggaggaggggatccgaggccg

SEQ ID NO: 787 - DNA, 116 bp, Homo sapiens
  source 1..116: mol_type = genomic DNA
cgtcccttccagcagcatcagtgggcgaaaagttaacatgctgagggccaccggcacccacacttgcttccaggaatacttgcggtacaggcttcctgacttattttctttagccg

SEQ ID NO: 788 - DNA, 116 bp, Homo sapiens
  source 1..116: mol_type = genomic DNA
cgaaaagttaacatgctgagggccaccggcacccacacttgcttccaggaatacttgcggtacaggcttcctgacttattttctttagccgctgggtgcccccccttcttcccccg

SEQ ID NO: 789 - DNA, 100 bp, Homo sapiens
  source 1..100: mol_type = genomic DNA
cggcacccacacttgcttccaggaatacttgcggtacaggcttcctgacttattttctttagccgctgggtgcccccccttcttcccccgttgggagacg

SEQ ID NO: 790 - DNA, 107 bp, Homo sapiens
  source 1..107: mol_type = genomic DNA
cgagtcgtgcccaccgcccaggccaggagatctggggctactgggagatctctcatgaaggtggctataaaaagagccctgaccgggacacaagcctttggagagcg

SEQ ID NO: 791 - DNA, 86 bp, Homo sapiens
  source 1..86: mol_type = genomic DNA
cgtgggcaggtgtgagcgctcgccctctctcctcccgagcctgctattttgtggtccaggacttgccatcagaacttgctttaacg

SEQ ID NO: 792 - DNA, 72 bp, Homo sapiens
  source 1..72: mol_type = genomic DNA
cgctcgccctctctcctcccgagcctgctattttgtggtccaggacttgccatcagaacttgctttaacgcg

SEQ ID NO: 793 - DNA, 41 bp, Homo sapiens
  source 1..41: mol_type = genomic DNA
cgaggcgcgggactggaaggacaggtaccaggctgcgggcg

SEQ ID NO: 794 - DNA, 36 bp, Homo sapiens
  source 1..36: mol_type = genomic DNA
cgggccagacgccgaagaggaaggtgaccgaacccg

SEQ ID NO: 795 - DNA, 50 bp, Homo sapiens
  source 1..50: mol_type = genomic DNA
cgtccacccagcgaggggcttgtgtgccctagagggcgctgggccccgcg

SEQ ID NO: 796 - DNA, 41 bp, Homo sapiens
  source 1..41: mol_type = genomic DNA
cgaggggcttgtgtgccctagagggcgctgggccccgcgcg

SEQ ID NO: 797 - DNA, 60 bp, Homo sapiens
  source 1..60: mol_type = genomic DNA
cgcgctttcctggctgtccccgccggctttccaccctccccaaagcccaggtgcccaccg

SEQ ID NO: 798 - DNA, 65 bp, Homo sapiens
  source 1..65: mol_type = genomic DNA
cgctttcctggctgtccccgccggctttccaccctccccaaagcccaggtgcccaccgtgggtcg

SEQ ID NO: 799 - DNA, 52 bp, Homo sapiens
  source 1..52: mol_type = genomic DNA
cgccggctttccaccctccccaaagcccaggtgcccaccgtgggtcgctgcg

SEQ ID NO: 800 - DNA, 75 bp, Homo sapiens
  source 1..75: mol_type = genomic DNA
cggctttccaccctccccaaagcccaggtgcccaccgtgggtcgctgcggcctttccccttcttggccaaatccg

SEQ ID NO: 801 - DNA, 49 bp, Homo sapiens
  source 1..49: mol_type = genomic DNA
cgtgggtcgctgcggcctttccccttcttggccaaatccgattacttcg

SEQ ID NO: 802 - DNA, 61 bp, Homo sapiens
  source 1..61: mol_type = genomic DNA
cgctgcggcctttccccttcttggccaaatccgattacttcgcagcctgcagatggcatcg

SEQ ID NO: 803 - DNA, 59 bp, Homo sapiens
  source 1..59: mol_type = genomic DNA
cggcctttccccttcttggccaaatccgattacttcgcagcctgcagatggcatcgccg

SEQ ID NO: 804 - DNA, 71 bp, Homo sapiens
  source 1..71: mol_type = genomic DNA
cgccggctaagggcagcctgcggcaggtccccgagcctgagcactcctcctatctggggcctgagaggacg

SEQ ID NO: 805 - DNA, 98 bp, Homo sapiens
  source 1..98: mol_type = genomic DNA
cggctaagggcagcctgcggcaggtccccgagcctgagcactcctcctatctggggcctgagaggacgctctgggctttttcccaggcccagggtgcg

SEQ ID NO: 806 - DNA, 78 bp, Homo sapiens
  source 1..78: mol_type = genomic DNA
cgcactggcgccctccgctcaacccaccttgcctttggggcgggcttcaaccctgggaagacaggcctgggggaagcg

SEQ ID NO: 807 - DNA, 84 bp, Homo sapiens
  source 1..84: mol_type = genomic DNA
cgccctccgctcaacccaccttgcctttggggcgggcttcaaccctgggaagacaggcctgggggaagcgagaggagaggcccg

SEQ ID NO: 808 - DNA, 66 bp, Homo sapiens
  source 1..66: mol_type = genomic DNA
cgggccctgggaagaccccggggacgcgctggagcccctgaagggggtcagggggaacaatgcccg

SEQ ID NO: 809 - DNA, 65 bp, Homo sapiens
  source 1..65: mol_type = genomic DNA
cggggacgcgctggagcccctgaagggggtcagggggaacaatgcccgaaaagggagcctccccg

SEQ ID NO: 810 - DNA, 129 bp, Homo sapiens
  source 1..129: mol_type = genomic DNA
cgattgcgaggggcctaggttagggacacgctcttctcgctgggtcccaagcccaatcacccaggccaatcaccagcctctaacctccttcccctctaaccccatccccacctaggaagttaaaaaacg

SEQ ID NO: 811 - DNA, 132 bp, Homo sapiens
  source 1..132: mol_type = genomic DNA
cgaggggcctaggttagggacacgctcttctcgctgggtcccaagcccaatcacccaggccaatcaccagcctctaacctccttcccctctaaccccatccccacctaggaagttaaaaaacgcccctgccg

SEQ ID NO: 812 - DNA, 117 bp, Homo sapiens
  source 1..117: mol_type = genomic DNA
cgggaagtgttatcaaacagtaatagctaccctcgactccgaatcacagcattccccccagaattcctgatcagtaaaacccaaatcgttttcaagacaaaggagcatcaatcagcg

SEQ ID NO: 813 - DNA, 124 bp, Homo sapiens
  source 1..124: mol_type = genomic DNA
cgtcttgccctgattgttctgggctgggattggattactgggactgcccagggtcccgcgccaggaaacaccaccttggactacagggaagagactgatctccctcctcctgccggcccccacg

SEQ ID NO: 814 - DNA, 79 bp, Homo sapiens
  source 1..79: mol_type = genomic DNA
cgcgccaggaaacaccaccttggactacagggaagagactgatctccctcctcctgccggcccccacgttgatttcccg

SEQ ID NO: 815 - DNA, 88 bp, Homo sapiens
  source 1..88: mol_type = genomic DNA
cgccaggaaacaccaccttggactacagggaagagactgatctccctcctcctgccggcccccacgttgatttcccgagaaacaagcg

SEQ ID NO: 816 - DNA, 101 bp, Homo sapiens
  source 1..101: mol_type = genomic DNA
cgccggccgtctccacaacccttgcttcctacaggcagaattgaatgcaggagctggctgtcttcccgcctggggtgggctctcaattcaactggacctcg

SEQ ID NO: 817 - DNA, 96 bp, Homo sapiens
  source 1..96: mol_type = genomic DNA
cgctctctctctgcatccccctcaccccctttctcggagactgaactaagtgaaaagttgtttcaataatcgcagctctctgctccgccagggccg

SEQ ID NO: 818 - DNA, 71 bp, Homo sapiens
  source 1..71: mol_type = genomic DNA
cggagactgaactaagtgaaaagttgtttcaataatcgcagctctctgctccgccagggccgagggaggcg

SEQ ID NO: 819 - DNA, 103 bp, Homo sapiens
  source 1..103: mol_type = genomic DNA
cgttcgcaggggctgggacttgataaaaggagacagttttctgaaaagatttgattgaaatggcgtgtgccagggctgatgggagccagcgagggacaaagcg

SEQ ID NO: 820 - DNA, 70 bp, Homo sapiens
  source 1..70: mol_type = genomic DNA
cgaggaaggcctcgggagacgagtcccacagtgagatatttgatgaaatgggtcggggctggaaagctcg

SEQ ID NO: 821 - DNA, 94 bp, Homo sapiens
  source 1..94: mol_type = genomic DNA
cgctgcccaggccgtccacatttccctccctggctgtccttcgagcccctaccagtccttcctctccctgccgcccaagcccaggcaagactcg

SEQ ID NO: 822 - DNA, 65 bp, Homo sapiens
  source 1..65: mol_type = genomic DNA
cgccctgccagcgggtggggtcctgggtggggcaggtgggacccagtgagggggtgcgcaccgcg

SEQ ID NO: 823 - DNA, 103 bp, Homo sapiens
  source 1..103: mol_type = genomic DNA
cgggtggggtcctgggtggggcaggtgggacccagtgagggggtgcgcaccgcgctgcctcctcccaggcaggggtccaggagaccagagctgagctctggcg

SEQ ID NO: 824 - DNA, 56 bp, Homo sapiens
  source 1..56: mol_type = genomic DNA
cgagggcgccattgaggtgcagattgggacctgccggctctggactgccgcccccg

SEQ ID NO: 825 - DNA, 43 bp, Homo sapiens
  source 1..43: mol_type = genomic DNA
cgaagggcgcacctgcagctctccgggattcaggcgggggtcg

SEQ ID NO: 826 - DNA, 59 bp, Homo sapiens
  source 1..59: mol_type = genomic DNA
cgcacctgcagctctccgggattcaggcgggggtcgctgtgctctcttaaaagtgagcg

SEQ ID NO: 827 - DNA, 46 bp, Homo sapiens
  source 1..46: mol_type = genomic DNA
cgggattcaggcgggggtcgctgtgctctcttaaaagtgagcggcg

SEQ ID NO: 828 - DNA, 52 bp, Homo sapiens
  source 1..52: mol_type = genomic DNA
cgggggtcgctgtgctctcttaaaagtgagcggcggtttcagcctgccaccg

SEQ ID NO: 829 - DNA, 50 bp, Homo sapiens
  source 1..50: mol_type = genomic DNA
cgctgtgctctcttaaaagtgagcggcggtttcagcctgccaccgcttcg

SEQ ID NO: 830 - DNA, 48 bp, Homo sapiens
  source 1..48: mol_type = genomic DNA
cgccagctcggaggaaactctggctggaggcgacctcgggcccagccg

SEQ ID NO: 831 - DNA, 44 bp, Homo sapiens
  source 1..44: mol_type = genomic DNA
cggaggaaactctggctggaggcgacctcgggcccagccggacg

SEQ ID NO: 832 - DNA, 27 bp, Homo sapiens
  source 1..27: mol_type = genomic DNA
cgacctcgggcccagccggacgggccg

SEQ ID NO: 833 - DNA, 26 bp, Homo sapiens
  source 1..26: mol_type = genomic DNA
cgggcccagccggacgggccgggccg

SEQ ID NO: 834 - DNA, 39 bp, Homo sapiens
  source 1..39: mol_type = genomic DNA
cggacgggccgggccgagcctaggaggggctggcagacg

SEQ ID NO: 835 - DNA, 58 bp, Homo sapiens
  source 1..58: mol_type = genomic DNA
cgggccgggccgagcctaggaggggctggcagacgtgtcccagggccagggtggggcg

SEQ ID NO: 836 - DNA, 60 bp, Homo sapiens
  source 1..60: mol_type = genomic DNA
cgagcctaggaggggctggcagacgtgtcccagggccagggtggggcgtagggagcgccg

SEQ ID NO: 837 - DNA, 55 bp, Homo sapiens
  source 1..55: mol_type = genomic DNA
cgccgtctccaccctcagtacttttggggtgggggacctgagcgtgcggagagcg

SEQ ID NO: 838 - DNA, 68 bp, Homo sapiens
  source 1..68: mol_type = genomic DNA
cgggtctcttttgaaacagagacccgcggcaccccgcttctccccctgggataggagagggagagccg

SEQ ID NO: 839 - DNA, 28 bp, Homo sapiens
  source 1..28: mol_type = genomic DNA
cgggacgccgtaacctacacggagcacg

SEQ ID NO: 840 - DNA, 47 bp, Homo sapiens
  source 1..47: mol_type = genomic DNA
cgggttcccagcccaagaaccttctcattcacctgcgcagcgccgcg

SEQ ID NO: 841 - DNA, 53 bp, Homo sapiens
  source 1..53: mol_type = genomic DNA
cggcagcggcaagaagtgggttcggaagccggctctctcctgccccagcctcg

SEQ ID NO: 842 - DNA, 61 bp, Homo sapiens
  source 1..61: mol_type = genomic DNA
cgtcggaaaggatttagtgtttatgtgaacggagcacctcggggccaagttgttgctgtcg

SEQ ID NO: 843 - DNA, 106 bp, Homo sapiens
  source 1..106: mol_type = genomic DNA
cgcgggggaaacagctcaaaagaaatgccaggtaagagctcaatattatgaagtattaaaaatcaactgctaaagcctagcgaatgtcccaaagcgagtccaggcg

SEQ ID NO: 844 - DNA, 114 bp, Homo sapiens
  source 1..114: mol_type = genomic DNA
cggccctgcagaactatctcaccgaggcgctcaaaggcatggacaagatgttcttgaacaacaccaccactaacaggcacacgtctggggaaggcccaggtagtaaaactggcg

SEQ ID NO: 845 - DNA, 105 bp, Homo sapiens
  source 1..105: mol_type = genomic DNA
cggctcctggagggagagtggcctagaaatatccaaggaatccgaagcttcccctcctccacctctgccagtacggaaacaccccaccgtcacaatcctaaagcg

SEQ ID NO: 846 - DNA, 121 bp, Homo sapiens
  source 1..121: mol_type = genomic DNA
cgaagcttcccctcctccacctctgccagtacggaaacaccccaccgtcacaatcctaaagcggggagatgggatgggaattgtcttacttggcaaggcagggtaccttcattgccagacg

SEQ ID NO: 847 - DNA, 120 bp, Homo sapiens
  source 1..120: mol_type = genomic DNA
cgaacagcctagtgtgatggcgacactgttcccccaagagtccctgccacttctggggcaacccaggatgctgccctcatggagcctgccccggaccccggaggccaggacatgcaggcg

SEQ ID NO: 848 - DNA, 105 bp, Homo sapiens
  source 1..105: mol_type = genomic DNA
cggtgactgtgcaatgaaattgtcaattatcctctctgccttcctgcgttgaaagagtttcaagaagttgtggtgagtggatgtattcccagacggatgcgggcg

SEQ ID NO: 849 - DNA, 66 bp, Homo sapiens
  source 1..66: mol_type = genomic DNA
cgttgaaagagtttcaagaagttgtggtgagtggatgtattcccagacggatgcgggcggtccccg

SEQ ID NO: 850 - DNA, 86 bp, Homo sapiens
  source 1..86: mol_type = genomic DNA
cgactgaactattccatctccaagttgtttttattttccactttaagcataagaacgatgagaaccaggagccaggcggccccgcg

SEQ ID NO: 851 - DNA, 37 bp, Homo sapiens
  source 1..37: mol_type = genomic DNA
cgcctgcggccaggggcaatcgggttctcggttcccg

SEQ ID NO: 852 - DNA, 46 bp, Homo sapiens
  source 1..46: mol_type = genomic DNA
cggccaggggcaatcgggttctcggttcccggagtcccagttcccg

SEQ ID NO: 853 - DNA, 35 bp, Homo sapiens
  source 1..35: mol_type = genomic DNA
cggtttccagtttccttatcgacgcgattgttccg

SEQ ID NO: 854 - DNA, 19 bp, Homo sapiens
  source 1..19: mol_type = genomic DNA
cgacgcgattgttccgtcg

SEQ ID NO: 855 - DNA, 66 bp, Homo sapiens
  source 1..66: mol_type = genomic DNA
cgtccgaattcccatgctactctttttgacgggtcactggtggctctataggcaggtgctgccgcg

SEQ ID NO: 856 - DNA, 78 bp, Homo sapiens
  source 1..78: mol_type = genomic DNA
cgaattcccatgctactctttttgacgggtcactggtggctctataggcaggtgctgccgcggggttgtaattacccg

SEQ ID NO: 857 - DNA, 131 bp, Homo sapiens
  source 1..131: mol_type = genomic DNA
cggcgattagaagagaaccaaatacatgtagcggtgcatgaataatgctcattgtaggaaactgacacttgctcaaggaaaaaaagtttgggtataaaatcacttcattatttgcttttaccgcagcttcg

SEQ ID NO: 858 - DNA, 70 bp, Homo sapiens
  source 1..70: mol_type = genomic DNA
cgagagccccgcaggcctgcggggtctgccaaacccggctctctaagcttcacctgcttggagaaggacg

SEQ ID NO: 859 - DNA, 76 bp, Homo sapiens
  source 1..76: mol_type = genomic DNA
cgcaggcctgcggggtctgccaaacccggctctctaagcttcacctgcttggagaaggacgtttcccccttgtccg

SEQ ID NO: 860 - DNA, 81 bp, Homo sapiens
  source 1..81: mol_type = genomic DNA
cggggtctgccaaacccggctctctaagcttcacctgcttggagaaggacgtttcccccttgtccgctttcttggggtccg

SEQ ID NO: 861 - DNA, 93 bp, Homo sapiens
  source 1..93: mol_type = genomic DNA
cgctttcttggggtccgggactggccctcttgaagtagcctgggacgtcccagccccttctcgatctggccctgctcatgcccaaggccctcg

SEQ ID NO: 862 - DNA, 80 bp, Homo sapiens
  source 1..80: mol_type = genomic DNA
cgggactggccctcttgaagtagcctgggacgtcccagccccttctcgatctggccctgctcatgcccaaggccctcgcg

SEQ ID NO: 863 - DNA, 27 bp, Homo sapiens
  source 1..27: mol_type = genomic DNA
cgtttccccgcccgggtgccggctccg

SEQ ID NO: 864 - DNA, 48 bp, Homo sapiens
  source 1..48: mol_type = genomic DNA
cgcccgggtgccggctccgggtctccagcctaaaggccctggggaccg

SEQ ID NO: 865 - DNA, 80 bp, Homo sapiens
  source 1..80: mol_type = genomic DNA
cgggtgccggctccgggtctccagcctaaaggccctggggaccgctgacctcctgctctctgttccagggaggctgtccg

SEQ ID NO: 866 - DNA, 109 bp, Homo sapiens
  source 1..109: mol_type = genomic DNA
cgagtgctgagcaaggccttctccagactcaagaccaccctgccctgggtgccccccgacaccaagctctccaagctggacacgctcaggctggcgtccagctacatcg

SEQ ID NO: 867 - DNA, 80 bp, Homo sapiens
  source 1..80: mol_type = genomic DNA
cgacaccaagctctccaagctggacacgctcaggctggcgtccagctacatcgcccacttgaggcagatcctggctaacg

SEQ ID NO: 868 - DNA, 42 bp, Homo sapiens
  source 1..42: mol_type = genomic DNA
cggggctactgcggggtgtctcgtgctgcgcagggggctgcg

SEQ ID NO: 869 - DNA, 52 bp, Homo sapiens
  source 1..52: mol_type = genomic DNA
cgtgctgcgcagggggctgcggccctggggcagacgacccaggtgctgagcg

SEQ ID NO: 870 - DNA, 50 bp, Homo sapiens
  source 1..50: mol_type = genomic DNA
cgcagggggctgcggccctggggcagacgacccaggtgctgagcgagacg

SEQ ID NO: 871 - DNA, 82 bp, Homo sapiens
  source 1..82: mol_type = genomic DNA
cggccctggggcagacgacccaggtgctgagcgagacgagagcctgggcagggggaagcttcactgggggccagaacaggcg

SEQ ID NO: 872 - DNA, 49 bp, Homo sapiens
  source 1..49: mol_type = genomic DNA
cgaggcgagttaaattccttttgccggtgcctggctgcgaggacaaacg

SEQ ID NO: 873 - DNA, 48 bp, Homo sapiens
  source 1..48: mol_type = genomic DNA
cgagttaaattccttttgccggtgcctggctgcgaggacaaacgtccg

SEQ ID NO: 874 - DNA, 42 bp, Homo sapiens
  source 1..42: mol_type = genomic DNA
cgtgagggcgctgggaccggggaggaaagagcacctgccgcg

SEQ ID NO: 875 - DNA, 61 bp, Homo sapiens
  source 1..61: mol_type = genomic DNA
cgcaggtgatgatgggcctcgttggactctgcctcccacgcatccttttgttttctgcgcg

SEQ ID NO: 876 - DNA, 61 bp, Homo sapiens
  source 1..61: mol_type = genomic DNA
cggggctgcactcgggaggacaggcccagatcctgcacggccgggaggacacctggccccg

SEQ ID NO: 877 - DNA, 79 bp, Homo sapiens
  source 1..79: mol_type = genomic DNA
cgggaggacaggcccagatcctgcacggccgggaggacacctggccccgctgtcttctcttcacagctcccctttctcg

SEQ ID NO: 878 - DNA, 77 bp, Homo sapiens
  source 1..77: mol_type = genomic DNA
cggtcccagaaacaaagatctagtgagcccagccgacgccggcctcattttccctttctaagggtcactttcttccg

SEQ ID NO: 879 - DNA, 64 bp, Homo sapiens
  source 1..64: mol_type = genomic DNA
cgacgccggcctcattttccctttctaagggtcactttcttccgccctctaaattaggtcatcg

SEQ ID NO: 880 - DNA, 73 bp, Homo sapiens
  source 1..73: mol_type = genomic DNA
cgccggcctcattttccctttctaagggtcactttcttccgccctctaaattaggtcatcgcctgggaaagcg

SEQ ID NO: 881 - DNA, 43 bp, Homo sapiens
  source 1..43: mol_type = genomic DNA
cgcaggccaggaggcttctggtagcaccggcgcgatgcccccg

SEQ ID NO: 882 - DNA, 46 bp, Homo sapiens
  source 1..46: mol_type = genomic DNA
cgatctcgccgaacatgcccggctgaaggcactcagttcccctccg

SEQ ID NO: 883 - DNA, 42 bp, Homo sapiens
  source 1..42: mol_type = genomic DNA
cgccgaacatgcccggctgaaggcactcagttcccctccgcg

SEQ ID NO: 884 - DNA, 46 bp, Homo sapiens
  source 1..46: mol_type = genomic DNA
cgccctagcaagggggctttcccgcggacatggtatctgcgccccg

SEQ ID NO: 885 - DNA, 26 bp, Homo sapiens
  source 1..26: mol_type = genomic DNA
cgcggacatggtatctgcgccccgcg

SEQ ID NO: 886 - DNA, 120 bp, Homo sapiens
  source 1..120: mol_type = genomic DNA
cgccgctctcattctcagcattgttttcagagaaggcgccttcgctgggttgtttttctctatcaactggaggagaaccacaagcatagtcagtcagggacaaagtgtgagtgtcaagcg

SEQ ID NO: 887 - DNA, 141 bp, Homo sapiens
  source 1..141: mol_type = genomic DNA
cgctctcattctcagcattgttttcagagaaggcgccttcgctgggttgtttttctctatcaactggaggagaaccacaagcatagtcagtcagggacaaagtgtgagtgtcaagcgtgggacagtcaccccttctggccg

SEQ ID NO: 888 - DNA, 114 bp, Homo sapiens
  source 1..114: mol_type = genomic DNA
cgccttcgctgggttgtttttctctatcaactggaggagaaccacaagcatagtcagtcagggacaaagtgtgagtgtcaagcgtgggacagtcaccccttctggccgacagcg

SEQ ID NO: 889 - DNA, 27 bp, Homo sapiens
  source 1..27: mol_type = genomic DNA
cgcctggcccgcccggcccgacccacg

SEQ ID NO: 890 - DNA, 52 bp, Homo sapiens
  source 1..52: mol_type = genomic DNA
cgagctccctgtaaataatccctgcggtccccgggagaggcgagtttacccg

SEQ ID NO: 891 - DNA, 64 bp, Homo sapiens
  source 1..64: mol_type = genomic DNA
cgagaaatgagaagccgccgggcctggagcggcctccacctcagctgctatcaccccctctccg

SEQ ID NO: 892 - DNA, 104 bp, Homo sapiens
  source 1..104: mol_type = genomic DNA
cgccgggcctggagcggcctccacctcagctgctatcaccccctctccgctgtcatgggattgcccaggcttaatggggttgtgcagggcttcacttgctctcg

SEQ ID NO: 893 - DNA, 125 bp, Homo sapiens
  source 1..125: mol_type = genomic DNA
cgcgtgcagattgaccctcgtgacagctcctaggcaggcattgctgccatgtggctgactctgtccctttcctggttaaatagacaaggggtgggcgtgggggaaggggatagagtgcctgtgcg

SEQ ID NO: 894 - DNA, 127 bp, Homo sapiens
  source 1..127: mol_type = genomic DNA
cgctgctgaaaggtatacgctgttaacagggacaattacttaaagggaagcgtttgtaaaatggaaaacaaatgccggggttggtaacaaatgggatcaaaagcagccattccaatctggctccccg

SEQ ID NO: 895 - DNA, 123 bp, Homo sapiens
  source 1..123: mol_type = genomic DNA
cgctgttaacagggacaattacttaaagggaagcgtttgtaaaatggaaaacaaatgccggggttggtaacaaatgggatcaaaagcagccattccaatctggctccccgagggaaaggagcg

SEQ ID NO: 896 - DNA, 94 bp, Homo sapiens
  source 1..94: mol_type = genomic DNA
cgtttgtaaaatggaaaacaaatgccggggttggtaacaaatgggatcaaaagcagccattccaatctggctccccgagggaaaggagcgggcg

SEQ ID NO: 897 - DNA, 91 bp, Homo sapiens
  source 1..91: mol_type = genomic DNA
cggggttggtaacaaatgggatcaaaagcagccattccaatctggctccccgagggaaaggagcgggcgtggccctgtaggattaggggcg

SEQ ID NO: 898 - DNA, 73 bp, Homo sapiens
  source 1..73: mol_type = genomic DNA
cgagggaaaggagcgggcgtggccctgtaggattaggggcgcttctgacctccccaagacccaggtgaggccg

SEQ ID NO: 899 - DNA, 70 bp, Homo sapiens
  source 1..70: mol_type = genomic DNA
cgggcgtggccctgtaggattaggggcgcttctgacctccccaagacccaggtgaggccgagggtccccg

SEQ ID NO: 900 - DNA, 69 bp, Homo sapiens
  source 1..69: mol_type = genomic DNA
cgtggccctgtaggattaggggcgcttctgacctccccaagacccaggtgaggccgagggtccccggcg

SEQ ID NO: 901 - DNA, 49 bp, Homo sapiens
  source 1..49: mol_type = genomic DNA
cgcttctgacctccccaagacccaggtgaggccgagggtccccggcgcg

SEQ ID NO: 902 - DNA, 94 bp, Homo sapiens
  source 1..94: mol_type = genomic DNA
cggcacaggttgctggtagcttctggactctggaggcttggccttccttctaagccgatggcggggaaagaacctcgtttccacagcttccccg

SEQ ID NO: 903 - DNA, 46 bp, Homo sapiens
  source 1..46: mol_type = genomic DNA
cgatggcggggaaagaacctcgtttccacagcttccccgacccccg

SEQ ID NO: 904 - DNA, 114 bp, Homo sapiens
  source 1..114: mol_type = genomic DNA
cgcttcacgtccctctgggccggagccctttccatggctggctcctctgggggcccttgggcctgtgagcagcgtctacttccctcagagaagaatcctttccttcccccatcg

SEQ ID NO: 905 - DNA, 100 bp, Homo sapiens
  source 1..100: mol_type = genomic DNA
cgtgaggaccccgtgcagttgagtccaggcgacaggtgcctccccaggtgtctacttgccctccctcaacctgtttagggaaagaccagaacgatgtgcg

SEQ ID NO: 906 - DNA, 98 bp, Homo sapiens
  source 1..98: mol_type = genomic DNA
cggtgcctttggattcctaaattaggtttacggaaaaggaaagacttcccaagaacagtgagaagaggaactgcagcccctccaaggccgctgcggcg

SEQ ID NO: 907 - DNA, 43 bp, Homo sapiens
  source 1..43: mol_type = genomic DNA
cgcgggttttagtttatcgagccaaagtggtcccggagaagcg

SEQ ID NO: 908 - DNA, 49 bp, Homo sapiens
  source 1..49: mol_type = genomic DNA
cgggttttagtttatcgagccaaagtggtcccggagaagcgctccctcg

SEQ ID NO: 909 - DNA, 58 bp, Homo sapiens
  source 1..58: mol_type = genomic DNA
cgagccaaagtggtcccggagaagcgctccctcgcagccaagctgcaagaagtggccg

SEQ ID NO: 910 - DNA, 52 bp, Homo sapiens
  source 1..52: mol_type = genomic DNA
cgttgcttcttctcccggacttgtttttacgcccaccctgtgctcctccgcg

SEQ ID NO: 911 - DNA, 43 bp, Homo sapiens
  source 1..43: mol_type = genomic DNA
cggacttgtttttacgcccaccctgtgctcctccgcgtttccg

SEQ ID NO: 912 - DNA, 63 bp, Homo sapiens
  source 1..63: mol_type = genomic DNA
cgccgcaggactcatagaagtgaggcggggccacagatttaagtttattccccgcaactgtcg

SEQ ID NO: 913 - DNA, 80 bp, Homo sapiens
  source 1..80: mol_type = genomic DNA
cgaggctgagagctgggcttggggcaggttcctttgcgttcgctcaggccacgatgaggccactaggacagatttctccg

SEQ ID NO: 914 - DNA, 53 bp, Homo sapiens
  source 1..53: mol_type = genomic DNA
cgggtgcccgccccagccgctgtgtccctggtgcttcctccgccagggccacg

SEQ ID NO: 915 - DNA, 55 bp, Homo sapiens
  source 1..55: mol_type = genomic DNA
cgctgtgtccctggtgcttcctccgccagggccacgtgccggggaagccaaagcg

SEQ ID NO: 916 - DNA, 136 bp, Homo sapiens
  source 1..136: mol_type = genomic DNA
cgctcatcaggaagtgtttggggtctttgagcgagtgatctacgcgttgaattgctgtttttccaaactttcatggcttaaaaattttttttcttctccctgtaaaagaaactcagagactggtgatctgttaccg

SEQ ID NO: 917 - DNA, 111 bp, Homo sapiens
  source 1..111: mol_type = genomic DNA
cgagtgatctacgcgttgaattgctgtttttccaaactttcatggcttaaaaattttttttcttctccctgtaaaagaaactcagagactggtgatctgttaccgaagacg

SEQ ID NO: 918 - DNA, 112 bp, Homo sapiens
  source 1..112: mol_type = genomic DNA
cgcgttgaattgctgtttttccaaactttcatggcttaaaaattttttttcttctccctgtaaaagaaactcagagactggtgatctgttaccgaagacgactttttaggcg

SEQ ID NO: 919 - DNA, 62 bp, Homo sapiens
  source 1..62: mol_type = genomic DNA
cgagagtgcgggacagagagggcctggcggggctccttggcgctgagagccctccttctccg

SEQ ID NO: 920 - DNA, 75 bp, Homo sapiens
  source 1..75: mol_type = genomic DNA
cgtaccttcgcttctgttgaaggcagtgagatccgcaggcctccgggacagctagaggacagcagacatgtcccg

SEQ ID NO: 921 - DNA, 99 bp, Homo sapiens
  source 1..99: mol_type = genomic DNA
cgcttctgttgaaggcagtgagatccgcaggcctccgggacagctagaggacagcagacatgtcccgtgggggaagcctgggcctgtctgaaaacaccg

SEQ ID NO: 922 - DNA, 89 bp, Homo sapiens
  source 1..89: mol_type = genomic DNA
cgcaggcctccgggacagctagaggacagcagacatgtcccgtgggggaagcctgggcctgtctgaaaacaccgcctcagctccctccg

SEQ ID NO: 923 - DNA, 92 bp, Homo sapiens
  source 1..92: mol_type = genomic DNA
cgggacagctagaggacagcagacatgtcccgtgggggaagcctgggcctgtctgaaaacaccgcctcagctccctccggggtccccctgcg

SEQ ID NO: 924 - DNA, 47 bp, Homo sapiens
  source 1..47: mol_type = genomic DNA
cgcggggtcagctggcggctcaggtgcgattggaggcctgggccccg

SEQ ID NO: 925 - DNA, 56 bp, Homo sapiens
  source 1..56: mol_type = genomic DNA
cggggtcagctggcggctcaggtgcgattggaggcctgggccccgagggaccaccg

SEQ ID NO: 926 - DNA, 50 bp, Homo sapiens
  source 1..50: mol_type = genomic DNA
cgagggacacgtgtcccgggacaggtgtgcaccaggcagccgggcatgcg

SEQ ID NO: 927 - DNA, 85 bp, Homo sapiens
  source 1..85: mol_type = genomic DNA
cgtgtcccgggacaggtgtgcaccaggcagccgggcatgcgctgacacctggccacaggtatttttgccaacacttcacattgcg

SEQ ID NO: 928 - DNA, 96 bp, Homo sapiens
  source 1..96: mol_type = genomic DNA
cgggacaggtgtgcaccaggcagccgggcatgcgctgacacctggccacaggtatttttgccaacacttcacattgcgactccccagtagcaggcg

SEQ ID NO: 929 - DNA, 74 bp, Homo sapiens
  source 1..74: mol_type = genomic DNA
cgggcatgcgctgacacctggccacaggtatttttgccaacacttcacattgcgactccccagtagcaggcgcg

SEQ ID NO: 930 - DNA, 97 bp, Homo sapiens
  source 1..97: mol_type = genomic DNA
cgctgacacctggccacaggtatttttgccaacacttcacattgcgactccccagtagcaggcgcggaggtggtcctgacacccacttgagactccg

SEQ ID NO: 931 - DNA, 73 bp, Homo sapiens
  source 1..73: mol_type = genomic DNA
cgccgagtagacaccctccccatcatttctacaagcaaattgtaatgccgcgggcataatcccccaaaactcg

SEQ ID NO: 932 - DNA, 76 bp, Homo sapiens
  source 1..76: mol_type = genomic DNA
cgagtagacaccctccccatcatttctacaagcaaattgtaatgccgcgggcataatcccccaaaactcgtgcacg

SEQ ID NO: 933 - DNA, 70 bp, Homo sapiens
  source 1..70: mol_type = genomic DNA
cgcgggcataatcccccaaaactcgtgcacgctgatccagtacaaaaccaatttattttacattccatcg

SEQ ID NO: 934 - DNA, 74 bp, Homo sapiens
  source 1..74: mol_type = genomic DNA
cgggcataatcccccaaaactcgtgcacgctgatccagtacaaaaccaatttattttacattccatcgtgggcg

SEQ ID NO: 935 - DNA, 89 bp, Homo sapiens
  source 1..89: mol_type = genomic DNA
cgtgcacgctgatccagtacaaaaccaatttattttacattccatcgtgggcgatgggagaagctaaataataatttagcatgcatacg

SEQ ID NO: 936 - DNA, 123 bp, Homo sapiens
  source 1..123: mol_type = genomic DNA
cgtgggcgatgggagaagctaaataataatttagcatgcatacgtagacgtttctttttatatataaatacaatatacaaaaataaagacagtacagttttgagatttccagcagtttgaacg

SEQ ID NO: 937 - DNA, 121 bp, Homo sapiens
  source 1..121: mol_type = genomic DNA
cgatgggagaagctaaataataatttagcatgcatacgtagacgtttctttttatatataaatacaatatacaaaaataaagacagtacagttttgagatttccagcagtttgaacgctcg

SEQ ID NO: 938 - DNA, 107 bp, Homo sapiens
  source 1..107: mol_type = genomic DNA
cgtttctttttatatataaatacaatatacaaaaataaagacagtacagttttgagatttccagcagtttgaacgctcgtgacataatccccccgaccccagagacg

SEQ ID NO: 939 - DNA, 89 bp, Homo sapiens
  source 1..89: mol_type = genomic DNA
cgtgacataatccccccgaccccagagacgtaagcataaacccttttttctttttccaaatactctgcagaatggcggctccagaggcg

SEQ ID NO: 940 - DNA, 113 bp, Homo sapiens
  source 1..113: mol_type = genomic DNA
cgaccccagagacgtaagcataaacccttttttctttttccaaatactctgcagaatggcggctccagaggcggtttcaagtttcataagtcaggtaacactgtgggtttccg

SEQ ID NO: 941 - DNA, 109 bp, Homo sapiens
  source 1..109: mol_type = genomic DNA
cgtaagcataaacccttttttctttttccaaatactctgcagaatggcggctccagaggcggtttcaagtttcataagtcaggtaacactgtgggtttccgccttctcg

SEQ ID NO: 942 - DNA, 66 bp, Homo sapiens
  source 1..66: mol_type = genomic DNA
cggctccagaggcggtttcaagtttcataagtcaggtaacactgtgggtttccgccttctcggacg

SEQ ID NO: 943 - DNA, 34 bp, Homo sapiens
  source 1..34: mol_type = genomic DNA
cgcggggtgggtcggttcttctcacacgcactcg

SEQ ID NO: 944 - DNA, 34 bp, Homo sapiens
  source 1..34: mol_type = genomic DNA
cggggtgggtcggttcttctcacacgcactcgcg

SEQ ID NO: 945 - DNA, 99 bp, Homo sapiens
  source 1..99: mol_type = genomic DNA
cggttcttctcacacgcactcgcgcctcccccaacaccaaccaaaattaattttaaaagaaccagagttcaagtttcagccccctgggtctccctctcg

SEQ ID NO: 946 - DNA, 27 bp, Homo sapiens
  source 1..27: mol_type = genomic DNA
cgccggggcctccgggagaagccgccg

SEQ ID NO: 947 - DNA, 28 bp, Homo sapiens
  source 1..28: mol_type = genomic DNA
cggggcctccgggagaagccgccggccg

SEQ ID NO: 948 - DNA, 24 bp, Homo sapiens
  source 1..24: mol_type = genomic DNA
cgggagaagccgccggccggggcg

SEQ ID NO: 949 - DNA, 20 bp, Homo sapiens
  source 1..20: mol_type = genomic DNA
cggccggggcgctccgtgcg

SEQ ID NO: 950 - DNA, 46 bp, Homo sapiens
  source 1..46: mol_type = genomic DNA
cgtcctcgtcctcctcggggtcactgtccctcaagtcccgcaggcg

SEQ ID NO: 951 - DNA, 43 bp, Homo sapiens
  source 1..43: mol_type = genomic DNA
cgtcctcctcggggtcactgtccctcaagtcccgcaggcggcg

SEQ ID NO: 952 - DNA, 38 bp, Homo sapiens
  source 1..38: mol_type = genomic DNA
cggggtcactgtccctcaagtcccgcaggcggcgtccg

SEQ ID NO: 953 - DNA, 44 bp, Homo sapiens
  source 1..44: mol_type = genomic DNA
cgctgcccttgtctccgggcgctggcggccccagcagctcctcg

SEQ ID NO: 954 - DNA, 36 bp, Homo sapiens
  source 1..36: mol_type = genomic DNA
cgggcgctggcggccccagcagctcctcggctcccg

SEQ ID NO: 955 - DNA, 41 bp, Homo sapiens
  source 1..41: mol_type = genomic DNA
cgctggcggccccagcagctcctcggctcccggctcctccg

SEQ ID NO: 956 - DNA, 37 bp, Homo sapiens
  source 1..37: mol_type = genomic DNA
cggccccagcagctcctcggctcccggctcctccgcg

SEQ ID NO: 957 - DNA, 33 bp, Homo sapiens
  source 1..33: mol_type = genomic DNA
cgccgccgcccttctgtttctccgcttcctgcg

SEQ ID NO: 958 - DNA, 33 bp, Homo sapiens
  source 1..33: mol_type = genomic DNA
cgccgcccttctgtttctccgcttcctgcgccg

SEQ ID NO: 959 - DNA, 55 bp, Homo sapiens
  source 1..55: mol_type = genomic DNA
cgcccttctgtttctccgcttcctgcgccgcctgctctttggcctttttgctgcg

SEQ ID NO: 960 - DNA, 54 bp, Homo sapiens
  source 1..54: mol_type = genomic DNA
cgcttcctgcgccgcctgctctttggcctttttgctgcgtttccatttcatccg

SEQ ID NO: 961 - DNA, 48 bp, Homo sapiens
  source 1..48: mol_type = genomic DNA
cgccgcctgctctttggcctttttgctgcgtttccatttcatccgccg

SEQ ID NO: 962 - DNA, 45 bp, Homo sapiens
  source 1..45: mol_type = genomic DNA
cgccggttctggaaccaaatcttcacctgcgggcacaagcgggcg

SEQ ID NO: 963 - DNA, 52 bp, Homo sapiens
  source 1..52: mol_type = genomic DNA
cggttctggaaccaaatcttcacctgcgggcacaagcgggcgtgagaaaccg

SEQ ID NO: 964 - DNA, 101 bp, Homo sapiens
  source 1..101: mol_type = genomic DNA
cgtagcttcctcctccccgcaaccccccactcagcagcaaaccccagagctgggctggagctccgtgggagctaggactatgtccccaacgacccaggccg

SEQ ID NO: 965 - DNA, 100 bp, Homo sapiens
  source 1..100: mol_type = genomic DNA
cgcaaccccccactcagcagcaaaccccagagctgggctggagctccgtgggagctaggactatgtccccaacgacccaggccggggacttttctacccg

SEQ ID NO: 966 - DNA, 125 bp, Homo sapiens
  source 1..125: mol_type = genomic DNA
cgcggtaaatctcaggatgttccctcctctctttctggaacaaaatttctcgaatgcggcctggggatcaccttcttcagaatgaaaggaggggtggttaagtgctgattcttgggccccacccg

SEQ ID NO: 967 - DNA, 137 bp, Homo sapiens
  source 1..137: mol_type = genomic DNA
cggtaaatctcaggatgttccctcctctctttctggaacaaaatttctcgaatgcggcctggggatcaccttcttcagaatgaaaggaggggtggttaagtgctgattcttgggccccacccgaagctactgaatcg

SEQ ID NO: 968 - DNA, 92 bp, Homo sapiens
  source 1..92: mol_type = genomic DNA
cgaatgcggcctggggatcaccttcttcagaatgaaaggaggggtggttaagtgctgattcttgggccccacccgaagctactgaatcggcg

SEQ ID NO: 969 - DNA, 110 bp, Homo sapiens
  source 1..110: mol_type = genomic DNA
cggcctggggatcaccttcttcagaatgaaaggaggggtggttaagtgctgattcttgggccccacccgaagctactgaatcggcgctctgggcaccttagatgaacccg

SEQ ID NO: 970 - DNA, 70 bp, Homo sapiens
  source 1..70: mol_type = genomic DNA
cgcagcgctagatgcctcagaccgcccgtgggtcacaagtgcaaaggtaacagtgtcccctgggaggccg

SEQ ID NO: 971 - DNA, 72 bp, Homo sapiens
  source 1..72: mol_type = genomic DNA
cgctagatgcctcagaccgcccgtgggtcacaagtgcaaaggtaacagtgtcccctgggaggccgggatgcg

SEQ ID NO: 972 - DNA, 58 bp, Homo sapiens
  source 1..58: mol_type = genomic DNA
cgcccgtgggtcacaagtgcaaaggtaacagtgtcccctgggaggccgggatgcgtcg

SEQ ID NO: 973 - DNA, 60 bp, Homo sapiens
  source 1..60: mol_type = genomic DNA
cgtgggtcacaagtgcaaaggtaacagtgtcccctgggaggccgggatgcgtcgggggcg

SEQ ID NO: 974 - DNA, 27 bp, Homo sapiens
  source 1..27: mol_type = genomic DNA
cgggatgcgtcgggggcggggagggcg

SEQ ID NO: 975 - DNA, 65 bp, Homo sapiens
  source 1..65: mol_type = genomic DNA
cggctccggtcctcgccccagcccctcccgttgctgcttgtaccactacactcaaggccccagcg

SEQ ID NO: 976 - DNA, 84 bp, Homo sapiens
  source 1..84: mol_type = genomic DNA
cggtcctcgccccagcccctcccgttgctgcttgtaccactacactcaaggccccagcgccaaggcctggccctgcagagggcg

SEQ ID NO: 977 - DNA, 120 bp, Homo sapiens
  source 1..120: mol_type = genomic DNA
cgccccagcccctcccgttgctgcttgtaccactacactcaaggccccagcgccaaggcctggccctgcagagggcggggctgttccctcactatcagtgcccactccccaaggaatgcg

SEQ ID NO: 978 - DNA, 79 bp, Homo sapiens
  source 1..79: mol_type = genomic DNA
cgcacgttctcacaaaaccatttgaaaacatgagctggagacgccaaattctgggacccacgaaaggctttggagctcg

SEQ ID NO: 979 - DNA, 79 bp, Homo sapiens
  source 1..79: mol_type = genomic DNA
cgttctcacaaaaccatttgaaaacatgagctggagacgccaaattctgggacccacgaaaggctttggagctcgctcg

SEQ ID NO: 980 - DNA, 54 bp, Homo sapiens
  source 1..54: mol_type = genomic DNA
cgctttccagcctcgcaaggttcttctctcaccttctccggcgaccccctcccg

SEQ ID NO: 981 - DNA, 51 bp, Homo sapiens
  source 1..51: mol_type = genomic DNA
cgcaaggttcttctctcaccttctccggcgaccccctcccgaggctccccg

SEQ ID NO: 982 - DNA, 16 bp, Homo sapiens
  source 1..16: mol_type = genomic DNA
cgcgcgcgggtcagcg

SEQ ID NO: 983 - DNA, 35 bp, Homo sapiens
  source 1..35: mol_type = genomic DNA
cgggaaaggcgtgtagtacgcggccaggttctgcg

SEQ ID NO: 984 - DNA, 29 bp, Homo sapiens
  source 1..29: mol_type = genomic DNA
cgctgaggacaccgcgcgggagcttcccg

SEQ ID NO: 985 - DNA, 24 bp, Homo sapiens
  source 1..24: mol_type = genomic DNA
cgcgcgggagcttcccggggtccg

SEQ ID NO: 986 - DNA, 54 bp, Homo sapiens
  source 1..54: mol_type = genomic DNA
cgcagccgggcttctgggctcctcggtgcggggaacctgaccctgtagccttcg

SEQ ID NO: 987 - DNA, 63 bp, Homo sapiens
  source 1..63: mol_type = genomic DNA
cggcccagggcctgaccaccccggctccccatctggctggtgcatggcgcggggaagggggcg

SEQ ID NO: 988 - DNA, 44 bp, Homo sapiens
  source 1..44: mol_type = genomic DNA
cggctccccatctggctggtgcatggcgcggggaagggggcgcg

SEQ ID NO: 989 - DNA, 26 bp, Homo sapiens
  source 1..26: mol_type = genomic DNA
cgcggggaagggggcgcgccaggacg

SEQ ID NO: 990 - DNA, 38 bp, Homo sapiens
  source 1..38: mol_type = genomic DNA
cggggaagggggcgcgccaggacgggccccctccttcg

SEQ ID NO: 991 - DNA, 28 bp, Homo sapiens
  source 1..28: mol_type = genomic DNA
cgcgccaggacgggccccctccttcgcg

SEQ ID NO: 992 - DNA, 79 bp, Homo sapiens
  source 1..79: mol_type = genomic DNA
cggctacagatggcgcaggccccaggtcgcagagaagccctatccaactctgcggactggccctgggagaaagggcccg

SEQ ID NO: 993 - DNA, 85 bp, Homo sapiens
  source 1..85: mol_type = genomic DNA
cgcaggccccaggtcgcagagaagccctatccaactctgcggactggccctgggagaaagggcccgggagttgcttctccagtcg

SEQ ID NO: 994 - DNA, 78 bp, Homo sapiens
  source 1..78: mol_type = genomic DNA
cgcagagaagccctatccaactctgcggactggccctgggagaaagggcccgggagttgcttctccagtcggtgaacg

SEQ ID NO: 995 - DNA, 99 bp, Homo sapiens
  source 1..99: mol_type = genomic DNA
cgcactgggggactatactgcagtgtgaccgccagtgtccccagggaggctgcggaaaagtagaggcaatgagacccgagcagaaatagggacttctcg

SEQ ID NO: 996 - DNA, 72 bp, Homo sapiens
  source 1..72: mol_type = genomic DNA
cgccagtgtccccagggaggctgcggaaaagtagaggcaatgagacccgagcagaaatagggacttctcgcg

SEQ ID NO: 997 - DNA, 64 bp, Homo sapiens
  source 1..64: mol_type = genomic DNA
cggcagtccgcacggaagcagctggggagcatccagctcgaccctccccacaggcccagggtcg

SEQ ID NO: 998 - DNA, 64 bp, Homo sapiens
  source 1..64: mol_type = genomic DNA
cgcacggaagcagctggggagcatccagctcgaccctccccacaggcccagggtcgggacaccg

SEQ ID NO: 999 - DNA, 70 bp, Homo sapiens
  source 1..70: mol_type = genomic DNA
cggaagcagctggggagcatccagctcgaccctccccacaggcccagggtcgggacaccgagggaaggcg

SEQ ID NO: 1000 - DNA, 46 bp, Homo sapiens
  source 1..46: mol_type = genomic DNA
cgaccctccccacaggcccagggtcgggacaccgagggaaggcgcg

SEQ ID NO: 1001 - DNA, 41 bp, Homo sapiens
  source 1..41: mol_type = genomic DNA
cgcattttcccgccttccgcgagcaacccaggggccctgcg

SEQ ID NO: 1002 - DNA, 50 bp, Homo sapiens
  source 1..50: mol_type = genomic DNA
cgccttccgcgagcaacccaggggccctgcgggaggaggagagggtcccg

SEQ ID NO: 1003 - DNA, 51 bp, Homo sapiens
  source 1..51: mol_type = genomic DNA
cgcgagcaacccaggggccctgcgggaggaggagagggtcccgggagtccg

SEQ ID NO: 1004 - DNA, 61 bp, Homo sapiens
  source 1..61: mol_type = genomic DNA
cgagcaacccaggggccctgcgggaggaggagagggtcccgggagtccgcccttccctgcg

SEQ ID NO: 1005 - DNA, 47 bp, Homo sapiens
  source 1..47: mol_type = genomic DNA
cgggaggaggagagggtcccgggagtccgcccttccctgcgccttcg

SEQ ID NO: 1006 - DNA, 34 bp, Homo sapiens
  source 1..34: mol_type = genomic DNA
cgggagtccgcccttccctgcgccttcgggaccg

SEQ ID NO: 1007 - DNA, 82 bp, Homo sapiens
  source 1..82: mol_type = genomic DNA
cgaattaaaagaaaaggaaaagctcgtagtggaggtgttaccgcatcctgcctttggacgctactcttagttgagtgacccg

SEQ ID NO: 1008 - DNA, 60 bp, Homo sapiens
  source 1..60: mol_type = genomic DNA
cgctactcttagttgagtgacccgattcggaccttaggggcgttagggtctcctccaccg

SEQ ID NO: 1009 - DNA, 26 bp, Homo sapiens
  source 1..26: mol_type = genomic DNA
cgagccggccgaatccgtgctcaccg

SEQ ID NO: 1010 - DNA, 48 bp, Homo sapiens
  source 1..48: mol_type = genomic DNA
cgctgggggctctgctcgcgtgctgctctgaagttgttccccgatgcg

SEQ ID NO: 1011 - DNA, 35 bp, Homo sapiens
  source 1..35: mol_type = genomic DNA
cgcgtgctgctctgaagttgttccccgatgcgccg

SEQ ID NO: 1012 - DNA, 78 bp, Homo sapiens
  source 1..78: mol_type = genomic DNA
cgggaagggaacccttgggcagcggctgctgcagagctgctctgggagtgggccaggaggccccggcagggtgcggcg

SEQ ID NO: 1013 - DNA, 78 bp, Homo sapiens
  source 1..78: mol_type = genomic DNA
cggccatttgtccaaatgccgcctggggagcggcccggcctggggagcataagactgccaggaggcctgaaggtgccg

SEQ ID NO: 1014 - DNA, 102 bp, Homo sapiens
  source 1..102: mol_type = genomic DNA
cggggaaagtagaaaccaaaaagaaaacggcagctggtcctaagaagatggcgtttgctccaaccaaatcagccttgctgcgttagaaaaagaacatgcacg

SEQ ID NO: 1015 - DNA, 67 bp, Homo sapiens
  source 1..67: mol_type = genomic DNA
cgggttgcgttgtagggggaggcaatcagccctgttacccctcctcaggcggccggactgagctgcg

SEQ ID NO: 1016 - DNA, 36 bp, Homo sapiens
  source 1..36: mol_type = genomic DNA
cgcctgggtcggatcagacgcgggtagtggcttgcg

SEQ ID NO: 1017 - DNA, 63 bp, Homo sapiens
  source 1..63: mol_type = genomic DNA
cgcagggaaagcgcgtggagccaacctagagcgggagcagggtctggcttgactctgtccccg

SEQ ID NO: 1018 - DNA, 57 bp, Homo sapiens
  source 1..57: mol_type = genomic DNA
cgcgtggagccaacctagagcgggagcagggtctggcttgactctgtccccgattcg

SEQ ID NO: 1019 - DNA, 43 bp, Homo sapiens
  source 1..43: mol_type = genomic DNA
cgctccccgctgctctgagaacactgagtgtcccgggcccgcg

SEQ ID NO: 1020 - DNA, 75 bp, Homo sapiens
  source 1..75: mol_type = genomic DNA
cgctgctctgagaacactgagtgtcccgggcccgcgcagactgggttgggagagggctctgtgggagggcttgcg

SEQ ID NO: 1021 - DNA, 56 bp, Homo sapiens
  source 1..56: mol_type = genomic DNA
cgggcccgcgcagactgggttgggagagggctctgtgggagggcttgcggggagcg

SEQ ID NO: 1022 - DNA, 82 bp, Homo sapiens
  source 1..82: mol_type = genomic DNA
cgcgcagactgggttgggagagggctctgtgggagggcttgcggggagcggggtaagaaaggctaggtgcttcttacctacg

SEQ ID NO: 1023 - DNA, 79 bp, Homo sapiens
  source 1..79: mol_type = genomic DNA
cgctgttcatagtgtttaagtggaccaacttaaagggagagagcgcttgccgagctctgggaaaccgaaagggtccacg

SEQ ID NO: 1024 - DNA, 40 bp, Homo sapiens
  source 1..40: mol_type = genomic DNA
cgcttgccgagctctgggaaaccgaaagggtccacgcccg

SEQ ID NO: 1025 - DNA, 35 bp, Homo sapiens
  source 1..35: mol_type = genomic DNA
cgagctctgggaaaccgaaagggtccacgcccgcg

SEQ ID NO: 1026 - DNA, 29 bp, Homo sapiens
  source 1..29: mol_type = genomic DNA
cgaaagggtccacgcccgcgggagcctcg

SEQ ID NO: 1027 - DNA, 35 bp, Homo sapiens
  source 1..35: mol_type = genomic DNA
cgcccgcgggagcctcggagactcaaaatcccccg

SEQ ID NO: 1028 - DNA, 64 bp, Homo sapiens
  source 1..64: mol_type = genomic DNA
cgggagcctcggagactcaaaatcccccgctagaccgcagaaaatgttacactttaatcaaccg

SEQ ID NO: 1029 - DNA, 70 bp, Homo sapiens
  source 1..70: mol_type = genomic DNA
cgcagaaaatgttacactttaatcaaccgctacttcttccgtggagaggggcggtgggtgggggtgtgcg

SEQ ID NO: 1030 - DNA, 53 bp, Homo sapiens
  source 1..53: mol_type = genomic DNA
cgctacttcttccgtggagaggggcggtgggtgggggtgtgcgcatctcatcg

SEQ ID NO: 1031 - DNA, 43 bp, Homo sapiens
  source 1..43: mol_type = genomic DNA
cgtggagaggggcggtgggtgggggtgtgcgcatctcatcgcg

SEQ ID NO: 1032 - DNA, 67 bp, Homo sapiens
  source 1..67: mol_type = genomic DNA
cggtgggtgggggtgtgcgcatctcatcgcggcctgtgagtggaacaggctagaattatccccagcg

SEQ ID NO: 1033 - DNA, 55 bp, Homo sapiens
  source 1..55: mol_type = genomic DNA
cggggagggacctggttgtgtgcttcaggtgccccaagggacacggagacgcgcg

SEQ ID NO: 1034 - DNA, 49 bp, Homo sapiens
  source 1..49: mol_type = genomic DNA
cgcgagggttgggatcaaggcgttgaagacagagcgacagcccttctcg

SEQ ID NO: 1035 - DNA, 49 bp, Homo sapiens
  source 1..49: mol_type = genomic DNA
cgggcggcccccttagcccgcgtgactgtgggatcctgagaacattccg

SEQ ID NO: 1036 - DNA, 40 bp, Homo sapiens
  source 1..40: mol_type = genomic DNA
cggccagcgctttggggaaagtgccccgaccgcagaggcg

SEQ ID NO: 1037 - DNA, 42 bp, Homo sapiens
  source 1..42: mol_type = genomic DNA
cgaccgcagaggcgacgacaggggagcaggaagctgctcacg

SEQ ID NO: 1038 - DNA, 45 bp, Homo sapiens
  source 1..45: mol_type = genomic DNA
cgcagaggcgacgacaggggagcaggaagctgctcacggtagtcg

SEQ ID NO: 1039 - DNA, 40 bp, Homo sapiens
  source 1..40: mol_type = genomic DNA
cgacgacaggggagcaggaagctgctcacggtagtcggcg

SEQ ID NO: 1040 - DNA, 87 bp, Homo sapiens
  source 1..87: mol_type = genomic DNA
cgttggcggcagcggtggccttcctcatctgggcgatgtgggctcctagaagagtaaggataacatcctggaaatgacttctgtacg

SEQ ID NO: 1041 - DNA, 36 bp, Homo sapiens
  source 1..36: mol_type = genomic DNA
cgtttcttccgaactggaagcgaagtcccatccgcg

SEQ ID NO: 1042 - DNA, 31 bp, Homo sapiens
  source 1..31: mol_type = genomic DNA
cgtgcaggaccgcatggagctcgccgcctcg

SEQ ID NO: 1043 - DNA, 28 bp, Homo sapiens
  source 1..28: mol_type = genomic DNA
cgccgcctcgctcaacctcaccgacacg

SEQ ID NO: 1044 - DNA, 117 bp, Homo sapiens
  source 1..117: mol_type = genomic DNA
cgcccccaggactcggtgtcccgaggttgaggctactggtcaccctggcttcagaaagcgaagaattcaggaacctctgatcaccccaccctgactcctgatcattcagttggggcg

SEQ ID NO: 1045 - DNA, 113 bp, Homo sapiens
  source 1..113: mol_type = genomic DNA
cggttactgggggcttctcagagcggcaggaaggtagagtaagaaccggacaccggggctctgacattacacagaaaaggtcagaattcctcccttcaagtcaccctaggccg

SEQ ID NO: 1046 - DNA, 97 bp, Homo sapiens
  source 1..97: mol_type = genomic DNA
cggcaggaaggtagagtaagaaccggacaccggggctctgacattacacagaaaaggtcagaattcctcccttcaagtcaccctaggccggttcccg

SEQ ID NO: 1047 - DNA, 27 bp, Homo sapiens
  source 1..27: mol_type = genomic DNA
cggtgcggaaggctaagcgagcccgcg

SEQ ID NO: 1048 - DNA, 30 bp, Homo sapiens
  source 1..30: mol_type = genomic DNA
cgcgctatgtgcccctgtgtgcccgcgccg

SEQ ID NO: 1049 - DNA, 25 bp, Homo sapiens
  source 1..25: mol_type = genomic DNA
cgccccgctcccgccccgcctgccg

SEQ ID NO: 1050 - DNA, 22 bp, Homo sapiens
  source 1..22: mol_type = genomic DNA
cgctcccgccccgcctgccgcg

SEQ ID NO: 1051 - DNA, 23 bp, Homo sapiens
  source 1..23: mol_type = genomic DNA
cgcctgccgcgcctcccggggcg

SEQ ID NO: 1052 - DNA, 58 bp, Homo sapiens
  source 1..58: mol_type = genomic DNA
cgcgtcccccacccgaacctggacctagcctcctctgaacgcagagggcagtgggccg

SEQ ID NO: 1053 - DNA, 60 bp, Homo sapiens
  source 1..60: mol_type = genomic DNA
cgtcccccacccgaacctggacctagcctcctctgaacgcagagggcagtgggccgcccg

SEQ ID NO: 1054 - DNA, 58 bp, Homo sapiens
  source 1..58: mol_type = genomic DNA
cgaacctggacctagcctcctctgaacgcagagggcagtgggccgcccggaaggggcg

SEQ ID NO: 1055 - DNA, 45 bp, Homo sapiens
  source 1..45: mol_type = genomic DNA
cgcagagggcagtgggccgcccggaaggggcggggaggggccccg

SEQ ID NO: 1056 - DNA, 72 bp, Homo sapiens
  source 1..72: mol_type = genomic DNA
cgtggacagtagcggtcaggaaggagattcccgtttccccttctcccaggaggaaaagaggtaacgctgccg

SEQ ID NO: 1057 - DNA, 90 bp, Homo sapiens
  source 1..90: mol_type = genomic DNA
cggtcaggaaggagattcccgtttccccttctcccaggaggaaaagaggtaacgctgccgccactgccctctctttccagggtgggaacg

SEQ ID NO: 1058 - DNA, 122 bp, Homo sapiens
  source 1..122: mol_type = genomic DNA
cggtggagaggcgaagtgatccagggtcccggaaagttggacaattccctggaccagatggctctcagtttcctcctctgtaaaatggggccggtaataagcaactctctggattcaccccg

SEQ ID NO: 1059 - DNA, 121 bp, Homo sapiens
  source 1..121: mol_type = genomic DNA
cgaagtgatccagggtcccggaaagttggacaattccctggaccagatggctctcagtttcctcctctgtaaaatggggccggtaataagcaactctctggattcaccccggtttgctgcg

SEQ ID NO: 1060 - DNA, 108 bp, Homo sapiens
  source 1..108: mol_type = genomic DNA
cgcagtgtgtacatccaaatctacacctcggaaagagcagggactgggcccccagccttgcaagatccctgtgaagactggggccgacgatccaaaaactaagagccg

SEQ ID NO: 1061 - DNA, 99 bp, Homo sapiens
  source 1..99: mol_type = genomic DNA
cggaaagagcagggactgggcccccagccttgcaagatccctgtgaagactggggccgacgatccaaaaactaagagccggggcaaggtgacccctccg

SEQ ID NO: 1062 - DNA, 90 bp, Homo sapiens
  source 1..90: mol_type = genomic DNA
cgacgatccaaaaactaagagccggggcaaggtgacccctccgcaagggtgcccaagctggggtacacactgtgttccctctgctcagcg

SEQ ID NO: 1063 - DNA, 71 bp, Homo sapiens
  source 1..71: mol_type = genomic DNA
cgcgccactctggtgggataggtcttccgagtaccacaggaagtctcctctcccagtagccccgagggtcg

SEQ ID NO: 1064 - DNA, 80 bp, Homo sapiens
  source 1..80: mol_type = genomic DNA
cgccactctggtgggataggtcttccgagtaccacaggaagtctcctctcccagtagccccgagggtcggagatcccacg

SEQ ID NO: 1065 - DNA, 36 bp, Homo sapiens
  source 1..36: mol_type = genomic DNA
cgagggtcggagatcccacgggacctcggacaagcg

SEQ ID NO: 1066 - DNA, 40 bp, Homo sapiens
  source 1..40: mol_type = genomic DNA
cggagatcccacgggacctcggacaagcgtctccttagcg

SEQ ID NO: 1067 - DNA, 86 bp, Homo sapiens
  source 1..86: mol_type = genomic DNA
cgaaaacgaaataaactatacaaatctgcctagcttttgagggggcggtggcccaggtatccttctcgcttgaggccaggtcaccg

SEQ ID NO: 1068 - DNA, 64 bp, Homo sapiens
  source 1..64: mol_type = genomic DNA
cggatccactcgacttacccaggccgctgccaatcccagctccttccccagcgcctcatttccg

SEQ ID NO: 1069 - DNA, 75 bp, Homo sapiens
  source 1..75: mol_type = genomic DNA
cgacttacccaggccgctgccaatcccagctccttccccagcgcctcatttccgattttttcatatgctaagtcg

SEQ ID NO: 1070 - DNA, 74 bp, Homo sapiens
  source 1..74: mol_type = genomic DNA
cggacccagaccctcaagctgccagcggttgtttaaagtcctggcagatgtcaggaagatgggcccggccgtcg

SEQ ID NO: 1071 - DNA, 42 bp, Homo sapiens; identical to SEQ ID NO: 58
  source 1..42: mol_type = genomic DNA

SEQ ID NO: 1072 - DNA, 38 bp, Homo sapiens
  source 1..38: mol_type = genomic DNA
cgcctctagcccggcgatgagtggttctgcggggaccg

SEQ ID NO: 1073 - DNA, 30 bp, Homo sapiens
  source 1..30: mol_type = genomic DNA
cggcgatgagtggttctgcggggaccgacg

SEQ ID NO: 1074 - DNA, 49 bp, Homo sapiens
  source 1..49: mol_type = genomic DNA
cgatgagtggttctgcggggaccgacggccccatttccttcttgcctcg

SEQ ID NO: 1075 - DNA, 56 bp, Homo sapiens
  source 1..56: mol_type = genomic DNA
cggcaactgcaggcccgaagctcccctcgcccagcggcccaaccacagtgctgtcg

SEQ ID NO: 1076 - DNA, 46 bp, Homo sapiens
  source 1..46: mol_type = genomic DNA
cgaagctcccctcgcccagcggcccaaccacagtgctgtcgtgtcg

SEQ ID NO: 1077 - DNA, 41 bp, Homo sapiens
  source 1..41: mol_type = genomic DNA
cgcccagcggcccaaccacagtgctgtcgtgtcggggatcg

SEQ ID NO: 1078 - DNA, 93 bp, Homo sapiens
  source 1..93: mol_type = genomic DNA
cgggactccagctgcccctgcctggagggagggaagggagaagagaatacgaattaattacgaaggaaacccaggtgtgaaaggcacccgccg

SEQ ID NO: 1079 - DNA, 43 bp, Homo sapiens
  source 1..43: mol_type = genomic DNA
cgaacccgggcctaggtcttcatgtcccggcggccagaggacg

SEQ ID NO: 1080 - DNA, 91 bp, Homo sapiens; identical to SEQ ID NO: 79
  source 1..91: mol_type = genomic DNA

SEQ ID NO: 1081 - DNA, 76 bp, Homo sapiens; identical to SEQ ID NO: 83
  source 1..76: mol_type = genomic DNA

SEQ ID NO: 1082 - DNA, 57 bp, Homo sapiens
  source 1..57: mol_type = genomic DNA
cgctcgctttctttggttttctttctcggtatttcgttgtcaaggccacccttgccg

SEQ ID NO: 1083 - DNA, 56 bp, Homo sapiens
  source 1..56: mol_type = genomic DNA
cgctttctttggttttctttctcggtatttcgttgtcaaggccacccttgccgtcg

SEQ ID NO: 1084 - DNA, 41 bp, Homo sapiens
  source 1..41: mol_type = genomic DNA
cggtatttcgttgtcaaggccacccttgccgtcggatcccg

SEQ ID NO: 1085 - DNA, 52 bp, Homo sapiens
  source 1..52: mol_type = genomic DNA
cgttgtcaaggccacccttgccgtcggatcccggggtgctgggtttctcccg

SEQ ID NO: 1086 - DNA, 35 bp, Homo sapiens; identical to SEQ ID NO: 84
  source 1..35: mol_type = genomic DNA

SEQ ID NO: 1087 - DNA, 64 bp, Homo sapiens; identical to SEQ ID NO: 99
  source 1..64: mol_type = genomic DNA

SEQ ID NO: 1088 - DNA, 67 bp, Homo sapiens
  source 1..67: mol_type = genomic DNA
cgagaggggcaaaaagttgagaaataggcgagcgggagagataagcaggaaggcccgggtgggcccg

SEQ ID NO: 1089 - DNA, 90 bp, Homo sapiens
  source 1..90: mol_type = genomic DNA
cgactggggtgctgggggggtgaaatgtcgcccgaaccagtgaggtctctctcctcctcttgtatcctctggtgctggtcagtcctcgcg

SEQ ID NO: 1090 - DNA, 126 bp, Homo sapiens
  source 1..126: mol_type = genomic DNA
cgatctcattgattccttcccacaaccccactttgcagataggaaagctgaggcacgagagggtaagtaacccaggccgcacaacgaggggaagctagggagggactggggccctgggcccctgcg

SEQ ID NO: 1091 - DNA, 92 bp, Homo sapiens
  source 1..92: mol_type = genomic DNA
cggaaacgatcaccaaactgtagtcaatattttcctagtcctttttaagcgaggctccggatggctgggatgtctttaggttagtagtcccg

SEQ ID NO: 1092 - DNA, 94 bp, Homo sapiens
  source 1..94: mol_type = genomic DNA
cgatcaccaaactgtagtcaatattttcctagtcctttttaagcgaggctccggatggctgggatgtctttaggttagtagtcccgggaggacg

SEQ ID NO: 1093 - DNA, 26 bp, Homo sapiens
  source 1..26: mol_type = genomic DNA
cgtagctggagggcgtgcgccgtgcg

SEQ ID NO: 1094 - DNA, 30 bp, Homo sapiens
  source 1..30: mol_type = genomic DNA
cgaaaagcagagatccgagaaccgcgtgcg

SEQ ID NO: 1095 - DNA, 26 bp, Homo sapiens
  source 1..26: mol_type = genomic DNA
cgcgcattttccgggagttgagcgcg

SEQ ID NO: 1096 - DNA, 43 bp, Homo sapiens
  source 1..43: mol_type = genomic DNA
cgcaggccgggaacctaccgctctcaggctcccagcccgggcg

SEQ ID NO: 1097 - DNA, 26 bp, Homo sapiens
  source 1..26: mol_type = genomic DNA
cggacgaaattcgaggccggagggcg

SEQ ID NO: 1098 - DNA, 137 bp, Homo sapiens
  source 1..137: mol_type = genomic DNA
cggagggcgttttcttttttgcaaaattgccccaaagccagggccgcatgtacctactgtctcctttgccccacatgctccaagaaaataagacacattctaccccgagtcctaattattgggccatttccttaacg

SEQ ID NO: 1099 - DNA, 132 bp, Homo sapiens
  source 1..132: mol_type = genomic DNA
cgttttcttttttgcaaaattgccccaaagccagggccgcatgtacctactgtctcctttgccccacatgctccaagaaaataagacacattctaccccgagtcctaattattgggccatttccttaacgcg

SEQ ID NO: 1100 - DNA, 108 bp, Homo sapiens
  source 1..108: mol_type = genomic DNA
cgactggtttcctgctctcatcctggcgcctgggcccagttgccatagtgtggatcccatgactcctcagggaacccctggactcaggcacgcgagaagaagacagcg

SEQ ID NO: 1101 - DNA, 121 bp, Homo sapiens
  source 1..121: mol_type = genomic DNA
cgataggtttatcttgaatagggggataccttgaacccaataccccaaggttagaggtttagaagacgaaggtcaagtgatttgaagaatactgctattccagtcgcgtatctgggcagcg

SEQ ID NO: 1102 - DNA, 30 bp, Homo sapiens
  source 1..30: mol_type = genomic DNA
cgagcgcaggcgggcggagagcagaccacg

SEQ ID NO: 1103 - DNA, 28 bp, Homo sapiens
  source 1..28: mol_type = genomic DNA
cgcaggcgggcggagagcagaccacgcg

SEQ ID NO: 1104 - DNA, 32 bp, Homo sapiens
  source 1..32: mol_type = genomic DNA
cgggcggagagcagaccacgcggaagaggccg

SEQ ID NO: 1105 - DNA, 40 bp, Homo sapiens
  source 1..40: mol_type = genomic DNA
cggagagcagaccacgcggaagaggccgagagagaaagcg

SEQ ID NO: 1106 - DNA, 35 bp, Homo sapiens
  source 1..35: mol_type = genomic DNA
cgcggaagaggccgagagagaaagcggggcacccg

SEQ ID NO: 1107 - DNA, 37 bp, Homo sapiens
  source 1..37: mol_type = genomic DNA
cggaagaggccgagagagaaagcggggcacccggccg

SEQ ID NO: 1108 - DNA, 61 bp, Homo sapiens
  source 1..61: mol_type = genomic DNA
cgctgggcacgaaggaggctggggcgaagatgagtccaggggttgggttgagagcgctgcg

SEQ ID NO: 1109 - DNA, 63 bp, Homo sapiens
  source 1..63: mol_type = genomic DNA
cgacaggctgttctttctcccttcaaccttgggcacccactcctgtcgccgttgcgggacccg

SEQ ID NO: 1110 - DNA, 72 bp, Homo sapiens
  source 1..72: mol_type = genomic DNA
cgctctgcgtttacttctgggcccgaagaatgccccgcagaggccctaggagctggttcatgggacttcacg

SEQ ID NO: 1111 - DNA, 67 bp, Homo sapiens
  source 1..67: mol_type = genomic DNA
cgtttacttctgggcccgaagaatgccccgcagaggccctaggagctggttcatgggacttcacgcg

SEQ ID NO: 1112 - DNA, 67 bp, Homo sapiens
  source 1..67: mol_type = genomic DNA
cgaagaatgccccgcagaggccctaggagctggttcatgggacttcacgcgcctccagtgtcttgcg

SEQ ID NO: 1113 - DNA, 79 bp, Homo sapiens
  source 1..79: mol_type = genomic DNA
cgcagaggccctaggagctggttcatgggacttcacgcgcctccagtgtcttgcggagccccagccccagcaagcagcg

SEQ ID NO: 1114 - DNA, 91 bp, Homo sapiens
  source 1..91: mol_type = genomic DNA
cgcaaactccgtcccaagtggggcggaggctggaggggaggggggctgcgagggtcacagtcaaggccaactactcacaacagaaagctcg

SEQ ID NO: 1115 - DNA, 75 bp, Homo sapiens
  source 1..75: mol_type = genomic DNA
cgaggagccgcccgcccggctaaatggacacaggaaaggcctgagtcaggcttctcataataaataaactgcacg

SEQ ID NO: 1116 - DNA, 85 bp, Homo sapiens
  source 1..85: mol_type = genomic DNA
cgcccgcccggctaaatggacacaggaaaggcctgagtcaggcttctcataataaataaactgcacgtgaataattcagcagtcg

SEQ ID NO: 1117 - DNA, 83 bp, Homo sapiens
  source 1..83: mol_type = genomic DNA
cgcccggctaaatggacacaggaaaggcctgagtcaggcttctcataataaataaactgcacgtgaataattcagcagtcgcg

SEQ ID NO: 1118 - DNA, 81 bp, Homo sapiens
  source 1..81: mol_type = genomic DNA
cggctaaatggacacaggaaaggcctgagtcaggcttctcataataaataaactgcacgtgaataattcagcagtcgcgcg

SEQ ID NO: 1119 - DNA, 71 bp, Homo sapiens
  source 1..71: mol_type = genomic DNA
cgcccaattccccgcggaggggagtagccaattaaggcacttgaaaagggagtcgggtggaagatcccccg

SEQ ID NO: 1120 - DNA, 71 bp, Homo sapiens
  source 1..71: mol_type = genomic DNA
cgagacggagccagaataagggatgcggacgtctgacctgtggaaaacccagcatctgcagtgcccagccg

SEQ ID NO: 1121 - DNA, 62 bp, Homo sapiens
  source 1..62: mol_type = genomic DNA
cgtctgacctgtggaaaacccagcatctgcagtgcccagccgcacagggaacgtgggcggcg

SEQ ID NO: 1122 - DNA, 103 bp, Homo sapiens
  source 1..103: mol_type = genomic DNA
cgtttgagcgaaacccggcccctttggctcttcccacttcccacaagtccctgggttcccgatcctgagggattgcattcatcccatctcacacccaccaacg

SEQ ID NO: 1123 - DNA, 129 bp, Homo sapiens
  source 1..129: mol_type = genomic DNA
cggcatgcaccagacctcaaaagggatcttcacgggaattccgcatagccagatcccaagcctccagggaggccaccagccctcccccatcttcttcttcctcctccgtctcctttcctcttccctccg

SEQ ID NO: 1124 - DNA, 91 bp, Homo sapiens
  source 1..91: mol_type = genomic DNA
cgtatttctttctattcgtttcttttcgtttggtgaggtgccacccgccacagccctgtcccctagggagaggcctggactaactttgccg

SEQ ID NO: 1125 - DNA, 81 bp, Homo sapiens
  source 1..81: mol_type = genomic DNA
cgtttcttttcgtttggtgaggtgccacccgccacagccctgtcccctagggagaggcctggactaactttgccgtcctcg

SEQ ID NO: 1126 - DNA, 84 bp, Homo sapiens
  source 1..84: mol_type = genomic DNA
cgtttggtgaggtgccacccgccacagccctgtcccctagggagaggcctggactaactttgccgtcctcgtggccttagcccg

SEQ ID NO: 1127 - DNA, 79 bp, Homo sapiens
  source 1..79: mol_type = genomic DNA
cgccacagccctgtcccctagggagaggcctggactaactttgccgtcctcgtggccttagcccggactgtgagtctcg

SEQ ID NO: 1128 - DNA, 123 bp, Homo sapiens
  source 1..123: mol_type = genomic DNA
cgggtgaatccagccctcgcagggcctggagtgacctaagccgagaggagctgggctgaatctccaggcgttcctttctttctgccctaggaaagctttgaaattcttattttcaggtaaacg

SEQ ID NO: 1129 - DNA, 111 bp, Homo sapiens
  source 1..111: mol_type = genomic DNA
cgggtcctcctcctgcagggcagcagagtggaagccagggcctgggaccctctggctgggcaaggagggactggagtccggctcccggtgaccccgtccccagtgccagcg

SEQ ID NO: 1130 - DNA, 42 bp, Homo sapiens
  source 1..42: mol_type = genomic DNA
cggctcccggtgaccccgtccccagtgccagcggtggggtcg

SEQ ID NO: 1131 - DNA, 44 bp, Homo sapiens
  source 1..44: mol_type = genomic DNA
cggtgaccccgtccccagtgccagcggtggggtcgcctgtggcg

SEQ ID NO: 1132 - DNA, 40 bp, Homo sapiens
  source 1..40: mol_type = genomic DNA
cgtccccagtgccagcggtggggtcgcctgtggcggcccg

SEQ ID NO: 1133 - DNA, 36 bp, Homo sapiens
  source 1..36: mol_type = genomic DNA
cggtggggtcgcctgtggcggcccggacaggccacg

SEQ ID NO: 1134 - DNA, 83 bp, Homo sapiens
  source 1..83: mol_type = genomic DNA
cggctgagagccgataaagccgagggggagaagggccgacatcagggtaatgggggagcagggccattgaggagctggagccg

SEQ ID NO: 1135 - DNA, 29 bp, Homo sapiens
  source 1..29: mol_type = genomic DNA
cggcgagagggccgcggaaggggggtgcg

SEQ ID NO: 1136 - DNA, 31 bp, Homo sapiens
  source 1..31: mol_type = genomic DNA
cgaaccggggccggaccccggggaggagccg

SEQ ID NO: 1137 - DNA, 52 bp, Homo sapiens
  source 1..52: mol_type = genomic DNA
cgggtcgaagtaccgcgagttggccctgactgtctgcaggatgagggtgtcg

SEQ ID NO: 1138 - DNA, 67 bp, Homo sapiens
  source 1..67: mol_type = genomic DNA
cgaagtaccgcgagttggccctgactgtctgcaggatgagggtgtcgaggagggttccaggccagcg

SEQ ID NO: 1139 - DNA, 70 bp, Homo sapiens
  source 1..70: mol_type = genomic DNA
cgcgagttggccctgactgtctgcaggatgagggtgtcgaggagggttccaggccagcgtgcctgcctcg

SEQ ID NO: 1140 - DNA, 79 bp, Homo sapiens
  source 1..79: mol_type = genomic DNA
cgagttggccctgactgtctgcaggatgagggtgtcgaggagggttccaggccagcgtgcctgcctcgcctccagcccg

SEQ ID NO: 1141 - DNA, 56 bp, Homo sapiens
  source 1..56: mol_type = genomic DNA
cgtgtccggaagtgtcctccccggccctcctcgcttcccaagatttgtttgcaacg

SEQ ID NO: 1142 - DNA, 60 bp, Homo sapiens
  source 1..60: mol_type = genomic DNA
cggaagtgtcctccccggccctcctcgcttcccaagatttgtttgcaacgtctacacccg

SEQ ID NO: 1143 - DNA, 54 bp, Homo sapiens
  source 1..54: mol_type = genomic DNA
cggccctcctcgcttcccaagatttgtttgcaacgtctacacccgtcaccaccg

SEQ ID NO: 1144 - DNA, 34 bp, Homo sapiens
  source 1..34: mol_type = genomic DNA
cgcccatcgccgcctctacagggagggagccgcg

SEQ ID NO: 1145 - DNA, 33 bp, Homo sapiens
  source 1..33: mol_type = genomic DNA
cgccgcctctacagggagggagccgcggccccg

SEQ ID NO: 1146 - DNA, 23 bp, Homo sapiens
  source 1..23: mol_type = genomic DNA
cgcggccccgagctggccacgcg

SEQ ID NO: 1147 - DNA, 27 bp, Homo sapiens
  source 1..27: mol_type = genomic DNA
cggccccgagctggccacgcgatggcg

SEQ ID NO: 1148 - DNA, 32 bp, Homo sapiens
  source 1..32: mol_type = genomic DNA
cgagctggccacgcgatggcgctgtgcacccg

SEQ ID NO: 1149 - DNA, 25 bp, Homo sapiens
  source 1..25: mol_type = genomic DNA
cgcgatggcgctgtgcacccggccg

SEQ ID NO: 1150 - DNA, 25 bp, Homo sapiens
  source 1..25: mol_type = genomic DNA
cgatggcgctgtgcacccggccgcg

SEQ ID NO: 1151 - DNA, 32 bp, Homo sapiens
  source 1..32: mol_type = genomic DNA
cgctgtgcacccggccgcggagcccctgcacg

SEQ ID NO: 1152 - DNA, 25 bp, Homo sapiens
  source 1..25: mol_type = genomic DNA
cggccgcggagcccctgcacgtccg

SEQ ID NO: 1153 - DNA, 42 bp, Homo sapiens
  source 1..42: mol_type = genomic DNA
cgagctccggactccaggctcggagcaggggttcgggctacg

SEQ ID NO: 1154 - DNA, 42 bp, Homo sapiens
  source 1..42: mol_type = genomic DNA
cggactccaggctcggagcaggggttcgggctacggttcccg

SEQ ID NO: 1155 - DNA, 35 bp, Homo sapiens
  source 1..35: mol_type = genomic DNA
cggagcaggggttcgggctacggttcccggctccg

SEQ ID NO: 1156 - DNA, 25 bp, Homo sapiens
  source 1..25: mol_type = genomic DNA
cggttcccggctccgagggaccgcg

SEQ ID NO: 1157 - DNA, 47 bp, Homo sapiens
  source 1..47: mol_type = genomic DNA
cggctccgagggaccgcggggctctctgcctgggaaggaagcttgcg

SEQ ID NO: 1158 - DNA, 51 bp, Homo sapiens
  source 1..51: mol_type = genomic DNA
cgagggaccgcggggctctctgcctgggaaggaagcttgcgggcctcctcg

SEQ ID NO: 1159 - DNA, 50 bp, Homo sapiens
  source 1..50: mol_type = genomic DNA
cgcggggctctctgcctgggaaggaagcttgcgggcctcctcgggcctcg

SEQ ID NO: 1160 - DNA, 82 bp, Homo sapiens
  source 1..82: mol_type = genomic DNA
cggggctctctgcctgggaaggaagcttgcgggcctcctcgggcctcgctgcagcccagctaggttaatcaatcagcagccg

SEQ ID NO: 1161 - DNA, 125 bp, Homo sapiens
  source 1..125: mol_type = genomic DNA
cgcaagttccatctacccctccagctacagagccccacctctaggcacaggaagcttcccggaaaaagaaaggctgtcccagaaagagaccgagagagactttccaaacttcgggcatagccacg

SEQ ID NO: 1162 - DNA, 91 bp, Homo sapiens
  source 1..91: mol_type = genomic DNA
cgtgctctctcccgcgccctgtaacagcgagtttaccacccctaccctgctgcaggcctgggggctcctctccccatcccttctaggcccg

SEQ ID NO: 1163 - DNA, 82 bp, Homo sapiens
  source 1..82: mol_type = genomic DNA
cgccgggttggtggagactttcagtcttggctaagcccaaaggggcctccttctcgctcaactaggagaagacttcgccccg

SEQ ID NO: 1164 - DNA, 68 bp, Homo sapiens
  source 1..68: mol_type = genomic DNA
cgctcaactaggagaagacttcgccccgccctgatcagcccagcccctctctccggggtcagagcccg

SEQ ID NO: 1165 - DNA, 76 bp, Homo sapiens
  source 1..76: mol_type = genomic DNA
cgcgcaccccgagcccgaagtcccagaaagaccagaggcacttactgttctgcatgctggctctggctgggggccg

SEQ ID NO: 1166 - DNA, 76 bp, Homo sapiens
  source 1..76: mol_type = genomic DNA
cgcaccccgagcccgaagtcccagaaagaccagaggcacttactgttctgcatgctggctctggctgggggccgcg

SEQ ID NO: 1167 - DNA, 79 bp, Homo sapiens
  source 1..79: mol_type = genomic DNA
cgagcccgaagtcccagaaagaccagaggcacttactgttctgcatgctggctctggctgggggccgcgggattccacg

SEQ ID NO: 1168 - DNA, 61 bp, Homo sapiens
  source 1..61: mol_type = genomic DNA
cgctctccatttgtcccgggcaaggaggctgggagggagggagccggctctggggtcgccg

SEQ ID NO: 1169 - DNA, 59 bp, Homo sapiens
  source 1..59: mol_type = genomic DNA
cgggcaaggaggctgggagggagggagccggctctggggtcgccgagggagaaggtgcg

SEQ ID NO: 1170 - DNA, 107 bp, Homo sapiens
  source 1..107: mol_type = genomic DNA
cgtagagccccaaatgccgagtgctacctgaggcaggctgtagctggggtgaaaaggaccggaagcgggaaagaaacctcttttctttcaggcctcccttcagcccg

SEQ ID NO: 1171 - DNA, 115 bp, Homo sapiens
  source 1..115: mol_type = genomic DNA
cgagtgctacctgaggcaggctgtagctggggtgaaaaggaccggaagcgggaaagaaacctcttttctttcaggcctcccttcagcccggggtagcacagactgcaaatttccg

SEQ ID NO: 1172 - DNA, 101 bp, Homo sapiens
  source 1..101: mol_type = genomic DNA
cgaaggtatgttttcccctcccggcctctgtcgcttttcctctctccccttaactactttatgcaaataaagccgcttccagactggaggagcccctaccg

SEQ ID NO: 1173 - DNA, 36 bp, Homo sapiens
  source 1..36: mol_type = genomic DNA
cggcgcgggcatcctggctccgggctgggccccccg

SEQ ID NO: 1174 - DNA, 35 bp, Homo sapiens
  source 1..35: mol_type = genomic DNA
cgcgggcatcctggctccgggctgggccccccgcg

SEQ ID NO: 1175 - DNA, 19 bp, Homo sapiens
  source 1..19: mol_type = genomic DNA
cggagccgcgcgccctgcg

SEQ ID NO: 1176 - DNA, 43 bp, Homo sapiens
  source 1..43: mol_type = genomic DNA
cgtcccgcaggtttacttcgggggccgggagccccaagatgcg

SEQ ID NO: 1177 - DNA, 47 bp, Homo sapiens
  source 1..47: mol_type = genomic DNA
cgcaggtttacttcgggggccgggagccccaagatgcggaaagtgcg

SEQ ID NO: 1178 - DNA, 57 bp, Homo sapiens
  source 1..57: mol_type = genomic DNA
cgggggccgggagccccaagatgcggaaagtgcgctcctccagtgtggagtcccccg

SEQ ID NO: 1179 - DNA, 99 bp, Homo sapiens
  source 1..99: mol_type = genomic DNA
cgttgagcccctgccagaactctgtgcctaggcagcctcgggtcctctatctcagtccagccgcttgcctcacttcttcccgcttgccttatctccccg

SEQ ID NO: 1180 - DNA, 67 bp, Homo sapiens
  source 1..67: mol_type = genomic DNA
cgggtcctctatctcagtccagccgcttgcctcacttcttcccgcttgccttatctccccgcagacg

SEQ ID NO: 1181 - DNA, 62 bp, Homo sapiens
  source 1..62: mol_type = genomic DNA
cgcttgcctcacttcttcccgcttgccttatctccccgcagacgtggttcccctgcagcccg

SEQ ID NO: 1182 - DNA, 59 bp, Homo sapiens
  source 1..59: mol_type = genomic DNA
cgcttgccttatctccccgcagacgtggttcccctgcagcccgaggtgagcagctaccg

SEQ ID NO: 1183 - DNA, 45 bp, Homo sapiens
  source 1..45: mol_type = genomic DNA
cgcagacgtggttcccctgcagcccgaggtgagcagctaccggcg

SEQ ID NO: 1184 - DNA, 41 bp, Homo sapiens
  source 1..41: mol_type = genomic DNA
cgtggttcccctgcagcccgaggtgagcagctaccggcgcg

SEQ ID NO: 1185 - DNA, 77 bp, Homo sapiens; identical to SEQ ID NO: 241
  source 1..77: mol_type = genomic DNA

SEQ ID NO: 1186 - DNA, 100 bp, Homo sapiens; identical to SEQ ID NO: 244
  source 1..100: mol_type = genomic DNA

SEQ ID NO: 1187 - DNA, 127 bp, Homo sapiens
  source 1..127: mol_type = genomic DNA
cgcccggaggcagagaggctccatggccgtgctgctgggccatccccaactccctatcccatccccagcctccacccccatccagatgggactcacgtggcttcaacagctttggaaatgggtcccg

SEQ ID NO: 1188 - DNA, 132 bp, Homo sapiens
  source 1..132: mol_type = genomic DNA
cggaggcagagaggctccatggccgtgctgctgggccatccccaactccctatcccatccccagcctccacccccatccagatgggactcacgtggcttcaacagctttggaaatgggtcccgagtgggccg

SEQ ID NO: 1189 - DNA, 113 bp, Homo sapiens; identical to SEQ ID NO: 245
  source 1..113: mol_type = genomic DNA

SEQ ID NO: 1190 - DNA, 80 bp, Homo sapiens
  source 1..80: mol_type = genomic DNA
cgaaagaccagtaaagaagcactatccaggcaccagggtctcctccacccactcccgggagttggaggcggggggcgccg

SEQ ID NO: 1191 - DNA, 96 bp, Homo sapiens
  source 1..96: mol_type = genomic DNA
cgggggctcaacccccagacctccagaaatgacgtcagaatcatttgcatcccgctgcctctacctgcctggtccagctgggaccctgcctcgccg

SEQ ID NO: 1192 - DNA, 29 bp, Homo sapiens
  source 1..29: mol_type = genomic DNA
cgcagctgcatttcccggaccgcgccccg

SEQ ID NO: 1193 - DNA, 16 bp, Homo sapiens
  source 1..16: mol_type = genomic DNA
cgggcccgccgcgccg

SEQ ID NO: 1194 - DNA, 31 bp, Homo sapiens
  source 1..31: mol_type = genomic DNA
cggagccgcccgcttccgctccctctttccg

SEQ ID NO: 1195 - DNA, 27 bp, Homo sapiens
  source 1..27: mol_type = genomic DNA
cgcccgcttccgctccctctttccgcg

SEQ ID NO: 1196 - DNA, 61 bp, Homo sapiens
  source 1..61: mol_type = genomic DNA
cgtgtgaaatatcgggagaagctaacccccaaagagtgacgggattcaatccgtggcttcg

SEQ ID NO: 1197 - DNA, 130 bp, Homo sapiens
  source 1..130: mol_type = genomic DNA
cgcggcctggttctcgtttatcttttccagacagcatttaggccaataaggtctttgaaaggaggcagccccaaagccctcaggccgacacctgagggcagggtctcaaacagctgtgcttgaagaaccg

SEQ ID NO: 1198 - DNA, 74 bp, Homo sapiens
  source 1..74: mol_type = genomic DNA
cgggtgatcggaaacaggcaggcggagaattaaaagcgggtttcagacagcaacaggcccctcccctgctctcg

SEQ ID NO: 1199 - DNA, 99 bp, Homo sapiens
  source 1..99: mol_type = genomic DNA
cgccctatcctcccatccacagtcatcactgacctgtagacccggaggagcaggcttccgggggtcggtccaggttggggatacagggaggggtcctcg

SEQ ID NO: 1200 - DNA, 49 bp, Homo sapiens
  source 1..49: mol_type = genomic DNA
cgcgttccgcacaacctgtagtgcttgggtctgacttcggcagccctcg

SEQ ID NO: 1201 - DNA, 61 bp, Homo sapiens
  source 1..61: mol_type = genomic DNA
cgttccgcacaacctgtagtgcttgggtctgacttcggcagccctcgggtacagtctgccg

SEQ ID NO: 1202 - DNA, 99 bp, Homo sapiens; identical to SEQ ID NO: 279
  source 1..99: mol_type = genomic DNA

SEQ ID NO: 1203 - DNA, 87 bp, Homo sapiens
  source 1..87: mol_type = genomic DNA
cgttcctcacagcccttcagccgggagcctaccaatgctctccccaccgtctccccagccttgctccccggggacccaggacacacg

SEQ ID NO: 1204 - DNA, 121 bp, Homo sapiens
  source 1..121: mol_type = genomic DNA
cggcttctggtaggttcctggggttttttggaattgagaagcccacgacagactgcagggaacacgaaccccggaatctaatctcagggcccatttaggagaagtagctgggaccacatcg

SEQ ID NO: 1205 - DNA, 83 bp, Homo sapiens
  source 1..83: mol_type = genomic DNA
cgacagactgcagggaacacgaaccccggaatctaatctcagggcccatttaggagaagtagctgggaccacatcgggcctcg

SEQ ID NO: 1206 - DNA, 57 bp, Homo sapiens
  source 1..57: mol_type = genomic DNA
cgaggggcccggagcccgcccagctccttcccggccctctgcagcagctccagtccg

SEQ ID NO: 1207 - DNA, 56 bp, Homo sapiens
  source 1..56: mol_type = genomic DNA
cggagcccgcccagctccttcccggccctctgcagcagctccagtccggagacccg

SEQ ID NO: 1208 - DNA, 59 bp, Homo sapiens
  source 1..59: mol_type = genomic DNA
cgcccagctccttcccggccctctgcagcagctccagtccggagacccgctggctgtcg

SEQ ID NO: 1209 - DNA, 63 bp, Homo sapiens
  source 1..63: mol_type = genomic DNA
cggccgcccccgaagcctttaatggggatggtggtgaggagggggtaacaaatgccccgggcg

SEQ ID NO: 1210 - DNA, 47 bp, Homo sapiens
  source 1..47: mol_type = genomic DNA
cgggcctggtccttgaggctgccggccctccggggcgccaggactcg

SEQ ID NO: 1211 - DNA, 79 bp, Homo sapiens
  source 1..79: mol_type = genomic DNA
cgttacaactaaacttgcgaattctcggagcatttcagtttggcccagtcctctttgtcctctggagacctctcgaccg

SEQ ID NO: 1212 - DNA, 70 bp, Homo sapiens
  source 1..70: mol_type = genomic DNA
cgaattctcggagcatttcagtttggcccagtcctctttgtcctctggagacctctcgaccgagacaacg

SEQ ID NO: 1213 - DNA, 91 bp, Homo sapiens
  source 1..91: mol_type = genomic DNA
cgaccacgggaaccagcggtgaggcctgacccagcaccacgttcttcttgctttgctttttcctaaggattttgctgcaaagtctccttcg

SEQ ID NO: 1214 - DNA, 63 bp, Homo sapiens; identical to SEQ ID NO: 297
  source 1..63: mol_type = genomic DNA

SEQ ID NO: 1215 - DNA, 63 bp, Homo sapiens; identical to SEQ ID NO: 298
  source 1..63: mol_type = genomic DNA

SEQ ID NO: 1216 - DNA, 94 bp, Homo sapiens
  source 1..94: mol_type = genomic DNA
cggctctctcccagctctcctcctcctccttctcgggctcagaaagccttccagccgcggccccagccctccccccagcctgtgctgggctccg

SEQ ID NO: 1217 - DNA, 86 bp, Homo sapiens
  source 1..86: mol_type = genomic DNA
cgggctcagaaagccttccagccgcggccccagccctccccccagcctgtgctgggctccgctttccctccatcaactccaagccg

SEQ ID NO: 1218 - DNA, 75 bp, Homo sapiens
  source 1..75: mol_type = genomic DNA
cgcggccccagccctccccccagcctgtgctgggctccgctttccctccatcaactccaagccgaattcaatccg

SEQ ID NO: 1219 - DNA, 119 bp, Homo sapiens
  source 1..119: mol_type = genomic DNA
cggccccagccctccccccagcctgtgctgggctccgctttccctccatcaactccaagccgaattcaatccgagaaggctcctttgagcttttgtgtttgctgggggagatgtgggcg

SEQ ID NO: 1220 - DNA, 96 bp, Homo sapiens
  source 1..96: mol_type = genomic DNA
cgctttccctccatcaactccaagccgaattcaatccgagaaggctcctttgagcttttgtgtttgctgggggagatgtgggcgcaggagggatcg

SEQ ID NO: 1221 - DNA, 56 bp, Homo sapiens; identical to SEQ ID NO: 302
  source 1..56: mol_type = genomic DNA

SEQ ID NO: 1222 - DNA, 30 bp, Homo sapiens
  source 1..30: mol_type = genomic DNA
cgaccccgccaccgggcagaacgggccccg

SEQ ID NO: 1223 - DNA, 46 bp, Homo sapiens
  source 1..46: mol_type = genomic DNA
cgggcagaacgggccccgggggctgtacggtgaggccacaggggcg

SEQ ID NO: 1224 - DNA, 42 bp, Homo sapiens
  source 1..42: mol_type = genomic DNA
cgggccccgggggctgtacggtgaggccacaggggcgggacg

SEQ ID NO: 1225 - DNA, 41 bp, Homo sapiens
  source 1..41: mol_type = genomic DNA
cgggggctgtacggtgaggccacaggggcgggacggggccg

SEQ ID NO: 1226 - DNA, 32 bp, Homo sapiens
  source 1..32: mol_type = genomic DNA
cgctcccccgggcccgggaatgcccgcaggcg

SEQ ID NO: 1227 - DNA, 59 bp, Homo sapiens
  source 1..59: mol_type = genomic DNA
cgggaatgcccgcaggcgggtcaccgcagtctcctggtggccacaaagtgtcctgggcg

SEQ ID NO: 1228 - DNA, 83 bp, Homo sapiens
  source 1..83: mol_type = genomic DNA
cgtcggtccctttccctctccagagtcctgggcaagcctctttcctcttcctctgaaactccgacttcttccggagggcttcg

SEQ ID NO: 1229 - DNA, 84 bp, Homo sapiens
  source 1..84: mol_type = genomic DNA
cggtccctttccctctccagagtcctgggcaagcctctttcctcttcctctgaaactccgacttcttccggagggcttcgctcg

SEQ ID NO: 1230 - DNA, 49 bp, Homo sapiens
  source 1..49: mol_type = genomic DNA
cgggagcgagagaaagaacgaaaatgcacagctctctccgagtaaggcg

SEQ ID NO: 1231 - DNA, 63 bp, Homo sapiens
  source 1..63: mol_type = genomic DNA
cgaaaatgcacagctctctccgagtaaggcgaacagatggagaggaagacgaacaattaaacg

SEQ ID NO: 1232 - DNA, 91 bp, Homo sapiens
  source 1..91: mol_type = genomic DNA
cgggtcatccgcgtctggttccagaacaagcgctgcaaggacaagaagaaatccattctcatgaagcagctgcagcagcagcagcacagcg

SEQ ID NO: 1233 - DNA, 90 bp, Homo sapiens
  source 1..90: mol_type = genomic DNA
cgcgtctggttccagaacaagcgctgcaaggacaagaagaaatccattctcatgaagcagctgcagcagcagcagcacagcgacaagacg

SEQ ID NO: 1234 - DNA, 99 bp, Homo sapiens
  source 1..99: mol_type = genomic DNA
cgtctggttccagaacaagcgctgcaaggacaagaagaaatccattctcatgaagcagctgcagcagcagcagcacagcgacaagacggtgagcagccg

SEQ ID NO: 1235 - DNA, 133 bp, Homo sapiens
  source 1..133: mol_type = genomic DNA
cggtatctgcgtgcccttttctgggcgagccctgggagatccagggagaactgggcgctccagatggtgtatgtctgtaccttcacagcaaggcttcccttggatttgaggcttcctattttgtctgggatcg

SEQ ID NO: 1236 - DNA, 117 bp, Homo sapiens
  source 1..117: mol_type = genomic DNA
cgtctctttggcgcaggagactcaggctgtaaccggagaaaacacttcaccctggaaccctaactcaggtcctggcaaaagatgcgagaggaagacttgctctcttaataaatctcg

SEQ ID NO: 1237 - DNA, 110 bp, Homo sapiens
  source 1..110: mol_type = genomic DNA
cgcaggagactcaggctgtaaccggagaaaacacttcaccctggaaccctaactcaggtcctggcaaaagatgcgagaggaagacttgctctcttaataaatctcggccg

SEQ ID NO: 1238 - DNA, 126 bp, Homo sapiens
  source 1..126: mol_type = genomic DNA
cgttccagccacaggggtcacacttgcatcaacatgcgggcacgctgccccttgggtaccccaaacattgagcaaaatattggaaaggtggaccgaggtgtctgtggtgcctgccagcattggtcg

SEQ ID NO: 1239 - DNA, 71 bp, Homo sapiens
  source 1..71: mol_type = genomic DNA
cgtttgctaagtgggggagatctcaccgatcgaacgtttaaaaatggctttgtcttcattcagctctcccg

SEQ ID NO: 1240 - DNA, 88 bp, Homo sapiens
  source 1..88: mol_type = genomic DNA
cgatcgaacgtttaaaaatggctttgtcttcattcagctctcccgatttattctgtgttttacaaatagaagctcagagcttctgtcg

SEQ ID NO: 1241 - DNA, 109 bp, Homo sapiens
  source 1..109: mol_type = genomic DNA
cgaacgtttaaaaatggctttgtcttcattcagctctcccgatttattctgtgttttacaaatagaagctcagagcttctgtcgcccagtccttgcatgactcatggcg

SEQ ID NO: 1242 - DNA, 136 bp, Homo sapiens
  source 1..136: mol_type = genomic DNA
cgctggaattcgctccaaacacagatgctcatttttggaatattctagaaaaataacaagatcttgtttgtcgttatgattcacgggaggtaactgatgggagggccatttacatgagggcagacactgtggggcg

SEQ ID NO: 1243 - DNA, 48 bp, Homo sapiens
  source 1..48: mol_type = genomic DNA
cgtggctttcgggctgcctccagcccacgcaggctcgtttagtccccg

SEQ ID NO: 1244 - DNA, 59 bp, Homo sapiens
  source 1..59: mol_type = genomic DNA
cggggcctctcgcacccaaagcgagaatcatacctctagaccaacaggcctcggtggcg

SEQ ID NO: 1245 - DNA, 86 bp, Homo sapiens
  source 1..86: mol_type = genomic DNA
cgagaatcatacctctagaccaacaggcctcggtggcgggaagcaccatctttgctcctccactccctcacccccctcccgccccg

SEQ ID NO: 1246 - DNA, 58 bp, Homo sapiens
  source 1..58: mol_type = genomic DNA
cggtggcgggaagcaccatctttgctcctccactccctcacccccctcccgccccgcg

SEQ ID NO: 1247 - DNA, 48 bp, Homo sapiens
  source 1..48: mol_type = genomic DNA
cgatcgcggcagcgagaagcccaaccagaggggaggagggagtccccg

SEQ ID NO: 1248 - DNA, 57 bp, Homo sapiens
  source 1..57: mol_type = genomic DNA
cgcggcagcgagaagcccaaccagaggggaggagggagtccccggagtgggacagcg

SEQ ID NO: 1249 - DNA, 96 bp, Homo sapiens
  source 1..96: mol_type = genomic DNA
cggcagcgagaagcccaaccagaggggaggagggagtccccggagtgggacagcgctcaggtctggggtggggttgatctagggcaaaatagggcg

SEQ ID NO: 1250 - DNA, 94 bp, Homo sapiens
  source 1..94: mol_type = genomic DNA
cgagaagcccaaccagaggggaggagggagtccccggagtgggacagcgctcaggtctggggtggggttgatctagggcaaaatagggcgggcg

SEQ ID NO: 1251 - DNA, 71 bp, Homo sapiens
  source 1..71: mol_type = genomic DNA
cgatgagctctatccacttcggaatcagccgatttgtgccggattggtggcaggtgtctgaaatgtcagcg

SEQ ID NO: 1252 - DNA, 62 bp, Homo sapiens
  source 1..62: mol_type = genomic DNA
cggaatcagccgatttgtgccggattggtggcaggtgtctgaaatgtcagcggaaatacacg

SEQ ID NO: 1253 - DNA, 41 bp, Homo sapiens
  source 1..41: mol_type = genomic DNA
cgcctcatgctggcgcagcccccgccggcctccccagcccg

SEQ ID NO: 1254 - DNA, 63 bp, Homo sapiens
  source 1..63: mol_type = genomic DNA
cgcaggctgagcctctgagcctccgcgggctgcccggttttcctcaaggactggatcatagcg

SEQ ID NO: 1255 - DNA, 91 bp, Homo sapiens
  source 1..91: mol_type = genomic DNA
cgggtcctgggggaggaaggctcgccaaggccccggctcatctgtcctttccctcttagccaccacgggcagtgaccccagtttgtctccg

SEQ ID NO: 1256 - DNA, 84 bp, Homo sapiens
  source 1..84: mol_type = genomic DNA
cgccaaggccccggctcatctgtcctttccctcttagccaccacgggcagtgaccccagtttgtctccggattcccaagacccg

SEQ ID NO: 1257 - DNA, 109 bp, Homo sapiens
  source 1..109: mol_type = genomic DNA
cgggccagccaggagggttctagctctgggatatcccttattagtgttgtctcagagttcaacaacagcgacaaaaaactcttatagcttcagaaacgccgacctgccg

SEQ ID NO: 1258 - DNA, 59 bp, Homo sapiens
  source 1..59: mol_type = genomic DNA
cggcccctttctttgcccgcacactttgtccgcgtggagagaagcagccccagcccccg

SEQ ID NO: 1259 - DNA, 56 bp, Homo sapiens
  source 1..56: mol_type = genomic DNA
cgcacactttgtccgcgtggagagaagcagccccagcccccgggacttggcctccg

SEQ ID NO: 1260 - DNA, 46 bp, Homo sapiens
  source 1..46: mol_type = genomic DNA
cgcgtggagagaagcagccccagcccccgggacttggcctccgccg

SEQ ID NO: 1261 - DNA, 49 bp, Homo sapiens
  source 1..49: mol_type = genomic DNA
cgtggagagaagcagccccagcccccgggacttggcctccgccgaaccg

SEQ ID NO: 1262 - DNA, 63 bp, Homo sapiens
  source 1..63: mol_type = genomic DNA
cggtgcccccgcttgtcctaaaagacacagaccaggtccgtgaacttgacccaggcgcccacg

SEQ ID NO: 1263 - DNA, 97 bp, Homo sapiens
  source 1..97: mol_type = genomic DNA
cgaaggcctggggatcgcaagttgaggaggagaagaccgcaaacccctttggttcaagcaagctctgcggcaggtaaaaggcaaattggtggggacg

SEQ ID NO: 1264 - DNA, 130 bp, Homo sapiens
  source 1..130: mol_type = genomic DNA
cgccttaggctaggagaggaaggacgggagcacagcactgggtgcccctctccctgtagagtctggggcggggctcagtaagaaggcctgcggttggtggctccccacctcatagctgcaagtaggggcg

SEQ ID NO: 1265 - DNA, 73 bp, Homo sapiens
  source 1..73: mol_type = genomic DNA
cgggcttcctcaccaaatccccaaaaaccgacgcaggcacagagggctgactgtgttttgagtaatgcacgcg

SEQ ID NO: 1266 - DNA, 89 bp, Homo sapiens
  source 1..89: mol_type = genomic DNA
cgacccccggcagcctctttcgctctaagcctcagcctagggcaaactcccctccgcaggtcatcagcctcccatgcctgagcctcacg

SEQ ID NO: 1267 - DNA, 79 bp, Homo sapiens
  source 1..79: mol_type = genomic DNA
cggctctgagtgctggctcggggttacagacctcggcatccggctgcaggggcagacagagacctcctctgctagggcg

SEQ ID NO: 1268 - DNA, 97 bp, Homo sapiens
  source 1..97: mol_type = genomic DNA
cgccctccgccagaatgaggcccaccttcccccgtctcatcttacactcccagactctggaagactgacaggggggctgtccactcccttcggggcg

SEQ ID NO: 1269 - DNA, 24 bp, Homo sapiens
  source 1..24: mol_type = genomic DNA
cgctggcaaaacgcggcccgggcg

SEQ ID NO: 1270 - DNA, 64 bp, Homo sapiens
  source 1..64: mol_type = genomic DNA
cggtgcctcagggctggccattggcagccgaggagacaggcacttccgggcggagtgtaagacg

SEQ ID NO: 1271 - DNA, 50 bp, Homo sapiens
  source 1..50: mol_type = genomic DNA
cggagtgtaagacgctggccaatcacagcctggcagcgggacttccgtcg

SEQ ID NO: 1272 - DNA, 126 bp, Homo sapiens
  source 1..126: mol_type = genomic DNA
cgttgacactgttgatggaagggattaaaggggtatcaggttattttaatttattgttgaagatgatgactgccccctccccgatccgcataccgtcctcactttcccaccccaaccctagacccg

SEQ ID NO: 1273 - DNA, 66 bp, Homo sapiens
  source 1..66: mol_type = genomic DNA
cgtcctcactttcccaccccaaccctagacccgacaacaccgttcagccctttgccccgggcagcg

SEQ ID NO: 1274 - DNA, 77 bp, Homo sapiens
  source 1..77: mol_type = genomic DNA
cgacaacaccgttcagccctttgccccgggcagcggaaatggttagcttcctctaggagtttctgctgaggaaggcg

SEQ ID NO: 1275 - DNA, 81 bp, Homo sapiens
  source 1..81: mol_type = genomic DNA
cgcgacctctaccacatccttgagaaccacaagttcaccaaggagtctcacggcaagctgcaggccatgtggctcgaggcg

SEQ ID NO: 1276 - DNA, 95 bp, Homo sapiens
  source 1..95: mol_type = genomic DNA
cgacctctaccacatccttgagaaccacaagttcaccaaggagtctcacggcaagctgcaggccatgtggctcgaggcgcactaccaggaggccg

SEQ ID NO: 1277 - DNA, 76 bp, Homo sapiens
  source 1..76: mol_type = genomic DNA
cgggttctgcctctcctccgaggccagcctctatctgagaagacttgggatgctcccgaaagcggaatggggagcg

SEQ ID NO: 1278 - DNA, 135 bp, Homo sapiens
  source 1..135: mol_type = genomic DNA
cgggaggcgggaaaggatagctctgtaatgctctcagagctgagccaaaaaggaaaagaaagcaaaacgacaaaaaactgatccgtgtcctgcatgttggcagcagacaaccttccttgctgctgagctgtcccg

SEQ ID NO: 1279 - DNA, 141 bp, Homo sapiens; identical to SEQ ID NO: 463
  source 1..141: mol_type = genomic DNA

SEQ ID NO: 1280 - DNA, 137 bp, Homo sapiens; identical to SEQ ID NO: 465
  source 1..137: mol_type = genomic DNA

SEQ ID NO: 1281 - DNA, 150 bp, Homo sapiens
  source 1..150: mol_type = genomic DNA
cgggtcagggggcgagctgcgagaagtaggaggggtcaagacccccagaaatccctccatgggcacacacacaatcaagaatagggttgagggtcttgagaggtagaactaccctaggcagggcttctccaactcggcctttggaccccg

SEQ ID NO: 1282 - DNA, 140 bp, Homo sapiens
  source 1..140: mol_type = genomic DNA
cgagctgcgagaagtaggaggggtcaagacccccagaaatccctccatgggcacacacacaatcaagaatagggttgagggtcttgagaggtagaactaccctaggcagggcttctccaactcggcctttggaccccgcg

SEQ ID NO: 1283 - DNA, 55 bp, Homo sapiens
  source 1..55: mol_type = genomic DNA
cggaaatccgttgggcactgaaggacttttcgaaccctgtagcgctgttgcttcg

SEQ ID NO: 1284 - DNA, 22 bp, Homo sapiens
  source 1..22: mol_type = genomic DNA
cgtctcgggccctccggcgccg

SEQ ID NO: 1285 - DNA, 19 bp, Homo sapiens
  source 1..19: mol_type = genomic DNA
cgggccctccggcgccgcg

SEQ ID NO: 1286 - DNA, 53 bp, Homo sapiens
  source 1..53: mol_type = genomic DNA
cgtcttaatgcaacaaacgaaaccccacacgaacgaaaaggaacatgtctgcg

SEQ ID NO: 1287 - DNA, 44 bp, Homo sapiens; identical to SEQ ID NO: 485
  source 1..44: mol_type = genomic DNA

SEQ ID NO: 1288 - DNA, 58 bp, Homo sapiens; identical to SEQ ID NO: 486
  source 1..58: mol_type = genomic DNA

SEQ ID NO: 1289 - DNA, 55 bp, Homo sapiens
  source 1..55: mol_type = genomic DNA
cgcatttcggtactgtttccccgagatcaggaactttccgggtctaggagcaacg

SEQ ID NO: 1290 - DNA, 80 bp, Homo sapiens
  source 1..80: mol_type = genomic DNA
cggtactgtttccccgagatcaggaactttccgggtctaggagcaacgcctggagggggctgtagagacccagccccccg

SEQ ID NO: 1291 - DNA, 77 bp, Homo sapiens
  source 1..77: mol_type = genomic DNA
cgtgaagttgctttgcacactcccgggaagcgtctgtagtctaggggttctggtcctggggagacggagtggcatcg

SEQ ID NO: 1292 - DNA, 70 bp, Homo sapiens
  source 1..70: mol_type = genomic DNA
cgggaagcgtctgtagtctaggggttctggtcctggggagacggagtggcatcgtccttgggaaacttcg

SEQ ID NO: 1293 - DNA, 75 bp, Homo sapiens
  source 1..75: mol_type = genomic DNA
cgtctgtagtctaggggttctggtcctggggagacggagtggcatcgtccttgggaaacttcgcccccaaccccg

SEQ ID NO: 1294 - DNA, 62 bp, Homo sapiens
  source 1..62: mol_type = genomic DNA
cgtccttgggaaacttcgcccccaaccccgtttggaagccccagatcccaaatcgacttgcg

SEQ ID NO: 1295 - DNA, 49 bp, Homo sapiens
  source 1..49: mol_type = genomic DNA
cgcccccaaccccgtttggaagccccagatcccaaatcgacttgcgccg

SEQ ID NO: 1296 - DNA, 52 bp, Homo sapiens
  source 1..52: mol_type = genomic DNA
cgtttggaagccccagatcccaaatcgacttgcgccgcaacctccttccccg

SEQ ID NO: 1297 - DNA, 30 bp, Homo sapiens
  source 1..30: mol_type = genomic DNA
cgacttgcgccgcaacctccttccccgtcg

SEQ ID NO: 1298 - DNA, 82 bp, Homo sapiens; identical to SEQ ID NO: 487
  source 1..82: mol_type = genomic DNA

SEQ ID NO: 1299 - DNA, 61 bp, Homo sapiens; identical to SEQ ID NO: 488
  source 1..61: mol_type = genomic DNA

SEQ ID NO: 1300 - DNA, 46 bp, Homo sapiens
  source 1..46: mol_type = genomic DNA
cggcccgagcgacccctcagagtttggcaccgagttcagggcaccg

SEQ ID NO: 1301 - DNA, 53 bp, Homo sapiens; identical to SEQ ID NO: 492
  source 1..53: mol_type = genomic DNA

SEQ ID NO: 1302 - DNA, 52 bp, Homo sapiens; identical to SEQ ID NO: 493
  source 1..52: mol_type = genomic DNA

SEQ ID NO: 1303 - DNA, 28 bp, Homo sapiens
  source 1..28: mol_type = genomic DNA
cgctgtgctgcccccgccccgagacgcg

SEQ ID NO: 1304 - DNA, 32 bp, Homo sapiens
  source 1..32: mol_type = genomic DNA
cgccgggtcaccggttttccgaaagggacccg

SEQ ID NO: 1305 - DNA, 49 bp, Homo sapiens
  source 1..49: mol_type = genomic DNA
cgggtcaccggttttccgaaagggacccggcagagacaaagtgccttcg

SEQ ID NO: 1306 - DNA, 44 bp, Homo sapiens
  source 1..44: mol_type = genomic DNA
cggttttccgaaagggacccggcagagacaaagtgccttcgccg

SEQ ID NO: 1307 - DNA, 41 bp, Homo sapiens
  source 1..41: mol_type = genomic DNA
cgaaagggacccggcagagacaaagtgccttcgccgctgcg

SEQ ID NO: 1308 - DNA, 58 bp, Homo sapiens
  source 1..58: mol_type = genomic DNA
cgccgctgcgataggttggttttactttgcaataaacagcccctaatgggaccgggcg

SEQ ID NO: 1309 - DNA, 58 bp, Homo sapiens
  source 1..58: mol_type = genomic DNA
cgctgcgataggttggttttactttgcaataaacagcccctaatgggaccgggcgccg

SEQ ID NO: 1310 - DNA, 57 bp, Homo sapiens
  source 1..57: mol_type = genomic DNA
cgataggttggttttactttgcaataaacagcccctaatgggaccgggcgccgggcg

SEQ ID NO: 1311 - DNA, 22 bp, Homo sapiens
  source 1..22: mol_type = genomic DNA
cgggcgccgggcggagagctcg

SEQ ID NO: 1312 - DNA, 23 bp, Homo sapiens
  source 1..23: mol_type = genomic DNA
cgccgggcggagagctcggcccg

SEQ ID NO: 1313 - DNA, 24 bp, Homo sapiens
  source 1..24: mol_type = genomic DNA
cggcccggggcgcggcctttgccg

SEQ ID NO: 1314 - DNA, 36 bp, Homo sapiens
  source 1..36: mol_type = genomic DNA
cgccccgccgggcgccaggttttggggggtggcccg

SEQ ID NO: 1315 - DNA, 37 bp, Homo sapiens
  source 1..37: mol_type = genomic DNA
cgccgggcgccaggttttggggggtggcccggccccg

SEQ ID NO: 1316 - DNA, 36 bp, Homo sapiens
  source 1..36: mol_type = genomic DNA
cgggcgccaggttttggggggtggcccggccccgcg

SEQ ID NO: 1317 - DNA, 36 bp, Homo sapiens
  source 1..36: mol_type = genomic DNA
cgccaggttttggggggtggcccggccccgcgtccg

SEQ ID NO: 1318 - DNA, 27 bp, Homo sapiens
  source 1..27: mol_type = genomic DNA
cggccccgcgtccgccactgcaggccg

SEQ ID NO: 1319 - DNA, 36 bp, Homo sapiens
  source 1..36: mol_type = genomic DNA
cgcgtccgccactgcaggccgctctcctccttcccg

SEQ ID NO: 1320 - DNA, 36 bp, Homo sapiens
  source 1..36: mol_type = genomic DNA
cgtccgccactgcaggccgctctcctccttcccgcg

SEQ ID NO: 1321 - DNA, 60 bp, Homo sapiens
  source 1..60: mol_type = genomic DNA
cgcgcacacagcggagaaaaaaggacgcaaacagcattttacacttttcccactttagcg

SEQ ID NO: 1322 - DNA, 62 bp, Homo sapiens; identical to SEQ ID NO: 497
  source 1..62: mol_type = genomic DNA

SEQ ID NO: 1323 - DNA, 20 bp, Homo sapiens
  source 1..20: mol_type = genomic DNA
cgggagggggcgcgggcgcg

SEQ ID NO: 1324 - DNA, 16 bp, Homo sapiens
  source 1..16: mol_type = genomic DNA
cgcgggcgcggccccg

SEQ ID NO: 1325 - DNA, 57 bp, Homo sapiens
  source 1..57: mol_type = genomic DNA
cggcggccctttggagacaccatttacagaaatgactttattgacggcttaacgtcg

SEQ ID NO: 1326 - DNA, 104 bp, Homo sapiens
  source 1..104: mol_type = genomic DNA
cggctggggcatggtggcaccggttgtctactccccacttgtgacaccgacagcttccaactcctcagacccaccccgtggaattctggactttgtgagggccg

SEQ ID NO: 1327 - DNA, 105 bp, Homo sapiens
  source 1..105: mol_type = genomic DNA
cgacagcttccaactcctcagacccaccccgtggaattctggactttgtgagggccgccggggtcctggccctggggtcagctgccatctgactaagccaggacg

SEQ ID NO: 1328 - DNA, 76 bp, Homo sapiens
  source 1..76: mol_type = genomic DNA
cgccggaggccgcatctccccttgttggtattgttgtcggcttgctttcttctggcttcccagctcagtgaccccg

SEQ ID NO: 1329 - DNA, 44 bp, Homo sapiens
  source 1..44: mol_type = genomic DNA
cggtggggcgcagggccggggagccaggggggtgccggggctcg

SEQ ID NO: 1330 - DNA, 42 bp, Homo sapiens
  source 1..42: mol_type = genomic DNA
cgcagggccggggagccaggggggtgccggggctcgaggccg

SEQ ID NO: 1331 - DNA, 36 bp, Homo sapiens
  source 1..36: mol_type = genomic DNA
cggggagccaggggggtgccggggctcgaggccgcg

SEQ ID NO: 1332 - DNA, 23 bp, Homo sapiens
  source 1..23: mol_type = genomic DNA
cggggctcgaggccgcggggccg

SEQ ID NO: 1333 - DNA, 18 bp, Homo sapiens
  source 1..18: mol_type = genomic DNA
cgaggccgcggggccgcg

SEQ ID NO: 1334 - DNA, 74 bp, Homo sapiens
  source 1..74: mol_type = genomic DNA
cgctccttccgaagtctcgctggaaacttcaagggctgatggttctgtgttcttgacgtaattaggaggccacg

SEQ ID NO: 1335 - DNA, 67 bp, Homo sapiens
  source 1..67: mol_type = genomic DNA
cgccgcccctcggggccttggtctttctaagctctttcttaggaagcggagagaggggcagaaaacg

SEQ ID NO: 1336 - DNA, 73 bp, Homo sapiens
  source 1..73: mol_type = genomic DNA
cgcccctcggggccttggtctttctaagctctttcttaggaagcggagagaggggcagaaaacgtgcacctcg

SEQ ID NO: 1337 - DNA, 76 bp, Homo sapiens
  source 1..76: mol_type = genomic DNA
cggggccttggtctttctaagctctttcttaggaagcggagagaggggcagaaaacgtgcacctcgccacccaccg

SEQ ID NO: 1338 - DNA, 71 bp, Homo sapiens
  source 1..71: mol_type = genomic DNA
cggtccccaattgcggtggggcctcgcctccccagggccacctcggggccccagaggcctgtgcctttccg

SEQ ID NO: 1339 - DNA, 128 bp, Homo sapiens
  source 1..128: mol_type = genomic DNA
cgatttatgcggaaaataaatctccaagctcaagagcaaatgaaaagtttcacctctggttcctgcttgaggaacaaagaccaactgggcttgccgcctaggggaaagtggggccgtgggtatgggcg

SEQ ID NO: 1340 - DNA, 129 bp, Homo sapiens
  source 1..129: mol_type = genomic DNA
cggtggccgggaaataaataagccaattcctttggtgactaccccccgcggatttccagacccttagctaaatctagccacccagaaaggggaaaggggaaaaagaaacaatcaacccagatgcccccg

SEQ ID NO: 1341 - DNA, 49 bp, Homo sapiens; identical to SEQ ID NO: 508
  source 1..49: mol_type = genomic DNA

SEQ ID NO: 1342 - DNA, 58 bp, Homo sapiens; identical to SEQ ID NO: 509
  source 1..58: mol_type = genomic DNA

SEQ ID NO: 1343 - DNA, 109 bp, Homo sapiens; identical to SEQ ID NO: 511
  source 1..109: mol_type = genomic DNA

SEQ ID NO: 1344 - DNA, 125 bp, Homo sapiens
  source 1..125: mol_type = genomic DNA
cggacagcgtgtgtgtataggaagggtctgctccaatgcctcttacctgtgtgaaatgcctttgccgggtaccagtgcacaaggtagggcaaattagctcactcggatttggggtctagaagtcg

SEQ ID NO: 1345 - DNA, 92 bp, Homo sapiens; identical to SEQ ID NO: 515
  source 1..92: mol_type = genomic DNA

SEQ ID NO: 1346 - DNA, 101 bp, Homo sapiens; identical to SEQ ID NO: 516
  source 1..101: mol_type = genomic DNA

SEQ ID NO: 1347 - DNA, 104 bp, Homo sapiens; identical to SEQ ID NO: 517
  source 1..104: mol_type = genomic DNA

SEQ ID NO: 1348 - DNA, 68 bp, Homo sapiens; identical to SEQ ID NO: 518
  source 1..68: mol_type = genomic DNA

SEQ ID NO: 1349 - DNA, 87 bp, Homo sapiens
  source 1..87: mol_type = genomic DNA
cggccaggctctggagcacgtgacccgagaggaggctgcggctcaaggccattttcaaatctcattggcttggttgtcatgtggtcg

SEQ ID NO: 1350 - DNA, 92 bp, Homo sapiens
  source 1..92: mol_type = genomic DNA
cgtgacccgagaggaggctgcggctcaaggccattttcaaatctcattggcttggttgtcatgtggtcggcagaggcatccacaattacacg

SEQ ID NO: 1351 - DNA, 46 bp, Homo sapiens
  source 1..46: mol_type = genomic DNA
cgatctgaacgccggctgggaggctcctggggggaacttgcggtcg

SEQ ID NO: 1352 - DNA, 125 bp, Homo sapiens
  source 1..125: mol_type = genomic DNA
cggttaaagtgaatcaagtcacagtgcttccttaacccaacctctctgttggggtcagccacagcctaaaccgcctgccgttcagcctgagaggctgctgctagcctgctcacgcatgcagcccg

SEQ ID NO: 1353 - DNA, 93 bp, Homo sapiens
  source 1..93: mol_type = genomic DNA
cgtgtcgggcttagaggacttcactgggcgggttggggtttattttcaataaattcagctgttcacatacacccccaccaccatttccacgcg

SEQ ID NO: 1354 - DNA, 90 bp, Homo sapiens
  source 1..90: mol_type = genomic DNA
cgggcttagaggacttcactgggcgggttggggtttattttcaataaattcagctgttcacatacacccccaccaccatttccacgcgcg

SEQ ID NO: 1355 - DNA, 69 bp, Homo sapiens
  source 1..69: mol_type = genomic DNA
cgggttggggtttattttcaataaattcagctgttcacatacacccccaccaccatttccacgcgcgcg

SEQ ID NO: 1356 - DNA, 88 bp, Homo sapiens
  source 1..88: mol_type = genomic DNA
cgcgtggcctactcgcctactcccttattattggctgggacctgttcctttccaaagtgtgatgcccggtgtcctaccctaggccacg

SEQ ID NO: 1357 - DNA, 88 bp, Homo sapiens
  source 1..88: mol_type = genomic DNA
cgtggcctactcgcctactcccttattattggctgggacctgttcctttccaaagtgtgatgcccggtgtcctaccctaggccacgcg

SEQ ID NO: 1358 - DNA, 95 bp, Homo sapiens
  source 1..95: mol_type = genomic DNA
cggacgctgctctgacccaagacgcgggagagaagtcccaaaggctacagccaggggctgggggactcctctgctcaccttagtccttgatttcg

SEQ ID NO: 1359 - DNA, 126 bp, Homo sapiens
  source 1..126: mol_type = genomic DNA
cgctgctctgacccaagacgcgggagagaagtcccaaaggctacagccaggggctgggggactcctctgctcaccttagtccttgatttcgaaggccccaattaactatctgttgtgtcagaatcg

SEQ ID NO: 1360 - DNA, 111 bp, Homo sapiens
  source 1..111: mol_type = genomic DNA
cgcgggagagaagtcccaaaggctacagccaggggctgggggactcctctgctcaccttagtccttgatttcgaaggccccaattaactatctgttgtgtcagaatcggcg

SEQ ID NO: 1361 - DNA, 126 bp, Homo sapiens
  source 1..126: mol_type = genomic DNA
cgggagagaagtcccaaaggctacagccaggggctgggggactcctctgctcaccttagtccttgatttcgaaggccccaattaactatctgttgtgtcagaatcggcgagtgcaaagtagctgcg

SEQ ID NO: 1362 - DNA, 26 bp, Homo sapiens
  source 1..26: mol_type = genomic DNA
cgcgggctccccggaaggcagcgccg

SEQ ID NO: 1363 - DNA, 45 bp, Homo sapiens
  source 1..45: mol_type = genomic DNA
cgggctccccggaaggcagcgccgcctgggagtggggaggctgcg

SEQ ID NO: 1364 - DNA, 124 bp, Homo sapiens
  source 1..124: mol_type = genomic DNA
cggctgatggaactcactgggggcgggaggaggaaggaagcaagggagcgcacatggttgagacaagcagggctgtgcgggaaggaccccccacccccaaacaccagccagggccctagagccg

SEQ ID NO: 1365 - DNA, 118 bp, Homo sapiens
  source 1..118: mol_type = genomic DNA
cgggaggaggaaggaagcaagggagcgcacatggttgagacaagcagggctgtgcgggaaggaccccccacccccaaacaccagccagggccctagagccgctgccctgcactgctcg

SEQ ID NO: 1366 - DNA, 81 bp, Homo sapiens
  source 1..81: mol_type = genomic DNA
cgactcattttctctcctttccggcctctttcttttctgccgagcgctctgagattcatatgtacccagatgatcagcccg

SEQ ID NO: 1367 - DNA, 139 bp, Homo sapiens
  source 1..139: mol_type = genomic DNA
cgggtgattccagccactctttctctgtctccgagtcttgggcctcccctttatttctttctgaagtctctccggagcccaagccaccccacacccaaaccccgcagctggatgggagtccaggccacttccctatccg

SEQ ID NO: 1368 - DNA, 114 bp, Homo sapiens
  source 1..114: mol_type = genomic DNA
cgagtcttgggcctcccctttatttctttctgaagtctctccggagcccaagccaccccacacccaaaccccgcagctggatgggagtccaggccacttccctatccgcagccg

SEQ ID NO: 1369 - DNA, 62 bp, Homo sapiens
  source 1..62: mol_type = genomic DNA
cgacacagaaacccatcgaaaggggccaccgagacccgttgattagctggagggagcatgcg

SEQ ID NO: 1370 - DNA, 130 bp, Homo sapiens
  source 1..130: mol_type = genomic DNA
cgggggactgaaccttctggtggaccggctgaggctcccttgagctctgttcctctcaccacctccaggggtagggtagctgtcactgttgacgtgccagaggtatgagcctcctcccttccggtgatcg

SEQ ID NO: 1371 - DNA, 106 bp, Homo sapiens
  source 1..106: mol_type = genomic DNA
cgggcggccttgggtcctttgcagtcggagaggctgcaggggaggaggaagacccagccagctccagggtgcaggattctgcccttccacccgagagcctcccccg

SEQ ID NO: 1372 - DNA, 128 bp, Homo sapiens
  source 1..128: mol_type = genomic DNA
cggccttgggtcctttgcagtcggagaggctgcaggggaggaggaagacccagccagctccagggtgcaggattctgcccttccacccgagagcctcccccggtgtcccagtggcagtggctgccccg

SEQ ID NO: 1373 - DNA, 114 bp, Homo sapiens
  source 1..114: mol_type = genomic DNA
cggagaggctgcaggggaggaggaagacccagccagctccagggtgcaggattctgcccttccacccgagagcctcccccggtgtcccagtggcagtggctgccccgggctgcg

SEQ ID NO: 1374 - DNA, 53 bp, Homo sapiens
  source 1..53: mol_type = genomic DNA
cggggcttcctcgagtctccaaggcccgggcttcaacttcccgggtctagacg

SEQ ID NO: 1375 - DNA, 56 bp, Homo sapiens
  source 1..56: mol_type = genomic DNA
cgagtctccaaggcccgggcttcaacttcccgggtctagacgtcagccctgaaccg

SEQ ID NO: 1376 - DNA, 52 bp, Homo sapiens
  source 1..52: mol_type = genomic DNA
cgtgagatccccatcaaccttccccgtgtggggtctgagcgacccgcatccg

SEQ ID NO: 1377 - DNA, 140 bp, Homo sapiens
  source 1..140: mol_type = genomic DNA
cgctaataagacactattgtactatatgaggacaaggaaagggagctgactccttaaaccggcctgcagtctccctggggatagatgtctgggaaaagcaaggcgatattttcatatccttagccgccaagtcctcctcg

SEQ ID NO: 1378 - DNA, 119 bp, Homo sapiens
  source 1..119: mol_type = genomic DNA
cggcctgcagtctccctggggatagatgtctgggaaaagcaaggcgatattttcatatccttagccgccaagtcctcctcgcccatcctggggtttcctggggtttgtggcatccaccg

SEQ ID NO: 1379 - DNA, 83 bp, Homo sapiens
  source 1..83: mol_type = genomic DNA
cgatattttcatatccttagccgccaagtcctcctcgcccatcctggggtttcctggggtttgtggcatccaccgagatttcg

SEQ ID NO: 1380 - DNA, 107 bp, Homo sapiens
  source 1..107: mol_type = genomic DNA
cgccaagtcctcctcgcccatcctggggtttcctggggtttgtggcatccaccgagatttcggagttaaatctgcatattcctctgggtcccaatctcccctaagcg

SEQ ID NO: 1381 - DNA, 109 bp, Homo sapiens
  source 1..109: mol_type = genomic DNA
cgcccatcctggggtttcctggggtttgtggcatccaccgagatttcggagttaaatctgcatattcctctgggtcccaatctcccctaagcgaactgcagccagaacg

SEQ ID NO: 1382 - DNA, 87 bp, Homo sapiens
  source 1..87: mol_type = genomic DNA
cgttggctgaccaggacacaacctccatcctgccccagagcgtctttctccgagccccaaccccgagattcttcctccactgaagcg

SEQ ID NO: 1383 - DNA, 67 bp, Homo sapiens
  source 1..67: mol_type = genomic DNA
cgtctttctccgagccccaaccccgagattcttcctccactgaagcgcaggcctcctaaaacccccg

SEQ ID NO: 1384 - DNA, 82 bp, Homo sapiens
  source 1..82: mol_type = genomic DNA
cggccccctctctgcacacctggcacacggctcctccctggcggcatgtgggtatttgggttgggcgagcctctccctcccg

SEQ ID NO: 1385 - DNA, 66 bp, Homo sapiens
  source 1..66: mol_type = genomic DNA
cgaattttgtttagagctggcaagggggacttagctcgcgccccaagacctgggcttgcagcgccg

SEQ ID NO: 1386 - DNA, 42 bp, Homo sapiens
  source 1..42: mol_type = genomic DNA
cgcgccccaagacctgggcttgcagcgccgccaacaggcccg

SEQ ID NO: 1387 - DNA, 30 bp, Homo sapiens
  source 1..30: mol_type = genomic DNA
cgccgccaacaggcccggggacacgaggcg

SEQ ID NO: 1388 - DNA, 91 bp, Homo sapiens
  source 1..91: mol_type = genomic DNA
cgcttcctgcgtttgcccctcaggtttcccgtttctccacaaaggcctaggggagcctcgcccacaggctgaccctgcaacctctggcccg

SEQ ID NO: 1389 - DNA, 78 bp, Homo sapiens
  source 1..78: mol_type = genomic DNA
cggcctttctcagagaaaacctcctaaacgtgtgaagaccgctttgggggaagcgagagggaggttggaggagccccg

SEQ ID NO: 1390 - DNA, 54 bp, Homo sapiens
  source 1..54: mol_type = genomic DNA
cgtgtgaagaccgctttgggggaagcgagagggaggttggaggagccccgggcg

SEQ ID NO: 1391 - DNA, 54 bp, Homo sapiens
  source 1..54: mol_type = genomic DNA
cgctttgggggaagcgagagggaggttggaggagccccgggcggggtctcagcg

SEQ ID NO: 1392 - DNA, 74 bp, Homo sapiens
  source 1..74: mol_type = genomic DNA
cgagagggaggttggaggagccccgggcggggtctcagcgcccaccagctgtgccttcagggcttgggtgttcg

SEQ ID NO: 1393 - DNA, 59 bp, Homo sapiens
  source 1..59: mol_type = genomic DNA
cgggcggggtctcagcgcccaccagctgtgccttcagggcttgggtgttcgctgcaacg

SEQ ID NO: 1394 - DNA, 132 bp, Homo sapiens
  source 1..132: mol_type = genomic DNA
cggcaccacctggtcgctggattcccaccctacaggagcacagctagtacactgaacagtctggaaggtgttggcacctgccatttcgcgggacactggcaccattttataaaacagtgatagaaagatgcg

SEQ ID NO: 1395 - DNA, 103 bp, Homo sapiens
  source 1..103: mol_type = genomic DNA
cggccattcccagaaggctggctcgttctcaggccagctctcaccaccgctggctctctgcctaccgcaaacttgctggtctaatttaggaacaattgggccg

SEQ ID NO: 1396 - DNA, 74 bp, Homo sapiens
  source 1..74: mol_type = genomic DNA
cgcccctcgctgcccctgggctcgggccaaaccctgcataaggtcccctggacagccaggtaatctccgtcccg

SEQ ID NO: 1397 - DNA, 75 bp, Homo sapiens
  source 1..75: mol_type = genomic DNA
cgctgcccctgggctcgggccaaaccctgcataaggtcccctggacagccaggtaatctccgtcccgcctgcccg

SEQ ID NO: 1398 - DNA, 64 bp, Homo sapiens
  source 1..64: mol_type = genomic DNA
cgggccaaaccctgcataaggtcccctggacagccaggtaatctccgtcccgcctgcccgaccg

SEQ ID NO: 1399 - DNA, 25 bp, Homo sapiens
  source 1..25: mol_type = genomic DNA
cgtcccgcctgcccgaccggggtcg

SEQ ID NO: 1400 - DNA, 59 bp, Homo sapiens
  source 1..59: mol_type = genomic DNA
cgtggaaacgcagactcgaccctaaacgcttaacccacagagatcaacaggttcaagcg

SEQ ID NO: 1401 - DNA, 60 bp, Homo sapiens
  source 1..60: mol_type = genomic DNA
cgcagactcgaccctaaacgcttaacccacagagatcaacaggttcaagcggaatattcg

SEQ ID NO: 1402 - DNA, 54 bp, Homo sapiens
  source 1..54: mol_type = genomic DNA
cgaccctaaacgcttaacccacagagatcaacaggttcaagcggaatattcgcg

SEQ ID NO: 1403 - DNA, 69 bp, Homo sapiens
  source 1..69: mol_type = genomic DNA
cgaaactggacaaggccgacccacccagccgggcccgatccctcaagacactttcagcctcagcccccg

SEQ ID NO: 1404 - DNA, 52 bp, Homo sapiens
  source 1..52: mol_type = genomic DNA
cgcaggcccactctcttggctctcctggcccgcacactcagctcggccgccg

SEQ ID NO: 1405 - DNA, 23 bp, Homo sapiens
  source 1..23: mol_type = genomic DNA
cgtcttcgctcccgctcgccccg

SEQ ID NO: 1406 - DNA, 19 bp, Homo sapiens
  source 1..19: mol_type = genomic DNA
cgctcccgctcgccccgcg

SEQ ID NO: 1407 - DNA, 19 bp, Homo sapiens
  source 1..19: mol_type = genomic DNA
cgctcgccccgcgcttccg

SEQ ID NO: 1408 - DNA, 77 bp, Homo sapiens
  source 1..77: mol_type = genomic DNA
cgccctccctcccggcctcagcctcctggttcgccaaaccggaggactgaatttatggctagctggtctctggggcg

SEQ ID NO: 1409 - DNA, 47 bp, Homo sapiens
  source 1..47: mol_type = genomic DNA
cgagagacgctgctcccaactcgcagagggagaaagcgtgtatcccg

SEQ ID NO: 1410 - DNA, 48 bp, Homo sapiens
  source 1..48: mol_type = genomic DNA
cgctgctcccaactcgcagagggagaaagcgtgtatcccgggctgccg

SEQ ID NO: 1411 - DNA, 49 bp, Homo sapiens
  source 1..49: mol_type = genomic DNA
cgccgaccgcagcgggagggggcaaggggctctctctgactcagctccg

SEQ ID NO: 1412 - DNA, 62 bp, Homo sapiens
  source 1..62: mol_type = genomic DNA
cgaccgcagcgggagggggcaaggggctctctctgactcagctccggatttgtgcatccccg

SEQ ID NO: 1413 - DNA, 81 bp, Homo sapiens
  source 1..81: mol_type = genomic DNA
cggatttgtgcatccccgcagtgcggcacccgcagtcttgacttccccagtctctgactgaacagatgtttttgtccctcg

SEQ ID NO: 1414 - DNA, 72 bp, Homo sapiens
  source 1..72: mol_type = genomic DNA
cgatatttggactacagagccaagcagttgcaggacgacgtgcaacctcaaaggaaagtgtcccgaaggccg

SEQ ID NO: 1415 - DNA, 74 bp, Homo sapiens
  source 1..74: mol_type = genomic DNA
cgacgtgcaacctcaaaggaaagtgtcccgaaggccgcttcctttttgggggttgtggatctcagagccaggcg

SEQ ID NO: 1416 - DNA, 95 bp, Homo sapiens
  source 1..95: mol_type = genomic DNA
cgcttcctttttgggggttgtggatctcagagccaggcgctcgcggggttcaggctgctgagaccctgccaagggttaggtttatttaccccacg

SEQ ID NO: 1417 - DNA, 66 bp, Homo sapiens
  source 1..66: mol_type = genomic DNA
cgctcgcggggttcaggctgctgagaccctgccaagggttaggtttatttaccccacgaacattcg

SEQ ID NO: 1418 - DNA, 69 bp, Homo sapiens
  source 1..69: mol_type = genomic DNA
cgcggggttcaggctgctgagaccctgccaagggttaggtttatttaccccacgaacattcgcccagcg

SEQ ID NO: 1419 - DNA, 78 bp, Homo sapiens
  source 1..78: mol_type = genomic DNA
cggggttcaggctgctgagaccctgccaagggttaggtttatttaccccacgaacattcgcccagcgtccactgagcg

SEQ ID NO: 1420 - DNA, 81 bp, Homo sapiens
  source 1..81: mol_type = genomic DNA
cggtattggcgctttcccagaactggcccagaggagccagttcagagtgtgaggtcgggtctgcattgaacgtacacaccg

SEQ ID NO: 1421 - DNA, 92 bp, Homo sapiens
  source 1..92: mol_type = genomic DNA
cgctttcccagaactggcccagaggagccagttcagagtgtgaggtcgggtctgcattgaacgtacacaccgaggtctatcagactcccccg

SEQ ID NO: 1422 - DNA, 55 bp, Homo sapiens
  source 1..55: mol_type = genomic DNA
cgggtctgcattgaacgtacacaccgaggtctatcagactcccccgattttagcg

SEQ ID NO: 1423 - DNA, 125 bp, Homo sapiens
  source 1..125: mol_type = genomic DNA
cgcagctgatgagtttcagcaactcgccacctgggcgcttttctttaaattttgggagtaaactgggaaaataaaaaaatctccacgtccactggctctctccccttctccaacttcctctttcg

SEQ ID NO: 1424 - DNA, 89 bp, Homo sapiens
  source 1..89: mol_type = genomic DNA
cggcagattgagggtggcagcaaaggcccggcctccaaggataatggggagccgttttccctcacgcctggtctctatggcccccttcg

SEQ ID NO: 1425 - DNA, 70 bp, Homo sapiens
  source 1..70: mol_type = genomic DNA
cgcccccccgccccctgctccccgcttcccgccagcatctctccttttctgctcaggagtgtttggcccg

SEQ ID NO: 1426 - DNA, 62 bp, Homo sapiens
  source 1..62: mol_type = genomic DNA
cgccagcatctctccttttctgctcaggagtgtttggcccggcggtccaccccggcttcccg

SEQ ID NO: 1427 - DNA, 65 bp, Homo sapiens
  source 1..65: mol_type = genomic DNA
cgccccactctggggcagcgtgtgtgacccaggccttcggaccgagaagggagaaaggaaatacg

SEQ ID NO: 1428 - DNA, 113 bp, Homo sapiens
  source 1..113: mol_type = genomic DNA
cggggcttaactggagccgaagccccagaacaggcggccattcatcttctgagcagagtctgggagaagcagcctccacttcccagggcagccagtacgtgggggccctttcg

SEQ ID NO: 1429 - DNA, 47 bp, Homo sapiens
  source 1..47: mol_type = genomic DNA
cgccgcacacacacgctccacttcctgcccgccagcaggccccagcg

SEQ ID NO: 1430 - DNA, 87 bp, Homo sapiens
  source 1..87: mol_type = genomic DNA
cgcagctccagtcctggcaggatgtgcaaggcctctcgaggttttcaaatgggcccccgaggcctgggagatccccgagtagccacg

SEQ ID NO: 1431 - DNA, 61 bp, Homo sapiens
  source 1..61: mol_type = genomic DNA
cgtttgtcttgaggccgtgttgaataccctttgccaactgtaaattcggcgtgtgtgcccg

SEQ ID NO: 1432 - DNA, 106 bp, Homo sapiens; identical to SEQ ID NO: 734
  source 1..106: mol_type = genomic DNA

SEQ ID NO: 1433 - DNA, 96 bp, Homo sapiens
  source 1..96: mol_type = genomic DNA
cgtgcctgaagtcgaggttagtacgcagccagcagggaccctgacaggaaaccagagcagccccggggttgggggagccctggtaggtgctggtcg

SEQ ID NO: 1434 - DNA, 69 bp, Homo sapiens
  source 1..69: mol_type = genomic DNA
cgtttgtcccttacttgggaggtcccgggtatctgaagcggatcccgggtctggggacatgaaggggcg

SEQ ID NO: 1435 - DNA, 71 bp, Homo sapiens
  source 1..71: mol_type = genomic DNA
cggatcccgggtctggggacatgaaggggcgccgtggccttagggaaggccccaaagaggcctaggccccg

SEQ ID NO: 1436 - DNA, 80 bp, Homo sapiens
  source 1..80: mol_type = genomic DNA
cgggtctggggacatgaaggggcgccgtggccttagggaaggccccaaagaggcctaggccccggaggaggcaagagccg

SEQ ID NO: 1437 - DNA, 40 bp, Homo sapiens
  source 1..40: mol_type = genomic DNA
cgggccaggggtgggaggcggttccctaccgcaggcggcg

SEQ ID NO: 1438 - DNA, 25 bp, Homo sapiens
  source 1..25: mol_type = genomic DNA
cgcgcaggtttcccaccgacgcccg

SEQ ID NO: 1439 - DNA, 27 bp, Homo sapiens
  source 1..27: mol_type = genomic DNA
cgcaggtttcccaccgacgcccgctcg

SEQ ID NO: 1440 - DNA, 20 bp, Homo sapiens
  source 1..20: mol_type = genomic DNA
cgacgcccgctcggaaggcg

SEQ ID NO: 1441 - DNA, 95 bp, Homo sapiens
  source 1..95: mol_type = genomic DNA
cgagtgtgcaaaatgactcctttctgaaccaaacggcatgctccgtgctggggaaatgagatgcacatttaaatctcatccatccaccgcctgcg

SEQ ID NO: 1442 - DNA, 21 bp, Homo sapiens
  source 1..21: mol_type = genomic DNA
cggtgcgcgggcacgcacacg

SEQ ID NO: 1443 - DNA, 19 bp, Homo sapiens
  source 1..19: mol_type = genomic DNA
cgcgggcacgcacacgccg

SEQ ID NO: 1444 - DNA, 20 bp, Homo sapiens
  source 1..20: mol_type = genomic DNA
cgggcacgcacacgccggcg

SEQ ID NO: 1445 - DNA, 89 bp, Homo sapiens
  source 1..89: mol_type = genomic DNA
cgggctcctgccttgggctggggggtctcccagaaggcccctttctgcagcggggccggcctccctccccgatcctgggatggcctgcg

SEQ ID NO: 1446 - DNA, 129 bp, Homo sapiens
  source 1..129: mol_type = genomic DNA
cggcagcttcaccctcctgcgggaaaatgcaaagtccatcaccaatagtcggtctaccgcagagaagtctgcaaatcccaggggcctggaactctaaaaaggccattcaagcctcaggtggactccacg

SEQ ID NO: 1447 - DNA, 31 bp, Homo sapiens
  source 1..31: mol_type = genomic DNA
cgcagcccgcccgaaggtcaggccgcatccg

SEQ ID NO: 1448 - DNA, 136 bp, Homo sapiens
  source 1..136: mol_type = genomic DNA
cgggggtggggggaagaagtggaagcaaatgcaaccctaagtcccatgagatccaggtttccagagtctttgaaacttggcctaggtttctcagtgtaaatccctctgctcaaagttccgaaaacgtccacgatcg

SEQ ID NO: 1449 - DNA, 44 bp, Homo sapiens
  source 1..44: mol_type = genomic DNA
cgagagcgtacccgtttgcaaattgctgcaggaagagcgaggcg

SEQ ID NO: 1450 - DNA, 53 bp, Homo sapiens
  source 1..53: mol_type = genomic DNA
cgtttgcaaattgctgcaggaagagcgaggcgggccttgcgctttttaatccg

SEQ ID NO: 1451 - DNA, 77 bp, Homo sapiens
  source 1..77: mol_type = genomic DNA
cgtaaaccttctcaaacctccgagttcaccttacagcgaacattaaatacccttgcgactacaatactaacaaatcg

SEQ ID NO: 1452 - DNA, 67 bp, Homo sapiens
  source 1..67: mol_type = genomic DNA
cgagttcaccttacagcgaacattaaatacccttgcgactacaatactaacaaatcgaacactgacg

SEQ ID NO: 1453 - DNA, 71 bp, Homo sapiens
  source 1..71: mol_type = genomic DNA
cgaacattaaatacccttgcgactacaatactaacaaatcgaacactgacgtaaaatcttaagaataaacg

SEQ ID NO: 1454 - DNA, 103 bp, Homo sapiens
  source 1..103: mol_type = genomic DNA
cgtccacgtctctccccaccccttgcctacacccccaaaactcagtggcgaaagaagcccctaaaatgcattgacattttttaaggggaattggggctcgccg

SEQ ID NO: 1455 - DNA, 103 bp, Homo sapiens
  source 1..103: mol_type = genomic DNA
cgtctctccccaccccttgcctacacccccaaaactcagtggcgaaagaagcccctaaaatgcattgacattttttaaggggaattggggctcgccgggggcg

SEQ ID NO: 1456 - DNA, 75 bp, Homo sapiens; identical to SEQ ID NO: 800
  source 1..75: mol_type = genomic DNA

SEQ ID NO: 1457 - DNA, 133 bp, Homo sapiens
  source 1..133: mol_type = genomic DNA
cgtggttttgtttaaaaagacgtaagaagggaagagaaagactgagatgatggggagatggtatttgcttgtttcatcttttatttttaaaagtaaatagggaagggggaaggagggggagccgtggcgggcg

SEQ ID NO: 1458 - DNA, 118 bp, Homo sapiens
  source 1..118: mol_type = genomic DNA
cgtaagaagggaagagaaagactgagatgatggggagatggtatttgcttgtttcatcttttatttttaaaagtaaatagggaagggggaaggagggggagccgtggcgggcgctacg

SEQ ID NO: 1459 - DNA, 124 bp, Homo sapiens; identical to SEQ ID NO: 813
  source 1..124: mol_type = genomic DNA

SEQ ID NO: 1460 - DNA, 79 bp, Homo sapiens; identical to SEQ ID NO: 814
  source 1..79: mol_type = genomic DNA

SEQ ID NO: 1461 - DNA, 88 bp, Homo sapiens; identical to SEQ ID NO: 815
  source 1..88: mol_type = genomic DNA

SEQ ID NO: 1462 - DNA, 106 bp, Homo sapiens
  source 1..106: mol_type = genomic DNA
cgttgatttcccgagaaacaagcgtgtcccaggtcttctcaaaaacttccctccttggtctccgcattagggaaactgaggtactgctggcatggctgtccatacg

SEQ ID NO: 1463 - DNA, 101 bp, Homo sapiens; identical to SEQ ID NO: 816
  source 1..101: mol_type = genomic DNA

SEQ ID NO: 1464 - DNA, 63 bp, Homo sapiens
  source 1..63: mol_type = genomic DNA
cggaccctaaaggcaagttgccggggaaggttccttaacaaacagcgggcttcccggtagccg

SEQ ID NO: 1465 - DNA, 77 bp, Homo sapiens
  source 1..77: mol_type = genomic DNA
cggggaaggttccttaacaaacagcgggcttcccggtagccgggttcccagcccaagaaccttctcattcacctgcg

SEQ ID NO: 1466 - DNA, 58 bp, Homo sapiens
  source 1..58: mol_type = genomic DNA
cgggcttcccggtagccgggttcccagcccaagaaccttctcattcacctgcgcagcg

SEQ ID NO: 1467 - DNA, 118 bp, Homo sapiens
  source 1..118: mol_type = genomic DNA
cggtgtgtttaatcgatgctgatgttttactctctagtttgaggacccagcgacagggctttagcccctcttcctgcagctgcggataacagacattggctctttgggaattgcagcg

SEQ ID NO: 1468 - DNA, 77 bp, Homo sapiens
  source 1..77: mol_type = genomic DNA
cgctccctgatcccggcttcaaggttcagcttgaagagagtcggtttacctttccacccacaccgggccctggtgcg

SEQ ID NO: 1469 - DNA, 60 bp, Homo sapiens
  source 1..60: mol_type = genomic DNA
cgggtcgaaggccacaggctgagaatttgctgggaggggtcgggaagaagcgtaggagcg

SEQ ID NO: 1470 - DNA, 56 bp, Homo sapiens
  source 1..56: mol_type = genomic DNA
cgggaagaagcgtaggagcgctgaccgcctccccttcctctttctctgtctttgcg

SEQ ID NO: 1471 - DNA, 45 bp, Homo sapiens
  source 1..45: mol_type = genomic DNA
cgctgaccgcctccccttcctctttctctgtctttgcgggcggcg

SEQ ID NO: 1472 - DNA, 43 bp, Homo sapiens
  source 1..43: mol_type = genomic DNA
cgagcccccctctccaggtcaaatgctcggcgcggcctttccg

SEQ ID NO: 1473 - DNA, 65 bp, Homo sapiens
  source 1..65: mol_type = genomic DNA
cgggagaggaggggacgggcggggagaggcccttggcccctctcattccctgagtccctgcggcg

SEQ ID NO: 1474 - DNA, 53 bp, Homo sapiens
  source 1..53: mol_type = genomic DNA
cggaagccggctctctcctgccccagcctcgggccgccttgccccagctctcg

SEQ ID NO: 1475 - DNA, 60 bp, Homo sapiens
  source 1..60: mol_type = genomic DNA
cggctctctcctgccccagcctcgggccgccttgccccagctctcgggccttgacccccg

SEQ ID NO: 1476 - DNA, 54 bp, Homo sapiens
  source 1..54: mol_type = genomic DNA
cgggccgccttgccccagctctcgggccttgacccccggagggactaccactcg

SEQ ID NO: 1477 - DNA, 116 bp, Homo sapiens
  source 1..116: mol_type = genomic DNA
cggaaaggatttagtgtttatgtgaacggagcacctcggggccaagttgttgctgtcgggaatacagaccacagaggtagagagctgaaaggagaaaagcagcaggcagcacaccg

SEQ ID NO: 1478 - DNA, 66 bp, Homo sapiens
  source 1..66: mol_type = genomic DNA
cgctttcctttcagtcccagctgctcgagaagggagggcgctgggaaaggaaacagagcggaatcg

SEQ ID NO: 1479 - DNA, 56 bp, Homo sapiens
  source 1..56: mol_type = genomic DNA
cgagaagggagggcgctgggaaaggaaacagagcggaatcgcccacattagcctcg

SEQ ID NO: 1480 - DNA, 78 bp, Homo sapiens
  source 1..78: mol_type = genomic DNA
cggcgcctctgggcttgtgtgagcgtctcctttctaatgccaatgacaacgacactgagggtgattttctgtgcctcg

SEQ ID NO: 1481 - DNA, 112 bp, Homo sapiens
  source 1..112: mol_type = genomic DNA
cgcctctgggcttgtgtgagcgtctcctttctaatgccaatgacaacgacactgagggtgattttctgtgcctcgcccacccctttgcaggcaactttgtaaagaatttacg

SEQ ID NO: 1482 - DNA, 104 bp, Homo sapiens
  source 1..104: mol_type = genomic DNA
cgtctcctttctaatgccaatgacaacgacactgagggtgattttctgtgcctcgcccacccctttgcaggcaactttgtaaagaatttacggatctactggcg

SEQ ID NO: 1483 - DNA, 86 bp, Homo sapiens
  source 1..86: mol_type = genomic DNA
cgacactgagggtgattttctgtgcctcgcccacccctttgcaggcaactttgtaaagaatttacggatctactggcgcaggaccg

SEQ ID NO: 1484 - DNA, 52 bp, Homo sapiens
  source 1..52: mol_type = genomic DNA
cgctcgccccgggacaggccctcgcctacacccctggaagtaaggagccccg

SEQ ID NO: 1485 - DNA, 58 bp, Homo sapiens
  source 1..58: mol_type = genomic DNA
cgccccgggacaggccctcgcctacacccctggaagtaaggagccccgggctctttcg

SEQ ID NO: 1486 - DNA, 62 bp, Homo sapiens
  source 1..62: mol_type = genomic DNA
cgggacaggccctcgcctacacccctggaagtaaggagccccgggctctttcgtccttttcg

SEQ ID NO: 1487 - DNA, 68 bp, Homo sapiens
  source 1..68: mol_type = genomic DNA
cggctgcgcccccgctgtggactaggaggcaggccaaccctccggactttgggggaaaaaccacagcg

SEQ ID NO: 1488 - DNA, 73 bp, Homo sapiens
  source 1..73: mol_type = genomic DNA
cgcccccgctgtggactaggaggcaggccaaccctccggactttgggggaaaaaccacagcgggctccttgcg

SEQ ID NO: 1489 - DNA, 80 bp, Homo sapiens
  source 1..80: mol_type = genomic DNA
cgctgtggactaggaggcaggccaaccctccggactttgggggaaaaaccacagcgggctccttgcggaaactttggccg

SEQ ID NO: 1490 - DNA, 48 bp, Homo sapiens
  source 1..48: mol_type = genomic DNA
cgccgctgcatttaggccctttccagaccggtggcggcagccaacccg

SEQ ID NO: 1491 - DNA, 54 bp, Homo sapiens
  source 1..54: mol_type = genomic DNA
cgctgcatttaggccctttccagaccggtggcggcagccaacccgagacttgcg

SEQ ID NO: 1492 - DNA, 36 bp, Homo sapiens
  source 1..36: mol_type = genomic DNA
cggtggcggcagccaacccgagacttgcgtccctcg

SEQ ID NO: 1493 - DNA, 36 bp, Homo sapiens
  source 1..36: mol_type = genomic DNA
cggcagccaacccgagacttgcgtccctcgggcccg

SEQ ID NO: 1494 - DNA, 29 bp, Homo sapiens
  source 1..29: mol_type = genomic DNA
cggggcagctaggaggtcggcgcgcagcg

SEQ ID NO: 1495 - DNA, 30 bp, Homo sapiens
  source 1..30: mol_type = genomic DNA
cgcgcagcgggccgggtcaggactgggtcg

SEQ ID NO: 1496 - DNA, 51 bp, Homo sapiens
  source 1..51: mol_type = genomic DNA
cgcagcgggccgggtcaggactgggtcgagcagacagagctgcagcccccg

SEQ ID NO: 1497 - DNA, 62 bp, Homo sapiens
  source 1..62: mol_type = genomic DNA
cggagctcgctcggcttgctaaatgagaggagctttgcaacggggtcaaccagcttgtctcg

SEQ ID NO: 1498 - DNA, 75 bp, Homo sapiens
  source 1..75: mol_type = genomic DNA
cggctcagaggtctgctcagaggcaggactcgcactggtggtggcctagagggcaacagtccggaagctcgggcg

SEQ ID NO: 1499 - DNA, 55 bp, Homo sapiens
  source 1..55: mol_type = genomic DNA
cgcactggtggtggcctagagggcaacagtccggaagctcgggcgggggaatccg

SEQ ID NO: 1500 - DNA, 69 bp, Homo sapiens
  source 1..69: mol_type = genomic DNA
cggaagctcgggcgggggaatccgaggaggggcccccaccctgcactggtcttccctccaccctcatcg

SEQ ID NO: 1501 - DNA, 117 bp, Homo sapiens
  source 1..117: mol_type = genomic DNA
cgaccaggaactcccttgggcgcagtgacaggcccgaaggagtggaggacaagacccgctgtgcccttgcctggccctctccagcccctccctcccacagcacaatctcccctcccg

SEQ ID NO: 1502 - DNA, 115 bp, Homo sapiens
  source 1..115: mol_type = genomic DNA
cgcagtgacaggcccgaaggagtggaggacaagacccgctgtgcccttgcctggccctctccagcccctccctcccacagcacaatctcccctcccgccctgtgggaggggggcg

SEQ ID NO: 1503 - DNA, 78 bp, Homo sapiens; identical to SEQ ID NO: 856
  source 1..78: mol_type = genomic DNA

SEQ ID NO: 1504 - DNA, 62 bp, Homo sapiens
  source 1..62: mol_type = genomic DNA
cggctcctccactcgattcctgcctacacgtgaagagcttggacccagcggggctgcactcg

SEQ ID NO: 1505 - DNA, 63 bp, Homo sapiens
  source 1..63: mol_type = genomic DNA
cgtgaagagcttggacccagcggggctgcactcgggaggacaggcccagatcctgcacggccg

SEQ ID NO: 1506 - DNA, 155 bp, Homo sapiens
  source 1..155: mol_type = genomic DNA
cgaagttaaatgttggtgtctgagcgatgtgctggtggggcagagggggttccagcgtcctcccagagtgaggaggcttttgcaatttctgctgaatgcgtcagtggaggcacaaaaccttgcttgacctagcagctgggaaaaaaagaagttcg

SEQ ID NO: 1507 - DNA, 97 bp, Homo sapiens
  source 1..97: mol_type = genomic DNA
cggggtttaatctggaatgaaaggatcgtcgtggtgtttccagattgaatgaaaaactaaacttgcaaaatttcgagaaagtgacaagtttacaacg

SEQ ID NO: 1508 - DNA, 128 bp, Homo sapiens
  source 1..128: mol_type = genomic DNA
cgcatccatctcaagaaaatatttacatttgttttgcacaaaagggatggccgtctttttccaaagcgagaacacagagtcagagaaagccaactcaaaagtcccttcgcaagtttttaactacaccg

SEQ ID NO: 1509 - DNA, 75 bp, Homo sapiens
  source 1..75: mol_type = genomic DNA
cgcttcctgcggctgctgcttgcccctcaggccaggaggtttctggaaggaccggtgctgtctccccgaacatcg

SEQ ID NO: 1510 - DNA, 31 bp, Homo sapiens
  source 1..31: mol_type = genomic DNA
cgtggtctccccgaacatcgcggcctctccg

SEQ ID NO: 1511 - DNA, 42 bp, Homo sapiens
  source 1..42: mol_type = genomic DNA
cggaaaaagacctggaggctccgcgggagcgcccagctggcg

SEQ ID NO: 1512 - DNA, 90 bp, Homo sapiens
  source 1..90: mol_type = genomic DNA
cgcctgcccagaccctggaagggtctctgtcccccgcaacagcctgccccgcggtggccttgtgggcaggactcagctatgagcagatcg

SEQ ID NO: 1513 - DNA, 95 bp, Homo sapiens
  source 1..95: mol_type = genomic DNA
cgcaacagcctgccccgcggtggccttgtgggcaggactcagctatgagcagatcgactctgcccaagtcttctctcacccaggtccagtgggcg

SEQ ID NO: 1514 - DNA, 146 bp, Homo sapiens
  source 1..146: mol_type = genomic DNA
cggccagcggagcccctcacataaaattagacaataattgaagccataaaaaagcagccaaatcgcattgtcgctctactgtatttaaatctatatttatgatatttcataaggagttattgtttcagaagccacacaggctggcg

SEQ ID NO: 1515 - DNA, 77 bp, Homo sapiens
  source 1..77: mol_type = genomic DNA
cgtttgcctcgccgtggctcccagctgtaaaaatttacgaggacttggaaaggttagactgttgtgtttggttggcg

SEQ ID NO: 1516 - DNA, 92 bp, Homo sapiens
  source 1..92: mol_type = genomic DNA
cgccgtggctcccagctgtaaaaatttacgaggacttggaaaggttagactgttgtgtttggttggcgagctccctgtaaataatccctgcg

SEQ ID NO: 1517 - DNA, 96 bp, Homo sapiens
  source 1..96: mol_type = genomic DNA
cgtggctcccagctgtaaaaatttacgaggacttggaaaggttagactgttgtgtttggttggcgagctccctgtaaataatccctgcggtccccg

SEQ ID NO: 1518 - DNA, 80 bp, Homo sapiens
  source 1..80: mol_type = genomic DNA
cgaggacttggaaaggttagactgttgtgtttggttggcgagctccctgtaaataatccctgcggtccccgggagaggcg

SEQ ID NO: 1519 - DNA, 52 bp, Homo sapiens; identical to SEQ ID NO: 890
  source 1..52: mol_type = genomic DNA

SEQ ID NO: 1520 - DNA, 31 bp, Homo sapiens
  source 1..31: mol_type = genomic DNA
cgcgcgtctgggggcctatgcccggcttccg

SEQ ID NO: 1521 - DNA, 44 bp, Homo sapiens
  source 1..44: mol_type = genomic DNA
cgcgtctgggggcctatgcccggcttccgattccatttaaaacg

SEQ ID NO: 1522 - DNA, 47 bp, Homo sapiens
  source 1..47: mol_type = genomic DNA
cgtctgggggcctatgcccggcttccgattccatttaaaacgacccg

SEQ ID NO: 1523 - DNA, 31 bp, Homo sapiens
  source 1..31: mol_type = genomic DNA
cggcttccgattccatttaaaacgacccgcg

SEQ ID NO: 1524 - DNA, 25 bp, Homo sapiens
  source 1..25: mol_type = genomic DNA
cgacccgcgcatcttatctccgtcg

SEQ ID NO: 1525 - DNA, 86 bp, Homo sapiens
  source 1..86: mol_type = genomic DNA
cgctgtcatgggattgcccaggcttaatggggttgtgcagggcttcacttgctctcggaatttaccttccttctcgagccgtgccg

SEQ ID NO: 1526 - DNA, 132 bp, Homo sapiens
  source 1..132: mol_type = genomic DNA
cgaataaccttcacgacacagtgataacgatgttattaaatactccccccacattcaccgcccaaaaaaccaaaacagtgttcttatccagtcctctctttttttgtcttctttcctttaaaaacccaaccg

SEQ ID NO: 1527 - DNA, 88 bp, Homo sapiens
  source 1..88: mol_type = genomic DNA
cgttctggcgggggtctcctactgtgttctggcattggcgggactgagggtgacagcagtgccttgagtgcggggtgctgagggggcg

SEQ ID NO: 1528 - DNA, 59 bp, Homo sapiens
  source 1..59: mol_type = genomic DNA
cgcggcgactacagggacagaatttggctttccttctctttccaatatgcagcgtttcg

SEQ ID NO: 1529 - DNA, 122 bp, Homo sapiens
  source 1..122: mol_type = genomic DNA
cgttgagaaaggtctggacccgatgtctgcaggaagtgtcaggacctccctgttctggggaccttcccagcccacaccaggtccgtgctctctagattcccccaacctcgctccacaacccg

SEQ ID NO: 1530 - DNA, 90 bp, Homo sapiens
  source 1..90: mol_type = genomic DNA
cgcgatcaccccacaggggctgttagtaatctccctgcttcctaggccgttgagaagcactcaggtcacccactctcggtgaccagagcg

SEQ ID NO: 1531 - DNA, 103 bp, Homo sapiens
  source 1..103: mol_type = genomic DNA
cgatcaccccacaggggctgttagtaatctccctgcttcctaggccgttgagaagcactcaggtcacccactctcggtgaccagagcggtcagtctttccccg

SEQ ID NO: 1532 - DNA, 76 bp, Homo sapiens
  source 1..76: mol_type = genomic DNA
cgttgagaagcactcaggtcacccactctcggtgaccagagcggtcagtctttccccgccatgaaatgtgagttcg

SEQ ID NO: 1533 - DNA, 96 bp, Homo sapiens
  source 1..96: mol_type = genomic DNA
cgacgggcttcagcagctaggcctgggagtgcctcgtttcaggcctcgaggtggcttcaggctgggacaactgatgcagcagagaagggcagagcg

SEQ ID NO: 1534 - DNA, 97 bp, Homo sapiens
  source 1..97: mol_type = genomic DNA
cgggcttcagcagctaggcctgggagtgcctcgtttcaggcctcgaggtggcttcaggctgggacaactgatgcagcagagaagggcagagcgcccg

SEQ ID NO: 1535 - DNA, 87 bp, Homo sapiens
  source 1..87: mol_type = genomic DNA
cgtttcaggcctcgaggtggcttcaggctgggacaactgatgcagcagagaagggcagagcgcccggggggagaaagcaaagaggcg

SEQ ID NO: 1536 - DNA, 88 bp, Homo sapiens
  source 1..88: mol_type = genomic DNA
cgcccagactgcaaatttttctttccttttgctggggccacgggacagagcacctgggcagagcaagcggagaggcgccacatccccg

SEQ ID NO: 1537 - DNA, 26 bp, Homo sapiens
  source 1..26: mol_type = genomic DNA
cgaacgcggccagctgcggggacccg

SEQ ID NO: 1538 - DNA, 120 bp, Homo sapiens
  source 1..120: mol_type = genomic DNA
cgatctcgagggattcacttcacctctctaagcctcagtttcccatttgcaaaatgggcagaatgactcgcacctctggggatgaagaaaatgcacatgtgagcacgtgggtacttggcg

SEQ ID NO: 1539 - DNA, 119 bp, Homo sapiens
  source 1..119: mol_type = genomic DNA
cgagggattcacttcacctctctaagcctcagtttcccatttgcaaaatgggcagaatgactcgcacctctggggatgaagaaaatgcacatgtgagcacgtgggtacttggcgacccg

SEQ ID NO: 1540 - DNA, 80 bp, Homo sapiens
  source 1..80: mol_type = genomic DNA
cggggaagccaaagcgggagtctttcgatcttggcccagcccttagccctcggtcctttgctctagcatctagattctcg

SEQ ID NO: 1541 - DNA, 102 bp, Homo sapiens
  source 1..102: mol_type = genomic DNA
cggtcgactttgacccccgagcaggtgaaacataagcgtccaccccagaggggtcccctacccaagtcagaggagaaggtggcctgccaggttaaaggatcg

SEQ ID NO: 1542 - DNA, 107 bp, Homo sapiens
  source 1..107: mol_type = genomic DNA
cgcaggccctggcgaggagcccctccggcccatgcggaactctgcacaggctgggtccctccagggttagaaacccagcttccccacccctacccagcagggacccg

SEQ ID NO: 1543 - DNA, 97 bp, Homo sapiens
  source 1..97: mol_type = genomic DNA
cgaggagcccctccggcccatgcggaactctgcacaggctgggtccctccagggttagaaacccagcttccccacccctacccagcagggacccgcg

SEQ ID NO: 1544 - DNA, 99 bp, Homo sapiens
  source 1..99: mol_type = genomic DNA
cggcccatgcggaactctgcacaggctgggtccctccagggttagaaacccagcttccccacccctacccagcagggacccgcgctgcatcccctaacg

SEQ ID NO: 1545 - DNA, 60 bp, Homo sapiens
  source 1..60: mol_type = genomic DNA
cgcgctgcatcccctaacgccccccaaccctctgcacccagacccaagagccggccttcg

SEQ ID NO: 1546 - DNA, 61 bp, Homo sapiens
  source 1..61: mol_type = genomic DNA
cgctgcatcccctaacgccccccaaccctctgcacccagacccaagagccggccttcgacg

SEQ ID NO: 1547 - DNA, 57 bp, Homo sapiens
  source 1..57: mol_type = genomic DNA
cgccccccaaccctctgcacccagacccaagagccggccttcgacgcaccacatgcg

SEQ ID NO: 1548 - DNA, 72 bp, Homo sapiens
  source 1..72: mol_type = genomic DNA
cgacgcaccacatgcgtggagcccgggtcccagccctgaggaccaggtttcagggctcagaagactccagcg

SEQ ID NO: 1549 - DNA, 80 bp, Homo sapiens
  source 1..80: mol_type = genomic DNA
cgcaccacatgcgtggagcccgggtcccagccctgaggaccaggtttcagggctcagaagactccagcgaggttccctcg

SEQ ID NO: 1550 - DNA, 84 bp, Homo sapiens
  source 1..84: mol_type = genomic DNA
cgtggagcccgggtcccagccctgaggaccaggtttcagggctcagaagactccagcgaggttccctcgcagattgtgtctgcg

SEQ ID NO: 1551 - DNA, 47 bp, Homo sapiens
  source 1..47: mol_type = genomic DNA
cgccgccccaggatgcaggtgctgcggcgctccctggtagcctcccg

SEQ ID NO: 1552 - DNA, 32 bp, Homo sapiens
  source 1..32: mol_type = genomic DNA
cggaagggggcgtggagccgccaagggcgccg

SEQ ID NO: 1553 - DNA, 27 bp, Homo sapiens
  source 1..27: mol_type = genomic DNA
cgtggagccgccaagggcgccggaccg

SEQ ID NO: 1554 - DNA, 83 bp, Homo sapiens
  source 1..83: mol_type = genomic DNA
cgccatggagggacagcttctatccagggccctaactgtagcgtgggttggggggactctttcgagagggtggggcgaggacg

SEQ ID NO: 1555 - DNA, 41 bp, Homo sapiens
  source 1..41: mol_type = genomic DNA
cgagagggtggggcgaggacgctggccccactgctgcggcg

SEQ ID NO: 1556 - DNA, 79 bp, Homo sapiens
  source 1..79: mol_type = genomic DNA
cgttttccaaagtgtgttgaattcgggaatcgatcgaaaatttcaaggccaattaaatgccctctgatgtagagctccg

SEQ ID NO: 1557 - DNA, 84 bp, Homo sapiens
  source 1..84: mol_type = genomic DNA
cggcctccagctgccggaaaggttggaagtcttcctccacggtggccaggctgtttctgtagtgtccataggacgggaaaggcg

SEQ ID NO: 1558 - DNA, 79 bp, Homo sapiens
  source 1..79: mol_type = genomic DNA
cggaaaggttggaagtcttcctccacggtggccaggctgtttctgtagtgtccataggacgggaaaggcgtgtagtacg

SEQ ID NO: 1559 - DNA, 56 bp, Homo sapiens
  source 1..56: mol_type = genomic DNA
cggtggccaggctgtttctgtagtgtccataggacgggaaaggcgtgtagtacgcg

SEQ ID NO: 1560 - DNA, 64 bp, Homo sapiens
  source 1..64: mol_type = genomic DNA
cggcctggcagcggcttggaagagagagggaaaggaggaacatctacccggctaagagacgccg

SEQ ID NO: 1561 - DNA, 136 bp, Homo sapiens
  source 1..136: mol_type = genomic DNA
cgtagtgacgctcaggtgtcctcgggttgttagtcttgacccaggagagtgcaagacaggcccaaggcctggggggattgcgttatgaatgtccaattctaaacacaaggtagagcacagacagtacatcctcacg

SEQ ID NO: 1562 - DNA, 143 bp, Homo sapiens
  source 1..143: mol_type = genomic DNA
cgacctctgcctcccgtccccagagcttacaaaattccttccagttgtattttagggcttgttttcctagagaaggttgagactagaggaaggattaaagtggtgggggaaggggcaagaccgaggggtccctaccccacgcg

SEQ ID NO: 1563 - DNA, 68 bp, Homo sapiens
  source 1..68: mol_type = genomic DNA
cgggtctgcgggctgtagcgagctcacccagtgagggcctggctcattttttgtgctcagggttcgcg

SEQ ID NO: 1564 - DNA, 67 bp, Homo sapiens
  source 1..67: mol_type = genomic DNA
cgggctgtagcgagctcacccagtgagggcctggctcattttttgtgctcagggttcgcgttatgcg

SEQ ID NO: 1565 - DNA, 72 bp, Homo sapiens
  source 1..72: mol_type = genomic DNA
cgggattttgtttccctaacttacgaatgcaggaaaaactcggcacatcactcgacaatattgctcttaacg

SEQ ID NO: 1566 - DNA, 118 bp, Homo sapiens
  source 1..118: mol_type = genomic DNA
cgcttatgtctttcaggcgaattaattcttcagtttcaattcgtgaatgggaaagaaacttgccttgtcgctcttgcccttcccccctcttgagccccagggaatagcactttactcg

SEQ ID NO: 1567 - DNA, 119 bp, Homo sapiens
  source 1..119: mol_type = genomic DNA
cgtttcctaattgaacttaagtccaccaccatggtagagactgaattagcaaaaacccgcaacagtgcgtttttcctgtcaccactatccgtcacctctctcccattttgaggtcctcg

SEQ ID NO: 1568 - DNA, 86 bp, Homo sapiens
  source 1..86: mol_type = genomic DNA
cgtttttcctgtcaccactatccgtcacctctctcccattttgaggtcctcgactaccatctaaggccaggaactcggtgtcagcg

SEQ ID NO: 1569 - DNA, 77 bp, Homo sapiens
  source 1..77: mol_type = genomic DNA
cgtcacctctctcccattttgaggtcctcgactaccatctaaggccaggaactcggtgtcagcggggtgggtgcccg

SEQ ID NO: 1570 - DNA, 52 bp, Homo sapiens
  source 1..52: mol_type = genomic DNA
cgactaccatctaaggccaggaactcggtgtcagcggggtgggtgcccgacg

SEQ ID NO: 1571 - DNA, 30 bp, Homo sapiens
  source 1..30: mol_type = genomic DNA
cggtgtcagcggggtgggtgcccgacggcg

SEQ ID NO: 1572 - DNA, 37 bp, Homo sapiens
  source 1..37: mol_type = genomic DNA
cggggtgggtgcccgacggcgcccccttaagtctgcg

SEQ ID NO: 1573 - DNA, 71 bp, Homo sapiens
  source 1..71: mol_type = genomic DNA
cgggaggccacagcctggggcctgggaccgcagagttcttggtgggtgaaagccggcggccaagctgctcg

SEQ ID NO: 1574 - DNA, 50 bp, Homo sapiens
  source 1..50: mol_type = genomic DNA
cgcagagttcttggtgggtgaaagccggcggccaagctgctcgagctgcg

SEQ ID NO: 1575 - DNA, 67 bp, Homo sapiens
  source 1..67: mol_type = genomic DNA
cgtgcatctctcctcccgggggctgcccagctctccccagcgcgtttcctagatactaggcaggccg

SEQ ID NO: 1576 - DNA, 138 bp, Homo sapiens
  source 1..138: mol_type = genomic DNA
cgtcttcacctgcaactggctcaggcccagggactcagcaagatctattctggaaagagcagggcgcaccctcagcaaggcaagtgaccacactcccacagcccgctggccccagccttgtaccgccctacctgtccg

SEQ ID NO: 1577 - DNA, 19 bp, Homo sapiens
  source 1..19: mol_type = genomic DNA
cgggcggcccgcgggcacg

SEQ ID NO: 1578 - DNA, 54 bp, Homo sapiens
  source 1..54: mol_type = genomic DNA
cggcccgcgggcacgaggcctggtttctgtgaggcctggagcttgcaccctccg

SEQ ID NO: 1579 - DNA, 56 bp, Homo sapiens
  source 1..56: mol_type = genomic DNA
cgcgggcacgaggcctggtttctgtgaggcctggagcttgcaccctccgcctcccg

SEQ ID NO: 1580 - DNA, 79 bp, Homo sapiens
  source 1..79: mol_type = genomic DNA
cgctgtggcatctctggccagaagcacctttggcggcctgttgggcccctcgcagcctctggctgagactctgccgccg

SEQ ID NO: 1581 - DNA, 121 bp, Homo sapiens
  source 1..121: mol_type = genomic DNA
cggaggatctgactcccacgggctccagggtcctcctgggtggatgtccctccgtcctgtcttgccactctctgtgccatcctccaggacaaaggaggctcgtatgggcaactggaacccg

SEQ ID NO: 1582 - DNA, 116 bp, Homo sapiens
  source 1..116: mol_type = genomic DNA
cgtcctgtcttgccactctctgtgccatcctccaggacaaaggaggctcgtatgggcaactggaacccggctggagtgtgcagctggccccctcacttccagcccgcaggctgccg

SEQ ID NO: 1583 - DNA, 70 bp, Homo sapiens
  source 1..70: mol_type = genomic DNA
cgtatgggcaactggaacccggctggagtgtgcagctggccccctcacttccagcccgcaggctgccgcg

SEQ ID NO: 1584 - DNA, 64 bp, Homo sapiens
  source 1..64: mol_type = genomic DNA
cggctggagtgtgcagctggccccctcacttccagcccgcaggctgccgcgggggtgactcccg

SEQ ID NO: 1585 - DNA, 146 bp, Homo sapiens
  source 1..146: mol_type = genomic DNA
cggctcttttgagattgtcttgcaaaccatggactctctccccagaaatgaacacccacaatctagtgtgtaaatttcaggtttccaagtgttccaaagccgcgcacaaatttcggaaccagaccaagaatcctggactctagacg

SEQ ID NO: 1586 - DNA, 85 bp, Homo sapiens
  source 1..85: mol_type = genomic DNA
cggaaccagaccaagaatcctggactctagacgttgcctgggggccgatcctgcgcccagggtcccagaatctaactgaaatgcg

SEQ ID NO: 1587 - DNA, 60 bp, Homo sapiens
  source 1..60: mol_type = genomic DNA
cgttgcctgggggccgatcctgcgcccagggtcccagaatctaactgaaatgcgcctgcg

SEQ ID NO: 1588 - DNA, 89 bp, Homo sapiens
  source 1..89: mol_type = genomic DNA
cgcagactgggttgggagagggctctgtgggagggcttgcggggagcggggtaagaaaggctaggtgcttcttacctacgtaagaaacg

SEQ ID NO: 1589 - DNA, 59 bp, Homo sapiens
  source 1..59: mol_type = genomic DNA
cggggagcggggtaagaaaggctaggtgcttcttacctacgtaagaaacgttggtggcg

SEQ ID NO: 1590 - DNA, 67 bp, Homo sapiens
  source 1..67: mol_type = genomic DNA
cggggtaagaaaggctaggtgcttcttacctacgtaagaaacgttggtggcgggatctaagcaaacg

SEQ ID NO: 1591 - DNA, 110 bp, Homo sapiens
  source 1..110: mol_type = genomic DNA
cgtaagaaacgttggtggcgggatctaagcaaacggggaggtgggaagcagaaggttccccttcctgggccccagttcctccatcctgtcccctgggtgacagtgattcg

SEQ ID NO: 1592 - DNA, 122 bp, Homo sapiens
  source 1..122: mol_type = genomic DNA
cgcagagcatagcagggctgaaaggctttaaataagcgctgccttgtaacaaagcctcctggagtgtcatgataaagccccgggaaggagaaagacagggtgggaaagagcggacattttcg

SEQ ID NO: 1593 - DNA, 112 bp, Homo sapiens
  source 1..112: mol_type = genomic DNA
cgctgccttgtaacaaagcctcctggagtgtcatgataaagccccgggaaggagaaagacagggtgggaaagagcggacattttcgtcccccattctacttgtcaacttgcg

SEQ ID NO: 1594 - DNA, 91 bp, Homo sapiens
  source 1..91: mol_type = genomic DNA
cggacattttcgtcccccattctacttgtcaacttgcgggacaggtaagattgtgccaaaggcgagggtcagcatcaggagagagaggacg

SEQ ID NO: 1595 - DNA, 86 bp, Homo sapiens
  source 1..86: mol_type = genomic DNA
cgtcccccattctacttgtcaacttgcgggacaggtaagattgtgccaaaggcgagggtcagcatcaggagagagaggacgctgcg

SEQ ID NO: 1596 - DNA, 103 bp, Homo sapiens
  source 1..103: mol_type = genomic DNA
cgggacaggtaagattgtgccaaaggcgagggtcagcatcaggagagagaggacgctgcggggagggacctggttgtgtgcttcaggtgccccaagggacacg

SEQ ID NO: 1597 - DNA, 77 bp, Homo sapiens
  source 1..77: mol_type = genomic DNA
cggggcccagcccagtcttcctagatctggcgtccgcccttccctcccctcccgcactggcaggagagaaatggccg

SEQ ID NO: 1598 - DNA, 59 bp, Homo sapiens
  source 1..59: mol_type = genomic DNA
cgtccgcccttccctcccctcccgcactggcaggagagaaatggccgcagtgtgggccg

SEQ ID NO: 1599 - DNA, 34 bp, Homo sapiens
  source 1..34: mol_type = genomic DNA
cgtgtgcgtgcagggttccgaccacggggacacg

SEQ ID NO: 1600 - DNA, 34 bp, Homo sapiens
  source 1..34: mol_type = genomic DNA
cggggccgcagcggcctcttcccgcagcccctcg

SEQ ID NO: 1601 - DNA, 34 bp, Homo sapiens
  source 1..34: mol_type = genomic DNA
cgagccaaggctcgaggcccgcggcctctccacg

SEQ ID NO: 1602 - DNA, 36 bp, Homo sapiens
  source 1..36: mol_type = genomic DNA
cgagccctcggggccgagggctgtgggcccggtgcg

SEQ ID NO: 1603 - DNA, 32 bp, Homo sapiens
  source 1..32: mol_type = genomic DNA
cggggccgagggctgtgggcccggtgcgaccg

SEQ ID NO: 1604 - DNA, 31 bp, Homo sapiens
  source 1..31: mol_type = genomic DNA
cgagggctgtgggcccggtgcgaccgggacg